(19) United States
(12) Patent Application Publication
(10) Pub. No.: US 2004/0265984 A1
(43) Pub. Date: Dec. 30, 2004

(54) Methods of growing crystals of free, antibiotic-complexed, and substrate-complexed large ribosomal subunits, and methods of rationally designing or identifying antibiotics using structure coordinate data derived from such crystals

(76) Inventors: Ada Yonath, Rehovot (IL); Anat Bashan, Nes Ziona (IL); Raz Zarivach, Yavne (IL); Francois Francheschi, Killingworth, CT (US); Frank Schluenzen, Wedel (DE); Joerg Harms, Hamburg (DE); Renate Albrecht, Berlin (DE); Rita Berisio, Napoli (IT)

Correspondence Address: Anthony Castorina, G E Ehrlich, Suite 207, 2001 Jefferson Davis Highway, Arlington, VA, 22202, US

(21) Appl. No.: 10/489,616

(22) PCT Filed:
(86) PCT No.: PCT/IL02/00786

Related U.S. Application Data
(60) Provisional application No. 60/324,060, filed on Sep. 24, 2001.
(60) Provisional application No. 60/358,728, filed on Feb. 25, 2002.

(30) Foreign Application Priority Data
Mar. 29, 2002 (US) 10/109,572

(51) Int. Cl. C12N 9/80; G06F 19/00; G01N 33/48; G01N 33/50

(52) U.S. Cl. 435/228; 702/19

(57) ABSTRACT

Methods of growing crystals of free, antibiotic complexed, or ribosomal substrate complexed large ribosomal subunits, coordinates defining the three-dimensional atomic structure thereof and methods of utilizing such coordinates for rational design or identification of antibiotics or large ribosomal subunits having desired characteristics are disclosed.

### FIELD AND BACKGROUND OF THE INVENTION

The present invention relates to methods of growing large ribosomal subunit (LRS) crystals and antibiotic-LRS complex crystals and to methods of identifying putative antibiotics. In particular, embodiments of the present invention relate to methods of growing crystals of eubacterial LRSs and to methods of rationally designing or selecting novel antibiotics using three-dimensional (3D) atomic structure data obtained via X-ray crystallographic analysis of such crystals.

The mesophilic bacterium Deinococcus radiodurans (D. radiodurans) is an extremely robust gram-positive eubacterium that shares extensive similarity throughout its genome with the bacterium E. coil and the thermophilic bacterium Thermus thermophilus (T. thermophilus; White O. et al., 1999. Science 286, 1571). The bacterium D. radiodurans was originally identified as a live contaminant of irradiated canned meat and has been found to survive in an extremely broad range of environments ranging from hypo- to hyper-nutritive conditions, in atomic pile wastes, in weathered granite in extremely cold and dry antarctic valleys and as a live contaminant of irradiated medical instruments. It is the most radiation-resistant organism known, possessing the ability to survive under conditions normally causing lethal levels of DNA damage, such as in the presence of lethal levels of hydrogen peroxide, ionizing radiation or ultraviolet radiation. The extreme adaptability of this organism is likely due to its specialized systems for DNA repair, DNA damage export, desiccation, temperature and starvation shock recovery, and genetic redundancy.

The ribosome, the largest known macromolecular enzyme and the focus of intense biochemical research for over four decades, is a universal intracellular ribonucleoprotein complex which translates the genetic code, in the form of mRNA, into proteins (reviewed in Garrett, R. A. et al. eds. The Ribosome. Structure, Function, Antibiotics and Cellular Interactions, (2000) ASM Press, Washington, D.C.).

Ribosomes of all species display great structural and functional similarities and are composed of two independent subunits, the small ribosomal subunit and the LRS, that associate upon initiation of protein biosynthesis. In prokaryotes, the small and large ribosomal subunits, which are respectively termed 30S and 50S, according to their sedimentation coefficients (forming the 70S ribosomal particle upon association), have a molecular weight of 0.85 and 1.45 MDa, respectively. The 30S subunit consists of one 16S ribosomal RNA (rRNA) chain, composed of about 1500 nucleotides, and about 20 proteins and the LRS consists of two rRNA chains, termed 23S and 5S, and over 30 proteins. The 23S rRNA molecule contains about 3000 nucleotides and is the major component of this subunit. The D. radiodurans LRS (D50S) is composed of 5S and 23S rRNA molecules and ribosomal proteins L1-L7, L9-L24, CTC, L27-L36.

The ribosome has three binding sites for transfer RNA (tRNA), designated the P—(peptidyl), A—(acceptor or aminoacyl) and E—(exit) sites which are partly located on both the small and large ribosomal subunits. During polypeptide assembly, the aminoacyl-tRNA stem region binds the LRS, where catalysis of peptide bond synthesis, a process involving addition of amino acids to the nascent polypeptide chain, occurs. Peptide bond formation, the principal reaction of protein biosynthesis, was localized in the LRS over three decades ago (Monro et al., 1968. Proc Natl Acad Sci USA. 61, 1042-9), however neither the detailed mechanisms, nor the structural basis of catalytic formation of the peptide bond by the peptidyl transferase center (PTC) of the LRS have been fully elucidated (Nissen P. et al., 2000. Science 289, 920; Polacek, N. et al., 2001. Nature 411, 498; Thompson J. et al., 2001. Proc Natl Acad Sci USA. 98, 9002; Barta A., 2001. et al. Science 291, 203; Bayfield Mass. et al., 2001. Proc Natl Acad Sci USA. 98, 10096).

The 30S subunit performs the process of decoding genetic information during translation. It initiates mRNA and tRNA anticodon stem loop engagement, and controls fidelity of codon-anticodon interactions by discriminating between corresponding and non-corresponding aminoacyl-tRNAs in the A-site during translation of the genetic code. The 30S subunit functions in conjunction with the LRS to move tRNAs and associated mRNA by precisely one codon with respect to the ribosome, in a process termed translocation. The entire process also depends on several extrinsic protein factors and the hydrolysis of GTP.

Mutations conferring drug resistance, as well as protection experiments implicated that the peptidyl transferase activity of the ribosome is associated predominantly with the 23s rRNA of the LRS (Cundliffe, 1990. Recognition Sites for Antibiotics within rRNA. In: The Ribosome: Structure, Function and Evolution, W. E. Hill, A. E. Dahlberg, R. A. Garrett, P. B. Moore, D. Schlessinger and J. R. Warner, eds. (Washington, D.C.: ASM), pp. 479-90; Moazed and Noller, 1991. Proc Natl Acad Sci USA. 88, 3725-8; Noller et al, 1992. Science 256, 1416-9; Garrett, R. A., and Rodriguez-Fonseca, C. (1995) The peptidyl transferase center. In: Ribosomal RNA: Structure, Evolution, Processing and Function. R. A. Zimmermann and A. E. Dahlberg, eds. (Boca Raton: CRC Press), pp. 327-55; Samaha et al, 1995. Nature 377, 309-14) and that the PTC contains two highly conserved RNA features, termed the A- and P-loops, which accommodate the 3′-termini of the A (aminoacyl)- and P (peptidyl)-tRNAs. The recent crystal structures of the T thermophilus 70S particle complexed with tRNA molecules (Yusupov et al, 2001. Science 292, 883-96), of the LRS of the halophilic archaea Haloarcula marismortui (H. marismortui) complexed with substrate analogs (Hansen, J. L. et al, 2002) Molecular Cell, 10,117-128; Nissen, P. et al, 2000. Science 289, 920-930; Schmeing, T. M. et al, 2002. Nat Struct Biol 9, 225-230), and of the D. radiodurans LRS complexed with peptidyl transferase antibiotics, as described in the present disclosure, below (Schlüenzen et al., 2001. Nature 413, 814-21), show that the PTC is located at the bottom of a cavity containing all of the nucleotides known to bind the 3′-ends of the A-site and P-site tRNA molecules.

The peptidyl transferase activity of the ribosome has specifically been linked to a multi branched loop in the secondary structure diagram of 23S rRNA known as the peptidyl transferase ring (PTR). The PTR contains the PTC and the entrance to the nascent protein exit tunnel. Both are highly conserved throughout all kingdoms. Although organisms can tolerate only a few modifications in the PTR without loss of viability, several mutations or post-translational modifications in the PTR, leading to antibiotic resistant microorganisms, have been identified (reviewed in Mankin, 2001. Molecular Biology 35, 509-520; Weisblum, 1998. Drug Resistance Updates 1, 29-41).

During the course of protein biosynthesis, once a peptide bond is formed, the P-site tRNA is deacylated and its acceptor end translocates to the E-site, while the A-site tRNA, carrying the nascent chain translocates into the P-site. Translocation may be performed either by a simple translation of entire tRNA molecules, according to the classical three-site model (Rheinberger et al, 1981. Proc Natl Acad Sci USA. 78, 5310-4; Lill and Wintermeyer, 1987. J Mol Biol 196, 137-48), or incorporate an additional intermediate hybrid state. According to the latter hypothesis, translocation occurs in two discrete steps. In the first, spontaneous step, occurring immediately following peptide bond formation, the tRNA acceptor end moves relative to the LRS. In the second step, which is promoted by EF-G, the anticodon moves relative to the small subunit (Moazed and Noller, 1989. Nature 342, 142-8; Wilson and Noller, 1998. Cell 92, 337-49). Translocation has been shown to require substantial motions of ribosomal features (Wilson and Noller, 1998. Cell 92, 337-49).

The entrance of the protein exit tunnel of the ribosome, located adjacent to the PTC, was first detected by low resolution electron microscopy (Milligan R A. and P. N. Unwin, 1986. Nature 319, 693-5; Yonath A. et al., 1987. Science 236, 813-6), was assumed to provide a passive path for exporting smoothly all protein sequences. However, recent evidence for tunnel participation in regulating intracellular cotranslational processes indicated that the tunnel may possess dynamic capabilities allowing it to function as a discriminating gate and to respond to signals from cellular factors or from nascent proteins (Walter and Johnson, 1994. Annu Rev Cell Biol 10, 87-119; Gabashvili et al., 2001. Mol Cell 8, 181-8; Tenson and Mankin, 2001. Peptides 22, 1661-8; Nakatogawa and Ito, 2002. Cell 108, 629-36; Tenson T. and Ehrenberg M., 2002. Cell 108, 591-4). Sequences that presumably interact with the tunnel interior and thereby arrest protein elongation cycle have been identified. This interactive elongation arrest was proposed to provide mechanisms to guarantee critical events, such as sub-cellular localizations (Walter P. and Johnson A. E., 1994. Annu Rev Cell Biol 10, 87-119) or subunit assembly (Young J C. and Andrews D W., 1996. Embo J 15, 172-81). The secM (secretion monitor) gene has been found to encode a secretory protein that monitors protein export (Sarker S.et al., 2000. J Bacteriol 182, 5592-5). It includes a sequence motif that causes arrest during translation in the absence of the protein export system (called also “pulling protein”), which can be bypassed by mutations in the ribosomal RNA or in ribosomal protein L22 (Nakatogawa H. and Ito K., 2002. Cell 108, 629-36), a constituent of the tunnel walls, as described in the present disclosure below (Harms J. et al., 2001. Cell 107, 679-88) and elsewhere (Nissen P. et al., 2000. Science 289, 920-30). Ribosomal protein L22 consists of a single globular domain and a well structured highly conserved beta-hairpin with a unique twisted conformation which maintains the same length in all species whereas insertions/deletions exist in other regions of L22. Within the ribosome L22 has a conformation similar to that seen in its crystal structure (Unge J. et al., 1998. Structure 6, 1577-86).

In contrast to the small ribosomal subunit, the structure of the LRS was reported to be compact and monolithic (Ban et al., 2000. Science 289, 905-20). Nevertheless, significant mobility was assigned to the LRS's features that are directly involved in the ribosomal functions. Large scale movements were detected by cryo-electron microscopy (Stark et al., 1997. Cell, 88, 19-28; Frank and Agrawal, 1998. Biophys J., 74, 589-94), by surface RNA probing (Alexander et al., 1994. Biochemistry 33, 12109-18) and by monitoring the ribosomal activity. Evidence for the motion of the small subunit were obtained by cryo electron microscopy (Frank and Agrawal, 2000. Nature 406, 318-22; VanLoock et al., 2000. Journal of Molecular Biology 304, 507-515) and X-ray crystallography (Brodersen et al., 2000. Cell 103, 1143-54; Carter et al., 2000. Nature 407, 340-8; Ogle et al., 2001. Science 292, 897-902; Schlüenzen et al., 2000. Nature 413, 814-21; Pioletti et al., 2001. EMBO J, 20, 1829-39). Comparison of the crystal structures of the entire ribosome with the structures of its LRS showed that most of the functionally relevant features of the large subunit assume different conformations in unbound, as described in Example 1 of the present disclosure below (Harms J. et al., 2001. Cell 107, 679-688; Yonath, 2002. Annu Rev Biophys Biomol Struct 31, 257-73), and tRNA-complexed states (Yusupov et al., 2001. Science 292, 883-96), or become completely disordered, as found in the 2.4 Å crystal structure of the H. marismortui LRS (Ban et al., 2000. Science 289, 905-20). Examples are the L1 stalk, a region of the LRS suggested to provide the pivotal movement required for the release of E-site tRNA, and several intersubunit bridges, including the bridge connecting the decoding site in the small subunit with the PTC in the LRS, as described in Example 1 of the present disclosure below and elsewhere (Harms J. et al., 2001. Cell 107, 679-688; Yonath, 2002. Annu Rev Biophys Biomol Struct 31, 257-73).

Thus, during protein synthesis, the ribosome performs an intricate multi-step process requiring smooth and rapid switches between different conformations. Both ribosomal subunits can undergo reversible alterations and contain structural elements that participate in global motions together with local rearrangements.

Importantly, the ribosomal subunits are the major molecular binding targets for a broad range of natural and synthetic antibiotics which prevent bacterial growth and/or survival by blocking subunit function, thereby preventing protein synthesis. In particular, the PTC of the LRS serves as the major binding target for many antibiotics, including chloramphenicol; lincosamides, such as clindamycin; streptogramins B; sparsomycin; and ribosomal substrate analogs, such as puromycin (Spahn CMT. and Prescott C D., 1996. J Mol Med.-Jmm. 74, 423).

The structural basis of the interactions between the PTC and antibiotics has been, until recently, unknown. Chloramphenicol is known to hamper binding of tRNA to the A-site, thereby inhibiting ribosome function by blocking peptidyl transferase activity (Rodriguez-Fonseca C. et al., 1995. Journal of Molecular Biology 247, 224; Moazed D. and Noller H F., 1987. Biochemie 69, 879); lincosamides, such as clindamycin-an antibiotic which is bactericidal to many gram-positive aerobic bacteria and many anaerobic microorganisms-inhibit ribosome function by interacting with the A- and P-sites (Kalliaraftopoulos S. et al., 1994. Molecular Pharmacology 46, 1009); and puromycin is also known to bind to the active site.

In contrast, macrolides do not block peptidyl transferase activity (Vazquez, D. in: Inhibitors of protein synthesis. Springer Verlag, Berlin, Germany, 1975), but rather inhibit ribosome function by binding to the entrance of the protein exit tunnel of the LRS, thereby blocking the tunnel that channels the nascent peptides away from the PTC (Milligan, R A. and Unwin, P N., 1986. Nature 319, 693; Nissen, P. et al., 2000. Science 289, 920; Yonath, A. et al., 1987. Science 236, 813).

The macrolide group of antibiotics includes compounds such as the original macrolide erythromycin; the second generation semi-synthetic erythromycin derivatives clarithromycin, roxithromycin, and troleandomycin; and the more recently developed erythromycin-derived ketolides and azalides, such as the azalide azithromycin and the ketolide ABT-773.

Antibiotics such as clarithromycin and roxithromycin are characterized by increased acid stability relative to erythromycin, (Steinmetz W E. et al., 1992. Journal of Medicinal Chemistry 35, 4842; Gasc J C. et al., 1991. Journal of Antibiotics 44, 313).

The development of the chemically modified azalide (Bright et al., 1988. Journal of Antibiotics 41, 1029-1047) and ketolide (Agouridas et al., 1998. Journal of Medicinal Chemistry 41, 4080-4100) macrolides resulted from an intensified the search for new antimicrobial agents prompted by the rapid emergence of drug resistance in many pathogenic bacteria. Azalide antimicrobials are semi-synthetic derivatives of erythromycin with a 15-membered lactone ring. The insertion of a methyl-substituted nitrogen in the lactone ring increases their basicity, resulting in an improved acid stability and oral bio-availability as compared to erythromycin. Ketolides are a novel class of antibiotics derived from 14-membered macrolides by substitution of the cladinose sugar by a 3-keto group.

Ketolides derive their name from the insertion of a ketone bond in the 3-position and the removal of the 1-cladinose sugar, which lead to a broader spectrum of activity against a number of erythromycin- or penicillin-resistant pathogens (Davies et al., 2000. Antimicrobial Agents and Chemotherapy 44, 1894-1899; Nagai et al., 2001. Antimicrobial Agents and Chemotherapy 45, 3242-3245; Rosato et al., 1998. Antimicrobial Agents and Chemotherapy 42, 1392-1396). In addition to the 3-ketone, the ketolide ABT-773 has a quinollyallyl at the 6-O position and a cyclic carbamate group at C-11, 12 inserted in the 14-membered lactone ring. The cladinose sugar was originally believed to be important for the binding of the macrolides, but previous studies of macrolide-ribosome interactions by the present inventors indicated the comparatively low contribution of the cladinose moiety to the binding of the macrolides on the 50S subunit (Schlüenzen et al., 2001. Nature 413, 814-821).

The macrolide azithromycin, a semi-synthetic derivative of erythromycin and the first member of the class of azalide antimicrobials, acts not only against atypical respiratory pathogens (for example, Legionella, C. pneumoniae, species of Mycoplasma), but also against Chlamydia trachomatis and nontuberculous mycobacteria. Both azithromycin and clarithromycin are active agents for patients with late-stage acquired immunodeficiency syndrome (AIDS). Among them, azithromycin is preferable due to its fewer inter-drug interactions. Azithromycin and clarithromycin have prolonged tissue levels, hence their pharmacokinetics allow shorter dosing schedules. Although both azithromycin and clarithromycin are well tolerated by children, azithromycin has the advantage of shorter treatment regimens and improved tolerance (Alvarez-Elcoro and Enzler, 1999. Mayo Clinic Proceedings 74, 613-634). It has been observed that ketolides as well as azalides are also active against a variety of resistant phenotypes, particularly those of the macrolide-lincosamide-streptogramin B (MLSB) phenotype, though the reason for the reduced resistance remains unknown. Clinically acquired resistance to macrolides is mostly due to the production of methylases by a group of genes termed the erm genes. The production of methylases, which modify the equivalent of E. coli A2058 in the 23S rRNA, can be induced by 14- and 15-membered macrolides, but apparently not by 16-membered macrolides like josamycin (Giovanetti et al., 1999; Hamiltonmiller, 1992) or ketolides like ABT-773 (Bonnefoy et al., 1997). Azithromycin as well as ABT-773 exhibit elevated activity against a number of penicillin-resistant and macrolide-resistant pathogenic bacteria.

The macrolide troleandomycin is a semi-synthetic compound that shares many structural features with erythromycin. In comparison to erythromycin, troleandomycin has an oxirane ring that replaces a methyl at position C8 of the lactone ring and lacks three hydroxyl groups, being fully acetylated (Chepkwony H. K. et al., 2001. J Chromatogr A 914, 53-8). Similar to other 14-member macrolides, troleandomycin is composed of a lactone ring and two sugar moieties; desosamine and cladinose. Troleandomycin has a unique chemical structure and displays high affinity for ribosomes, and pentapeptides have been discovered conferring resistance to this antibiotic (Tenson T. and Mankin A. S., 2001. Peptides 22, 1661-8; Verdier L. et al., 2002. Biochemistry 41, 4218-29).

Puromycin and sparsomycin are universal inhibitors of protein biosynthesis that exert their effects by direct interactions with the PTC. Unlike other antibiotics, puromycin does not lead to drug-resistance by mutations in the PTC [Garrett, R. A., and Rodriguez-Fonseca, C. (1995) The peptidyl transferase center. In: Ribosomal RNA: Structure, Evolution, Processing and Function. R. A. Zimmermann and A. E. Dahlberg, eds. (Boca Raton: CRC Press), pp. 327-55]. Puromycin is partially co-structural with the 3′ terminus of aminoacyl-tRNA (Harms J. et al., 2001. Cell 107, 679-688), but its aminoacyl residue is linked via an amide bridge rather than an ester bond. Puromycin is known to bind weakly to the large subunit. Puromycin probing in the presence of an active donor substrate can result in peptide bond formation (Odom et al., 1990. Biochemistry 29, 10734-44), which is uncoupled from movement of the A-site tRNA (Green et al., 1998. Science 280, 286-9). No further synthesis can take place since the amide bond of puromycin cannot be cleaved; hence the peptidyl-puromycin so obtained falls off the ribosome. Puromycin played a central role in biochemical experiments aimed at the understanding of the mechanism of peptide bond formation [Pestka, S. (1977) Inhibitors of protein synthesis, Weissbach H, Pestka S, eds Edition (New York: Weissbach H); Vazquez, 1979. Mol Biol Biochem Biophys. 30, 1-312; Gale et al., 1981. The molecular basis of antibiotic action (London: Wiley); Porse and Garrett, 1995; Kirillov et al., 1997; Rodriguez-Fonseca et al., 2000. Rna 6, 744-54] since it can bind to the A-site (Moazed and Noller, 1991. Proc Natl Acad Sci USA. 88, 3725-8; Monro et al., 1969. Nature 222, 356-8; Smith et al., 1965. J Mol Biol. 13, 617-28; Traut and Monro, 1964. J Mol Biol. 10, 63-72) as well as to the P-site, albeit to a lower extent (Bourd et al., 1983. Eur J Biochem. 135, 465-70; Kirillov et al., 1997. RNA 5, 1003-13).

Sparsomycin is a potent ribosome-targeted inhibitor with a strong activity on all cell types, including Gram-positive bacteria and highly resistant archaea [Goldberg and Mitsugi, 1966. Biochem Biophys Res Commun. 23, 453-9; Vazquez, 1979. Mol Biol Biochem Biophys. 30, 1-312; Cundliffe, E. (1981) Antibiotic inhibitors of ribosome function, E. F. Gale, E. Cundliffe, P. E. Reynolds, M. H. Richmond and M. H. Waring, eds. (London, New York, Sydney, Toronto: Wiley)], but despite its universality, ribosomes from different kingdoms show differences in binding affinities to it (Lazaro et al., 1991. J Mol Biol 261, 231-8). Sparsomycin was shown to bind weakly to the ribosome (Monro et al., 1969), and donor substrates like N-blocked aminoacyl-tRNA appear to enhance its binding (Monro et al., 1968. Proc Natl Acad Sci USA. 61, 1042-9; Vazquez, 1979. Mol Biol Biochem Biophys. 30, 1-312; Hornig et al., 1987. Biochimie 69, 803-13; Lazaro et al., 1991. J Mol Biol 261, 231-8; Moazed and Noller, 1991. Proc Natl Acad Sci USA. 88, 3725-8; Porse et al., 1999. Proc Natl Acad Sci USA. 96, 9003-8; Theocharis and Coutsogeorgopoulos, 1992. Biochemistry 31, 5861-8). In contrast to most of the ribosome-targeted drugs that protect certain rRNA bases from chemical modification, sparsomycin does not produce clear footprints on the 23S rRNA (Moazed and Noller, 1991. Proc Natl Acad Sci USA. 88, 3725-8). Though not competitively inhibiting the binding of acceptor substrates to the A-site, A-site antibiotics such as chloramphenicol and lincomycin compete with sparsomycin for binding to bacterial ribosomes. Hence it was suggested that it binds to the A-site. However, since results from competition studies revealed discrepancies between the drug affinity and its biological effects, conformational rearrangements were suggested (Lazaro et al., 1991. J Mol Biol 261, 231-8; Porse et al., 1999. Proc Natl Acad Sci USA. 96, 9003-8). The A-site was implicated as a sparsomycin binding site also because mutations in A-site nucleotides leading to sparsomycin tolerance have been identified (Lazaro et al., 1996. J Mol Biol 261, 231-8; Tan et al., 1996. Journal of Molecular Biology 261, 222-30). More recent studies, however, localized crosslinks between sparsomycin and nucleotide 2602 and pointed to its association with the P-site (Porse et al., 1999. Proc Natl Acad Sci USA. 96, 9003-8).

Bacterial resistance to currently available antibiotics is responsible for the presently expanding global epidemics of increasing numbers of lethal or debilitating diseases caused by antibiotic resistant or multi-resistant strains of bacterial pathogens. Such diseases include, for example, bacteremia, pneumonia, endocarditis, bone infections, joint infections and nocosomial infections caused by Staphylococcus aureus (Bradley S F., 2002. Clin Infect Dis. 34, 211), and pulmonary infections caused by Haemophilus influenzae or Streptococcus pneumoniae (S. pneunoniae; Mlynarczyk G. et al., 2001. Int J Antimicrob Agents 18, 497).

The molecular mechanisms whereby bacteria become antibiotic-resistant usually involve drug efflux, drug inactivation, or alterations in the antibiotic target site. Although ribosomal proteins can affect the binding and action of ribosome-targeted antibiotics, the primary target of these antibiotics is rRNA (Cundliffe, E. in The Ribosome: Structure, Function and Evolution (1990) pp. 479-490, ASM Press, Washington, D.C. eds. Hill W E. et al.) and many cases of antibiotic resistance in clinical strains can be linked to alterations of specific nucleotides of the 23S rRNA within the PTC or around the entrance of the exit tunnel of the LRS (Vester B. and Douthwaite S., 2001. Antimicrobial Agents and Chemotherapy 45, 1).

Large ribosomal subunit-targeting antibiotics to which bacterial resistance has become problematic include lincosamides, such as clindamycin, an effective antibiotic in the treatment of most infections involving anaerobes and gram-positive cocci (Kasten M J., 1999. Mayo Clin Proc. 74, 825); chloramphenicol, an effective antibiotic in the treatment of a wide variety of bacterial infections, including serious anaerobic infections (Johnson A W. et al., 1992. Acta Paediatr. 81, 941); and macrolides, antibiotics offering coverage against a broad spectrum of pathogens and to which there has been reported a global increase in resistance among respiratory pathogens, particularly S. pneumoniae (Douthwaite S., 2001. Clin Microbiol Infect. Suppl. 3, 11). Indeed, resistance to macrolides and lincosamides, among other antibiotics, appears in almost all streptococcal species that attack humans (Horaud T. et al., 1985. J Antimicrob Chemother. 16 Suppl A, 111).

In gram-negative bacteria, resistance to macrolides is frequently related to the impermeability of the cellular outer membrane as a consequence of the hydrophobic nature of macrolide antibiotics such as erythromycin (Montenez et al., 1999. Toxicology and Applied Pharmacology 156, 129-140; Savage, 2001. Annals of Medicine 33, 167-171; Vaara, 1993. Antimicrobial Agents and Chemotherapy 37, 354-356). While structurally modified macrolides, such as the azalides, display higher efficiency against some gram-negative respiratory pathogens, such as Haemophilus influenzae, and enteric bacilli, including Shigella and Salmonella species (Gordillo et al., 1993. Antimicrobial Agents and Chemotherapy 37, 1203-1205; Gur et al., 1993. Journal of Chemotherapy 5, 151-152; Rakita et al., 1994. Antimicrobial Agents and Chemotherapy 38, 1915-1921), the effectiveness of such modified antibiotics remains unsatisfactory.

There is therefore a vital need for antibiotics being effective against bacterial pathogens responsible for diseases for which no satisfactory therapies have been elaborated, and/or against antibiotic-resistant bacterial pathogens.

One ideal strategy for facilitating the development of such antibiotics would be to generate three-dimensional atomic structure models of LRSs complexed with antibiotics, inhibitors of ribosomal activity, and/or ribosomal substrates, so as to elucidate the mechanisms of antibiotic activity, antibiotic resistance, or ribosomal function. Such models would constitute a potent tool for the identification or rational design of putative antibiotics having desired properties. Such models could also find utility in facilitating the rational design or identification of ribosomes having desired characteristics, for example, for enhancing production of recombinant proteins, such proteins being currently difficult and costly to produce in industrial quantities and being uniquely useful and in great demand in various biomedical, pharmacological, and industrial applications.

Numerous prior art approaches have been employed in attempts to generate three-dimensional atomic structure models of the LRS, whether alone or complexed to antibiotics, inhibitors and/or substrates.

One approach has attempted to crystallize ribosomal components of the eubacterium E. coli, the preferred model organism for such studies, for structural analysis thereof by X-ray crystallography. However, this approach has been hindered by the fact that E. coil ribosomal components are too fragile to resist deterioration during attempts at satisfactory crystallization thereof.

Another approach utilizing E. coli has used cryo-electron microscopy of the 70S ribosomal particle thereof complexed with formyl-methionyl initiator tRNAf(Met) (Gabashvili, I. S. et al., 2000. Cell 100, 537). This approach, however, failed to yield high resolution structures of the ribosome.

Thus, approaches employing X-ray crystallography of ribosomal subunits of bacterial species adapted to extreme environmental conditions have been employed since such organisms appear to express robust ribosomal components, more easily crystallized, than those of E. coli.

One approach has employed X-ray crystallography of the 30S subunit of T. thermophilus (T30S) (Schlüenzen, F. et al., 2000. Cell 102, 615; Wimberly B T. et al., 2000. Nature 407, 327; Clemons W M. et al., 2001. J Mol Biol. 310, 827).

Another approach has used X-ray crystallography of a complex of T30S with the initiation factor IF1 (Carter, A. P. et al., 2001. Science 291, 498).

Yet another approach has employed X-ray crystallography of T30S in complex with mRNA and cognate tRNA in the A-site, both in the presence and absence of the antibiotic paromomycin (Ogle, J. M. et al., 2001. Science 292, 897).

Still another approach has utilized X-ray crystallography of complexes of T30S with the antibiotics paromomycin, streptomycin or spectinomycin which interfere with decoding and translocation (Carter A. P. et al., 2000. Nature 407, 340).

A further approach has employed X-ray crystallography of T30S in complexes with the antibiotics tetracycline, pactamycin or hygromycin (Brodersen D. E. et al., 2000. Cell 103, 1143).

Yet a further approach has used X-ray crystallography of T30S in complexes with tetracycline, the universal initiation inhibitor edeine or the C-terminal domain of the translation initiation factor IF3 (Pioletti, M. et al., 2001. Embo J. 20, 1829).

Still a further approach, similar to that described above utilizing E. coli 70S ribosomal particle, has employed X-ray crystallography of the T. thermophilus 70S ribosomal particle (T70S) complexed with mRNAs and tRNAs (Yusupov MM. et al., 2001. Science 292, 883).

An additional approach has utilized X-ray crystallography of the H. marismortui LRS (Ban, N. et al., 2000. Science 289, 905; Nissen et al., 2001. Proc Natl Acad Sci USA. 98, 4899-903), of complexes thereof with substrate analogs (Nissen P. et al., 2000. Science 289, 920; Schmeing et al., 2002. Nat Struct Biol 9, 225-30), and of complexes thereof with the antibiotics anisomycin, blasticidin, carbomycin, sparsomycin, spiramycin, tylosin and virginiamycin (Eur Pat No.: EP1188769A2).

All of the aforementioned prior art approaches, however, suffer from critical disadvantages.

Most significantly, all of the structural features involved in the non-catalytic functional aspects of protein biosynthesis were found to be disordered in the atomic structure of the H. marismortui 50S LRS (H50S), the only LRS whose structure has been partially determined at high resolution. The use of H50S is a poor model for numerous reasons. For example, since ribosomes of such halophilic bacteria are resistant to antibiotic agents (Mankin A S. and Garrett R A., 1991. J Bacteriol. 173, 3559), the ribosomes thereof are less suitable models for generating ribosome-antibiotic complexes, and, in any case, H. marismortui is an archaea having eukaryotic properties and hence constitutes a clearly suboptimal model organism to elucidate ribosomal structure and function since biomedically, pharmacologically and industrially relevant bacterial strains are in large majority eubacteria which are evolutionarily and biologically divergent organisms (Willumeit R. et al., 2001. Biochim Biophys Acta. 1520, 7).

In the case of approaches in which LRSs were modeled via determining the lower resolution structure of whole 70S ribosomal particles, none were capable of generating high resolution three-dimensional atomic structure models, nor did these provide models of the atomic interactions between the LRS and any antibiotic molecule.

Thus, all prior art approaches have failed to provide satisfactory high resolution three-dimensional atomic structure models of the LRS, whether alone or complexed with an antibiotic or ribosomal substrate.

There is thus a widely recognized need for, and it would be highly advantageous to have, high resolution three-dimensional atomic structure models of the LRS in the free stat, and complexed with antibiotics or ribosomal substrates devoid of the above limitation.

### SUMMARY OF THE INVENTION

According to one aspect of the present invention there is provided a composition-of-matter comprising a crystallized complex of an antibiotic bound to a large ribosomal subunit of a eubacterium.

According to further features in preferred embodiments of the invention described below, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the antibiotic is clindamycin and the crystallized complex is characterized by unit cell dimensions of a=170.286±10 Å, b=410.134±15 Å and c=697.201±25 Å.

According to still further features in preferred embodiments, the antibiotic is erythromycin and the crystallized complex is characterized by unit cell dimensions of a=169.194±10 Å, b=409.975±15 Å and c=695.049±25 Å.

According to still further features in preferred embodiments, the antibiotic is clarithromycin and the crystallized complex is characterized by unit cell dimensions of a=169.871±10Å, b=412.705±15 Å and c=697.008±25 Å.

According to still further features in preferred embodiments, the antibiotic is roxithromycin and the crystallized complex is characterized by unit cell dimensions of a=170.357±10 Å, b=410.713±15 Å and c=694.810±25 Å.

According to still further features in preferred embodiments, the antibiotic is chloramphenicol and the crystallized complex is characterized by unit cell dimensions of a=171.066±10 Å, b=409.312±15 Å and c=696.946±25 Å.

According to still further features in preferred embodiments, the antibiotic is ACCP and the crystallized complex is characterized by unit cell dimensions of a=169.9 Å, b=410.4 and c=697.1 Å.

According to still further features in preferred embodiments, the antibiotic is ASM and the crystallized complex is characterized by unit cell dimensions of a=169.9 Å, b=409.9 Å and c=695.9 Å.

According to still further features in preferred embodiments, the antibiotic is ASMS and the crystallized complex is characterized by unit cell dimensions of a=169.6 Å, b=409.4 Å and c=695.1 Å.

According to still further features in preferred embodiments, the antibiotic is sparsomycin and the crystallized complex is characterized by unit cell dimensions of a=169.1 Å, b=409.9 Å and c=696.3 Å.

According to still further features in preferred embodiments, the antibiotic is troleandomycin and the crystallized complex is characterized by unit cell dimensions of a=170.3 Å, b=411.1 Å and c=695.5 Å.

According to still further features in preferred embodiments, the crystallized complex is characterized by having a crystal space group of I222.

According to still further features in preferred embodiments, the antibiotic is selected from the group consisting of chloramphenicol, a lincosamide antibiotic, a macrolide antibiotic, a puromycin conjugate, ASMS, and sparsomycin.

According to still further features in preferred embodiments, the lincosamide antibiotic is clindamycin.

According to still further features in preferred embodiments, the macrolide antibiotic is selected from the group consisting of erythromycin, clarithromycin, roxithromycin, troleandomycin, a ketolide antibiotic and an azalide antibiotic.

According to still further features in preferred embodiments, the ketolide antibiotic is ABT-773.

According to still further features in preferred embodiments, the azalide antibiotic is azithromycin.

According to still further features in preferred embodiments, the puromycin conjugate is ACCP or ASM.

According to still further features in preferred embodiments, the antibiotic is chloramphenicol and the large ribosomal subunit comprises a nucleic acid molecule, a segment of which including nucleotides being associated with the chloramphenicol, wherein a three-dimensional atomic structure of the nucleotides is defined by the set of structure coordinates corresponding to nucleotide coordinates 2044, 2430, 2431, 2479 and 2483-2485 set forth in Table 7.

According to still further features in preferred embodiments, the antibiotic is chloramphenicol and the large ribosomal subunit comprises a nucleic acid molecule, a segment of which including nucleotides being associated with the chloramphenicol, wherein a three-dimensional atomic structure of the nucleotides is defined by a set of structure coordinates having a root mean square deviation of not more than 2.0 Å from the set of structure coordinates corresponding to nucleotide coordinates 2044, 2430, 2431, 2479 and 2483-2485 set forth in Table 7.

According to still further features in preferred embodiments, a three-dimensional atomic structure of the segment is defined by the set of structure coordinates corresponding to nucleotide coordinates 2044-2485 set forth in Table 7.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 2044-2485 set forth in Table 7.

According to still further features in preferred embodiments, the antibiotic is chloramphenicol and a three-dimensional atomic structure of the chloramphenicol is defined by the set of structure coordinates corresponding to HETATM coordinates 59925-59944 set forth in Table 7.

According to still further features in preferred embodiments, the antibiotic is chloramphenicol and a three-dimensional atomic structure of the chloramphenicol is defined by a set of structure coordinates having a root mean square deviation of not more than 2.0 Å from the set of structure coordinates corresponding to HETATM coordinates 59925-59944 set forth in Table 7.

According to still further features in preferred embodiments, the antibiotic is clindamycin and the large ribosomal subunit comprises a nucleic acid molecule, a segment of which including nucleotides being associated with the clindamycin, wherein a three-dimensional atomic structure of the nucleotides is defined by the set of structure coordinates corresponding to nucleotide coordinates 2040-2042, 2044, 2482, 2484 and 2590 set forth in Table 8.

According to still further features in preferred embodiments, the antibiotic is clindamycin and the large ribosomal subunit comprises a nucleic acid molecule, a segment of which including nucleotides being associated with the clindamycin, wherein a three-dimensional atomic structure of the nucleotides is defined by a set of structure coordinates having a root mean square deviation of not more than 2.0 Å from the set of structure coordinates corresponding to nucleotide coordinates 2040-2042, 2044, 2482, 2484 and 2590 set forth in Table 8.

According to still further features in preferred embodiments, a three-dimensional atomic structure of the segment is defined by the set of structure coordinates corresponding to nucleotide coordinates 2040-2590 set forth in Table 8.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 2040-2590 set forth in Table 8.

According to still further features in preferred embodiments, the antibiotic is clindamycin and a three-dimensional atomic structure of the clindamycin is defined by the set of structure coordinates corresponding to HETATM coordinates 59922-59948 set forth in Table 8.

According to still further features in preferred embodiments, the antibiotic is clindamycin and a three-dimensional atomic structure of the clindamycin is defined by a set of structure coordinates having a root mean square deviation of not more than 2.0 Å from the set of structure coordinates corresponding to HETATM coordinates 59922-59948 set forth in Table 8.

According to still further features in preferred embodiments, the antibiotic is clarithromycin and the large ribosomal subunit comprises a nucleic acid molecule, a segment of which including nucleotides being associated with the clarithromycin, wherein a three-dimensional atomic structure of the nucleotides is defined by the set of structure coordinates corresponding to nucleotide coordinates 2040-2042, 2045, 2484, 2588 and 2589 set forth in Table 9.

According to still further features in preferred embodiments, the antibiotic is clarithromycin and the large ribosomal subunit comprises a nucleic acid molecule, a segment of which including nucleotides being associated with the clarithromycin, wherein a three-dimensional atomic structure of the nucleotides is defined by a set of structure coordinates having a root mean square deviation of not more than 2.0 Å from the set of structure coordinates corresponding to nucleotide coordinates 2040-2042, 2045, 2484, 2588 and 2589 set forth in Table 9.

According to still further features in preferred embodiments, a three-dimensional atomic structure of the segment is defined by the set of structure coordinates corresponding to nucleotide coordinates 2040-2589 set forth in Table 9.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 2040-2589 set forth in Table 9.

According to still further features in preferred embodiments, the antibiotic is clarithromycin and a three-dimensional atomic structure of the clarithromycin is defined by the set of structure coordinates corresponding to HETATM coordinates 59922-59973 set forth in Table 9.

According to still further features in preferred embodiments, the antibiotic is clarithromycin and a three-dimensional atomic structure of the clarithromycin is defined by a set of structure coordinates having a root mean square deviation of not more than 2.0 Å from the set of structure coordinates corresponding to HETATM coordinates 59922-59973 set forth in Table 9.

According to still further features in preferred embodiments, the antibiotic is erythromycin and the large ribosomal subunit comprises a nucleic acid molecule, a segment of which including nucleotides being associated with the erythromycin, wherein a three-dimensional atomic structure of the nucleotides is defined by the set of structure coordinates corresponding to nucleotide coordinates 2040-2042, 2045, 2484, 2588 and 2589 set forth in Table 10.

According to still further features in preferred embodiments, the antibiotic is erythromycin and the large ribosomal subunit comprises a nucleic acid molecule, a segment of which including nucleotides being associated with the erythromycin, wherein a three-dimensional atomic structure of the nucleotides is defined by a set of structure coordinates having a root mean square deviation of not more than 2.0 Å from the set of structure coordinates corresponding to nucleotide coordinates 2040-2042, 2045, 2484, 2588 and 2589 set forth in Table 10.

According to still further features in preferred embodiments, a three-dimensional atomic structure of the segment is defined by the set of structure coordinates corresponding to nucleotide coordinates 2040-2589 set forth in Table 10.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 2040-2589 set forth in Table 10.

According to still further features in preferred embodiments, the antibiotic is erythromycin and a three-dimensional atomic structure of the erythromycin is defined by the set of structure coordinates corresponding to HETATM coordinates 59922-59972 set forth in Table 10.

According to still further features in preferred embodiments, the antibiotic is erythromycin and a three-dimensional atomic structure of the erythromycin is defined by a set of structure coordinates having a root mean square deviation of not more than 2.0 Å from the set of structure coordinates corresponding to HETATM coordinates 59922-59972 set forth in Table 10.

According to still further features in preferred embodiments, the antibiotic is roxithromycin and the large ribosomal subunit comprises a nucleic acid molecule, a segment of which including nucleotides being associated with the roxithromycin, wherein a three-dimensional atomic structure of the nucleotides is defined by the set of structure coordinates corresponding to nucleotide coordinates 2040-2042, 2045, 2484, 2588 and 2589 set forth in Table 11.

According to still further features in preferred embodiments, the antibiotic is roxithromycin and the large ribosomal subunit comprises a nucleic acid molecule, a segment of which including nucleotides being associated with the roxithromycin, wherein a three-dimensional atomic structure of the nucleotides is defined by a set of structure coordinates having a root mean square deviation of not more than 2.0 Å from the set of structure coordinates corresponding to nucleotide coordinates 2040-2042, 2045, 2484, 2588 and 2589 set forth in Table 11.

According to still further features in preferred embodiments, a three-dimensional atomic structure of the segment is defined by the set of structure coordinates corresponding to nucleotide coordinates 2040-2589 set forth in Table 11.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 2040-2589 set forth in Table 11.

According to still further features in preferred embodiments, the antibiotic is roxithromycin and a three-dimensional atomic structure of the roxithromycin is defined by the set of structure coordinates corresponding to HETATM coordinates 59922-59979 set forth in Table 11.

According to still further features in preferred embodiments, the antibiotic is roxithromycin and a three-dimensional atomic structure of the roxithromycin is defined by a set of structure coordinates having a root mean square deviation of not more than 2.0 Å from the set of structure coordinates corresponding to HETATM coordinates 59922-59979 set forth in Table 11.

According to still further features in preferred embodiments, the antibiotic is ABT-773 and the large ribosomal subunit comprises a nucleic acid molecule, a segment of which including nucleotides being associated with the ABT-773, wherein a three-dimensional atomic structure of the nucleotides is defined by the set of structure coordinates corresponding to nucleotide coordinates 803, 1773, 2040-2042, 2045, 2484, and 2588-2590 set forth in Table 18.

According to still further features in preferred embodiments, the antibiotic is ABT-773 and the large ribosomal subunit comprises a nucleic acid molecule, a segment of which including nucleotides being associated with the ABT-773, wherein a three-dimensional atomic structure of the nucleotides is defined by a set of structure coordinates having a root mean square deviation of not more than 2.0 Å from the set of structure coordinates corresponding to nucleotide coordinates 803, 1773, 2040-2042, 2045, 2484, and 2588-2590 set forth in Table 18.

According to still further features in preferred embodiments, a three-dimensional atomic structure of the segment is defined by the set of structure coordinates corresponding to nucleotide coordinates 803-2590 set forth in Table 18.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 803-2590 set forth in Table 18.

According to still further features in preferred embodiments, the antibiotic is ABT-773 and a three-dimensional atomic structure of the ABT-773 is defined by the set of structure coordinates corresponding to HETATM coordinates 1-55 set forth in Table 18.

According to still further features in preferred embodiments, the antibiotic is ABT-773 and a three-dimensional atomic structure of the ABT-773 is defined by a set of structure coordinates having a root mean square deviation of not more than 2.0 Å from the set of structure coordinates corresponding to HETATM coordinates 1-55 set forth in Table 18.

According to still further features in preferred embodiments, the antibiotic is azithromycin and the large ribosomal subunit comprises a nucleic acid molecule, a segment of which including nucleotides being associated with the azithromycin, wherein a three-dimensional atomic structure of the nucleotides is defined by the set of structure coordinates corresponding to nucleotide coordinates 764, 2041, 2042, 2045, A2482, 2484, 2565, and 2588-2590 set forth in Table 19.

According to still further features in preferred embodiments, the antibiotic is azithromycin and the large ribosomal subunit comprises a nucleic acid molecule, a segment of which including nucleotides being associated with the azithromycin, wherein a three-dimensional atomic structure of the nucleotides is defined by a set of structure coordinates having a root mean square deviation of not more than 2.0 Å from the set of structure coordinates corresponding to nucleotide coordinates 764, 2041, 2042, 2045, A2482, 2484, 2565, and 2588-2590 set forth in Table 19.

According to still further features in preferred embodiments, the antibiotic is azithromycin and the large ribosomal subunit comprises amino acid residues being associated with the azithromycin, wherein a three-dimensional atomic structure of the amino acid residues is defined by the set of structure coordinates corresponding to amino acid residue coordinates Y59, G60, G63, T64 and R111 set forth in Table 19.

According to still further features in preferred embodiments, the antibiotic is azithromycin and the large ribosomal subunit comprises amino acid residues being associated with the azithromycin, wherein a three-dimensional atomic structure of the amino acid residues is defined by a set of structure coordinates having a root mean square deviation of not more than 2.0 Å from the set of structure coordinates corresponding to amino acid residue coordinates Y59, G60, G63, T64 and R111 set forth in Table 19.

According to still further features in preferred embodiments, a three-dimensional atomic structure of the segment is defined by the set of structure coordinates corresponding to nucleotide coordinates 764-2590 set forth in Table 19.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 764-2590 set forth in Table 19.

According to still further features in preferred embodiments, the antibiotic is azithromycin and a three-dimensional atomic structure of the azithromycin is defined by the set of structure coordinates corresponding to HETATM coordinates 79705-79808 set forth in Table 19.

According to still further features in preferred embodiments, the antibiotic is azithromycin and a three-dimensional atomic structure of the azithromycin is defined by a set of structure coordinates having a root mean square deviation of not more than 2.0 Å from the set of structure coordinates corresponding to HETATM coordinates 79705-79808 set forth in Table 19.

According to still further features in preferred embodiments, the antibiotic is ACCP and the large ribosomal subunit comprises a nucleic acid molecule, a segment of which including nucleotides being associated with the ACCP, wherein a three-dimensional atomic structure of the nucleotides is defined by the set of structure coordinates corresponding to nucleotide coordinates 1924, 2430, 2485, 2532-2534, 2552, 2562, and 2583 set forth in Table 20.

According to still further features in preferred embodiments, the antibiotic is ACCP and the large ribosomal subunit comprises a nucleic acid molecule, a segment of which including nucleotides being associated with the ACCP, wherein a three-dimensional atomic structure of the nucleotides is defined by a set of structure coordinates having a root mean square deviation of not more than 2.0 Å from the set of structure coordinates corresponding to nucleotide coordinates 1924, 2430, 2485, 2532-2534, 2552, 2562, and 2583 set forth in Table 20.

According to still further features in preferred embodiments, a three-dimensional atomic structure of the segment is defined by the set of structure coordinates corresponding to nucleotide coordinates 1924-2583 set forth in Table 20.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 1924-2583 set forth in Table 20.

According to still further features in preferred embodiments, the antibiotic is ACCP and a three-dimensional atomic structure of the ACCP is defined by the set of structure coordinates corresponding to atom coordinates 78760-78855 set forth in Table 20.

According to still further features in preferred embodiments, the antibiotic is ACCP and a three-dimensional atomic structure of the ACCP is defined by a set of structure coordinates having a root mean square deviation of not more than 2.0 Å from the set of structure coordinates corresponding to atom coordinates 78760-78855 set forth in Table 20.

According to still further features in preferred embodiments, the antibiotic is ASM and the large ribosomal subunit comprises: a nucleic acid molecule, a segment of which including nucleotides being associated with the ASM, wherein a three-dimensional atomic structure of the nucleotides is defined by the set of structure coordinates corresponding to nucleotide coordinates 1892, 1896, 1899, 1925, 1926, 2431, 2485, 2486, 2532, 2534, 2552, 2562, and 2581 set forth in Table 21; and a polypeptide including amino acid residues being associated with the ASM, wherein a three-dimensional atomic structure of the amino acid residues is defined by the set of structure coordinates corresponding to amino acid residue coordinates 79-81 set forth in Table 21.

According to still further features in preferred embodiments, the antibiotic is ASM and the large ribosomal subunit comprises: a nucleic acid molecule, a segment of which including nucleotides being associated with the ASM, wherein a three-dimensional atomic structure of the nucleotides is defined by a set of structure coordinates having a root mean square deviation of not more than 2.0 Å from the set of structure coordinates corresponding to nucleotide coordinates 1892, 1896, 1899, 1925, 1926, 2431, 2485, 2486, 2532, 2534, 2552, 2562, and 2581 set forth in Table 21; and a polypeptide including amino acid residues being associated with the ASM, wherein a three-dimensional atomic structure of the amino acid residues is defined by a set of structure coordinates having a root mean square deviation of not more than 2.0 Å from the set of structure coordinates corresponding to amino acid residue coordinates 79-81 set forth in Table 21.

According to still further features in preferred embodiments, a three-dimensional atomic structure of the segment is defined by the set of structure coordinates corresponding to nucleotide coordinates 1892-2581 set forth in Table 21.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 1892-2581 set forth in Table 21.

According to still further features in preferred embodiments, the antibiotic is ASM and a three-dimensional atomic structure of the ASM is defined by the set of structure coordinates corresponding to atom coordinates 78747-79289 set forth in Table 21.

According to still further features in preferred embodiments, the antibiotic is ASM and a three-dimensional atomic structure of the ASM is defined by a set of structure coordinates having a root mean square deviation of not more than 2.0 Å from the set of structure coordinates corresponding to atom coordinates 78747-79289 set forth in Table 21.

According to still further features in preferred embodiments, the antibiotic is ASMS and the large ribosomal subunit comprises: a nucleic acid molecule, a segment of which including nucleotides being associated with the ASMS, wherein a three-dimensional atomic structure of the nucleotides is defined by the set of structure coordinates corresponding to nucleotide coordinates 1899, 1924, 1926, 2430, 2472, 2485, 2486, 2532, 2534, 2552, 2562, 2563, and 2581; set forth in Table 22; a polypeptide including amino acid residues being associated with the ASM, wherein a three-dimensional atomic structure of the amino acid residues is defined by the set of structure coordinates corresponding to amino acid residue coordinates 79-81 set forth in Table 22; and magnesium ions being associated with the ASM, wherein a three-dimensional positioning of the magnesium ions is defined by the set of structure coordinates corresponding to atom coordinates 79393 and 79394 set forth in Table 22.

According to still further features in preferred embodiments, the antibiotic is ASMS and the large ribosomal subunit comprises a nucleic acid molecule, a segment of which including nucleotides being associated with the ASMS, wherein a three-dimensional atomic structure of the nucleotides is defined by a set of structure coordinates having a root mean square deviation of not more than 2.0 Å from the set of structure coordinates corresponding to nucleotide coordinates 1899, 1924, 1926, 2430, 2472, 2485, 2486, 2532, 2534, 2552, 2562, 2563, and 2581 set forth in Table 22; a polypeptide including amino acid residues being associated with the ASM, wherein a three-dimensional atomic structure of the amino acid residues is defined by a set of structure coordinates having a root mean square deviation of not more than 2.0 Å from the set of structure coordinates corresponding to amino acid residue coordinates 79-81 set forth in Table 22; and magnesium ions being associated with the ASM, wherein a three-dimensional positioning of the magnesium ions is defined by a set of structure coordinates having a root mean square deviation of not more than 2.0 Å from the set of structure coordinates corresponding to atom coordinates 79393 and 79394 set forth in Table 22.

According to still further features in preferred embodiments, a three-dimensional atomic structure of the segment is defined by the set of structure coordinates corresponding to nucleotide coordinates 1924-2581 set forth in Table 22.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 1924-2581 set forth in Table 22.

According to still further features in preferred embodiments, the antibiotic is ASMS and a three-dimensional atomic structure of the ASMS is defined by the set of structure coordinates corresponding to atom coordinates 78758-79322 set forth in Table 22.

According to still further features in preferred embodiments, the antibiotic is ASMS and a three-dimensional atomic structure of the ASMS is defined by a set of structure coordinates having a root mean square deviation of not more than 2.0 Å from the set of structure coordinates corresponding to coordinates 78758-79322 set forth in Table 22.

According to still further features in preferred embodiments, the antibiotic is sparsomycin and the large ribosomal subunit comprises a nucleic acid molecule, a nucleotide of which being associated with the sparsomycin, wherein a three-dimensional atomic structure of the nucleotide is defined by a set of structure coordinates corresponding to nucleotide coordinate 2581 set forth in Table 23.

According to still further features in preferred embodiments, the antibiotic is sparsomycin and the large ribosomal subunit comprises a nucleic acid molecule, a nucleotide of which being associated with the sparsomycin, wherein a three-dimensional atomic structure of the nucleotide is defined by a set of structure coordinates having a root mean square deviation of not more than 2.0 Å from the structure coordinate corresponding to nucleotide coordinate 2581 set forth in Table 23.

According to still further features in preferred embodiments, the antibiotic is sparsomycin and a three-dimensional atomic structure of the sparsomycin is defined by the set of structure coordinates corresponding to atom coordinates 78757-78778 set forth in Table 23.

According to still further features in preferred embodiments, the antibiotic is sparsomycin and a three-dimensional atomic structure of the sparsomycin is defined by a set of structure coordinates having a root mean square deviation of not more than 2.0 Å from the set of structure coordinates corresponding to atom coordinates 78757-78778 set forth in Table 23.

According to still further features in preferred embodiments, the antibiotic is troleandomycin and the large ribosomal subunit comprises a nucleic acid molecule, a segment of which including nucleotides being associated with the troleandomycin, wherein a three-dimensional atomic structure of the nucleotides is defined by the set of structure coordinates corresponding to nucleotide coordinates 759, 761, 803, 2041, 2042, 2045, 2484, and 2590 set forth in Table 38.

According to still further features in preferred embodiments, the antibiotic is troleandomycin and the large ribosomal subunit comprises a nucleic acid molecule, a segment of which including nucleotides being associated with the troleandomycin, wherein a three-dimensional atomic structure of the nucleotides is defined by a set of structure coordinates having a root mean square deviation of not more than 2.0 Å from the set of structure coordinates corresponding to nucleotide coordinates 759, 761, 803, 2041, 2042, 2045, 2484, and 2590 set forth in Table 38.

According to still further features in preferred embodiments, a three-dimensional atomic structure of the segment is defined by the set of structure coordinates corresponding to nucleotide coordinates 759-2590 set forth in Table 38.

According to still further features in preferred embodiments, a three-dimensional atomic structure of the segment is defined by a set of structure coordinates having a root mean square deviation of not more than 2.0 Å from the set of structure coordinates corresponding to nucleotide coordinates 759-2590 set forth in Table 38.

According to still further features in preferred embodiments, the antibiotic is troleandomycin and the large ribosomal subunit comprises an amino acid residue being associated with the troleandomycin, wherein a three-dimensional atomic structure of the amino acid residue is defined by the set of structure coordinates corresponding to amino acid residue coordinate Ala2 set forth in Table 38.

According to still further features in preferred embodiments, the antibiotic is troleandomycin and the large ribosomal subunit comprises an amino acid residue being associated with the troleandomycin, wherein a three-dimensional atomic structure of the nucleotides is defined by a set of structure coordinates having a root mean square deviation of not more than 2.0 Å from the set of structure coordinates corresponding to amino acid residue coordinate Ala2 set forth in Table 38.

According to still further features in preferred embodiments, the antibiotic is troleandomycin and a three-dimensional atomic structure of the troleandomycin is defined by the set of structure coordinates corresponding to atom coordinates 1-57 set forth in Table 38.

According to still further features in preferred embodiments, the antibiotic is troleandomycin and a three-dimensional atomic structure of the troleandomycin is defined by a set of structure coordinates having a root mean square deviation of not more than 2.0 Å from the set of structure coordinates corresponding to atom coordinates 1-57 set forth in Table 38.

According to another aspect of the present invention there is provided a composition-of-matter comprising a crystallized LRS of a eubacterium.

According to further features in preferred embodiments of the invention described below, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the crystallized large ribosomal subunit is characterized by unit cell dimensions of a=170.827±10 Å, b=409.430±15 Å and c=695.597±25 Å.

According to still further features in preferred embodiments, the crystallized large ribosomal subunit is characterized by having a crystal space group of I222.

According to still further features in preferred embodiments, a three-dimensional atomic structure of at least a portion of the crystallized large ribosomal subunit is defined by a set of structure coordinates corresponding to a set of coordinates set forth in Table 3, the set of coordinates set forth in Table 3 being selected from the group consisting of: nucleotide coordinates 2044, 2430, 2431, 2479 and 2483-2485; nucleotide coordinates 2044-2485; nucleotide coordinates 2040-2042, 2044, 2482, 2484 and 2590; nucleotide coordinates 2040-2590; nucleotide coordinates 2040-2042, 2045, 2484, 2588 and 2589; nucleotide coordinates 2040-2589; atom coordinates 1-59360; atom coordinates 59361-61880; atom coordinates 1-61880; atom coordinates 61881-62151; atom coordinates 62152-62357; atom coordinates 62358-62555; atom coordinates 62556-62734; atom coordinates 62735-62912; atom coordinates 62913-62965; atom coordinates 62966-63109; atom coordinates 63110-63253; atom coordinates 63254-63386; atom coordinates 63387-63528; atom coordinates 63529-63653; atom coordinates 63654-63768; atom coordinates 63769-63880; atom coordinates 63881-64006; atom coordinates 64007-64122; atom coordinates 64123-64223; atom coordinates 64224-64354; atom coordinates 64355-64448; atom coordinates 64449-64561; atom coordinates 64562-64785; atom coordinates 64786-64872; atom coordinates 64873-64889; atom coordinates 64890-64955; atom coordinates 64956-65011; atom coordinates 65012-65085; atom coordinates 65086-65144; atom coordinates 65145-65198; atom coordinates 65199-65245; atom coordinates 65246-65309; atom coordinates 65310-65345; atom coordinates 61881-65345; and atom coordinates 1-65345.

According to still further features in preferred embodiments, a three-dimensional atomic structure of at least a portion of the crystallized large ribosomal subunit is defined by a set of structure coordinates having a root mean square deviation of not more than 2.0 Å from a set of structure coordinates corresponding to a set of coordinates set forth in Table 3, the set of coordinates set forth in Table 3 being selected from the consisting of nucleotide coordinates 2044, 2430, 2431, 2479 and 2483-2485; nucleotide coordinates 2044-2485; nucleotide coordinates 2040-2042, 2044, 2482, 2484 and 2590; nucleotide coordinates 2040-2590; nucleotide coordinates 2040-2042, 2045, 2484, 2588 and 2589; nucleotide coordinates 2040-2589; atom coordinates 1-59360; atom coordinates 59361-61880; atom coordinates 1-61880; atom coordinates 61881-62151; atom coordinates 62152-62357; atom coordinates 62358-62555; atom coordinates 62556-62734; atom coordinates 62735-62912; atom coordinates 62913-62965; atom coordinates 62966-63109; atom coordinates 63110-63253; atom coordinates 63254-63386; atom coordinates 63387-63528; atom coordinates 63529-63653; atom coordinates 63654-63768; atom coordinates 63769-63880; atom coordinates 63881-64006; atom coordinates 64007-64122; atom coordinates 64123-64223; atom coordinates 64224-64354; atom coordinates 64355-64448; atom coordinates 64449-64561; atom coordinates 64562-64785; atom coordinates 64786-64872; atom coordinates 64873-64889; atom coordinates 64890-64955; atom coordinates 64956-65011; atom coordinates 65012-65085; atom coordinates 65086-65144; atom coordinates 65145-65198; atom coordinates 65199-65245; atom coordinates 65246-65309; atom coordinates 65310-65345; atom coordinates 61881-65345; and atom coordinates 1-65345.

According to still further features in preferred embodiments, the crystallized large ribosomal subunit comprises a nucleic acid molecule, a segment of which including nucleotides or amino acid residues being capable of specifically associating with an antibiotic selected from the group consisting of chloramphenicol, a lincosamide antibiotic, a macrolide antibiotic, a puromycin conjugate, ASMS, and sparsomycin.

According to still further features in preferred embodiments, the lincosamide antibiotic is clindamycin.

According to still further features in preferred embodiments, the macrolide antibiotic is selected from the group consisting of erythromycin, clarithromycin, roxithromycin, troleandomycin, a ketolide antibiotic and an azalide antibiotic.

According to still further features in preferred embodiments, the ketolide antibiotic is ABT-773.

According to still further features in preferred embodiments, the azalide antibiotic is azithromycin.

According to still further features in preferred embodiments, the puromycin conjugate is ACCP or ASM.

According to still further features in preferred embodiments, a three-dimensional atomic structure of the nucleic acid molecule is defined by the set of structure coordinates corresponding to atom coordinates 1-59360 set forth in Table 3.

According to still further features in preferred embodiments, the antibiotic is chloramphenicol and a three-dimensional atomic structure of the nucleotides being capable of specifically associating with the chloramphenicol is defined by the set of structure coordinates corresponding to nucleotide coordinates 2044, 2430, 2431, 2479 and 2483-2485 set forth in Table 3.

According to still further features in preferred embodiments, the antibiotic is chloramphenicol and a three-dimensional atomic structure of the nucleotides being capable of specifically associating with the chloramphenicol is defined by a set of structure coordinates having a root mean square deviation of not more than 2.0 Å from the set of structure coordinates corresponding to nucleotide coordinates 2044, 2430, 2431, 2479 and 2483-2485 set forth in Table 3.

According to still further features in preferred embodiments, the antibiotic is chloramphenicol and a three-dimensional atomic structure of the segment including the nucleotides being capable of specifically associating with the chloramphenicol is defined by the set of structure coordinates corresponding to nucleotide coordinates 2044-2485 set forth in Table 3.

According to still further features in preferred embodiments, the antibiotic is chloramphenicol and a three-dimensional atomic structure of the segment including the nucleotides being capable of specifically associating with the chloramphenicol is defined by a set of structure coordinates having a root mean square deviation of not more than 2.0 Å from the set of structure coordinates corresponding to nucleotide coordinates 2044-2485 set forth in Table 3.

According to still further features in preferred embodiments, the antibiotic is clindamycin and a three-dimensional atomic structure of the nucleotides being capable of specifically associating with the clindamycin is defined by the set of structure coordinates corresponding to nucleotide coordinates 2040-2042, 2044, 2482, 2484 and 2590 set forth in Table 3.

According to still further features in preferred embodiments, the antibiotic is clindamycin and a three-dimensional atomic structure of the nucleotides being capable of specifically associating with the clindamycin is defined by a set of structure coordinates having a root mean square deviation of not more than 2.0 Å from the set of structure coordinates corresponding to nucleotide coordinates 2040-2042, 2044, 2482, 2484 and 2590 set forth in Table 3.

According to still further features in preferred embodiments, the antibiotic is clindamycin and a three-dimensional atomic structure of the segment including the nucleotides being capable of specifically associating with the clindamycin is defined by the set of structure coordinates corresponding to nucleotide coordinates 2040-2590 set forth in Table 3.

According to still further features in preferred embodiments, the antibiotic is clindamycin and a three-dimensional atomic structure of the segment including the nucleotides being capable of specifically associating with the clindamycin is defined by a set of structure coordinates having a root mean square deviation of not more than 2.0 Å from the set of structure coordinates corresponding to nucleotide coordinates 2040-2590 set forth in Table 3.

According to still further features in preferred embodiments, the antibiotic is clarithromycin, erythromycin or roxithromycin, and a three-dimensional atomic structure of the nucleotides being capable of specifically associating with the antibiotic is defined by the set of structure coordinates corresponding to nucleotide coordinates 2040-2042, 2045, 2484, 2588 and 2589 set forth in Table 3.

According to still further features in preferred embodiments, the antibiotic is clarithromycin, erythromycin or roxithromycin, and a three-dimensional atomic structure of the nucleotides being capable of specifically associating with the antibiotic is defined by a set of structure coordinates having a root mean square deviation of not more than 2.0 Å from the set of structure coordinates corresponding to nucleotide coordinates 2040-2042, 2045, 2484, 2588 and 2589 set forth in Table 3.

According to still further features in preferred embodiments, the antibiotic is clarithromycin, erythromycin or roxithromycin, and a three-dimensional atomic structure of the segment including the nucleotides being capable of specifically associating with the antibiotic is defined by the set of structure coordinates corresponding to nucleotide coordinates 2040-2589 set forth in Table 3.

According to still further features in preferred embodiments, the antibiotic is clarithromycin, erythromycin or roxithromycin, and a three-dimensional atomic structure of the segment including the nucleotides being capable of specifically associating with the antibiotic is defined by a set of structure coordinates having a root mean square deviation of not more than 2.0 Å from the set of structure coordinates corresponding to nucleotide coordinates 2040-2589 set forth in Table 3.

According to still further features in preferred embodiments, the antibiotic is ABT-773, and a three-dimensional atomic structure of the nucleotides being capable of specifically associating with the antibiotic is defined by the set of structure coordinates corresponding to nucleotide coordinates 803, 1773, 2040-2042, 2045, 2484, and 2588-2590 set forth in Table 18.

According to still further features in preferred embodiments, the antibiotic is ABT-773, and a three-dimensional atomic structure of the nucleotides being capable of specifically associating with the antibiotic is defined by a set of structure coordinates having a root mean square deviation of not more than 2.0 Å from the set of structure coordinates corresponding to nucleotide coordinates 803, 1773, 2040-2042, 2045, 2484, and 2588-2590 set forth in Table 18.

According to still further features in preferred embodiments, the antibiotic is ABT-773, and a three-dimensional atomic structure of the segment including the nucleotides being capable of specifically associating with the antibiotic is defined by the set of structure coordinates corresponding to nucleotide coordinates 803-2590 set forth in Table 18.

According to still further features in preferred embodiments, the antibiotic is ABT-773, and a three-dimensional atomic structure of the segment including the amino acid residues being capable of specifically associating with the antibiotic is defined by a set of structure coordinates having a root mean square deviation of not more than 2.0 Å from the set of structure coordinates corresponding to amino acid residue coordinates 803-2590 set forth in Table 18.

According to still further features in preferred embodiments, the antibiotic is azithromycin, and a three-dimensional atomic structure of the nucleotides being capable of specifically associating with the antibiotic is defined by the set of structure coordinates corresponding to nucleotide coordinates 764, 2041, 2042, 2045, A2482, 2484, 2565, and 2588-2590 set forth in Table 19.

According to still further features in preferred embodiments, the antibiotic is azithromycin, and a three-dimensional atomic structure of the nucleotides being capable of specifically associating with the antibiotic is defined by a set of structure coordinates having a root mean square deviation of not more than 2.0 Å from the set of structure coordinates corresponding to nucleotide coordinates 764, 2041, 2042, 2045, A2482, 2484, 2565, and 2588-2590 set forth in Table 19.

According to still further features in preferred embodiments, the antibiotic is azithromycin, and a three-dimensional atomic structure of the segment including the nucleotides being capable of specifically associating with the antibiotic is defined by the set of structure coordinates corresponding to nucleotide coordinates 764-2590 set forth in Table 19.

According to still further features in preferred embodiments, the antibiotic is azithromycin, and a three-dimensional atomic structure of the segment including the nucleotides being capable of specifically associating with the antibiotic is defined by a set of structure coordinates having a root mean square deviation of not more than 2.0 Å from the set of structure coordinates corresponding to nucleotide coordinates 764-2590 set forth in Table 19.

According to still further features in preferred embodiments, the antibiotic is azithromycin, and a three-dimensional atomic structure of the segment including the amino acid residues being capable of specifically associating with the antibiotic is defined by the set of structure coordinates corresponding to amino acid residue coordinates Y59, G60, G63, T64 and R111 set forth in Table 19.

According to still further features in preferred embodiments, the antibiotic is azithromycin, and a three-dimensional atomic structure of the segment including the amino acid residues being capable of specifically associating with the antibiotic is defined by a set of structure coordinates having a root mean square deviation of not more than 2.0 Å from the set of structure coordinates corresponding to amino acid residue coordinates Y59, G60, G63, T64 and R111 set forth in Table 19.

According to still further features in preferred embodiments, the antibiotic is ACCP, and a three-dimensional atomic structure of the nucleotides being capable of specifically associating with the antibiotic is defined by the set of structure coordinates corresponding to nucleotide coordinates 1924, 2430, 2485, 2532, 2533, 2534, 2552, 2562, and 2583 set forth in Table 20.

According to still further features in preferred embodiments, the antibiotic is ACCP, and a three-dimensional atomic structure of the nucleotides being capable of specifically associating with the antibiotic is defined by a set of structure coordinates having a root mean square deviation of not more than 2.0 Å from the set of structure coordinates corresponding to nucleotide coordinates 1924, 2430, 2485, 2532, 2533, 2534, 2552, 2562, and 2583 set forth in Table 20.

According to still further features in preferred embodiments, the antibiotic is ACCP, and a three-dimensional atomic structure of the segment including the nucleotides being capable of specifically associating with the antibiotic is defined by the set of structure coordinates corresponding to nucleotide coordinates 1924-2583 set forth in Table 20.

According to still further features in preferred embodiments, the antibiotic is ACCP, and a three-dimensional atomic structure of the segment including the nucleotides being capable of specifically associating with the antibiotic is defined by a set of structure coordinates having a root mean square deviation of not more than 2.0 Å from the set of structure coordinates corresponding to nucleotide coordinates 1924-2583 set forth in Table 20.

According to still further features in preferred embodiments, the antibiotic is ASM, and a three-dimensional atomic structure of the nucleotides being capable of specifically associating with the antibiotic is defined by the set of structure coordinates corresponding to nucleotide coordinates 1892, 1896, 1899, 1925, 1926, 2431, 2485, 2486, 2532, 2534, 2552, 2562, and 2581 set forth in Table 21.

According to still further features in preferred embodiments, the antibiotic is ASM, and a three-dimensional atomic structure of the nucleotides being capable of specifically associating with the antibiotic is defined by a set of structure coordinates having a root mean square deviation of not more than 2.0 Å from the set of structure coordinates corresponding to nucleotide coordinates 1892, 1896, 1899, 1925, 1926, 2431, 2485, 2486, 2532, 2534, 2552, 2562, and 2581 set forth in Table 21.

According to still further features in preferred embodiments, the antibiotic is ASM, and a three-dimensional atomic structure of the segment including the nucleotides being capable of specifically associating with the antibiotic is defined by the set of structure coordinates corresponding to nucleotide coordinates 1892-2581 set forth in Table 21.

According to still further features in preferred embodiments, the antibiotic is ASM, and a three-dimensional atomic structure of the segment including the nucleotides being capable of specifically associating with the antibiotic is defined by a set of structure coordinates having a root mean square deviation of not more than 2.0 Å from the set of structure coordinates corresponding to nucleotide coordinates 1892-2581 set forth in Table 21.

According to still further features in preferred embodiments, the antibiotic is ASM, and a three-dimensional atomic structure of the segment including the amino acid residues being capable of specifically associating with the antibiotic is defined by the set of structure coordinates corresponding to amino acid residue coordinates 79-81 set forth in Table 21.

According to still further features in preferred embodiments, the antibiotic is ASM, and a three-dimensional atomic structure of the segment including the amino acid residues being capable of specifically associating with the antibiotic is defined by a set of structure coordinates having a root mean square deviation of not more than 2.0 Å from the set of structure coordinates corresponding to amino acid residue coordinates 79-81 set forth in Table 21.

According to still further features in preferred embodiments, the antibiotic is ASMS, and a three-dimensional atomic structure of the nucleotides being capable of specifically associating with the antibiotic is defined by the set of structure coordinates corresponding to nucleotide coordinates 1899, 1924, 1926, 2430, 2472, 2485, 2486, 2532, 2534, 2552, 2562, 2563, and 2581 set forth in Table 22.

According to still further features in preferred embodiments, the antibiotic is ASMS, and a three-dimensional atomic structure of the nucleotides being capable of specifically associating with the antibiotic is defined by a set of structure coordinates having a root mean square deviation of not more than 2.0 Å from the set of structure coordinates corresponding to nucleotide coordinates 1899, 1924, 1926, 2430, 2472, 2485, 2486, 2532, 2534, 2552, 2562, 2563, and 2581 set forth in Table 22.

According to still further features in preferred embodiments, the antibiotic is ASMS, and a three-dimensional atomic structure of the segment including the nucleotides being capable of specifically associating with the antibiotic is defined by the set of structure coordinates corresponding to nucleotide coordinates 1899-2581 set forth in Table 22.

According to still further features in preferred embodiments, the antibiotic is ASMS, and a three-dimensional atomic structure of the segment including the nucleotides being capable of specifically associating with the antibiotic is defined by a set of structure coordinates having a root mean square deviation of not more than 2.0 Å from the set of structure coordinates corresponding to nucleotide coordinates 1899-2581 set forth in Table 22.

According to still further features in preferred embodiments, the antibiotic is ASMS, and a three-dimensional atomic structure of the segment including the amino acid residues being capable of specifically associating with the antibiotic is defined by the set of structure coordinates corresponding to amino acid residue coordinates 79-81 set forth in Table 22.

According to still further features in preferred embodiments, the antibiotic is ASMS, and a three-dimensional atomic structure of the segment including the amino acid residues being capable of specifically associating with the antibiotic is defined by a set of structure coordinates having a root mean square deviation of not more than 2.0 Å from the set of structure coordinates corresponding to amino acid residue coordinates 79-81 set forth in Table 22.

According to still further features in preferred embodiments, the antibiotic is sparsomycin, and a three-dimensional atomic structure of the nucleotides being capable of specifically associating with the antibiotic is defined by the set of structure coordinates corresponding to nucleotide coordinate 2581set forth in Table 23.

According to still further features in preferred embodiments, the antibiotic is sparsomycin, and a three-dimensional atomic structure of the nucleotides being capable of specifically associating with the antibiotic is defined by a set of structure coordinates having a root mean square deviation of not more than 2.0 Å from the set of structure coordinates corresponding to nucleotide coordinate 2581set forth in Table 23.

According to still further features in preferred embodiments, the antibiotic is troleandomycin, and a three-dimensional atomic structure of the nucleotides being capable of specifically associating with the antibiotic is defined by the set of structure coordinates corresponding to nucleotide coordinates 759, 761, 803, 2041, 2042, 2045, 2484, and 2590 set forth in Table 38.

According to still further features in preferred embodiments, the antibiotic is troleandomycin, and a three-dimensional atomic structure of the nucleotides being capable of specifically associating with the antibiotic is defined by a set of structure coordinates having a root mean square deviation of not more than 2.0 Å from the set of structure coordinates corresponding to nucleotide coordinates 759, 761, 803, 2041, 2042, 2045, 2484, and 2590 set forth in Table 38.

According to still further features in preferred embodiments, the antibiotic is troleandomycin, and a three-dimensional atomic structure of the segment including the nucleotides being capable of specifically associating with the antibiotic is defined by the set of structure coordinates corresponding to nucleotide coordinates 759-2590 set forth in Table 38.

According to still further features in preferred embodiments, the antibiotic is troleandomycin, and a three-dimensional atomic structure of the segment including the nucleotides being capable of specifically associating with the antibiotic is defined by a set of structure coordinates having a root mean square deviation of not more than 2.0 Å from the set of structure coordinates corresponding to nucleotide coordinates 759-2590 set forth in Table 38.

According to still further, features in preferred embodiments, the antibiotic is troleandomycin, and a three-dimensional atomic structure of the segment including the amino acid residues being capable of specifically associating with the antibiotic is defined by the set of structure coordinates corresponding to amino acid residue coordinate Ala2 set forth in Table 38.

According to still further features in preferred embodiments, the antibiotic is troleandomycin, and a three-dimensional atomic structure of the segment including the amino acid residues being capable of specifically associating with the antibiotic is defined by a set of structure coordinates having a root mean square deviation of not more than 2.0 Å from the set of structure coordinates corresponding to amino acid residue coordinate Ala2 set forth in Table 38.

According to yet another aspect of the present invention there is provided a method of identifying a putative antibiotic comprising: (a) obtaining a set of structure coordinates defining a three-dimensional atomic structure of a crystallized antibiotic-binding pocket of a large ribosomal subunit of a eubacterium; and (b) computationally screening a plurality of compounds for a compound capable of specifically binding the antibiotic-binding pocket,

thereby identifying the putative antibiotic.

According to further features in preferred embodiments of the invention described below, the method of identifying a putative antibiotic further comprises: (i) contacting the putative antibiotic with the antibiotic-binding pocket; and (ii) detecting specific binding of the putative antibiotic to the antibiotic-binding pocket, thereby qualifying the putative antibiotic.

According to still further features in preferred embodiments, step (a) is effected by co-crystallizing at least the antibiotic-binding pocket with an antibiotic.

According to still further features in preferred embodiments, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the antibiotic-binding pocket is a clindamycin-binding pocket and the structure coordinates define the three-dimensional atomic structure at a resolution higher than or equal to 3.1 Å.

According to still further features in preferred embodiments, the antibiotic-binding pocket is an erythromycin-binding pocket and the structure coordinates define the three-dimensional atomic structure at a resolution higher than or equal to 3.4 Å.

According to still further features in preferred embodiments, the antibiotic-binding pocket is a clarithromycin-binding pocket and the structure coordinates define the three-dimensional atomic structure at a resolution higher than or equal to 3.5 Å.

According to still further features in preferred embodiments, the antibiotic-binding pocket is a roxithromycin-binding pocket and the structure coordinates define the three-dimensional atomic structure at a resolution higher than or equal to 3.8 Å.

According to still further features in preferred embodiments, the antibiotic-binding pocket is a chloramphenicol-binding pocket and the structure coordinates define the three-dimensional atomic structure at a resolution higher than or equal to 3.5 Å.

According to still further features in preferred embodiments, the antibiotic-binding pocket is an ABT-773-binding pocket and the structure coordinates define the three-dimensional atomic structure at a resolution higher than or equal to 3.5 Å.

According to still further features in preferred embodiments, the antibiotic-binding pocket is an azithromycin-binding pocket and the structure coordinates define the three-dimensional atomic structure at a resolution higher than or equal to 3.2 Å.

According to still further features in preferred embodiments, the antibiotic-binding pocket is an ACCP-binding pocket and the structure coordinates define the three-dimensional atomic structure at a resolution higher than or equal to 3.7 Å.

According to still further features in preferred embodiments, the antibiotic-binding pocket is a puromycin conjugate-binding pocket and the structure coordinates define the three-dimensional atomic structure at a resolution higher than or equal to 3.5 Å.

According to still further features in preferred embodiments, the puromycin conjugate is ASM.

According to still further features in preferred embodiments, the antibiotic-binding pocket is an ASMS-binding pocket and the structure coordinates define the three-dimensional atomic structure at a resolution higher than or equal to 3.6 Å.

According to still further features in preferred embodiments, the antibiotic-binding pocket is a sparsomycin-binding pocket and the structure coordinates define the three-dimensional atomic structure at a resolution higher than or equal to 3.7 Å.

According to still further features in preferred embodiments, the antibiotic-binding pocket is a troleandomycin-binding pocket and the structure coordinates define the three-dimensional atomic structure at a resolution higher than or equal to 3.4 Å.

According to still further features in preferred embodiments, the antibiotic-binding pocket is selected from the group consisting of a chloramphenicol-specific antibiotic-binding pocket, a lincosamide antibiotic-specific antibiotic-binding pocket, a macrolide antibiotic-specific antibiotic-binding pocket, a puromycin conjugate-specific antibiotic-binding pocket, and a sparsomycin-specific antibiotic-binding pocket.

According to still further features in preferred embodiments, the lincosamide-specific binding pocket is a clindamycin-specific antibiotic-binding pocket.

According to still further features in preferred embodiments, the macrolide antibiotic-specific binding pocket is an erythromycin-specific antibiotic-binding pocket, a roxithromycin-specific antibiotic-binding pocket, a troleandomycin-specific antibiotic-binding pocket, a ketolide antibiotic-specific binding pocket, and an azalide antibiotic-specific binding pocket.

According to still further features in preferred embodiments, the ketolide-specific binding pocket is an ABT-773-specific antibiotic-binding pocket.

According to still further features in preferred embodiments, the azalide-specific binding pocket is an azithromycin-specific antibiotic-binding pocket.

According to still further features in preferred embodiments, the puromycin conjugate-specific antibiotic-binding pocket is an ACCP-specific antibiotic-binding pocket or an ASM-specific antibiotic-binding pocket.

According to still further features in preferred embodiments, the antibiotic comprises at least two non-covalently associated molecules.

According to still further features in preferred embodiments, the antibiotic-binding pocket forms a part of a component of the large ribosomal subunit selected from the group consisting of a polynucleotide component, a polypeptide component and a magnesium ion component.

According to still another aspect of the present invention there is provided a computing platform for generating a three-dimensional model of at least a portion of a large ribosomal subunit of a eubacterium, the computing platform comprising: (a) a data-storage device storing data comprising a set of structure coordinates defining at least a portion of a three-dimensional structure of the large ribosomal subunit; and (b) a processing unit being for generating the three-dimensional model from the data stored in the data-storage device.

According to further features in preferred embodiments of the invention described below, the computing platform for generating a three-dimensional model of at least a portion of a large ribosomal subunit of a eubacterium further comprises a display being for displaying the three-dimensional model generated by the processing unit.

According to still further features in preferred embodiments, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the set of structure coordinates define the portion of a three-dimensional structure of a large ribosomal subunit at a resolution higher than or equal to a resolution selected from the group consisting of 5.4 Å, 5.3 Å, 5.2 Å, 5.1 Å, 5.0 Å, 4.9 Å, 4.8 Å, 4.7 Å, 4.6 Å, 4.5 Å, 4.4 Å, 4.3 Å, 4.2 Å, 4.1 Å, 4.0 Å, 3.9 Å, 3.8 Å, 3.7 Å, 3.6 Å, 3.5 Å, 3.4 Å, 3.3 Å, 3.2 Å and 3.1 Å.

According to still further features in preferred embodiments, the set of structure coordinates define the portion of a three-dimensional structure of the large ribosomal subunit at a resolution higher than or equal to 3.1 Å.

According to still further features in preferred embodiments, the set of structure coordinates defining at least a portion of a three-dimensional structure of the large ribosomal subunit is a set of structure coordinates corresponding to a set of coordinates set forth in Table 3, the set of coordinates set forth in Table 3 being selected from the group consisting of: nucleotide coordinates 2044, 2430, 2431, 2479 and 2483-2485; nucleotide coordinates 2044-2485; nucleotide coordinates 2040-2042, 2044, 2482, 2484 and 2590; nucleotide coordinates 2040-2590; nucleotide coordinates 2040-2042, 2045, 2484, 2588 and 2589; nucleotide coordinates 2040-2589; atom coordinates 1-59360; atom coordinates 59361-61880; atom coordinates 1-61880; atom coordinates 61881-62151; atom coordinates 62152-62357; atom coordinates 62358-62555; atom coordinates 62556-62734; atom coordinates 62735-62912; atom coordinates 62913-62965; atom coordinates 62966-63109; atom coordinates 63110-63253; atom coordinates 63254-63386; atom coordinates 63387-63528; atom coordinates 63529-63653; atom coordinates 63654-63768; atom coordinates 63769-63880; atom coordinates 63881-64006; atom coordinates 64007-64122; atom coordinates 64123-64223; atom coordinates 64224-64354; atom coordinates 64355-64448; atom coordinates 64449-64561; atom coordinates 64562-64785; atom coordinates 64786-64872; atom coordinates 64873-64889; atom coordinates 64890-64955; atom coordinates 64956-65011; atom coordinates 65012-65085; atom coordinates 65086-65144; atom coordinates 65145-65198; atom coordinates 65199-65245; atom coordinates 65246-65309; atom coordinates 65310-65345; atom coordinates 61881-65345; and atom coordinates 1-65345.

According to still further features in preferred embodiments, the set of structure coordinates defining at least a portion of a three-dimensional structure of the large ribosomal subunit is a set of structure coordinates having a root mean square deviation of not more than 2.0 Å from a set of structure coordinates corresponding to a set of coordinates set forth in Table 3, the set of coordinates set forth in Table 3 being selected from the group consisting of: nucleotide coordinates 2044, 2430, 2431, 2479 and 2483-2485; nucleotide coordinates 2044-2485; nucleotide coordinates 2040-2042, 2044, 2482, 2484 and 2590; nucleotide coordinates 2040-2590; nucleotide coordinates 2040-2042, 2045, 2484, 2588 and 2589; nucleotide coordinates 2040-2589; atom coordinates 1-59360; atom coordinates 59361-61880; atom coordinates 1-61880; atom coordinates 61881-62151; atom coordinates 62152-62357; atom coordinates 62358-62555; atom coordinates 62556-62734; atom coordinates 62735-62912; atom coordinates 62913-62965; atom coordinates 62966-63109; atom coordinates 63110-63253; atom coordinates 63254-63386; atom coordinates 63387-63528; atom coordinates 63529-63653; atom coordinates 63654-63768; atom coordinates 63769-63880; atom coordinates 63881-64006; atom coordinates 64007-64122; atom coordinates 64123-64223; atom coordinates 64224-64354; atom coordinates 64355-64448; atom coordinates 64449-64561; atom coordinates 64562-64785; atom coordinates 64786-64872; atom coordinates 64873-64889; atom coordinates 64890-64955; atom coordinates 64956-65011; atom coordinates 65012-65085; atom coordinates 65086-65144; atom coordinates 65145-65198; atom coordinates 65199-65245; atom coordinates 65246-65309; atom coordinates 65310-65345; atom coordinates 61881-65345; and atom coordinates 1-65345.

According to a further aspect of the present invention there is provided a computing platform for generating a three-dimensional model of at least a portion of a complex of an antibiotic and a large ribosomal subunit of a eubacterium, the computing platform comprising: (a) a data-storage device storing data comprising a set of structure coordinates defining at least a portion of a three-dimensional structure of the complex of an antibiotic and a large ribosomal subunit; and (b) a processing unit being for generating the three-dimensional model from the data stored in the data-storage device.

According to further features in preferred embodiments of the invention described below, the computing platform for generating a three-dimensional model of at least a portion of a complex of an antibiotic and a large ribosomal subunit of a eubacterium further comprises a display being for displaying the three-dimensional model generated by the processing unit.

According to still further features in preferred embodiments, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the antibiotic is clindamycin and the set of structure coordinates define the portion of a three-dimensional structure at a resolution higher than or equal to 3.1 Å.

According to still further features in preferred embodiments, the antibiotic is erythromycin and the set of structure coordinates define the portion of a three-dimensional structure at a resolution higher than or equal to of 3.4 Å.

According to still further features in preferred embodiments, the antibiotic is clarithromycin and the set of structure coordinates define the portion of a three-dimensional structure at a resolution higher than or equal to 3.5 Å.

According to still further features in preferred embodiments, the antibiotic is roxithromycin and the set of structure coordinates define the portion of a three-dimensional structure at a resolution higher than or equal to 3.8 Å.

According to still further features in preferred embodiments, the antibiotic is chloramphenicol and the set of structure coordinates define the portion of a three-dimensional structure at a resolution higher than or equal to 3.5 Å.

According to still further features in preferred embodiments, the antibiotic is ABT-773 and the set of structure coordinates define the portion of a three-dimensional structure at a resolution higher than or equal to 3.5 Å.

According to still further features in preferred embodiments, the antibiotic is azithromycin and the set of structure coordinates define the portion of a three-dimensional structure at a resolution higher than or equal to 3.2 Å.

According to still further features in preferred embodiments, the antibiotic is ACCP and the set of structure coordinates define the portion of a three-dimensional structure at a resolution higher than or equal to 3.7 Å.

According to still further features in preferred embodiments, the antibiotic is ASM and the set of structure coordinates define the portion of a three-dimensional structure at a resolution higher than or equal to 3.5 Å.

According to still further features in preferred embodiments, the antibiotic is ASMS and the set of structure coordinates define the portion of a three-dimensional structure at a resolution higher than or equal to 3.6 Å.

According to still further features in preferred embodiments, the antibiotic is sparsomycin and the set of structure coordinates define the portion of a three-dimensional structure at a resolution higher than or equal to 3.7 Å.

According to still further features in preferred embodiments, the antibiotic is troleandomycin and the set of structure coordinates define the portion of a three-dimensional structure at a resolution higher than or equal to 3.4 Å.

According to still further features in preferred embodiments, the antibiotic is selected from the group consisting of chloramphenicol, a lincosamide antibiotic, a macrolide antibiotic, a puromycin conjugate, ASMS, and sparsomycin.

According to still further features in preferred embodiments, the lincosamide antibiotic is clindamycin.

According to still further features in preferred embodiments, the macrolide antibiotic is selected from the group consisting of erythromycin, clarithromycin, roxithromycin, troleandomycin, a ketolide antibiotic and an azalide antibiotic.

According to still further features in preferred embodiments, the ketolide antibiotic is ABT-773.

According to still further features in preferred embodiments, the azalide antibiotic is azithromycin.

According to still further features in preferred embodiments, the puromycin conjugate is ACCP or ASM.

According to still further features in preferred embodiments, the antibiotic is chloramphenicol and the set of structure coordinates defining at least a portion of a three-dimensional structure of the complex of the chloramphenicol and the large ribosomal subunit corresponds to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

According to still further features in preferred embodiments, the antibiotic is clindamycin and the set of structure coordinates defining at least a portion of a three-dimensional structure of the complex of the clindamycin and the large ribosomal subunit corresponds to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

According to still further features in preferred embodiments, the antibiotic is clarithromycin and the set of structure coordinates defining at least a portion of a three-dimensional structure of the complex of the clarithromycin and the large ribosomal subunit corresponds to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

According to still further features in preferred embodiments, the antibiotic is erythromycin and the set of structure coordinates defining at least a portion of a three-dimensional structure of the complex of the erythromycin and the large ribosomal subunit corresponds to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

According to still further features in preferred embodiments, the antibiotic is roxithromycin and the set of structure coordinates defining at least a portion of a three-dimensional structure of the complex of the roxithromycin and the large ribosomal subunit corresponds to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

According to still further features in preferred embodiments, the antibiotic is ABT-773 and the set of structure coordinates defining at least a portion of a three-dimensional structure of the complex of the ABT-773 and the large ribosomal subunit corresponds to a set of coordinates selected from the group consisting of nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

According to still further features in preferred embodiments, the antibiotic is azithromycin and the set of structure coordinates defining at least a portion of a three-dimensional structure of the complex of the azithromycin and the large ribosomal subunit corresponds to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

According to still further features in preferred embodiments, the antibiotic is ACCP and the set of structure coordinates defining at least a portion of a three-dimensional structure of the complex of the ACCP and the large ribosomal subunit corresponds to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

According to still further features in preferred embodiments, the antibiotic is ASM and the set of structure coordinates defining at least a portion of a three-dimensional structure of the complex of the ASM and the large ribosomal subunit corresponds to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

According to still further features in preferred embodiments, the antibiotic is ASMS and the set of structure coordinates defining at least a portion of a three-dimensional structure of the complex of the ASMS and the large ribosomal subunit corresponds to a set of coordinates selected from the group consisting of nucleotide coordinates 1899, 1924, 1926, 2430, 2472, 2485, 2486, 2532, 2534, 2552, 2562, 2563, and 2581 set forth in Table 22; nucleotide coordinates 1899-2581set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

According to still further features in preferred embodiments, the antibiotic is sparsomycin and the set of structure coordinates defining at least a portion of a three-dimensional structure of the complex of the sparsomycin and the large ribosomal subunit corresponds to a set of coordinates selected from the group consisting of nucleotide coordinate 2581 set forth in Table 23; atom coordinates 78757-78778 set forth in Table 23; the set of atom coordinates set forth in Table 23; and the set of atom coordinates set forth in Table 28.

According to still further features in preferred embodiments, the antibiotic is troleandomycin and the set of structure coordinates defining at least a portion of a three-dimensional structure of the complex of the troleandomycin and the large ribosomal subunit corresponds to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

According to still further features in preferred embodiments, the antibiotic is chloramphenicol and the set of structure coordinates defining at least a portion of a three-dimensional structure of the complex of the chloramphenicol and the large ribosomal subunit is a set of structure coordinates having a root mean square deviation of not more than 2.0 Å from a set of structure coordinates corresponding to a set of coordinates selected from the group consisting of nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

According to still further features in preferred embodiments, the antibiotic is clindamycin and the set of structure coordinates defining at least a portion of a three-dimensional structure of the complex of the clindamycin and the large ribosomal subunit is a set of structure coordinates having a root mean square deviation of not more than 2.0 Å from a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

According to still further features in preferred embodiments, the antibiotic is clarithromycin and the set of structure coordinates defining at least a portion of a three-dimensional structure of the complex of the clarithromycin and the large ribosomal subunit is a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

According to still further features in preferred embodiments, the antibiotic is erythromycin and the set of structure coordinates defining at least a portion of a three-dimensional structure of the complex of the erythromycin and the large ribosomal subunit is a set of structure coordinates having a root mean square deviation of not more than 2.0 Å from a set of structure coordinates corresponding to a set of coordinates selected from the group consisting of nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

According to still further features in preferred embodiments, the antibiotic is roxithromycin and the set of structure coordinates defining at least a portion of a three-dimensional structure of the complex of the roxithromycin and the large ribosomal subunit is a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

According to still further features in preferred embodiments, the antibiotic is ABT-773 and the set of structure coordinates defining at least a portion of a three-dimensional structure of the complex of the ABT-773 and the large ribosomal subunit is a set of structure coordinates having a root mean square deviation of not more than 2.0 Å from a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

According to still further features in preferred embodiments, the antibiotic is azithromycin and the set of structure coordinates defining at least a portion of a three-dimensional structure of the complex of the azithromycin and the large ribosomal subunit is a set of structure coordinates having a root mean square deviation of not more than 2.0 Å from a set of structure coordinates corresponding to a set of coordinates selected from the group consisting of nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

According to still further features in preferred embodiments, the antibiotic is ACCP and the set of structure coordinates defining at least a portion of a three-dimensional structure of the complex of the ACCP and the large ribosomal subunit is a set of structure coordinates having a root mean square deviation of not more than 2.0 Å from a set of structure coordinates corresponding to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

According to still further features in preferred embodiments, the antibiotic is ASM and the set of structure coordinates defining at least a portion of a three-dimensional structure of the complex of the ASM and the large ribosomal subunit is a set of structure coordinates having a root mean square deviation of not more than 2.0 Å from a set of structure coordinates corresponding to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

According to still further features in preferred embodiments, the antibiotic is ASMS and the set of structure coordinates defining at least a portion of a three-dimensional structure of the complex of the ASMS and the large ribosomal subunit is a set of structure coordinates having a root mean square deviation of not more than 2.0 Å from a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

According to still further features in preferred embodiments, the antibiotic is sparsomycin and the set of structure coordinates defining at least a portion of a three-dimensional structure of the complex of the sparsomycin and the large ribosomal subunit is a set of structure coordinates having a root mean square deviation of not more than 2.0 Å from a set of structure coordinates corresponding to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

According to still further features in preferred embodiments, the antibiotic is troleandomycin and the set of structure coordinates defining at least a portion of a three-dimensional structure of the complex of the troleandomycin and the large ribosomal subunit is a set of structure coordinates having a root mean square deviation of not more than 2.0 Å from a set of structure coordinates corresponding to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

According to a yet a further aspect of the present invention there is provided a computer generated model representing at least a portion of a large ribosomal subunit of a eubacterium, the computer generated model having a three-dimensional atomic structure defined by a set of structure coordinates corresponding to a set of coordinates set forth in Table 3, the set of coordinates set forth in Table 3 being selected from the group consisting of: nucleotide coordinates 2044, 2430, 2431, 2479 and 2483-2485; nucleotide coordinates 2044-2485; nucleotide coordinates 2040-2042, 2044, 2482, 2484 and 2590; nucleotide coordinates 2040-2590; nucleotide coordinates 2040-2042, 2045, 2484, 2588 and 2589; nucleotide coordinates 2040-2589; atom coordinates 1-59360; atom coordinates 59361-61880; atom coordinates 1-61880; atom coordinates 61881-62151; atom coordinates 62152-62357; atom coordinates 62358-62555; atom coordinates 62556-62734; atom coordinates 62735-62912; atom coordinates 62913-62965; atom coordinates 62966-63109; atom coordinates 63110-63253; atom coordinates 63254-63386; atom coordinates 63387-63528; atom coordinates 63529-63653; atom coordinates 63654-63768; atom coordinates 63769-63880; atom coordinates 63881-64006; atom coordinates 64007-64122; atom coordinates 64123-64223; atom coordinates 64224-64354; atom coordinates 64355-64448; atom coordinates 64449-64561; atom coordinates 64562-64785; atom coordinates 64786-64872; atom coordinates 64873-64889; atom coordinates 64890-64955; atom coordinates 64956-65011; atom coordinates 65012-65085; atom coordinates 65086-65144; atom coordinates 65145-65198; atom coordinates 65199-65245; atom coordinates 65246-65309; atom coordinates 65310-65345; atom coordinates 61881-65345; and atom coordinates 1-65345.

According to further features in preferred embodiments of the invention described below, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still a further aspect of the present invention there is provided a computer generated model representing at least a portion of a large ribosomal subunit of a eubacterium, the computer generated model having a three-dimensional atomic structure defined by a set of structure coordinates having a root mean square deviation of not more than 2.0 Å from a set of structure coordinates corresponding to a set of coordinates set forth in Table 3, the set of coordinates set forth in Table 3 being selected from the group consisting of: nucleotide coordinates 2044, 2430, 2431, 2479 and 2483-2485; nucleotide coordinates 2044-2485; nucleotide coordinates 2040-2042, 2044, 2482, 2484 and 2590; nucleotide coordinates 2040-2590; nucleotide coordinates 2040-2042, 2045, 2484, 2588 and 2589; nucleotide coordinates 2040-2589; atom coordinates 1-59360; atom coordinates 59361-61880; atom coordinates 1-61880; atom coordinates 61881-62151; atom coordinates 62152-62357; atom coordinates 62358-62555; atom coordinates 62556-62734; atom coordinates 62735-62912; atom coordinates 62913-62965; atom coordinates 62966-63109; atom coordinates 63110-63253; atom coordinates 63254-63386; atom coordinates 63387-63528; atom coordinates 63529-63653; atom coordinates 63654-63768; atom coordinates 63769-63880; atom coordinates 63881-64006; atom coordinates 64007-64122; atom coordinates 64123-64223; atom coordinates 64224-64354; atom coordinates 64355-64448; atom coordinates 64449-64561; atom coordinates 64562-64785; atom coordinates 64786-64872; atom coordinates 64873-64889; atom coordinates 64890-64955; atom coordinates 64956-65011; atom coordinates 65012-65085; atom coordinates 65086-65144; atom coordinates 65145-65198; atom coordinates 65199-65245; atom coordinates 65246-65309; atom coordinates 65310-65345; atom coordinates 61881-65345; and atom coordinates 1-65345.

According to further features in preferred embodiments of the invention described below, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to an additional aspect of the present invention there is provided a computer generated model representing at least a portion of a complex of an antibiotic and a large ribosomal subunit of a eubacterium.

According to further features in preferred embodiments of the invention described below, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the antibiotic is selected from the group consisting of chloramphenicol, a lincosamide antibiotic, a macrolide antibiotic, a puromycin conjugate, ASMS, and sparsomycin.

According to still further features in preferred embodiments, the lincosamide antibiotic is clindamycin.

According to still further features in preferred embodiments, the macrolide antibiotic is selected from the group consisting of erythromycin, clarithromycin, roxithromycin, troleandomycin, a ketolide antibiotic and an azalide antibiotic.

According to still further features in preferred embodiments, the ketolide antibiotic is ABT-773.

According to still further features in preferred embodiments, the azalide antibiotic is azithromycin.

According to still further features in preferred embodiments, the puromycin conjugate is ACCP or ASM.

According to still further features in preferred embodiments, the antibiotic is clindamycin and the set of structure coordinates define the three-dimensional structure of the computer generated model at a resolution higher than or equal to 3.1 Å.

According to still further features in preferred embodiments, the antibiotic is erythromycin and the set of structure coordinates define the three-dimensional structure of the computer generated model at a resolution higher than or equal to 3.4 Å.

According to still further features in preferred embodiments, the antibiotic is clarithromycin and the set of structure coordinates define the three-dimensional structure of the computer generated model at a resolution higher than or equal to 3.5 Å.

According to still further features in preferred embodiments, the antibiotic is roxithromycin and the set of structure coordinates define the three-dimensional structure of the computer generated model at a resolution higher than or equal to 3.8 Å.

According to still further features in preferred embodiments, the antibiotic is chloramphenicol and the set of structure coordinates define the three-dimensional structure of the computer generated model at a resolution higher than or equal to 3.5 Å.

According to still further features in preferred embodiments, the antibiotic is chloramphenicol and a three-dimensional atomic structure of the portion of a complex of the chloramphenicol and the large ribosomal subunit is defined by a set of structure coordinates corresponding to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

According to still further features in preferred embodiments, the antibiotic is clindamycin and a three-dimensional atomic structure of the portion of a complex of the clindamycin and the large ribosomal subunit is defined by a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

According to still further features in preferred embodiments, the antibiotic is clarithromycin and a three-dimensional atomic structure of the portion of a complex of the clarithromycin and the large ribosomal subunit is defined by a set of structure coordinates corresponding to a set of coordinates selected from the group consisting of nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

According to still further features in preferred embodiments, the antibiotic is erythromycin and a three-dimensional atomic structure of the portion of a complex of the erythromycin and the large ribosomal subunit is defined by a set of structure coordinates corresponding to a set of coordinates selected from the group consisting of nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

According to still further features in preferred embodiments, the antibiotic is roxithromycin and a three-dimensional atomic structure of the portion of a complex of the roxithromycin and the large ribosomal subunit is defined by a set of structure coordinates corresponding to a set of coordinates selected from the group consisting of nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

According to still further features in preferred embodiments, the antibiotic is ABT-773 and a three-dimensional atomic structure of the portion of a complex of the ABT-773 and the large ribosomal subunit is defined by a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

According to still further features in preferred embodiments, the antibiotic is azithromycin and a three-dimensional atomic structure of the portion of a complex of the azithromycin and the large ribosomal subunit is defined by a set of structure coordinates corresponding to a set of coordinates selected from the group consisting of nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

According to still further features in preferred embodiments, the antibiotic is ACCP and a three-dimensional atomic structure of the portion of a complex of the ACCP and the large ribosomal subunit is defined by a set of structure coordinates corresponding to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

According to still further features in preferred embodiments, the antibiotic is ASM and a three-dimensional atomic structure of the portion of a complex of the ASM and the large ribosomal subunit is defined by a set of structure coordinates corresponding to a set of coordinates selected from the group consisting of nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

According to still further features in preferred embodiments, the antibiotic is ASMS and a three-dimensional atomic structure of the portion of a complex of the ASMS and the large ribosomal subunit is defined by a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

According to still further features in preferred embodiments, the antibiotic is sparsomycin and a three-dimensional atomic structure of the portion of a complex of the sparsomycin and the large ribosomal subunit is defined by a set of structure coordinates corresponding to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

According to still further features in preferred embodiments, the antibiotic is troleandomycin and a three-dimensional atomic structure of the portion of a complex of the troleandomycin and the large ribosomal subunit is defined by a set of structure coordinates corresponding to a set of coordinates selected from the group consisting of nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

According to still further features in preferred embodiments, the antibiotic is chloramphenicol and a three-dimensional atomic structure of the portion of a complex of the chloramphenicol and the large ribosomal subunit is defined by a set of structure coordinates having a root mean square deviation of not more than 2.0 Å from a set of structure coordinates corresponding to a set of coordinates selected from the group consisting of nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

According to still further features in preferred embodiments, the antibiotic is clindamycin and a three-dimensional atomic structure of the portion of a complex of the clindamycin and the large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

According to still further features in preferred embodiments, the antibiotic is clarithromycin and a three-dimensional atomic structure of the portion of a complex of the clarithromycin and the large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

According to still further features in preferred embodiments, the antibiotic is erythromycin and a three-dimensional atomic structure of the portion of a complex of the erythromycin and the large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

According to still further features in preferred embodiments, the antibiotic is roxithromycin and a three-dimensional atomic structure of the portion of a complex of the roxithromycin and the large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

According to still further features in preferred embodiments, the antibiotic is ABT-773 and a three-dimensional atomic structure of the portion of a complex of the ABT-773 and the large ribosomal subunit is defined by a set of structure coordinates having a root mean square deviation of not more than 2.0 Å from a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

According to still further features in preferred embodiments, the antibiotic is azithromycin and a three-dimensional atomic structure of the portion of a complex of the azithromycin and the large ribosomal subunit is defined by a set of structure coordinates having a root mean square deviation of not more than 2.0 Å from a set of structure coordinates corresponding to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

According to still further features in preferred embodiments, the antibiotic is ACCP and a three-dimensional atomic structure of the portion of a complex of the ACCP and the large ribosomal subunit is defined by a set of structure coordinates having a root mean square deviation of not more than 2.0 Å from a set of structure coordinates corresponding to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

According to still further features in preferred embodiments, the antibiotic is ASM and a three-dimensional atomic structure of the portion of a complex of the ASM and the large ribosomal subunit is defined by a set of structure coordinates having a root mean square deviation of not more than 2.0 Å from a set of structure coordinates corresponding to a set of coordinates selected from the group consisting of nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

According to still further features in preferred embodiments, the antibiotic is ASMS and a three-dimensional atomic structure of the portion of a complex of the ASMS and the large ribosomal subunit is defined by a set of structure coordinates having a root mean square deviation of not more than 2.0 Å from a set of structure coordinates corresponding to a set of coordinates selected from the group consisting of nucleotide coordinates 1899, 1924, 1926, 2430, 2472, 2485, 2486, 2532, 2534, 2552, 2562, 2563, and 2581 set forth in Table 22; nucleotide coordinates 1899-2581set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

According to still further features in preferred embodiments, the antibiotic is sparsomycin and a three-dimensional atomic structure of the portion of a complex of the sparsomycin and the large ribosomal subunit is defined by a set of structure coordinates having a root mean square deviation of not more than 2.0 Å from a set of structure coordinates corresponding to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

According to still further features in preferred embodiments, the antibiotic is troleandomycin and a three-dimensional atomic structure of the portion of a complex of the troleandomycin and the large ribosomal subunit is defined by a set of structure coordinates having a root mean square deviation of not more than 2.0 Å from a set of structure coordinates corresponding to a set of coordinates selected from the group consisting of nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

According to yet an additional aspect of the present invention there is provided a computer readable medium comprising, in a retrievable format, data including a set of structure coordinates defining at least a portion of a three-dimensional structure of a crystallized large ribosomal subunit of a eubacterium.

According to further features in preferred embodiments of the invention described below, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the set of structure coordinates define the portion of a three-dimensional structure of a crystallized large ribosomal subunit at a resolution higher than or equal to a resolution selected from the group consisting of 5.4 Å, 5.3 Å, 5.2 Å, 5.1 Å, 5.0 Å, 4.9 Å, 4.8 Å, 4.7 Å, 4.6 Å, 4.5 Å, 4.4 Å, 4.3 Å, 4.2 Å, 4.1 Å, 4.0 Å, 3.9 Å, 3.8 Å, 3.7 Å, 3.6 Å, 3.5 Å, 3.4 Å, 3.3 Å, 3.2 Å and 3.1 Å.

According to still further features in preferred embodiments, the set of structure coordinates define the portion of a three-dimensional structure of a crystallized large ribosomal subunit at a resolution higher than or equal to 3.1 Å.

According to still further features in preferred embodiments, the structure coordinates defining at least a portion of a three-dimensional structure of a crystallized large ribosomal subunit correspond to a set of coordinates set forth in Table 3, the set of coordinates set forth in Table 3 being selected from the group consisting of nucleotide coordinates 2044, 2430, 2431, 2479 and 2483-2485; nucleotide coordinates 2044-2485; nucleotide coordinates 2040-2042, 2044, 2482, 2484 and 2590; nucleotide coordinates 2040-2590; nucleotide coordinates 2040-2042, 2045, 2484, 2588 and 2589; nucleotide coordinates 2040-2589; atom coordinates 1-59360; atom coordinates 59361-61880; atom coordinates 1-61880; atom coordinates 61881-62151; atom coordinates 62152-62357; atom coordinates 62358-62555; atom coordinates 62556-62734; atom coordinates 62735-62912; atom coordinates 62913-62965; atom coordinates 62966-63109; atom coordinates 63110-63253; atom coordinates 63254-63386; atom coordinates 63387-63528; atom coordinates 63529-63653; atom coordinates 63654-63768; atom coordinates 63769-63880; atom coordinates 63881-64006; atom coordinates 64007-64122; atom coordinates 64123-64223; atom coordinates 64224-64354; atom coordinates 64355-64448; atom coordinates 64449-64561; atom coordinates 64562-64785; atom coordinates 64786-64872; atom coordinates 64873-64889; atom coordinates 64890-64955; atom coordinates 64956-65011; atom coordinates 65012-65085; atom coordinates 65086-65144; atom coordinates 65145-65198; atom coordinates 65199-65245; atom coordinates 65246-65309; atom coordinates 65310-65345; atom coordinates 61881-65345; and atom coordinates 1-65345.

According to still further features in preferred embodiments, the structure coordinates defining at least a portion of a three-dimensional structure of a crystallized large ribosomal subunit have a root mean square deviation of not more than 2.0 Å from a set of structure coordinates corresponding to a set of coordinates set forth in Table 3, the set of coordinates set forth in Table 3 being selected from the group consisting of: nucleotide coordinates 2044, 2430, 2431, 2479 and 2483-2485; nucleotide coordinates 2044-2485; nucleotide coordinates 2040-2042, 2044, 2482, 2484 and 2590; nucleotide coordinates 2040-2590; nucleotide coordinates 2040-2042, 2045, 2484, 2588 and 2589; nucleotide coordinates 2040-2589; atom coordinates 1-59360; atom coordinates 59361-61880; atom coordinates 1-61880; atom coordinates 61881-62151; atom coordinates 62152-62357; atom coordinates 62358-62555; atom coordinates 62556-62734; atom coordinates 62735-62912; atom coordinates 62913-62965; atom coordinates 62966-63109; atom coordinates 63110-63253; atom coordinates 63254-63386; atom coordinates 63387-63528; atom coordinates 63529-63653; atom coordinates 63654-63768; atom coordinates 63769-63880; atom coordinates 63881-64006; atom coordinates 64007-64122; atom coordinates 64123-64223; atom coordinates 64224-64354; atom coordinates 64355-64448; atom coordinates 64449-64561; atom coordinates 64562-64785; atom coordinates 64786-64872; atom coordinates 64873-64889; atom coordinates 64890-64955; atom coordinates 64956-65011; atom coordinates 65012-65085; atom coordinates 65086-65144; atom coordinates 65145-65198; atom coordinates 65199-65245; atom coordinates 65246-65309; atom coordinates 65310-65345; atom coordinates 61881-65345; and atom coordinates 1-65345.

According to still an additional aspect of the present invention there is provided a computer readable medium comprising, in a retrievable format, data including a set of structure coordinates defining at least a portion of a three-dimensional structure of a crystallized complex of an antibiotic and a large ribosomal subunit of a eubacterium.

According to further features in preferred embodiments of the invention described below, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the antibiotic is selected from the group consisting of chloramphenicol, a lincosamide antibiotic, a macrolide antibiotic, a puromycin conjugate, ASMS, and sparsomycin.

According to still further features in preferred embodiments, the lincosamide antibiotic is clindamycin.

According to still further features in preferred embodiments, the macrolide antibiotic is selected from the group consisting of erythromycin, clarithromycin, roxithromycin, troleandomycin, a ketolide antibiotic and an azalide antibiotic.

According to still further features in preferred embodiments, the ketolide antibiotic is ABT-773.

According to still further features in preferred embodiments, the azalide antibiotic is azithromycin.

According to still further features in preferred embodiments, the puromycin conjugate is ACCP or ASM.

According to still further features in preferred embodiments, the antibiotic is clindamycin and the set of structure coordinates define the portion of a three-dimensional structure of a crystallized complex of an antibiotic and a large ribosomal subunit at a resolution higher than or equal to 3.1 Å.

According to still further features in preferred embodiments, the antibiotic is erythroinycin and the set of structure coordinates define the portion of a three-dimensional structure of a crystallized complex of an antibiotic and a large ribosomal subunit at a resolution higher than or equal to 3.4 Å.

According to still further features in preferred embodiments, the antibiotic is clarithromycin and the set of structure coordinates define the portion of a three-dimensional structure of a crystallized complex of an antibiotic and a large ribosomal subunit at a resolution higher than or equal to 3.5 Å.

According to still further features in preferred embodiments, the antibiotic is roxithromycin and the set of structure coordinates define the portion of a three-dimensional structure of a crystallized complex of an antibiotic and a large ribosomal subunit at a resolution higher than or equal to 3.8 Å.

According to still further features in preferred embodiments, the antibiotic is chloramphenicol and the set of structure coordinates define the portion of a three-dimensional structure of a crystallized complex of an antibiotic and a large ribosomal subunit at a resolution higher than or equal to 3.5 Å.

According to still further features in preferred embodiments, the antibiotic is ABT-773 and the set of structure coordinates define the portion of a three-dimensional structure of a crystallized complex of an antibiotic and a large ribosomal subunit at a resolution higher than or equal to 3.5 Å.

According to still further features in preferred embodiments, the antibiotic is azithromycin and the set of structure coordinates define the portion of a three-dimensional structure of a crystallized complex of an antibiotic and a large ribosomal subunit at a resolution higher than or equal to 3.2 Å.

According to still further features in preferred embodiments, the antibiotic is ACCP and the set of structure coordinates define the portion of a three-dimensional structure of a crystallized complex of an antibiotic and a large ribosomal subunit at a resolution higher than or equal to 3.7 Å.

According to still further features in preferred embodiments, the antibiotic is ASM and the set of structure coordinates define the portion of a three-dimensional structure of a crystallized complex of an antibiotic and a large ribosomal subunit at a resolution higher than or equal to 3.5 Å.

According to still further features in preferred embodiments, the antibiotic is ASMS and the set of structure coordinates define the portion of a three-dimensional structure of a crystallized complex of an antibiotic and a large ribosomal subunit at a resolution higher than or equal to 3.6 Å.

According to still further features in preferred embodiments, the antibiotic is sparsomycin and the set of structure coordinates define the portion of a three-dimensional structure of a crystallized complex of an antibiotic and a large ribosomal subunit at a resolution higher than or equal to 3.7 Å.

According to still further features in preferred embodiments, the antibiotic is troleandomycin and the set of structure coordinates define the portion of a three-dimensional structure of a crystallized complex of an antibiotic and a large ribosomal subunit at a resolution higher than or equal to 3.4 Å.

According to still further features in preferred embodiments, the antibiotic is chloramphenicol and the three-dimensional structure of a crystallized complex of an antibiotic and a large ribosomal subunit is defined by a set of structure coordinates corresponding to a set of coordinates selected from the group consisting of nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

According to still further features in preferred embodiments, the antibiotic is clindamycin and the three-dimensional structure of a crystallized complex of an antibiotic and a large ribosomal subunit is defined by a set of structure coordinates corresponding to a set of coordinates selected from the group consisting of nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

According to still further features in preferred embodiments, the antibiotic is clarithromycin and the three-dimensional structure of a crystallized complex of an antibiotic and a large ribosomal subunit is defined by a set of structure coordinates corresponding to a set of coordinates selected from the group consisting of nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

According to still further features in preferred embodiments, the antibiotic is erythromycin and the three-dimensional structure of a crystallized complex of an antibiotic and a large ribosomal subunit is defined by a set of structure coordinates corresponding to a set of coordinates selected from the group consisting of nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

According to still further features in preferred embodiments, the antibiotic is roxithromycin and the three-dimensional structure of a crystallized complex of an antibiotic and a large ribosomal subunit is defined by a set of structure coordinates corresponding to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

According to still further features in preferred embodiments, the antibiotic is ABT-773 and the three-dimensional structure of a crystallized complex of an antibiotic and a large ribosomal subunit is defined by a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

According to still further features in preferred embodiments, the antibiotic is azithromycin and the three-dimensional structure of a crystallized complex of an antibiotic and a large ribosomal subunit is defined by a set of structure coordinates corresponding to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

According to still further features in preferred embodiments, the antibiotic is ACCP and the three-dimensional structure of a crystallized complex of an antibiotic and a large ribosomal subunit is defined by a set of structure coordinates corresponding to a set of coordinates selected from the group consisting of nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

According to still further features in preferred embodiments, the antibiotic is ASM and the three-dimensional structure of a crystallized complex of an antibiotic and a large ribosomal subunit is defined by a set of structure coordinates corresponding to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

According to still further features in preferred embodiments, the antibiotic is ASMS and the three-dimensional structure of a crystallized complex of an antibiotic and a large ribosomal subunit is defined by a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581 set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

According to still further features in preferred embodiments, the antibiotic is sparsomycin and the three-dimensional structure of a crystallized complex of an antibiotic and a large ribosomal subunit is defined by a set of structure coordinates corresponding to a set of coordinates selected from the group consisting of nucleotide coordinate 2581 set forth in Table 23; atom coordinates 78757-78778 set forth in Table 23; the set of atom coordinates set forth in Table 23; and the set of atom coordinates set forth in Table 28.

According to still further features in preferred embodiments, the antibiotic is troleandomycin and the three-dimensional structure of a crystallized complex of an antibiotic and a large ribosomal subunit is defined by a set of structure coordinates corresponding to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

According to still further features in preferred embodiments, the antibiotic is chloramphenicol and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

According to still further features in preferred embodiments, the antibiotic is clindamycin and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

According to still further features in preferred embodiments, the antibiotic is clarithromycin and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

According to still further features in preferred embodiments, the antibiotic is erythromycin and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

According to still further features in preferred embodiments, the antibiotic is roxithromycin and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

According to still further features in preferred embodiments, the antibiotic is ABT-773 and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

According to still further features in preferred embodiments, the antibiotic is azithromycin and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

According to still further features in preferred embodiments, the antibiotic is ACCP and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

According to still further features in preferred embodiments, the antibiotic is ASM and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group- consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

According to still further features in preferred embodiments, the antibiotic is ASMS and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581 set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

According to still further features in preferred embodiments, the antibiotic is sparsomycin and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of nucleotide coordinate 2581 set forth in Table 23; atom coordinates 78757-78778 set forth in Table 23; the set of atom coordinates set forth in Table 23; and the set of atom coordinates set forth in Table 28.

According to still further features in preferred embodiments, the antibiotic is troleandomycin and the three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

According to yet still an additional aspect of the present invention there is provided a method of crystallizing a large ribosomal subunit of a eubacterium comprising: (a) suspending a purified preparation of the large ribosomal subunit in a crystallization solution, the crystallization solution comprising a buffer component and a volatile component, the volatile component being at a first concentration in the crystallization solution, thereby forming a crystallization mixture; and (b) equilibrating the crystallization mixture with an equilibration solution, the equilibration solution comprising the buffer component and the volatile component, the volatile component being at a second concentration in the equilibration solution, the second concentration being a fraction of the first concentration, thereby crystallizing the large ribosomal subunit.

According to further features in preferred embodiments of the invention described below, the eubacterium is D. radiodurans.

According to still further features in preferred embodiments, the eubacterium is a gram-positive bacterium.

According to still further features in preferred embodiments, the eubacterium is a coccus.

According to still further features in preferred embodiments, the eubacterium is a Deinococcus-Thermophilus group bacterium.

According to still further features in preferred embodiments, the volatile component is an alcohol component.

According to still further features in preferred embodiments, the volatile component comprises at least one monovalent alcohol and at least one polyvalent alcohol.

According to still further features in preferred embodiments, the volumetric ratio of the at least one multivalent alcohol to the at least one monovalent alcohol is selected from the range consisting of 1:3.0-1:4.1.

According to still further features in preferred embodiments, the volumetric ratio of the at least one multivalent alcohol to the at least one monovalent alcohol is 1:3.5.

According to still further features in preferred embodiments, the at least one monovalent alcohol is ethanol.

According to still further features in preferred embodiments, the at least one polyvalent alcohol is dimethylhexandiol.

According to still further features in preferred embodiments, the first concentration is selected from a range consisting of 0.1-10% (v/v).

According to still further features in preferred embodiments, the fraction is selected from a range consisting of 0.33-0.67.

According to still further features in preferred embodiments, the fraction is 0.5.

According to still further features in preferred embodiments, the buffer component is an optimal buffer for the functional activity of the large ribosomal subunit.

According to still further features in preferred embodiments, the buffer component is an aqueous solution comprising: MgCl₂ in such a quantity as to yield a final concentration of the MgCl₂ in the crystallization solution, the equilibration solution, or both selected from a range consisting of 3-12 mM; NH₄Cl in such a quantity as to yield a final concentration of the NH₄Cl in the crystallization solution, the equilibration solution, or both selected from a range consisting of 20-70 mM; KCl in such a quantity as to yield a final concentration of the KCl in the crystallization solution, the equilibration solution, or both selected from a range consisting of 0-15 mM; and HEPES in such a quantity as to yield a final concentration of the HEPES in the crystallization solution, the equilibration solution, or both selected from a range consisting of 8-20 mM.

According to still further features in preferred embodiments, the crystallization solution, the equilibration solution, or both have a pH selected from the range consisting of 6.0-9.0 pH units.

According to still further features in preferred embodiments, the equilibrating is effected by vapor diffusion.

According to still further features in preferred embodiments, the equilibrating is effected at a temperature selected from a range consisting of 15-25 degrees centigrade.

According to still further features in preferred embodiments, the equilibrating is effected at a temperature selected from a range consisting of 17-20 degrees centigrade.

According to still further features in preferred embodiments, the equilibrating is effected using a hanging drop of the crystallization mixture.

According to still further features in preferred embodiments, the equilibrating is effected using Linbro dishes.

According to still further features in preferred embodiments, the crystallization solution, the equilibration solution, or both comprise 10 mM MgCl₂, 60 mM NH₄Cl, 5 mM KCl and 10 mM HEPES.

According to still further features in preferred embodiments, the crystallization solution, the equilibration solution, or both have a pH of 7.8.

According to still further features in preferred embodiments, the crystallization solution comprises an antibiotic.

According to still further features in preferred embodiments, the antibiotic is selected from the group consisting of chloramphenicol, a lincosamide antibiotic, a macrolide antibiotic, a puromycin conjugate, ASMS, and sparsomycin.

According to still further features in preferred embodiments, the lincosamide antibiotic is clindamycin.

According to still further features in preferred embodiments, the macrolide antibiotic is selected from the group consisting of erythromycin, clarithromycin, roxithromycin, troleandomycin, a ketolide antibiotic and an azalide antibiotic.

According to still further features in preferred embodiments, the ketolide antibiotic is ABT-773.

According to still further features in preferred embodiments, the azalide antibiotic is azithromycin.

According to still further features in preferred embodiments, the puromycin conjugate is ACCP or ASM.

According to still further features in preferred embodiments, the antibiotic is selected from the group consisting of chloramphenicol, clindamycin, roxithromycin, and erythromycin, and the crystallization solution comprises the antibiotic at a concentration selected from the range consisting of 0.8-3.5 mM.

According to still further features in preferred embodiments, the antibiotic is ABT-773 or azithromycin, and wherein the concentration of the antibiotic in the crystallization solution is about 5.5 to 8.5 times higher than the concentration of the large ribosomal subunit in the crystallization solution.

According to still further features in preferred embodiments, the antibiotic is sparsomycin, and wherein the concentration of the antibiotic in the crystallization solution is about 8 to 12 times higher than the concentration of the large ribosomal subunit in the crystallization solution.

According to still further features in preferred embodiments, the method of crystallizing a large ribosomal subunit of a eubacterium further comprises soaking the crystallized ribosomal subunit in a soaking solution containing an antibiotic.

According to still further features in preferred embodiments, the antibiotic is clarithromycin, and the soaking solution comprises the antibiotic at a concentration selected from the range consisting of 0.004-0.025 mM.

According to still further features in preferred embodiments, the soaking solution comprises the antibiotic at a concentration of 0.01 mM.

According to still further features in preferred embodiments, the antibiotic is ACCP, and the soaking solution comprises the antibiotic at a concentration selected from the range consisting of 0.0150-0.0100 mM.

According to still further features in preferred embodiments, the antibiotic is ASM, and the soaking solution comprises the antibiotic at a concentration selected from the range consisting of 0.020-0.030 mM.

According to still further features in preferred embodiments, the antibiotic is troleandomycin, and the soaking solution comprises the antibiotic at a concentration selected from the range consisting of 0.080-0.120 mM.

According to still further features in preferred embodiments, the antibiotic is ASMS, further wherein the crystallized ribosomal subunit is co-crystallized with sparsomycin, and the soaking solution comprises ASM at a concentration selected from the range consisting of 0.020-0.030 mM.

The present invention successfully addresses the shortcomings of the presently known configurations by providing compositions-of-matter comprising crystallized complexes of free or antibiotic-complexed large ribosomal subunits of a eubacterium, which compositions-of-matter being suitable for generating coordinate data defining the high-resolution three-dimensional atomic structure thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a photograph depicting D50S crystals grown according to the teachings of the present invention.

FIG. 2 is a photograph depicting two-dimensional polyacrylamide gel electrophoretic separation and identification of D50S proteins.

FIGS. 3a-c are atomic structure diagrams depicting a crown view representation of the D50S structure, shown from the side facing the small subunit within the 70S particle (FIG. 3a). The RNA chains are shown as ribbons (in cyan) and the proteins main chains in different colors. For orientation, the L12 stalk is on the right, the L1 stalk is on the left, and the central protuberance (CP), including the 5S rRNA is in the middle of the upper part of the particle. FIGS. 3b and 3c depict typical map segments of rRNA helices and proteins, respectively.

FIG. 4 is an atomic structure diagram depicting the location of protein CTC and its domain organization. CTC is shown in colored ribbons on the upper part of the D50S structure, shown in gray ribbons in the orientation of FIG. 3a. The N-terminal domain (Dom1) is located at the solvent side, shown in this figure behind the CP. The middle domain (Dom2) wraps around the CP and fills the gap extending to the L11 arm. The C-terminal domain (Dom3) is located at the rim of the intersubunit interface and reaches the site of docked A-site tRNA position (marked by a star).

FIG. 5 is an atomic structure diagram depicting the D50S L1-arm and its possible rotation. The adjacent part of the D50S structure is shown in gray, the L1-arm of D50S is highlighted in gold, and also shown is the L1-arm of the T70S structure in green. In T70S the L1-arm and protein L1 block the exit of the E-tRNA (magenta). Whereas, in the D50S structure, the L1-arm is displaced by about 30 Å, and can also rotate around a pivot point (marked by a red dot) by about 30 degrees, thus clearing the E-tRNA exit.

FIG. 6 is an atomic structure diagram depicting the intersubunit bridge to the decoding side of D50S. Shown is an overlay of H69 of D50S (cyan) and the corresponding feature in the structure of the T70S ribosome (gold). The figure indicates the proposed movement of H69 towards the decoding center of H44 (gray) in T30S.

FIGS. 7a-f are atomic structure diagrams depicting novel structural features identified in D50S. FIG. 7a depicts the inter-protein β-sheet, made by proteins L14-L19 in D50S, overlaid on the H50S counterparts, L14 and HL24e. Note the differences in structure and size between L19 in D50S to HL24e. FIG. 7b depicts the opening of the nascent polypeptide tunnel. The D50S protein L23 (gold) and its substitutes in H50S, L29e (red) and the HL23 (purple), are highlighted. FIG. 7c depicts an overlay of H25 in D50S (blue) and in H50S (red). In D50S H25 is significantly shorter, and proteins L20 (yellow) and L21 (green) are attached to it. Their space is occupied by part of the long helix of H25 in H550S. FIG. 7d depicts isolated views of L21 (green) and L23e (gray) that are related by an approximate 2-fold and which display similar extensions. FIG. 7e depicts an overlay of D50S protein L33 (purple) and H50S protein L44e (green) which shows similar globular domain folds in both proteins, but no extension for L33. Part of the space of the H50S L44e loop is occupied by the extension of D50S L31 (yellow). The E-site tRNA (red) is shown interacting with D50S L33 and H50S L44e and D50S L31 (gold) loop. FIG. 7f depicts a tweezers-like structure formed by proteins L32 (gold) and L22 (red), presumably stabilizing a helical structure generated from three RNA domains: H26 (green), the junction H61, H72 (blue) and the junction H26, H47 (cyan).

FIGS. 8a-c are structure diagrams depicting interaction of chloramphenicol with the peptidyl transferase cavity of D50S. FIG. 8a is a chemical structure diagram depicting interaction of chloramphenicol with 23S rRNA nucleotides in the peptidyl transferase cavity. Arrows depict interacting chemical moieties positioned less than 4.5 Å apart. FIG. 8b is a diagram depicting the secondary structure of the peptidyl transferase ring of D. radiodurans 23S rRNA, showing nucleotides (colored) interacting with chloramphenicol. Matching nucleotide color-coding schemes are used in FIGS. 8a and 8b. FIG. 8c is a stereo diagram depicting chloramphenicol binding sites in the peptidyl transferase cavity. The difference electron density map (2Fo-Fc) is contoured at 1.2 sigma. Chloramphenicol and portions of 23S rRNA which do not interact therewith are depicted in green and blue, respectively, and 23S rRNA nucleotides interacting with chloramphenicol are shown in the form of chemical structure models. Nucleotide numbering is according to the E. coli sequence. Mg2+ ions are indicated (Mg).

FIGS. 9a-c are structure diagrams depicting interaction of clindamycin with the peptidyl transferase cavity of D50S. FIG. 9a is a chemical structure diagram depicting interaction of clindamycin with 23S rRNA nucleotides in the peptidyl transferase cavity. Arrows depict interacting chemical moieties positioned less than 4.5 Å apart. FIG. 9b is a diagram depicting the secondary structure of the peptidyl transferase ring of D50S 23S rRNA showing nucleotides (colored) interacting with clindamycin. Matching nucleotide color-coding schemes are used in FIGS. 9a and 9b. FIG. 9c is a stereo diagram depicting clindamycin binding sites in the peptidyl transferase cavity. The difference electron density map (2Fo-Fc) is contoured at 1.2 sigma. Clindamycin and portions of 23S rRNA which do not interact therewith are depicted in green and blue, respectively, and 23S rRNA nucleotides interacting with clindamycin are shown as chemical structure models. Nucleotide numbering is according to the E. coli sequence.

FIGS. 10a-d are structure diagrams depicting interaction of the macrolide antibiotics erythromycin, clarithromycin and roxithromycin with the peptidyl transferase cavity of D50S. FIG. 10a is a chemical structure diagram depicting the interactions (colored arrows) of the reactive groups of the macrolides with the nucleotides of the peptidyl transferase cavity (colored). Colored arrows between two chemical moieties indicate that the two groups are less than 4.5 Å apart. Groups previously implicated in antibiotic interactions, namely proteins L4, L22, and domain II of the 23S rRNA are shown in black with their corresponding distances to the macrolide moieties. FIG. 10b is a diagram depicting secondary structure of the peptidyl transferase ring of D50S showing the nucleotides involved in the interaction with clindamycin (colored nucleotides). Matching nucleotide color-coding schemes are used in FIGS. 10a and 10b. FIG. 10c is a stereo diagram depicting the erythromycin binding site at the entrance of the tunnel of D50S. The stereo view of clarithromycin is identical to that of erythromycin. FIG. 10d is a stereo diagram depicting the roxithromycin binding site at the entrance of the tunnel of D50S. In FIGS. 10c and 10d, the difference electron density map (2Fo-Fc) is contoured at 1.2 sigma. Green, antibiotic; blue, 23S rRNA; yellow, part of ribosomal protein L4; light green, part of ribosomal protein L22. Nucleotides that interact with the antibiotic are shown with their chemical structure. Nucleotide numbering is according to the E. coli sequence.

FIG. 11 is a stereo diagram depicting the relative positions of chloramphenicol, clindamycin, and macrolides with respect to CC-puromycin and the 3′-CA end of P-site and A-site tRNAs. The location of CC-puromycin was obtained by docking the previously reported position thereof (Nissen, P. et al., 2000. Science 289, 920) into the PTC of D50S. The location of the 3′-CA end of P- and A-site tRNAs were obtained by docking the previously reported position (Yusupov, M M. et al., 2001. Science 292, 883) into the PTC of D50S. Light blue, 3′-CA end of A-site tRNA; light yellow, 3′ CA end of P-site tRNA; gray, puromycin; gold, chloramphenicol; green, clindamycin; cyan, macrolides (erythromycin). Oxygen atoms are shown in red and nitrogen atoms in dark blue.

FIG. 12 is an atomic structure diagram depicting the view of D50S from the 30S side showing erythromycin (red) bound at the entrance of the tunnel. Yellow, ribosomal proteins; gray, 23S rRNA; dark gray, 5S rRNA.

FIG. 13 is a diagram depicting a stereo view of the local environment surrounding ABT-773 together with its electron density map. For clarity, only the density of ABT-773 is shown, contoured at 1.2 sigma. Nucleotides contributing to hydrophobic interactions are labelled in black; those contributing to hydrogen bonds or electrostatic interactions are labelled in red.

FIG. 14 is a two-dimensional structure diagram depicting the interactions between ABT-773 and 23S rRNA. Nucleotides contributing to hydrophobic interactions are indicated; those contributing to hydrogen bonds or electrostatic interactions are represented by their structure.

FIGS. 15a-c are secondary structure diagrams depicting the parts of domains II, IV and V of 23S rRNA (FIGS. 15a-c, respectively) contributing to the binding of ABT-773 or azithromycin. Black circles indicate changes in nucleotides accessibility affected by macrolides according to biochemical data. Modifications or mutations affecting the macrolide susceptibility or resistance are indicated by arrows. The contacts sites of ABT-773 and azithromycin are indicated by large and coloured letters in the diagram. The colours of the letters correspond to the colours of the ligands in FIGS. 14a-b.

FIG. 16a is a stereo diagram depicting the local environment of the two azithromycin molecules complexed with D50S. For clarity, only the density of the azithromycin molecules is shown, contoured at 1.5 sigma. The colours of the letters correspond to the colours of the ligands in FIGS. 14a-b and 15a-c.

FIG. 16b is a two-dimensional structure diagram depicting the interactions between the two azithromycin molecules with the 23S rRNA molecule and the two ribosomal proteins L4 and L22. Nucleotides or amino acids contributing to hydrophobic interactions are indicated, with those contributing to hydrogen bonds or electrostatic interactions being represented by their structure. The primary (Azi-1) and secondary (Azi-2) binding sites of azithromycin are shown.

FIGS. 17a-c are diagrams depicting ASM (FIG. 17a), ACCP (FIG. 17b), and sparsomycin (FIG. 17c).

FIGS. 18a-g are atomic structure diagrams depicting D50S in complex with ASM, ACCP, sparsomycin, or ASMS. The same color code as in FIGS. 19a-j, below, has been used. Unless otherwise specified, the 23s rRNA is shown in green. The A-site and P-site tRNA (Yusupov et al., 2001. Science 292, 883-96) are shown in cyan and olive green, respectively, ASM is shown in red, ASMS is shown in pink-violet, ACCP is shown in blue, sparsomycin (SPAG) is shown in gold, protein L16 is shown in green, and protein CTC in native conformation is shown in dark blue. Protein CTC in occupied A-site conformation is shown in light green (FIG. 18b) or yellow (FIG. 18c). Nucleotides interacting with the substrates or inhibitors are numbered according to E. coli numbering system, and shown with their chemical structure. FIG. 18a depicts the location of the PTC within D50S. The modeled A- and P-site tRNAs, ASM and sparsomycin are also shown. FIG. 18b depicts the PTC and its environment, including ASM and the modeled A- and P-site tRNAs. FIG. 18c depicts domain 3 of protein CTC, the domain which undergoes substantial conformational rearrangements upon A-site occupation by ASM. Dark blue—the native conformation, yellow the conformation in ASM crystals. FIGS. 18d-g are stereo views depicting ASM, ACCP, sparsomycin and ASMS, respectively, in their binding sites within the peptidyl transferase cavity of D. radiodurans, together with their electron density maps, contoured at 1.0 sigma. The structures of ASM, ACCP, sparsomycin and ASMS are superposed on their corresponding electron densities.

FIGS. 19a-j. are atomic structure diagrams depicting D50S in complex with ASM, ACCP, sparsomycin, or ASMS. The same color code as in FIGS. 18a-g, above, has been used. Nucleotides interacting with the substrates or inhibitors are numbered according to E. coli numbering system, and shown with their chemical structure. FIGS. 19a-b depict side and front (relative to FIG. 18a) views of ASM, sparsomycin (SPAG) and ASMS within the PTC. The modeled A- and P-site tRNAs, ASM and sparsomycin are also shown. The view shown in FIG. 19a highlights the contributions of H69 and protein L16 to the precise positioning of ASM and ASMS. The view in FIG. 19b depicts the relative orientations of sparsomycin (SPAG) and the modeled P-site. FIGS. 19c-d depict H69 in D50S and T70S, respectively, in views highlighting their different conformation and supporting the possible participation of H69 in A- to P-site translocation. FIG. 19e-g depict three views, showing A2602 and a part of the backbone of H93 in the same orientation, together with sparsomycin (SPAG) and ASM (FIG. 19e), sparsomycin and ASMS (FIG. 19f), and sparsomycin and ACCP (FIG. 19g). Note the hydrated Mg2+ ions, shown as pink dots, near ASMS and the various conformations of A2602. FIG. 19h depicts the conformations of the PTC in complex with sparsomycin (SPAG; gold) and chloramphenicol (CAM; red) complexes. For clarity the 23s rRNA is colored blue relative to sparsomycin structures and green relative to chloramphenicol structures. FIG. 19i depicts ACCP, ASM and ASMS together with additional substrate analogs in the PTC, highlighting the positions of sparsomycin (SPAG) and base A2602. FIG. 19j depicts ten of the fifteen bases related by the two-fold symmetry (five bases are not shown for clarity). A2602 (in orange-brown) is located in the middle, near the two-fold axis.

FIG. 20a is a chemical structure diagram depicting the chemical structure of troleandomycin.

FIGS. 20b-c: are atomic structure diagrams depicting the atomic structure of crystallized LRS-troleandomycin complex. FIG. 20b depicts an interface view of the whole LRS-troleandomycin complex showing the location of troleandomycin (dark pink) within it. D50S is represented by metal-blue ribbons showing its RNA trace. FIG. 20c depicts a superposition of the structures of troleandomycin (TAO; dark pink) and erythromycin (ERY; green) bound within the tunnel of D50S (metal blue). The bases of the three nucleotides common to troleandomycin and erythromycin interactions with the ribosome are shown.

FIG. 20d is a stereo diagram depicting the atomic structure of the troleandomycin binding site in the LRS and the electron density of troleandomycin (TAO).

FIG. 20e is a two-dimensional chemical structure diagram depicting the interactions between troleandomycin and LRS atoms.

FIGS. 21a-c are atomic structure diagrams depicting the global conformational changes in the L22 hairpin triggered by troleandomycin binding. FIG. 21a depicts an electron density map (2Fo-Fc) for the swung conformation (magenta) of L22 beta-hairpin. For comparison, the native conformation is also shown (cyan). Note the hinge region at the bottom of the diagram. FIG. 21b depicts a view into the ribosomal tunnel from the active site, showing the hindrance of the tunnel by L22 swung conformation (magenta) compared to the native (cyan). Note how the native and swung double-hooks interact with two sides of the tunnel wall. The LRS is represented by a gray ribbon trace of the rRNA. FIG. 21c depicts a side view of the region of the ribosome exit tunnel, showing the contacts of the native (cyan) and swung (magenta) conformations of L22 hairpin tip. Note the collision between troleandomycin (TAO; dark yellow) and the tip of L22 hairpin at its native conformation. The RNA moieties constructing the tunnel wall at this region are shown in gray. The main interactions of the double hook arginines of the native and the swung conformations are indicated with their respective colors.

FIG. 22: is an atomic structure diagram depicting the putative progression of the SecM arrest sequence in the exit tunnel. The modeled poly-glycine chain together with the native (cyan) and the swung (magenta) L22 beta-hairpin conformations are shown. The region highlighted in pink corresponds to Trp155 and Ile156 in the SecM sequence (Nakatogawa H. and Ito K., 2002. Cell 108, 629-36). The walls of the exit tunnel are represented by the RNA backbone (gray).

FIG. 23 is a schematic diagram depicting a computing platform for generating a three-dimensional model of at least a portion of a LRS or of a complex of an antibiotic and a LRS.

### DESCRIPTION OF THE PREFERRED EMBODIMENTS

The present invention is of a crystallized large ribosomal subunit (LRS), crystallized co-complexes of a eubacterial LRS and an antibiotic and/or a ribosomal substrate, compositions-of-matter comprising such crystals and methods of using structural data derived from such crystals for generating three-dimensional (3D) models of the LRS or LRS-ligand complexes, which models can be used for rational design or identification of novel antibiotics and LRSs having desired characteristics.

The principles and operation of the present invention may be better understood with reference to the accompanying descriptions.

Before explaining at least one embodiment of the invention in detail, it is to be understood that the invention is not limited in its application to the details of construction and the arrangement of the components set forth in the following description or exemplified in the Examples. The invention is capable of other embodiments or of being practiced or carried out in various ways. Also, it is to be understood that the phraseology and terminology employed herein is for the purpose of description and should not be regarded as limiting.

For the past few years, the rational design or identification of novel antibiotics has gained importance mostly due to the emergence of pathogenic bacterial strains resistant to known antibiotics.

One major binding target of antibiotics is the LRS, the universal and central macromolecular catalyst of protein synthesis, thus the LRS has been the center of numerous studies due to its pivotal role in protein synthesis and antibiotic therapy.

Various approaches for generating three-dimensional atomic structure models of free, antibiotic-complexed or ribosomal substrate-complexed ribosomal subunits have been described by the prior art. All such approaches have, however, proven to be suboptimal for the various reasons.

For example, attempts to generate structure models of the LRS of the eubacterium E. coli have failed since this molecule is too fragile to generate X-ray crystallography grade crystals.

Information derived from attempts to determine the structure of the 30S subunit from the thermophilic bacterium Thermus thermophilus (T. thermophilus) (T30S) alone or in complex with various combinations of RNA molecules, initiation factors and small ribosomal subunit-specific antibiotics cannot be used for modeling free or complexed LRSs.

Approaches attempting to determine the structure of the T. thermophilus 70S ribosomal particle (T70S) in complex with mRNA and tRNA have failed to yield structures at resolutions higher than 5.5 Å nor have these provided structure models of the LRS in complex with an antibiotic molecule.

Attempts to determine the structure of the LRS of the archaea Haloarcula marismortui (H. marismortui) have not provided satisfactory coverage of the structural features involved in the non-catalytic functional aspects of protein biosynthesis. Critically, there are significant differences between such archaeal LRS and eubacterial LRSs, the latter being of vastly greater significance, in the biomedical and industrial fields, than the former. Archaeal ribosomes have not only bacterial but also eukaryotic properties and are therefore suboptimal for modeling the eubacterial LRS.

Thus, all prior art approaches have failed to provide satisfactory three-dimensional atomic structure models of free, antibiotic complexed, or substrate-complexed eubacterial LRSs.

While reducing the present invention to practice, the present inventors have generated an essentially complete high resolution three-dimensional atomic structure model of a eubacterial LRS, and high resolution three-dimensional atomic structure models of such a LRS in complex with a range of antibiotics.

As used herein, an “essentially complete” structure of a LRS refers to a high resolution structure whose RNA component is at least 96% complete and which includes the features involved in both catalytic and non-catalytic functional aspects of protein biosynthesis at high resolution.

As used herein, the term “high resolution” refers to a resolution higher than or equal to 5.4 Å.

As used herein, the, the term “antibiotic” refers to any compound or combination of compounds capable of killing bacteria, inhibiting the growth or reproduction of bacteria, inhibiting bacterial infection, or inhibiting the function of bacterial ribosomes.

As described in Example 1 of the Examples section below, an essentially complete structure model of a free LRS at a resolution of 3.1 Å was generated for the first time. Due to its high resolution, this model is superior to all prior art LRS models. Furthermore, this model is also superior to all prior art LRS models in that it represents the most complete bacterial, including archaeal, LRS model generated at a resolution of 3.1 Å, or higher. In addition, as is shown in Examples 2-5 of the Examples section which follows, structure models of the interaction between the LRS and a range of antibiotics and/or substrates were also generated at resolutions as high as 3.1 Å.

The structures of the LRS-antibiotic complexes described in Example 2 of the Examples section below represent the first three-dimensional atomic structure models of the interaction between LRSs and antibiotics. Such novel and highly resolved crystallography data, which were obtained using the crystallography method of the present invention, represents a breakthrough of historical proportions in structure determination of free and antibiotic-complexed LRSs (Examples 1 and 2, respectively) and, as such, these data have been recently published, after the earliest priority date of this application, in both Cell and Nature (Harms J. et al., 2001. Cell 107, 679; and Schlüenzen F. et al., 2001. Nature 413, 814, respectively).

Furthermore, the atomic structures of the LRS complexed with an antibiotic and/or a substrate, as described in Examples 3-5 of the Examples section which follows, constitute the first structures of the eubacterial LRS complexed with such antibiotics and/or substrates, as further described hereinbelow.

Due to the completeness and highly resolved nature of the data obtained, the models of the present invention constitute a unique and powerful tool capable of greatly facilitating the rational design or identification of LRS-targeting antibiotics, or of LRSs having desired characteristics, and of providing profound insights into the crucial and universal mechanisms of protein production which are performed by the ribosome.

Thus, according to one aspect of the present invention there are provided compositions including crystallized eubacterial LRSs.

According to one embodiment, such compositions comprise crystallized free LRSs.

As used herein, the term “free LRSs” refers to LRSs which are not complexed with an antibiotic or a substrate.

The crystallized free LRSs of the present invention are suitable for generating, preferably via X-ray crystallography, coordinate data defining the high resolution three-dimensional atomic structure of essentially complete crystallized free LRSs, or portions of crystallized free LRSs, as shown in Example 1 of the Examples section, below.

As used herein, the terms “coordinate data”, “structure coordinate data”, and “structure coordinate” are used interchangeably.

X-ray crystallography is effected by exposing crystals to an X-ray beam and collecting the resultant X-ray diffraction data. This process usually involves the measurements of many tens of thousands of data points over a period of one to several days depending on the crystal form and the resolution of the data required. The crystals diffract the rays, creating a geometrically precise pattern of spots recorded on photographic film or electronic detectors. The distribution of atoms within the crystal influences the pattern of spots. The quality of protein crystals is determined by the ability of the crystal to scatter X-rays of wavelengths (typically 1.0-1.6 Å) suitable to determine the atomic coordinates of the macromolecule. The measure of the quality is determined as a function of the highest angle of scatter (the ultimate or intrinsic resolution) and according to Bragg's Law: nλ=2d sin θ (where θ is the angle of incidence of the reflected X-ray beam, d is the distance between atomic layers in a crystal, λ is the wavelength of the incident X-ray beam, and n is an integer), d may be determined, and represents the resolution of the crystal form in angstroms. Thus, this measurement is routinely used to judge the ultimate usefulness of protein crystals. Group theory shows that there are 230 unique ways in which chemical substances, proteins or otherwise, may assemble in three-dimensional to form crystals. These are called the 230 “space groups.” The designation of the space group in addition to the unit cell constants (which define the explicit size and shape of the cell which repeats periodically within the crystal) is routinely used to uniquely identify a crystalline substance. Certain conventions have been established to ensure the proper identification of crystalline materials and these conventions have been set forth and documented in the International Tables for Crystallography, incorporated herein by reference.

The crystallized free LRSs of the present invention can be used to generate coordinate data defining essentially complete structures of crystallized free LRSs, or structures of portions of crystallized free LRSs, at a resolution preferably higher than or equal to 5.4 Å, more preferably higher than or equal to 5.3 Å, more preferably higher than or equal to 5.2 Å, more preferably higher than or equal to 5.1 Å, more preferably higher than or equal to 5.0 Å, more preferably higher than or equal to 4.9 Å, more preferably higher than or equal to 4.8 Å, more preferably higher than or equal to 4.7 Å, more preferably higher than or equal to 4.6 Å, more preferably higher than or equal to 4.5 Å, more preferably higher than or equal to 4.4 Å, more preferably higher than or equal to 4.3 Å, more preferably higher than or equal to 4.2 Å, more preferably higher than or equal to 4.1 Å, more preferably higher than or equal to 4.0 Å, more preferably higher than or equal to 3.9 Å, more preferably higher than or equal to 3.8 Å, more preferably higher than or equal to 3.7 Å, more preferably higher than or equal to 3.6 Å, more preferably higher than or equal to 3.5 Å, more preferably higher than or equal to 3.4 Å, more preferably higher than or equal to 3.3 Å, more preferably higher than or equal to 3.2 Å, and most preferably higher than or equal to 3.1 Å.

Preferably, the coordinate data used to define the structure of the crystallized free ribosomal subunit or a portion thereof is the set of coordinate data set forth in Table 3 (refer to attached CD-ROM) or a portion thereof.

As described in Example 1 of the Examples section which follows, the coordinate data set forth in Table 3 (refer to attached CD-ROM) can be used to define the structure of the essentially complete LRS at high resolution.

Preferably, the portion of the coordinate data set forth in Table 3 (refer to attached CD-ROM) used to define the structure of a portion of the crystallized free ribosomal subunit corresponds to:

nucleotide coordinates 2044, 2430, 2431, 2479 and 2483-2485;

nucleotide coordinates 2044-2485;

nucleotide coordinates 2040-2042, 2044, 2482, 2484 and 2590;

nucleotide coordinates 2040-2590;

nucleotide coordinates 2040-2042, 2045, 2484, 2588 and 2589;

nucleotide coordinates 2040-2589;

atom coordinates 1-59360;

atom coordinates 59361-61880;

atom coordinates 1-61880;

atom coordinates 61881-62151;

atom coordinates 62152-62357;

atom coordinates 62358-62555;

atom coordinates 62556-62734;

atom coordinates 62735-62912;

atom coordinates 62913-62965;

atom coordinates 62966-63109;

atom coordinates 63110-63253;

atom coordinates 63254-63386;

atom coordinates 63387-63528;

atom coordinates 63529-63653;

atom coordinates 63654-63768;

atom coordinates 63769-63880;

atom coordinates 63881-64006;

atom coordinates 64007-64122;

atom coordinates 64123-64223;

atom coordinates 64224-64354;

atom coordinates 64355-64448;

atom coordinates 64449-64561;

atom coordinates 64562-64785;

atom coordinates 64786-64872;

atom coordinates 64873-64889;

atom coordinates 64890-64955;

atom coordinates 64956-65011;

atom coordinates 65012-65085;

atom coordinates 65086-65144;

atom coordinates 65145-65198;

atom coordinates 65199-65245;

atom coordinates 65246-65309;

atom coordinates 65310-65345;

atom coordinates 61881-65345; or

atom coordinates 1-65345.

As described in Example 1 of the Examples section below, the following atomic coordinates set forth in Table 3 (refer to attached CD-ROM) can be used to define the indicated LRS portions:

23S rRNA: 1-59360,

5S rRNA: 59361-61880,

ribosomal protein L2: 61881-62151,

ribosomal protein L3: 62152-62357,

ribosomal protein L4: 62358-62555,

ribosomal protein L5: 62556-62734,

ribosomal protein L6: 62735-62912,

ribosomal protein L9: 62913-62965,

ribosomal protein L11: 62966-63109,

ribosomal protein L13: 63110-63253,

ribosomal protein L14: 63254-63386,

ribosomal protein L15: 63387-63528,

ribosomal protein L16: 63529-63653,

ribosomal protein L17: 63654-63768,

ribosomal protein L18: 63769-63880,

ribosomal protein L19: 63881-64006,

ribosomal protein L20: 64007-64122,

ribosomal protein L21: 64123-64223,

ribosomal protein L22: 64224-64354,

ribosomal protein L23: 64355-64448,

ribosomal protein L24: 64449-64561,

ribosomal protein CTC: 64562-64785,

ribosomal protein L27: 64786-64872,

ribosomal protein L28: 64873-64889,

ribosomal protein L29: 64890-64955,

ribosomal protein L30: 64956-65011,

ribosomal protein L31: 65012-65085,

ribosomal protein L32: 65086-65144,

ribosomal protein L33: 65145-65198,

ribosomal protein L34: 65199-65245,

ribosomal protein L35: 65246-65309, and

ribosomal protein L36: 65310-65345.

Thus, the present invention provides coordinate data which define the three-dimensional atomic structure of essentially whole crystallized free LRSs, or components thereof, at resolutions as high as 3.1 Å.

As used herein, the term “three-dimensional atomic structure” refers to the positioning and structure of atoms or groups of atoms, including sets of atoms or sets of groups of atoms which are not directly associated with each other such as, for example, sets of non-contiguous nucleotides from the same polynucleotide molecule.

Those of ordinary skill in the art will understand that a set of atomic structure coordinates is a relative set of points that define a shape in three dimensions. Thus, it is possible that a different set of coordinates, for example a set of coordinates utilizing a different frame of reference and/or different units, could define a similar or identical shape. Moreover, it will be understood that slight variations in the individual coordinates will have little effect on overall shape.

The variations in coordinates discussed above may be generated because of mathematical manipulations of the structure coordinates. For example, structure coordinates can be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, or other changes in any of the components that make up the crystal could also account for variations in structure coordinates. If such variations are within an acceptable standard error as compared to the original coordinates, the resulting three-dimensional shape is considered to be the same.

The LRS of D. radiodurans is a very large macromolecular complex comprising the following components: 5S and 23S rRNA molecules, and ribosomal proteins L1-L7, L9-L24, CTC, and L27-L36.

As shown in Example 1 of the Examples section below, crystal-derived coordinate data can be used to define the structure of such LRS components, of portions of such LRS components, of combinations of such LRS components, or essentially of the entirety of the LRS (refer to Table 3).

While reducing the present invention to practice, the present inventors succeeded, following extensive experimentation, as described in Example 1 of the Examples section below, in crystallizing free eubacterial LRSs.

Thus, according to another aspect of the present invention, there is provided a method of crystallizing eubacterial LRSs.

Preferably, the method of the present invention is used to crystallize LRSs of Deinococcus radiodurans (D. radiodurans), more preferably of Deinococcus-Thermophilus group bacteria, more preferably of gram-positive bacteria, and most preferably of cocci.

According to the teachings of the present invention, crystallized free LRSs are obtained by isolating LRSs, preferably as previously described (Noll, M. et al., 1973. J Mol Biol. 75, 281) and suspending them in an aqueous crystallization solution preferably supplemented with 0.1-10% (v/v) of a volatile component and equilibrating the resulting crystallization mixture, preferably by vapor diffusion, preferably using standard Linbro dishes, preferably at 15-25 degrees centigrade, most preferably at 17-20 degrees centigrade, against an equilibration solution supplemented with the aforementioned volatile component at a concentration preferably 0.33-0.67 times, most preferably 0.5 times that thereof in the crystallization solution.

Typically in the vapor diffusion method, a small drop of crystallization mixture containing a macromolecule to be crystallized is placed on a cover slip or glass plate which is inverted over a well of equilibration solution such that the cover slip or glass plate forms a seal over the well. The equilibration solution is initially at a lower volatile component vapor pressure than the crystallization mixture so that evaporation of the volatile component from the crystallization mixture to the equilibration mixture progresses at a rate fixed by the difference in the vapor pressures therebetween and by the distance between the crystallization mixture and the equilibration solution. Thus, as evaporation proceeds, the crystallization mixture becomes supersaturated with the macromolecule to be crystallized and, under the appropriate crystallization mixture conditions-including pH, solute composition and/or concentration, and temperature-crystallization occurs.

Suitable crystallization solutions and equilibration solutions according to the present invention comprise, via the buffer component thereof MgCl₂, preferably at a concentration of 3-12 mM, most preferably 10 mM; NH₄Cl, preferably at a concentration of 20-70 mM, most preferably 60 mM; KCl, preferably at a concentration of 0-15 mM, most preferably 5 mM; and HEPES, preferably at a concentration of 8-20 mM, most preferably 10 mM.

Preferably crystallization solutions and equilibration solutions are at a pH of 6.0-9.0, most preferably at a pH of 7.8.

Preferably, the buffer component of crystallization solutions and equilibration solutions are optimized for enabling in-vitro functional activity of LRSs.

According to a preferred embodiment of the present invention, the buffer component of crystallization solutions and equilibration solutions is H-I buffer (10 mM MgCl₂, 60 mM NH₄Cl, 5 mM KCl, 10 mM HEPES pH 7.8).

Preferably, the volatile component is composed of a mixture of multivalent and monovalent alcohols, the multivalent to monovalent alcohol ratio preferably being 1:3.0 to 1:4.1, most preferably 1:3.56, the multivalent alcohol preferably being dimethylhexandiol and the monovalent alcohol preferably being ethanol.

The crystallized free LRSs of the present invention are preferably characterized by unit cell dimensions of about a=170.827±10 Å, b=409.430±15 Å and c=695.597±25 Å; more preferably a=170.827±5 Å, b=409.430±7.5 Å and c=695.597±12.5 Å; and most preferably a=170.827±1 Å, b=409.430±1.5 Å and c=695.597±2.5 Å, as shown in Example 1 of the Examples section, which follows.

As used herein the term “about” refers to ±10%.

It will be appreciated by one of ordinary skill in the art that, due to the high level of conservation between LRSs of different eubacteria, the method of crystallizing LRSs of the present invention can be generally applied to crystallizing LRSs of different types/species of eubacteria.

Examples of types/species of eubacteria include Aquifex, Thermotogales group bacteria (e.g., Thermotoga, Fervidobacterium), Thermodesulfobacterium group bacteria (e.g., Thermodesulfobacterium), Green nonsulfur group bacteria (e.g., Chloroflexus, Herpetosiphon, Thermomicrobium), Deinococcus-Thermus group bacteria (e.g., Deinococcus, Thermus), Thermodesulfovibrio group bacteria (e.g., Thermodesulfovibrio), Synergistes group bacteria (e.g. Synergistes), low G+C Gram positive group bacteria (e.g. Bacillus, Clostridium, Eubacterium, Heliobacterium, Lactobacillus, Mycoplasma, Spiroplasma), high G+C Gram positive group bacteria (e.g., Bifidobacterium, Mycobacterium, Propionibacterium, Streptomyces), Cyanobacteria (e.g., Oscillatoria, Prochlorococcus, Synechococcus, chloroplasts), Planctomycetales group bacteria (e.g., Planctomyces), Chiamydiales group bacteria (e.g., Chlamydia), Green sulfur group bacteria (e.g., Chlorobium), Cytophaga group bacteria (e.g., Bacteriodes, Cytophaga, Flexibacter, Flavobacterium, Rhodothermus), Fibrobacter group bacteria (e.g., Fibrobacter), Spirochetes group bacteria (e.g., Borrelia, Leptonema, Spirochaeta (including Spirochaeta sp. str. Antarctic), Treponema), and Proteobacteria group bacteria (e.g., alpha Proteobacteria, beta Proteobacteria, gamma Proteobacteria, delta/epsilon Proteobacteria, Agrobacterium, Anaplasma, Rhodobacter, Rhodospirillum, Rickettsia, ritochondria, Neisseria, Rhodocyclus, Beggiatoa, Chromatium, Escherichia, Haemophilus, Legionella, Pseudomonas, Salmonella, Vibrio, Yersinia, Bdellovibrio, Campylobacter, Desulfovibrio, Helicobacter, Myxococcus, and Wolinella).

Preferably, the method according to this aspect of the present invention is used to crystallize free LRSs of Deinococcus-Thermus group bacteria.

Examples of Deinococcus-Thermus group bacteria include Thermus, such as, for example, T. thermophihus, T. aquaticus, and T. flavus; and Deinococcus such as, for example, D. radiodurans, D. geothermalis, D. radiophilus, D. murrayi, D. proteolyticus, D. radiopugnans, and D. erythromyxa.

Most preferably the method of the present invention is used to crystallize D. radiodurans free LRSs.

Thus, the present invention also provides a method which can be used to crystallize LRSs in a manner which enables fine resolution of the crystal structure.

Although the method of the present invention is most preferably used to crystallize D. radiodurans LRS-antibiotic complexes or LRS-substrate complexes, as described in Examples 2-5 of the Examples section below, the method is generally suitable for crystallizing eubacterial LRS-antibiotic or LRS-substrate complexes.

As mentioned hereinabove, LRSs are one of the main targets for antibiotics. As such, the present crystallization method was also used to crystallize LRS-antibiotic complexes in efforts of gaining insight into LRS-antibiotic interactions.

Thus, according to another aspect of the present invention there are provided compositions including crystallized antibiotic-LRS complexes.

Preferably, the antibiotic is chloramphenicol, a lincosamide antibiotic, a macrolide antibiotic, a puromycin conjugate, ASMS, and sparsomycin.

Examples of lincosamide antibiotics include lincomycin, pirlimycin and clindamycin.

Preferably, the lincosamide antibiotic is clindamycin.

Examples of macrolide antibiotics include erythromycin, carbomycin, clarithromycin, josamycin, leucomycin, midecamycin, mikamycin, miokamycin, oleandomycin, pristinamycin, rokitamycin, rosaramicin, roxithromycin, spiramycin, tylosin, troleandomycin, virginiamycin, ketolides and azalides.

Preferably, the macrolide antibiotic is erythromycin, clarithromycin, roxithromycin, troleandomycin, a ketolide antibiotic or an azalide antibiotic.

Examples of ketolide antibiotics include telithromycin and ABT-773.

Preferably, the ketolide is ABT-773.

Preferably, the azalide antibiotic is azithromycin.

Preferably, the puromycin conjugate is ACCP or ASM.

As described in Example 4 of the following Examples section, ACCP and ASM are composed of puromycin conjugated to short and long tRNA acceptor stem portions, respectively. As such, ACCP and ASM further constitute ribosomal substrate analogs. As also described in Example 4 of the following Examples section, ASMS is a combination of ASM and sparsomycin, and as such constitutes a combination of a ribosomal substrate analog and sparsomycin.

As is shown in the Examples section which follows, the present invention provides compositions-of-matter including crystallized LRS-chloramphenicol, LRS-clindamycin, LRS-clarithromycin, LRS-roxithromycin, LRS-erythromycin, LRS-ABT-773, LRS-azithromycin, LRS-ACCP, LRS-ASM, LRS-ASMS, LRS-sparsomycin, and LRS-troleandomycin complexes.

Crystallization of LRS-antibiotic complexes may be achieved via various methods, depending on the antibiotic to be co-crystallized. Preferably, the antibiotic is either co-crystallized with the LRS by including the antibiotic in the crystallization solution, or is co-crystallized by soaking the crystallized free LRS in a soaking solution containing the antibiotic, for example at a concentration of about 0.01 mM.

Crystallization of LRSs in complex with chloramphenicol, clindamycin, roxithromycin, or erythromycin, is preferably effected by including the antibiotic in the crystallization solution at a concentration selected from the range of 0.8-3.5 mM.

Crystallization of LRSs in complex with ABT-773 or azithromycin is preferably effected by including the antibiotic in the crystallization solution at a concentration about 5.5 to 8.5 times higher, more preferably about 7 times higher than the concentration of the LRS in the crystallization solution.

Crystallization of LRSs in complex with sparsomycin is preferably effected by including the antibiotic in the crystallization solution at a concentration about 8 to 12 times higher, more preferably about 10 times higher than the concentration of the LRS in the crystallization solution.

Crystallization of LRSs in complex with clarithromycin is preferably effected by soaking the crystallized LRS in a soaking solution comprising the antibiotic at a concentration selected from the range of about 0.004-0.025 mM.

Crystallization of LRSs in complex with ACCP is preferably effected by soaking the crystallized LRS in a soaking solution comprising the antibiotic at a concentration selected from the range of about 0.0150-0.0100 mM, more preferably about 0.0125 mM.

Crystallization of LRSs in complex with ASM is preferably effected by soaking the crystallized LRS in a soaking solution comprising the antibiotic at a concentration selected from the range of about 0.020-0.030 mM, more preferably about 0.025 mM.

Crystallization of LRSs in complex with troleandomycin is preferably effected by soaking the crystallized LRS in a soaking solution comprising the antibiotic at a concentration selected from the range of about 0.020-0.030 mM, more preferably about 0.025 mM.

Crystallization of LRSs in complex with troleandomycin is preferably effected by soaking the crystallized LRS in a soaking solution comprising the antibiotic at a concentration selected from the range of about 0.080-0.120 mM, more preferably about 0.100 mM.

Crystallization of LRSs in complex with ASMS is preferably effected by first co-crystallizing the LRS with sparsomycin, as described hereinabove, and soaking the crystallized LRS-ASM complex in a soaking solution comprising ASM at a concentration selected from the range of about 0.020-0.030 mM, more preferably about 0.025 mM.

The crystallized antibiotic-LRS complexes of the present invention are suitable for generating, preferably via X-ray crystallography, coordinate data defining high resolution structures of crystallized antibiotic-LRS complexes, or portions thereof comprising antibiotic-binding pockets of LRSs and/or antibiotics. Hence, the crystallized antibiotic-LRS complexes of the present invention are suitable for generating coordinate data defining high resolution three-dimensional atomic structures of the atomic interactions between antibiotic-binding pockets of LRSs and antibiotics.

As used herein, an “antibiotic-binding pocket” is defined as the set of LRS atoms or nucleotides which specifically associate with, or are capable of specifically associating with, an antibiotic.

Thus, the present invention provides coordinate data which define, at a resolution higher than or equal to 3.1 Å, the structure of crystallized antibiotic-LRS complexes, or portions thereof, including portions comprising the antibiotic-binding pocket of the LRS and/or the antibiotic, as demonstrated in Example 2 of the Examples section, below.

As is further described in the Examples section which follows, the present methodology was used to crystallize chloramphenicol, LRS-clindamycin, LRS-clarithromycin, LRS-erythromycin, LRS-roxithromycin, LRS-ABT-773-LRS, LRS-azithromycin, LRS-ACCP, LRS-ASM, LRS-ASMS- LRS, and LRS-troleandomycin complexes.

The crystallized chloramphenicol-LRS complexes of the present invention are preferably characterized by unit cell dimensions of about a=171.066±10 Å b=409.312±15 Å, and c=696.946±25 Å; more preferably a=171.066±5 Å, b=409.312±7.5 Å, and c=696.946±12.5 Å; and most preferably a=171.066±1 Å, b=409.312±1.5 Å, and c=696.946±2.5 Å, as shown in Example 2 of the Examples section, which follows.

The crystallized clindamycin-LRS complexes of the present invention are preferably characterized by unit cell dimensions of about: a=170.286±10 Å, b=410.134±15 Å and, c=697.201±25 Å; more preferably a=170.286±5 Å, b=410.134±7.5 Å, and c=697.201±12.5 Å; and most preferably a=170.286 ±1 Å, b=410.134±1.5 Å, and c=697.201±2.5 Å, as shown in Example 2 of the Examples section, below.

The crystallized clarithromycin-LRS complexes of the present invention are preferably characterized by unit cell dimensions of about: a=169.871±10 Å, b=412.705±15 Å and c=697.008±25 Å; more preferably a=169.871±5 Å, b=412.705±7.5 Å and c=697.008±12.5 Å; and most preferably a=169.871±1 Å, b=412.705±1.5 Å and c=697.008±2.5 Å, as shown in Example 2 of the Examples section, which follows.

The crystallized erythromycin-LRS complexes of the present invention are preferably characterized by unit cell dimensions of about: a=169.194±10 Å, b=409.975±15 Å, and c=695.049±25 Å; more preferably a=169.194±5 Å, b=409.975±7.5 Å, and c=695.049±12.5 Å; and most preferably a=169.194±1 Å, b=409.975±1.5 Å, and c=695.049±2.5 Å, as shown in Example 2 of the Examples section, below.

The crystallized roxithromycin-LRS complexes of the present invention are preferably characterized by unit cell dimensions of about: a=170.357±10 Å, b=410.713±15 Å, and c=694.810±25 Å; more preferably a=170.357±5 Å, b=410.713±7.5 Å, and c=694.810±12.5 Å; and most preferably a=170.357±1 Å, b=410.713±1.5 Å, and c=694.810±2.5 Å, as shown in Example 2 of the Examples section, which follows.

The crystallized ACCP-LRS complexes of the present invention are preferably characterized by unit cell dimensions of about: a=169.9 Å, b=410.4 and c=697.1 Å, as shown in Example 4 of the Examples section, which follows.

The crystallized ASM-LRS complexes of the present invention are preferably characterized by unit cell dimensions of about: a=169.9 Å, b=409.9 Å and c=695.9 Å, as shown in Example 4 of the Examples section, which follows.

The crystallized ASMS-LRS complexes of the present invention are preferably characterized by unit cell dimensions of about: a=169.6 Å, b=409.4 Å, and c=695.1 Å, as shown in Example 4 of the Examples section, which follows.

The crystallized sparsomycin-LRS complexes of the present invention are preferably characterized by unit cell dimensions of about: a=169.6 Å, b=409.4 Å and c=695.1 Å, as shown in Example 4 of the Examples section, which follows.

The crystallized troleandomycin-LRS complexes of the present invention are preferably characterized by unit cell dimensions of about: a=170.3 Å, b=411.1 Å and c=695.5 Å, as shown in Example 5 of the Examples section, which follows.

The crystallized antibiotic-LRS complexes of the present invention can be used to generate coordinate data defining structures thereof at characteristic resolutions, depending on the antibiotic, as described hereinbelow.

The crystallized antibiotic-LRS complexes of the present invention can be used to generate coordinate data defining structures of crystallized chloramphenicol-LRS, clarithromycin-LRS, ABT-773-LRS or puromycin conjugate-LRS complexes, preferably at a resolution higher than or equal to 7 Å, more preferably at a resolution higher than or equal to 6 Å, more preferably at a resolution higher than or equal to 5 Å, more preferably at a resolution higher than or equal to 4 Å, and most preferably at a resolution higher than or equal to 3.5 Å. As shown in Examples 2, 3, and 4, the crystallized antibiotic-LRS complexes of the present invention can be used to generate coordinate data defining structures of: crystallized chloramphenicol-LRS or clarithromycin-LRS complexes; crystallized ABT-773-LRS complexes; or crystallized puromycin conjugate-LRS complexes at a resolution higher than or equal to 3.5 Å, respectively.

The crystallized antibiotic-LRS complexes of the present invention can further used to generate coordinate data defining structures of crystallized clindamycin-LRS complexes, preferably at a resolution higher than or equal to 6.2 Å, more preferably at a resolution higher than or equal to 5 Å, more preferably at a resolution higher than or equal to 4 Å, more preferably at a resolution higher than or equal to 3.5 Å, and most preferably at a resolution higher than or equal to 3.1 Å. As shown in Example 2 of the Examples section below, the crystallized antibiotic-LRS complexes of the present invention can be used to generate coordinate data defining the structure of clindamycin-LRS complexes at a resolution of 3.1 Å.

The crystallized antibiotic-LRS complexes of the present invention can yet further be used to generate coordinate data defining structures of crystallized erythromycin-LRS or troleandomycin-LRS complexes, preferably at a resolution higher than or equal to 6.8 Å, more preferably at a resolution higher than or equal to 6 Å, more preferably at a resolution higher than or equal to 5 Å, more preferably at a resolution higher than or equal to 4 Å, and most preferably at a resolution higher than or equal to 3.4 Å. As shown in Examples 2 and 5 of the Examples section which follows, the crystallized antibiotic-LRS complexes of the present invention can be used to generate coordinate data defining the structures of crystallized erythromycin-LRS or troleandomycin-LRS complexes at a resolution of 3.4 Å.

The crystallized antibiotic-LRS complexes of the present invention can still further be used to generate coordinate data defining the structure of crystallized clarithromycin-LRS complexes, preferably at a resolution higher than or equal to 7.4 Å, more preferably at a resolution higher than or equal to 7 Å, more preferably at a resolution higher than or equal to 6 Å, more preferably at a resolution higher than or equal to 5 Å, more preferably at a resolution higher than or equal to 4 Å, and most preferably at a resolution higher than or equal to 3.8 Å. As shown in Example 2 of the Examples section below, the crystallized antibiotic-LRS complexes of the present invention can be used to generate coordinate data defining the structure of crystallized clarithromycin-LRS complexes at a resolution of 3.8 Å.

The crystallized antibiotic-LRS complexes of the present invention can additionally used to generate coordinate data defining structures of crystallized azithromycin-LRS complexes, preferably at a resolution higher than or equal to 6.4 Å, more preferably at a resolution higher than or equal to 6 Å, more preferably at a resolution higher than or equal to 5 Å, more preferably at a resolution higher than or equal to 4 Å, and most preferably at a resolution higher than or equal to 3.2 Å. As shown in Example 3 of the Examples section which follows, the crystallized antibiotic-LRS complexes of the present invention can be used to generate coordinate data defining the structure of crystallized azithromycin-LRS complexes at a resolution of 3.2 Å.

The crystallized antibiotic-LRS complexes of the present invention can yet additionally used to generate coordinate data defining structures of crystallized ACCP-LRS or sparsomycin-LRS complexes, preferably at a resolution higher than or equal to 7.4 Å, more preferably at a resolution higher than or equal to 6 Å, more preferably at a resolution higher than or equal to 5 Å, more preferably at a resolution higher than or equal to 4 Å, and most preferably at a resolution higher than or equal to 3.7 Å. As shown in Example 4 of the Examples section below, the crystallized antibiotic-LRS complexes of the present invention can be used to generate coordinate data defining the structure of crystallized ACCP-LRS or sparsomycin-LRS complexes at a resolution of 3.7 Å.

The crystallized antibiotic-LRS complexes of the present invention can still additionally used to generate coordinate data defining structures of crystallized ASMS-LRS complexes, preferably at a resolution higher than or equal to 7.2 Å, more preferably at a resolution higher than or equal to 6 Å, more preferably at a resolution higher than or equal to 5 Å, more preferably at a resolution higher than or equal to 4 Å, and most preferably at a resolution higher than or equal to 3.6 Å. As shown in Example 4 of the following Examples section, the crystallized antibiotic-LRS complexes of the present invention can be used to generate coordinate data defining the structure of crystallized ASMS-LRS complexes at a resolution of 3.6 Å.

Since the crystallized antibiotic-LRS complexes of the present invention can be used to generate coordinate data defining the structure, thereof at high resolution, these can be used to define the structure of portions of such complexes so as to identify atoms associated between the LRS and an antibiotic, thereby generating models of the interaction between portions of the LRS, such as the antibiotic binding pocket thereof, and an antibiotic.

As used herein, atoms which are “associated” are positioned less than 4.5 Å apart.

As shown in Examples 2, 3, and 4, respectively, of the Examples section which follows, atoms of LRSs associated with: clindamycin, erythromycin, clarithromycin, roxithromycin, chloramphenicol; ABT-773; and ACCP and sparsomycin, atoms in crystallized antibiotic-LRS complexes are 23S rRNA nucleotide atoms.

As shown in Examples 3, 4, and 5 of the Examples section which follows, respectively, atoms of LRSs associated with: azithromycin; ASM and ASMS; and troleandomycin atoms in crystallized antibiotic-LRS complexes are 23S rRNA nucleotide atoms and ribosomal protein amino acid residue atoms.

Preferably, the coordinate data used to define the structure of a crystallized LRS-chloramphenicol complex, or of a portion thereof, is the set of coordinate data set forth in Table 7 (refer to attached CD-ROM), or a portion thereof.

As described in Example 2 of the Examples section which follows, the coordinate data set forth in Table 7 (refer to attached CD-ROM) can be used to define the structure of the essentially complete LRS-chloramphenicol complex at high resolution.

Preferably, the portion of the coordinate data set forth in Table 7 (refer to attached CD-ROM) used to define the structure of a portion of a crystallized LRS-chloramphenicol complex corresponds to:

nucleotide coordinates 2044, 2430, 2431, 2479 and 2483-2485;

nucleotide coordinates 2044-2485;

HETATM coordinates 59925-59944; and

the coordinates set forth in Table 12 (derived from Table 7; refer to enclosed CD-ROM).

As described in Example 2 of the Examples section below, the following Table 7 (refer to attached CD-ROM) coordinates can be used to define the structure of the indicated LRS-chloramphenicol complex portions:

23S rRNA nucleotides associated with chloramphenicol: nucleotide coordinates 2044, 2430, 2431, 2479 and 2483-2485;

23S rRNA segment spanning nucleotides associated with chloramphenicol: nucleotide coordinates 2044-2485;

chloramphenicol: HETATM coordinates 59925-59944; and

the portion of the LRS-chloramphenicol complex, including the chloramphenicol-binding site of the LRS, contained within a 20 Å-radius sphere centered on a chloramphenicol atom: the set of coordinates set forth in Table 12.

Preferably, the coordinate data used to define the structure of a crystallized LRS-clindamycin complex, or of a portion thereof, is the set of coordinate data set forth in Table 8 (refer to attached CD-ROM), or a portion thereof

As described in Example 2 of the Examples section which follows, the coordinate data set forth in Table 8 (refer to attached CD-ROM) can be used to define the structure of the essentially complete LRS-clindamycin complex at high resolution.

Preferably, the portion of the coordinate data set forth in Table 8 (refer to attached CD-ROM) used to define the structure of a portion of a crystallized LRS-clindamycin complex corresponds to:

nucleotide coordinates 2040-2042, 2044, 2482, 2484 and 2590;

nucleotide coordinates 2040-2590;

HETATM coordinates 59922-59948; and

the coordinates set forth in Table 13 (derived from Table 8; refer to enclosed CD-ROM).

As described in Example 2 of the Examples section below, the following Table 8 (refer to attached CD-ROM) coordinates can be used to define the structure of the indicated LRS-clindamycin complex portions:

23S rRNA nucleotides associated with clindamycin: nucleotide coordinates 2040-2042, 2044, 2482, 2484 and 2590;

23S rRNA segment spanning nucleotides associated with clindamycin: nucleotide coordinates 2040-2590;

clindamycin: HETATM coordinates 59922-59948; and

portions of the LRS-clindamycin complex, including the clindamycin-binding site of the LRS, contained within a 20 Å-radius sphere centered on a clindamycin atom: the set of coordinates set forth in Table 13.

Preferably, the coordinate data used to define the structure of a crystallized LRS-clarithromycin complex, or of a portion thereof, is the set of coordinate data set forth in Table 9 (refer to attached CD-ROM), or a portion thereof.

As described in Example 2 of the Examples section which follows, the coordinate data set forth in Table 9 (refer to attached CD-ROM) can be used to define the structure of the essentially complete LRS-clarithromycin complex at high resolution.

Preferably, the portion of the coordinate data set forth in Table 9 (refer to attached CD-ROM) used to define the structure of a portion of a crystallized LRS-clarithromycin complex corresponds to:

nucleotide coordinates 2040-2042, 2045, 2484, 2588 and 2589;

nucleotide coordinates 2040-2589;

HETATM coordinates 59922-59973; and

the coordinates set forth in Table 14 (derived from Table 9; refer to enclosed CD-ROM).

As described in Example 2 of the Examples section below, the following Table 9 (refer to attached CD-ROM) coordinates can be used to define the structure of the indicated LRS-clarithromycin complex portions:

23S rRNA nucleotides associated with clarithromycin: nucleotide coordinates 2040-2042, 2045, 2484, 2588 and 2589;

23S rRNA segment spanning nucleotides associated with clarithromycin: nucleotide coordinates 2040-2589;

clarithromycin: HETATM coordinates 59922-59973; and

portions of the LRS-clarithromycin complex, including the clarithromycin-binding site of the LRS, contained within a 20 Å-radius sphere centered on a clarithromycin atom: the set of coordinates set forth in Table 14.

Preferably, the coordinate data used to define the structure of a crystallized LRS-erythromycin complex, or of a portion thereof, is the set of coordinate data set forth in Table 10 (refer to attached CD-ROM), or a portion thereof.

As described in Example 2 of the Examples section which follows, the coordinate data set forth in Table 10 (refer to attached CD-ROM) can be used to define the structure of the essentially complete LRS-erythromycin complex at high resolution.

Preferably, the portion of the coordinate data set forth in Table 10 (refer to attached CD-ROM) used to define the structure of a portion of a crystallized LRS-erythromycin complex corresponds to:

nucleotide coordinates 2040-2042, 2045, 2484, 2588 and 2589;

nucleotide coordinates 2040-2589;

HETATM coordinates 59922-59972; and

the coordinates set forth in Table 15 (derived from Table 10; refer to enclosed CD-ROM).

As described in Example 2 of the Examples section below, the following Table 10 (refer to attached CD-ROM) coordinates can be used to define the structure of the indicated LRS-erythromycin complex portions:

23S rRNA nucleotides associated with erythromycin: nucleotide coordinates 2040-2042, 2045, 2484, 2588 and 2589;

23S rRNA segment spanning nucleotides associated with erythromycin: nucleotide coordinates 2040-2589;

erythromycin: HETATM coordinates 59922-59972; and

portions of the LRS-erythromycin complex, including the erythromycin-binding site of the LRS, contained within a 20 Å-radius sphere centered on a erythromycin atom: the set of coordinates set forth in Table 15.

Preferably, the coordinate data used to define the structure of a crystallized LRS-roxithromycin complex, or of a portion thereof, is the set of coordinate data set forth in Table 11 (refer to attached CD-ROM), or a portion thereof.

As described in Example 2 of the Examples section which follows, the coordinate data set forth in Table 11 (refer to attached CD-ROM) can be used to define the structure of the essentially complete LRS-roxithromycin complex at high resolution.

Preferably, the portion of the coordinate data set forth in Table 11 (refer to attached CD-ROM) used to define the structure of a portion of a crystallized LRS-roxithromycin complex corresponds to:

nucleotide coordinates 2040-2042, 2045, 2484, 2588 and 2589;

nucleotide coordinates 2040-2589;

HETATM coordinates 59922-59979; and

the coordinates set forth in Table 16 (derived from Table 11; refer to enclosed CD-ROM).

As described in Example 2 of the Examples section below, the following Table 11 (refer to attached CD-ROM) coordinates can be used to define the structure of the indicated LRS-roxithromycin complex portions:

23S rRNA nucleotides associated with roxithromycin: nucleotide coordinates 2040-2042, 2045, 2484, 2588 and 2589;

23S rRNA segment spanning nucleotides associated with roxithromycin: nucleotide coordinates 2040-2589;

roxithromycin: HETATM coordinates 59922-59979; and

portions of the LRS-roxithromycin complex, including the roxithromycin-binding site of the LRS, contained within a 20 Å-radius sphere centered on a roxithromycin atom: the set of coordinates set forth in Table 16.

Preferably, the coordinate data used to define the structure of a crystallized LRS-ABT-773 complex, or of a portion thereof, is the set of coordinate data set forth in Table 18 (refer to attached CD-ROM), or a portion thereof.

As described in Example 3 of the Examples section which follows, the coordinate data set forth in Table 18 (refer to attached CD-ROM) can be used to define the structure of the essentially complete LRS-ABT-773 complex at high resolution.

Preferably, the portion of the coordinate data set forth in Table 18 (refer to attached CD-ROM) used to define the structure of a portion of a crystallized LRS-ABT-773 complex corresponds to:

nucleotide coordinates 803, 1773, 2040-2042, 2045, 2484, and 2588-2590;

nucleotide coordinates 803-2590;

HETATM coordinates 1-55; and

the coordinates set forth in Table 21 (derived from Table 18; refer to enclosed CD-ROM).

As described in Example 3 of the Examples section below, the following Table 18 (refer to attached CD-ROM) coordinates can be used to define the structure of the indicated LRS-ABT-773 complex portions:

23S rRNA nucleotides associated with ABT-773: nucleotide coordinates 803, 1773, 2040-2042, 2045, 2484, and 2588-2590;

23S rRNA segment spanning nucleotides associated with ABT-773: nucleotide coordinates 803-2590;

ABT-773: HETATM coordinates 1-55; and

portions of the LRS-ABT-773 complex, including the ABT-773-binding site of the LRS, contained within a 20 Å-radius sphere centered on a ABT-773 atom: the set of coordinates set forth in Table 21.

Preferably, the coordinate data used to define the structure of a crystallized LRS-azithromycin complex, or of a portion thereof, is the set of coordinate data set forth in Table 19 (refer to attached CD-ROM), or a portion thereof.

As described in Example 3 of the Examples section which follows, the coordinate data set forth in Table 19 (refer to attached CD-ROM) can be used to define the structure of the essentially complete LRS-azithromycin complex at high resolution.

Preferably, the portion of the coordinate data set forth in Table 19 (refer to attached CD-ROM) used to define the structure of a portion of a crystallized LRS-azithromycin complex corresponds to:

nucleotide coordinates 764, 2041, 2042, 2045, A2482, 2484, 2565, and 2588-2590;

nucleotide coordinates 764-2590;

amino acid residue coordinates Y59, G60, G63, T64, and R111;

HETATM coordinates 79705-79808; and

the coordinates set forth in Table 22 (derived from Table 19; refer to enclosed CD-ROM).

As described in Example 3 of the Examples section below, the following Table 19 (refer to attached CD-ROM) coordinates can be used to define the structure of the indicated LRS-azithromycin complex portions:

23S rRNA nucleotides associated with azithromycin: nucleotide coordinates 764, 2041, 2042, 2045, A2482, 2484, 2565, and 2588-2590;

ribosomal protein amino acid residues associated with azithromycin: Y59, G60, G63, T64, and R111

23S rRNA segment spanning nucleotides associated with azithromycin: nucleotide coordinates 764-2590;

azithromycin: HETATM coordinates 79705-79808; and

portions of the LRS-azithromycin complex, including the azithromycin-binding site of the LRS, contained within a 20 Å-radius sphere centered on a azithromycin atom: the set of coordinates set forth in Table 22.

Preferably, the coordinate data used to define the structure of a crystallized LRS-ACCP complex, or of a portion thereof, is the set of coordinate data set forth in Table 20 (refer to attached CD-ROM), or a portion thereof.

As described in Example 4 of the Examples section which follows, the coordinate data set forth in Table 20 (refer to attached CD-ROM) can be used to define the structure of the essentially complete LRS-ACCP complex at high resolution.

Preferably, the portion of the coordinate data set forth in Table 20 (refer to attached CD-ROM) used to define the structure of a portion of a crystallized LRS-ACCP complex corresponds to:

nucleotide coordinates 1924, 2430, 2485, 2532, 2533, 2534, 2552, 2562, and 2583;

nucleotide coordinates 1924-2583;

atom coordinates 78760-78855; and

the coordinates set forth in Table 25 (derived from Table 20; refer to enclosed CD-ROM).

As described in Example 4 of the Examples section below, the following Table 20 (refer to attached CD-ROM) coordinates can be used to define the structure of the indicated LRS-ACCP complex portions:

23S rRNA nucleotides associated with ACCP: nucleotide coordinates 1924, 2430, 2485, 2532, 2533, 2534, 2552, 2562, and 2583;

23S rRNA segment spanning nucleotides associated with ACCP: nucleotide coordinates 1924-2583;

ACCP: atom coordinates 78760-78855; and

portions of the LRS-ACCP complex, including the ACCP-binding site of the LRS, contained within a 20 Å-radius sphere centered on a ACCP atom: the set of coordinates set forth in Table 25.

Preferably, the coordinate data used to define the structure of a crystallized LRS-ASM complex, or of a portion thereof, is the set of coordinate data set forth in Table 21 (refer to attached CD-ROM), or a portion thereof.

As described in Example 4 of the Examples section which follows, the coordinate data set forth in Table 21 (refer to attached CD-ROM) can be used to define the structure of the essentially complete LRS-ASM complex at high resolution.

Preferably, the portion of the coordinate data set forth in Table 21 (refer to attached C D-ROM) used to define the structure of a portion of a crystallized LRS-ASM complex corresponds to:

nucleotide coordinates 1892, 1896, 1899, 1925, 1926, 2431, 2485, 2486, 2532, 2534, 2552, 2562, and 2581;

nucleotide coordinates 1892-2581;

amino acid residue coordinates 79-81;

atom coordinates 78747-79289; and

the coordinates set forth in Table 26 (derived from Table 21; refer to enclosed CD-ROM).

As described in Example 4 of the Examples section below, the following Table 21 (refer to attached CD-ROM) coordinates can be used to define the structure of the indicated LRS-ASM complex portions:

23S rRNA nucleotides associated with ASM: nucleotide coordinates 1892, 1896, 1899, 1925, 1926, 2431, 2485, 2486, 2532, 2534, 2552, 2562, and 2581;

23S rRNA segment spanning nucleotides associated with ASM: nucleotide coordinates 1892-2581;

ribosomal protein L16 amino acid residues associated with ASM: amino acid residue coordinates: 79-81

ASM: atom coordinates 78747-79289; and

portions of the LRS-ASM complex, including the ASM-binding site of the LRS, contained within a 40 Å-radius sphere centered on a ASM atom: the set of coordinates set forth in Table 26.

Preferably, the coordinate data used to define the structure of a crystallized LRS-ASMS complex, or of a portion thereof, is the set of coordinate data set forth in Table 22 (refer to attached CD-ROM), or a portion thereof.

As described in Example 4 of the Examples section which follows, the coordinate data set forth in Table 22 (refer to attached CD-ROM) can be used to define the structure of the essentially complete LRS-ASMS complex at high resolution.

Preferably, the portion of the coordinate data set forth in Table 22 (refer to attached CD-ROM) used to define the structure of a portion of a crystallized LRS-ASMS complex corresponds to:

nucleotide coordinates 1899, 1924, 1926, 2430, 2472, 2485, 2486, 2532, 2534, 2552, 2562, 2563, and 2581;

nucleotide coordinates 1924-2581;

amino acid residue coordinates 79-81;

atom coordinates 78758-79322; and

the coordinates set forth in Table 27 (derived from Table 22; refer to enclosed CD-ROM).

As described in Example 4 of the Examples section below, the following Table 22 (refer to attached CD-ROM) coordinates can be used to define the structure of the indicated LRS-ASMS complex portions:

23S rRNA nucleotides associated with ASMS: nucleotide coordinates 1899, 1924, 1926, 2430, 2472, 2485, 2486, 2532, 2534, 2552, 2562, 2563, and 2581;

23S rRNA segment spanning nucleotides associated with ASMS: nucleotide coordinates 1924-2581;

ribosomal protein L16 amino acid residues associated with ASM: amino acid residue coordinates: 79-81

ASMS: atom coordinates 78758-79322; and

portions of the LRS-ASMS complex, including the ASMS-binding site of the LRS, contained within a 40 Å-radius sphere centered on a ASMS atom: the set of coordinates set forth in Table 27.

Preferably, the coordinate data used to define the structure of a crystallized LRS-sparsomycin complex, or of a portion thereof, is the set of coordinate data set forth in Table 23 (refer to attached CD-ROM), or a portion thereof.

As described in Example 4 of the Examples section which follows, the coordinate data set forth in Table 23 (refer to attached CD-ROM) can be used to define the structure of the essentially complete LRS-sparsomycin complex at high resolution.

Preferably, the portion of the coordinate data set forth in Table 23 (refer to attached CD-ROM) used to define the structure of a portion of a crystallized LRS-sparsomycin complex corresponds to:

nucleotide coordinate 2581;

atom coordinates 78757-78778, and

the coordinates set forth in Table 28 (derived from Table 23; refer to enclosed CD-ROM).

As described in Example 4 of the Examples section below, the following Table 23 (refer to attached CD-ROM) coordinates can be used to define the structure of the indicated LRS-sparsomycin complex portions:

23S rRNA nucleotides associated with sparsomycin: nucleotide coordinate 2581;

sparsomycin: atom coordinates 78757-78778; and

portions of the LRS-sparsomycin complex, including the sparsomycin-binding site of the LRS, contained within a 20 Å-radius sphere centered on a sparsomycin atom: the set of coordinates set forth in Table 28.

Preferably, the coordinate data used to define the structure of a crystallized LRS-troleandomycin complex, or of a portion thereof, is the set of coordinate data set forth in Table 38 (refer to attached CD-ROM), or a portion thereof.

As described in Example 5 of the Examples section which follows, the coordinate data set forth in Table 38 (refer to attached CD-ROM) can be used to define the structure of the essentially complete LRS-troleandomycin complex at high resolution.

Preferably, the portion of the coordinate data set forth in Table 38 (refer to attached CD-ROM) used to define the structure of a portion of a crystallized LRS-troleandomycin complex corresponds to:

nucleotide coordinates 759, 761, 803, 2041, 2042, 2045, 2484, and 2590;

nucleotide coordinates 759-2590;

amino acid residue coordinate Ala2;

atom coordinates 1-57; and

the coordinates set forth in Table 40 (derived from Table 38; refer to enclosed CD-ROM).

As described in Example 5 of the Examples section below, the following Table 38 (refer to attached CD-ROM) coordinates can be used to define the structure of the indicated LRS-troleandomycin complex portions:

23S rRNA nucleotides associated with troleandomycin: nucleotide coordinates 759, 761, 803, 2041, 2042, 2045, 2484, and 2590;

23S rRNA segment spanning nucleotides associated with troleandomycin: nucleotide coordinates 759-2590;

ribosomal protein L32 amino acid residues associated with ASM: amino acid residue coordinate Ala2;

troleandomycin: atom coordinates 1-57; and

portions of the LRS-troleandomycin complex, including the troleandomycin-binding site of the LRS, contained within a 20 Å-radius sphere centered on a troleandomycin atom: the set of coordinates set forth in Table 40.

Thus, the LRS-antibiotic crystals of the present invention can be used to derive coordinate data used for modeling the high resolution structures of antibiotic-binding pockets of LRSs and for modeling the high resolution structures of the interactions between LRSs and antibiotics.

Since, as described above, the coordinate data of the present invention can be used to define structures of free LRSs, or portions thereof, such coordinate data can be used to generate models of the structure of free LRSs, or portions thereof, as described in Example 1 of the Examples section below.

Thus, according to still another aspect of the present invention, there are provided models of the three-dimensional atomic structures of free LRSs, or portions thereof.

It will be understood by one of ordinary skill in the art that such models can be used to represent selected three-dimensional structures of the free LRSs of the present invention and to thereby assign particular functions to particular structures represented by such models and to perform comparative structure/function analyses of different LRSs, or portions thereof, or to design or identify a molecule binding a desired portion thereof.

As described hereinabove, the coordinate data of the present invention define the essentially complete structure of crystallized free eubacterial LRSs at a resolution as high as 3.1 Å, whereas the highest prior art such resolution was 5.5 Å, and whereas no satisfactorily complete prior art structures of LRSs of any type have been defined at a resolution of 3.1 Å or higher.

Thus, it will be appreciated that the highly resolved coordinate data of the present invention define significantly more accurately the three-dimensional structure of free LRSs, or portions thereof than the prior art. As such, the free LRS structure models of the present invention are distinctly superior to such prior art models in representing the structure of free LRSs. Thus, the free LRS structure models of the present invention are thereby also distinctly superior relative to such prior art models with regards to enabling elucidation of structural-functional relationships of free LRS. This is abundantly demonstrated by the wealth of novel structural-functional features of free LRSs which can now be described for the first time using the highly resolved data of the present invention, examples of which are described at length in the Examples section below, for example in Tables 4 and 5.

Thus, the models of the present invention can be used to provide novel and far-reaching insights into the crucial and universal mechanisms of protein production which are performed by the ribosome.

The models of the structures of free LRSs, or portions thereof, of the present invention can be exploited in several ways.

For example, data relating to free LRSs uncovered by the present invention can be used to design LRSs having desired characteristics, such as the capacity to drive high levels of recombinant protein synthesis. Such modified LRSs could thus be of value, for example, for enhancing recombinant protein production by bacteria, an area of potentially great economic and scientific benefit. Such functional modification could be achieved, for example, by using the models of the present invention to identify features of the LRS which negatively regulate protein synthesis and designing and modeling desired functional alterations. For example, features which sterically hinder growth of the nascent polypeptide or features which sterically hinder or limit tRNA processes could be altered so as not to cause such steric hindrance, thereby potentially enhancing protein production by ribosomes comprising such modified LRSs.

Coordinate data defining structures of antibiotic-LRS complexes, or portions thereof, at high resolution can be used to generate models of the structure of antibiotic-LRS complexes, or portions thereof, as described in Examples 2-5 of the Examples section below.

Thus, according to a further aspect of the present invention, there are provided models of the structures of antibiotic-eubacterial LRS complexes, or portions thereof.

Such models, being completely novel and unprecedented in the prior art, enable, for the first time, elucidation of the precise structural-functional basis of the interactions between antibiotics and LRSs, as described in extensive detail in Examples 2-5 of the Examples section below, for a broad range of antibiotics, as detailed hereinabove.

By virtue of illuminating the precise structural-functional interactions between antibiotics and LRSs, the high resolution models of the three-dimensional structure of the antibiotic-LRS complexes of the present invention constitute a unique and highly potent tool enabling the rational design or identification of putative antibiotics, such as antibiotics effective against antibiotic resistant pathogens.

Thus, according to yet a further aspect of the present invention, there is provided a method of identifying a putative antibiotic.

The method of identifying a putative antibiotic is effected by obtaining a set of structure coordinates defining the structure of a crystallized antibiotic-binding pocket of a free LRS or, more preferably, of a crystallized antibiotic-complexed LRS and, preferably computationally, screening a plurality of compounds for a compound capable of specifically binding the antibiotic-binding pocket, thereby identifying the putative antibiotic. Preferably, the method further comprises the steps of contacting the putative antibiotic with the antibiotic-binding pocket and detecting specific binding of the putative antibiotic to the antibiotic-binding pocket, thereby qualifying the putative antibiotic.

It will be appreciated, in this case, that qualification of a large number of putative antibiotic compounds can be used to provide data as to particular structural regions thereof which contribute to binding, thus enabling design of compounds which exhibit efficient binding.

Various methods of computationally screening compounds capable of specifically binding a set of atoms whose atomic positioning and structure is modeled, such as the antibiotic-binding pockets of the present invention, are well known to skilled artisans (see, for example, Bugg et al., 1993. Scientific American December:92; West et al., 1995. TIPS. 16, 67; Dunbrack et al., 1997. Folding and Design 2, 27).

For example, potential antibiotic-binding pocket-binding compounds can be examined through the use of computer modeling using a docking program such as GRAM, DOCK, or AUTODOCK (Dunbrack et al., 1997. Folding and Design 2, 27). Using such programs, one may predict or calculate the orientation, binding constant or relative affinity of a given compound to an antibiotic-binding pocket, and use that information to design or select compounds of the desired affinity. Using such methods, a database of chemical structures is searched and computational fitting of compounds to LRSs is performed to identify putative antibiotics containing one or more functional groups suitable for the desired interaction with the nucleotides comprising the antibiotic-binding pocket. Compounds having structures which best fit the points of favorable interaction with the three-dimensional structure are thus identified. Thus, these methods ascertain how effectively candidate compounds mimic the binding of antibiotics to antibiotic-binding pockets. Generally the tighter the fit (e.g., the lower the steric hindrance, and/or the greater the attractive force) the more potent the putative antibiotic will be.

Molecular docking programs may also be effectively used in conjunction with structure modeling programs (see hereinbelow). One important advantage of using computational techniques when selecting putative antibiotics is that such techniques can provide antibiotics with high binding specificity which are less likely to interfere with mammalian protein synthesis and/or cause side-effects.

Using computational approaches, compounds can furthermore be systematically modified by molecular modeling programs until promising putative antibiotics are generated. This technique has been shown, for example, to be effective in the development of HIV protease inhibitors (Lam et al., 1994. Science 263, 380; Wlodawer et al., 1993. Ann Rev Biochem. 62, 543; Appelt, 1993. Perspectives in Drug Discovery and Design 1, 23; Erickson, 1993. Perspectives in Drug Discovery and Design 1, 109) and hence of providing for the first time an apparent cure for AIDS.

Thus, the use of computational screening enables large numbers of compounds to be rapidly screened and produces small numbers of putative antibiotics without the requirement of resorting to the laborious synthesis of large numbers of compounds inherent to chemical synthesis techniques.

Once putative antibiotics are computationally identified they can either be obtained from chemical libraries, such as those held by most large chemical companies, including Merck, Glaxo Welcome, Bristol Meyers Squib, Monsanto/Searle, Eli Lilly, Novartis and Pharmacia UpJohn. Alternatively, putative antibiotics may be synthesized de novo, a feasible practice in rational drug design due to the small numbers of promising compounds produced via computer modeling.

Putative antibiotics can be tested for their ability to bind antibiotic-binding pockets in any standard, preferably high throughput, binding assay, via contact with target LRSs or portions thereof comprising antibiotic-binding pockets. Alternatively putative antibiotics can be functionally qualified for antibiotic activity, for example, via testing of their ability to inhibit growth of target bacterial strains in-vitro or in-vivo or to inhibit protein synthesis by target LRSs in-vitro. When suitable putative antibiotics are identified, further NMR structural analysis can optionally be performed on binding complexes formed between the antibiotic-binding pocket and the putative antibiotic.

For all of the putative antibiotic screening assays described herein, further necessary refinements to the structure of the putative antibiotic may be performed by successive iteration of any and/or all of the steps provided by the particular screening assay.

Putative antibiotics may also be generated in-vitro, for example, by screening random peptide libraries produced by recombinant bacteriophages (Scott and Smith, 1990. Science 249, 386; Cwirla et al., 1990. Proc Natl Acad Sci USA. 87, 6378; Devlin et al., 1990. Science 249, 404) or chemical libraries.

Phage libraries for screening have been constructed such that when infected therewith, host E. coli produce large numbers of random peptide sequences of about 10-15 amino acids (Parmley and Smith, 1988. Gene 73, 305, Scott and Smith, 1990. Science 249, 386). In one such method, phages are mixed at low dilution with permissive E. coli strains in low melting point LB agar which is then overlayed on LB agar plates. Following incubation at 37 degrees centigrade, small clear plaques in a lawn of E. coli form representing active phage growth. These phages are then adsorbed in their original positions onto nylon filters which are placed in washing solutions to block any remaining adsorbent sites. The filters can then be placed in a solution containing, for example, a radiolabelled LRS, or portion thereof, comprising an antibiotic-binding pocket. Following incubation, filters are thoroughly washed and developed for autoradiography. Plaques containing phages that bind to radiolabelled LRSs can then be conveniently identified and the phages further cloned and retested for LRS binding capacity. Following isolation and purification of LRS-binding phages, amino acid sequences of putative antibiotics can be deduced via DNA sequencing and employed to produce synthetic peptides.

Promising putative antibiotic peptides can then be easily synthesized in large quantities for clinical use. It is a significant advantage of this method that synthetic peptide production is relatively non-labor intensive, facile, and easily quality-controlled, such that large quantities of peptide antibiotics could be produced economically (see, for example, Patarroyo, 1990. Vaccine, 10, 175).

In another screening assay, a LRS, or portion thereof, comprising an antibiotic-binding pocket is bound to a solid support, for example, via biotin-avidin linkage and a candidate compound is allowed to equilibrate therewith to test for binding thereto. Generally, the solid support is washed and compounds that are retained are selected as putative antibiotics. In order to facilitate visualization, compounds may be labeled, for example, by radiolabeling or with fluorescent markers.

Another highly effective means of testing binding interactions is via surface plasmon resonance analysis, using, for example, commercially available BIAcore chips (Pharmacia). Such chips may be coated with either the LRS, or portion thereof comprising an antibiotic-binding pocket, or with the putative antibiotic, and changes in surface conductivity are then measured as a function of binding affinity upon exposure of one member of the putative binding pair to the other member of the pair.

It will be understood by one of ordinary skill in the art that rational design of LRSs can be easily effected, for example, via recombinant DNA technology or via chemical techniques.

Thus, the antibiotic-LRS complex three-dimensional structure models of the present invention can be efficiently used by one of ordinary skill in the art to obtain novel antibiotics.

The antibiotic-LRS complexes of the present invention can further be advantageously employed to elucidate the structural basis for the effectiveness of novel antibiotics, such antibiotics derived by chemical modification from established classes of antibiotics. As such, the antibiotic-LRS complexes of the present invention generate vital information required for designing antibiotics effective against bacterial pathogens having developed antibiotic resistance, of which the emergence represents an expanding epidemiological threat, as described above.

As shown in Example 3 of the Examples section below, the models of the present invention can be used to elucidate the structural basis for the ability of antibiotics (e.g., ABT-773 and azithromycin) derived by modification of a class of antibiotics (e.g. macrolides) to exert antibacterial activity against bacteria having developed resistance to the unmodified class antibiotics.

As well as providing a means whereby novel antibiotics can be obtained, the antibiotic-LRS complex three-dimensional structure models of the present invention provide novel information illuminating the mechanisms of LRS function per se, since antibiotics function as inhibitors of, and hence as probes of LRS function, as described in extensive ground-breaking detail in Examples 25 of the Examples section below. For example, as shown in Examples 4 and 5, respectively, of the following Examples section, the models of the present invention can be used to elucidate critical aspects of the structural basis of peptidyl transferase activity, or to elucidate the structural basis underlying the regulatory functions of portions of the ribosome, such as the protein exit tunnel.

The antibiotic-LRS complex three-dimensional structure models of the present invention further provide novel information illuminating the mechanisms of LRS function since, as described above and in Example 4 of the Examples section which follows, antibiotics such as ACCP, ASM or ASMS constitute ribosomal substrate inhibitors. As is illustrated in Example 4 of the Examples section which follows, ACCP-, ASM- and ASMS-LRS models of the present invention enable, for the first time elucidation of the precise structural-functional basis for the interaction between ribosomal substrates and eubacterial LRSs at high resolution.

The models of the present invention also serve as a valuable tool for solving related atomic structures.

In particular, the models of the structure of free LRSs and of antibiotic-LRS complexes of the present invention can be utilized, respectively, to facilitate solution of the three-dimensional structures of free LRSs, of antibiotic-LRS complexes, and of ribosomal substrate-LRS complexes, or portions thereof, which are similar to those of the present invention.

This can be effected, preferably computationally via molecular replacement. In molecular replacement, all or part of a model of a free LRSs or of an antibiotic-LRS complex of the present invention is used to determine the structure of a crystallized macromolecule or macromolecular complex having a closely related but unknown structure. This method is more rapid and efficient than attempting to determine such information ab initio. Solution of an unknown structure by molecular replacement involves obtaining X-ray diffraction data for crystals of the macromolecule or macromolecular complex for which one wishes to determine the three-dimensional structure. The three-dimensional structure of a macromolecule or macromolecular complex whose structure is unknown is obtained by analyzing X-ray diffraction data derived therefrom using molecular replacement techniques with reference to the structural coordinates of the present invention as a starting point to model the structure thereof, as described in U.S. Pat. No. 5,353,236, for instance. The molecular replacement technique is based on the principle that two macromolecules which have similar structures, orientations and positions in the unit cell diffract similarly. Molecular replacement involves positioning the known structure in the unit cell in the same location and orientation as the unknown structure. Once positioned, the atoms of the known structure in the unit cell are used to calculate the structure factors that would result from a hypothetical diffraction experiment. This involves rotating the known structure in the six dimensions (three angular and three spatial dimensions) until alignment of the known structure with the experimental data is achieved. This approximate structure can be fine-tuned to yield a more accurate and often higher resolution structure using various refinement techniques. For instance, the resultant model for the structure defined by the experimental data may be subjected to rigid body refinement in which the model is subjected to limited additional rotation in the six dimensions yielding positioning shifts of under about 5%. The refined model may then be further refined using other known refinement methods.

According to one embodiment, the coordinates of the present invention can be used to model atomic structures defined by a set of structure coordinates having a root mean square deviation of not more than 2.0 Å, more preferably of not more than 1.0 Å, and most preferably of not more than 0.5 Å from a set of structure coordinates of the present invention.

Further utilities of the models of the present invention include computational identification or rational design of potentially antibiotic resistant forms of LRSs followed by identification or rational design of putative antibiotics effective against such modified LRSs, thereby providing banks of antibiotics potentially useful against future outbreaks of bacterial pathogens bearing such antibiotic resistant LRSs. The structure models of the present invention, or portions thereof, can further be utilized to computationally identify RNA bases or amino acids within the three-dimensional structure thereof, preferably within or adjacent to an antibiotic-binding pocket; to generate and visualize a molecular surface, such as a water-accessible surface or a surface comprising the space-filling van der Waals surface of all atoms; to calculate and visualize the size and shape of surface features of free LRSs or antibiotic-LRS complexes, to locate potential H-bond donors and acceptors within the three-dimensional structure, preferably within or adjacent to an antibiotic-binding pocket; to calculate regions of hydrophobicity and hydrophilicity within the three-dimensional structure, preferably within or adjacent to an antibiotic-binding pocket; and to calculate and visualize regions on or adjacent to the protein surface of favorable interaction energies with respect to selected functional groups of interest (e.g., amino, hydroxyl, carboxyl, methylene, alkyl, alkenyl, aromatic carbon, aromatic rings, heteroaromatic rings, substituted and unsubstituted phosphates, substituted and unsubstituted phosphonates, substituted and unsubstituted fluoro and difluorophosphonates; etc.).

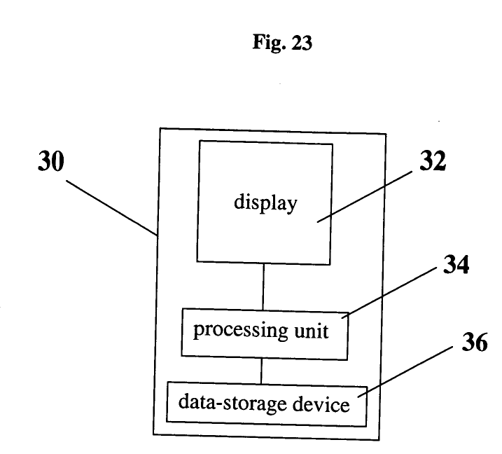

The structure models of the present invention are preferably generated by a computing platform 30 (FIG. 23) which generates a graphic output of the models via display 32. The computing platform generates graphic representations of atomic structure models via processing unit 34 which processes structure coordinate data stored in a retrievable format in data storage device 36. Examples of computer readable media which can be used to store coordinate data include conventional computer hard drives, floppy disks, DAT tape, CD-ROM, and other magnetic, magneto-optical, optical, floptical, and other media which may be adapted for use with computing platform 30.

Suitable software applications, well known to those of skill in the art, which may be used by processing unit 34 to process structure coordinate data so as to provide a graphic output of three-dimensional structure models generated therewith via display 32 include RIBBONS (Carson, M., 1997. Methods in Enzymology 277, 25), O (Jones, T A. et al., 1991. Acta Crystallogr A47, 110), DINO (DINO: Visualizing Structural Biology (2001) http://www.dino3d.org); and QUANTA, CHARMM, INSIGHT, SYBYL, MACROMODE, ICM, MOLMOL, RASMOL and GRASP (reviewed in Kraulis, J., 1991. Appl Crystallogr. 24, 946).

Preferably, the software application used to process coordinate data is RIBBONS.

Preferably the structure coordinates of the present invention are in PDB format for convenient processing by such software applications. Most or all of these software applications, and others as well, are downloadable from the World Wide Web.

Thus, the present invention provides novel and highly resolved three-dimensional structural data of a bacterial LRS, thereby providing for the first time, tools enabling the design and testing of novel antibiotic compounds as well as tools which can be used to predict the effect of changes in LRS structure on antibiotic-binding efficiencies and the like.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al., (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al., (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Growth of Eubacterial LRS Crystals and Solution of the Complete Three-Dimensional Atomic Structure of the Eubacterial LRS at 3.1 Å Resolution

The ability to generate three-dimensional models of bacterial LRS structure and function at the atomic level would be extremely useful since the ribosome is responsible for the central biological process of protein production and serves as the main binding target for a broad range of antibiotics. Three-dimensional atomic structure models of the LRS could be of significant utility for elucidating mechanisms of ribosome function. Such models could constitute a powerful tool for the rational design or identification of antibiotics, a vital need in light of expanding epidemics of diseases caused by antibiotic resistant microorganisms. Furthermore these models could be employed to rationally design or select ribosomes having desired characteristics, such as, for example, enhanced protein production capacity when expressed in bacterial strains which would be of great benefit, for example, for improving recombinant protein production. All prior art approaches, however, have failed to produce satisfactory three-dimensional atomic models of LRS such as, for example, for drug design or drug improvement. In order to fulfill these important needs, therefore, high resolution three-dimensional atomic structure models of bacterial LRSs were generated while reducing the present invention to practice, as follows.

Materials and Methods:

Cell culture: D. radiodurans bacteria were cultured as recommended by the American Tissue-Type Culture Collection (ATCC), using ATCC Medium No. 679 with minor modifications.

Amino acid sequencing of D. radiodurans large ribosomal subunit proteins: D. radiodurans LRS proteins were separated by two-dimensional polyacrylamide gel electrophoresis and identified via sequencing analysis of their five N-terminal amino acids.

Determination of D. radiodurans 23S and 5S rRNA secondary structure: Secondary structure diagrams were constructed for the 23S and 5S rRNA chains of D50S, guided by their sequences and by the available diagram for the RNA of the 50S subunit from T. thermophilus (T50S; Gutell, R. (1996) In Ribosomal RNA: Structure, Evolution, Processing and Function in Protein Biosynthesis., A. E. Z. Dahlberg, R. A., eds, ed. (FL, USA: CRC Press, Boca Raton), pp. 111-128).

Crystallization of the large ribosomal subunit of D. radiodurans: Ribosomes and their subunits were prepared as previously described (Noll, M. et al., 1973. J Mol Biol. 75, 281) and suspended in solutions containing H-I buffer (10 mM MgCl₂, 60 mM NH₄Cl, 5 mM KCI, 10 mM HEPES pH 7.8), a buffer optimized for testing in-vitro functional activity of ribosomes, supplemented with 0.1-1% (v/v) of a solution comprising monovalent and multivalent alcohols (typically comprising the monovalent and multivalent alcohols dimethylhexandiol and ethanol, respectively, at a ratio dimethylhexandiol to ethanol of 1:3.56) as a precipitant. Ribosome suspensions were equilibrated against equilibration solutions comprising the same buffer components as the solutions in which the ribosomes were suspended but containing half the amount of alcohols thereof, as previously described (Yonath A. et al., 1983. FEBS Lett. 163, 69; Yonath, A. et al., 1982. Journal of Cellular Biochemistry 19, 145). Crystals were grown in hanging drops using standard Linbro dishes, via vapor diffusion at 18 degrees centigrade. For optimizing crystal growth, it was necessary to determine the exact conditions for every preparation individually. The same, or similar, divalent alcohols (e.g. ethyleneglycol) were used as cryoprotectants for flash-freezing of the crystals in liquid propane.

Heavy-atom derivatives of D50S were prepared by soaking D50S crystals in 1-2 mM of iridium pentamide or K₅H(PW₁₂O₄₀)12H₂O for 24 hours.

Collection of X-ray diffraction data: Experimental MIRAS phases were obtained from anomalous data using heavy atom derivatives of D50S crystals. The tungsten and iridium sites were obtained from difference Patterson, residual and difference Fourier maps. To remove potential bias of W₁₂, the density modification procedure was altered to gradually include the low-resolution terms.

X-ray diffraction data was collected at 95 K with well collimated X-ray beams at high brightness synchrotron (SR) stations (ID14/European Synchrotron Radiation Facility (ESRF)/European Molecular Biology Laboratory (EMBO) and ID19/Argonne Photon Source (APS)). Data was recorded on image-plates (MAR 345) or CCD (Mar, Quantum 4, or APS2), processed with DENZO and reduced with SCALEPACK (Otwinowski, Z. and Minor, W., 1997. Macromolecular Crystallography Pt A. 276, 307) and the CCP4 package (Bailey, S., 1994. Acta Cryst D. 50, 760). Crystals were screened at BW6/MPG and BW7/EMBL at Deutsches Elektronen-Synchrotron (DESY).

Phase determination: The initial electron density maps were obtained by molecular replacement searches using AmoRe (Navazza J., 1994. Acta Crystallogr. A50, 57), using the structure of H50S determined by us and others (Yonath et al., 1998. Acta Crystallogr. A, 54, 945; Ban, N. et al., 2000. Science 289, 905) as a basis for the search model. The phases obtained from the MR solution (correlation coefficient, calculated using intensities=45.8% and contrast to next solution=1.5) were subjected to several cycles of density modification using SOLOMON (Abrahams, J. P. and Leslie, A. G. W., 1996. Acta Cryst D. 52, 30). The resulting map was sufficiently clear to model a significant part of D50S but MIRAS phasing by heavy metal atoms was still required for tracing of the RNA chains and of the proteins. The inclusion of phase information obtained from the two heavy-atom derivatives substantially improved the quality of the electron density.

Some of the D50S proteins were localized using structural homology with the available high resolution structure of H50S as a guide. The high level of homology existing between D. radiodurans and E. coli LRSs and existing knowledge concerning their relative positions (Wittmann, H. G., 1983. Annu Rev Biochem. 52, 35; Walleczek, J. et al., 1989. Biochemistry 28, 4099) were used for initial placement of most of the proteins, including L7, L10, L11, L17, CTC (L25 in E. coli), L27, L28 and L31-L36 that do not exist in H50S and to model their interactions with rRNA (Ostergaard, P. et al., 1998. J Mol Biol. 284, 227). Coordinates determined for the isolated proteins were also used (Golden, B. L. et al., 1993. Embo J. 12, 4901; Wimberly, B. T. et al., 1999. Cell 97, 491; GuhaThakurta, D., and Draper, D. E., 2000. J Mol Biol. 295, 569; Fedorov, R. et al., 1999. Acta Cryst D. 55, 1827; Fedorov, R. et al., 2001. Acta Cryst D. 57, 968; Worbs, M. et al., 2000. Embo J. 19, 807; Unge, J. et al., 1998. Structure 6, 1577; Nikonov, S. et al., 1996. Embo J. 15, 1350; Hoffman, D. W. et al., 1996. J Mol Biol. 264, 1058; Davies, C. et al., 1996. Structure 4, 55; Wahl, M. C. et al., 2000. Embo J. 19, 174; Hard, T. et al., 2000. J Mol Biol. 296, 169).

Docking procedure: The A-, P- and E-site tRNA molecules were positioned on the LRS as previously described (Schlüenzen, F. et al., 2000. Cell 102, 615) in the same relative orientation as was observed in the 5.5 Å resolution structure of the T70S ribosome (Yusupov M M., 2001. Science 292, 883).

Experimental Results:

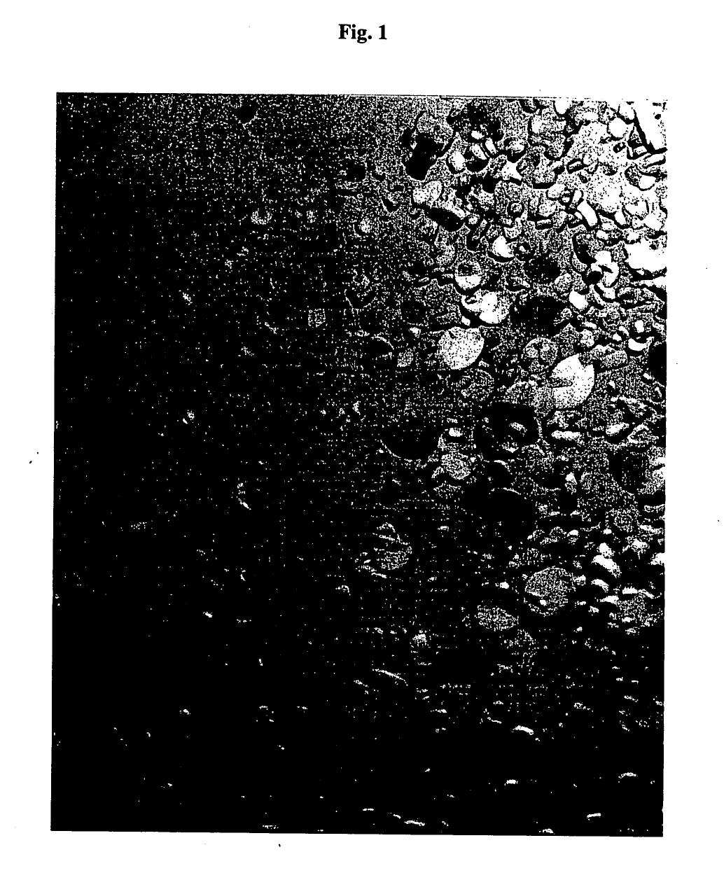

Generation of eubacterial large ribosomal subunit crystals: D50S crystals, having an ovo-discoidal shape were grown (FIG. 1).

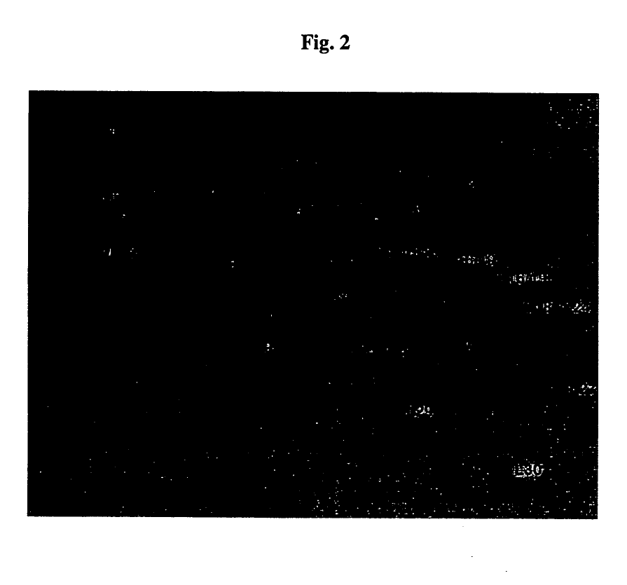

Amino acid sequences of D. radiodurans large ribosomal subunit proteins: Ribosomal proteins identified via two-dimensional PAGE and by sequencing the five N-terminal amino acids of each protein are shown in FIG. 2. Upon comparison to reported sequences in the TIGR database (White, O. et al., 1999. Science 286, 1571), two discrepancies were identified; one in the sequence of CTC starting at amino acid residue 19 and the other in L6 starting at amino acid residue 30 of the predicted sequence. These results constituted the first such amino acid-based sequencing of D. radiodurans LRS proteins.

Eubacterial large ribosomal subunit structure determination: The three-dimensional atomic structure of D50S was determined and refined to 3.1 Å by generating structural coordinates using data derived from X-ray crystallography of native D50S (Table 1) and heavy atom derivatives thereof (Table 2).

TABLE 1
Native D. radiodurans LRS crystallographic data.

| Data | Resolution | No. of unique | Rsym | Completeness | |
|---|---|---|---|---|---|
| set | (Å) | reflections | (%) | (%) | <I/sig (I)> |
| 1 | 50-3.0 | 178,685 | 11.8 | 49.9 | 4.5 |
| | | | (69.6) | (42.1) | (1.5) |
| 2 | 50-3.1 | 400,658 | 15.9 | 92.3 | 12.0 |
| | | | (44.4) | (77.3) | (1.5) |

The three-dimensional atomic structure of portions, or combination of portions, of crystallized D50S are defined by atom coordinates (D. radiodurans numbering system) set forth in Table 3 (refer to enclosed CD-ROM), as follows:

D50S: 1-65345;

23S rRNA: 1-59360;

5S rRNA: 59361-61880;

ribosomal protein L2: 61881-62151;

ribosomal protein L3: 62152-62357;

ribosomal protein L4: 62358-62555;

ribosomal protein L5: 62556-62734;

ribosomal protein L6: 62735-62912;

ribosomal protein L9: 62913-62965;

ribosomal protein L11: 62966-63109;

ribosomal protein L13: 63110-63253;

ribosomal protein L14: 63254-63386;

ribosomal protein L15: 63387-63528;

ribosomal protein L16: 63529-63653;

ribosomal protein L17: 63654-63768;

ribosomal protein L18: 63769-63880;

ribosomal protein L19: 63881-64006;

ribosomal protein L20: 64007-64122;

ribosomal protein L21: 64123-64223;

ribosomal protein L22: 64224-64354;

ribosomal protein L23: 64355-64448;

ribosomal protein L24: 64449-64561;

ribosomal protein CTC: 64562-64785;

ribosomal protein L27: 64786-64872;

ribosomal protein L28: 64873-64889;

ribosomal protein L29: 64890-64955;

ribosomal protein L30: 64956-65011;

ribosomal protein L31: 65012-65085;

ribosomal protein L32: 65086-65144;

ribosomal protein L33: 65145-65198;

ribosomal protein L34: 65199-65245;

ribosomal protein L35: 65246-65309; and

ribosomal protein L36: 65310-65345.

TABLE 2
D50S heavy atom derivative crystallographic data.

| | | | Unit cell | | Complete- | |
|---|---|---|---|---|---|---|
| Heavy | No. of | Resolution | dimensions | Rsym | ness | <I/sig |
| atom | sites | (Å) | (Å) | (%) | (%) | (I)> |
| Penta-Ir | 56 | 50-4.0 | 170.24 × | 14.5 | 93.9 | 5.8 |
| | | | 410.54 × | (47.2) | (90.9) | (1.9) |
| | | | 696.52 | | | |
| W₁₂ | 4 × 12 | 30-6.0 | 170.15 × | 8.1 | 71.9 | 14.1 |
| | | | 408.65 × | (12.9) | (69.2) | (5.0) |
| | | | 696.52 | | | |

FOM = 0.58 post-phasing (SHARP), and 0.78 post-density modification with SOLOMON.
Space group = I222; Unit cell dimensions: 170.827 × 409.430 × 695.597 Å;
R_{factor} = 25.6%;
R_{free} = 28.3%; Highest resolution bins are in parentheses.

Atomic coordinates defining the three-dimensional atomic structure of D50S were deposited in the protein data bank (PDB) under accession code 1LNR.

The fold of the 23S and 5S rRNA chains manually traced in electron density maps was found to be in remarkable agreement to that predicted. Only in a few instances did the base-pairing system deviate from the predicted scheme, one example being the predicted base pair near the loop of H81 (C2243-G2255, in the D. radiodurans numbering system) which was found to be flipped out in the three-dimensional structure of D50S.

Essentially all (96%) of the 23S rRNA nucleotides and 30 of the 33 D50S proteins were traced in the electron density map and found to be mostly ordered. The remaining three proteins were successfully placed, and portions thereof resolved, and the locations of several metals and hydrated Mg2+ ions were identified.

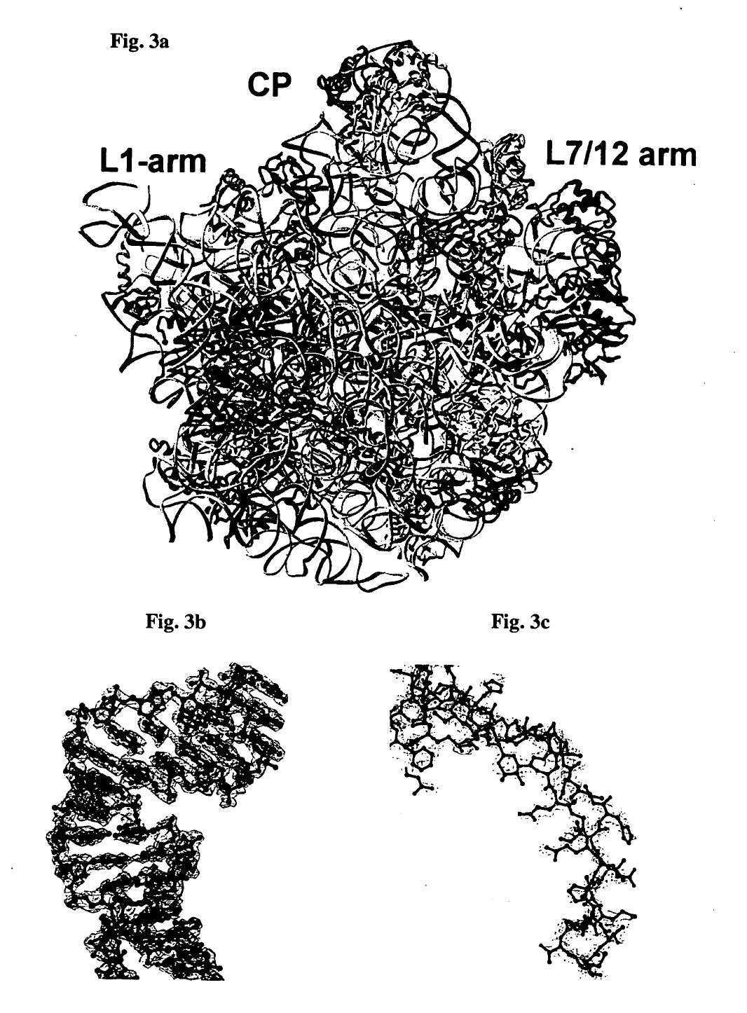

Overall structure of D50S: The traditional shape of the LRS, as seen by electron microscopy, contains a massive core with a central elongated feature and two lateral protuberances, termed the L1 and L7/L12 stalks. The view referred to as the “crown view”, has the appearance of a halved pear with two protuberances. Its “flat” surface, facing the viewer in FIG. 3, is adjacent to the small subunit in the 70S ribosome and its globular distal side faces the solvent. The overall shape of D50S is generally similar to this consensus view. Typical map segments of an rRNA helix and of a protein chain are shown in FIGS. 1b and 1c, respectively.

As in all other models of the LRS, the 23S rRNA molecule forms the bulk of the structure and the small 5S rRNA molecule forms most of an elongated feature in the center of the “crown”. At the secondary structure level the two RNA chains form seven domains and, although each of the domains has a unique three-dimensional shape, together they produce a compact single intertwined core, in contrast to the domain-like design of the 30S subunit (Schlüenzen, F. et al., 2000. Cell 102, 615; Wimberly, B. T. et al., 2000. Nature 407, 327).

Superimposition of the D50S structure onto that of H50S (Ban, N. et al., 2000. Science 289, 905) and onto that of the 50S subunit within the 5.5 Å structure of the T70S ribosome (Yusupov, M. M. et al., 2001. Science 292, 883) revealed similarities between the rRNA folds thereof, as previously observed between all known 50S structures. Despite these similarities significant structural differences were detected. It was discovered that the central regions of all three structures were similar in all structures, however, even within conserved regions structural differences were detected in RNA bulges, hairpin loops, structural motifs such as the loop-E (Leontis, N. B. and Westhof, E., 1998. J Mol Biol. 283, 571) and the base-pairing scheme, which cannot be explained by the expected phylogenetic variations. Some of these features are located at strategic locations and cause significant changes in the local architecture which may propagate towards the periphery. Thus, several surface regions in D50S appear to be unique relative to other 50S structures, even when the local environment of individual features is similar. In order to pinpoint the meaningful differences, the model was divided into individual structural elements, each containing a few neighboring helices and junctions, and the environment of each element was analyzed separately, both visually and by computing the internal distances separating features therein (Tables 4 and 5). In this way, significant differences unrelated to the sequences were discovered, of which some can be related to ribosomal function.

TABLE 4
Characterization of D50S proteins

| | DNA [symbol] | | Contacts with | | | | | | |
|---|---|---|---|---|---|---|---|---|---|
| | seq. | | rRNA and | Proteins | | Comparison to H50S | | | |
| D50S | length | General | docked tRNA | separated | H50S | proteins | | | |
| protein | (bp) | fold^{@} | (<4.5 Å) | by <4.5 Å | counterpart | D¹ | E² | Ct³ | Nt° |
| L2 | 275 | αβ + Ct | II, III, IV, V | — | L2 | + | | ± | |
| L3 | 211 | αβ + ext. | II, III, IV, V, | L13, L14, | L3 | + | + | | − |
| | | | VI | L17, L19 | | | | | |
| L4 | 205 | αβ + ext. | I, II, III, IV, V | L15, L20, | L4 | + | ± | | |
| | | | | L34 | | | | | |
| L5 | 180 | αβ + small ext. | V, 5S, | — | L5 | ± | − | | |
| | | | P-tRNA | | | | | | |
| L6 | 212 | 2αβ + Ct | V, VI | L36 | L6 | + | | − | |
| L9 | 146 | αβ | I, V | L31 | | | | | |
| L11 | 144 | 2αβ | II | CTC | | | | | |
| L13 | 174 | αβ + Nt + ext. | II, V, VI | L3, L20 | L13 | + | − | | − |
| L14 | 134 | β barrel, αβ | III, IV, V, VI | L3, L19 | L14 | + | − | | |
| L15 | 156 | α + Nt | I, II, III, V | L4, L21, L35 | L15 | + | | − | ± |
| L16 | 142 | αβ | 5S, II, V, A | CTC, L27 | L10e | + | | | |
| | | | and P-tRNA | | | | | | |
| L17 | 116 | αβ + Nt | III, IV, VI | L22, L32, L3 | L31e | − | − | | − |
| L18 | 114 | αβ | 5S, V | L27 | L18 | + | | − | − |
| L19 | 166 | β barrel + Ct + Nt | IV, VI | L3, L14 | L24e | − | | − | − |
| L20 | 118 | extended α- | I, II, IV | L13, L21, L4 | — | | | | |
| | | helix | | | | | | | |
| L21 | 169 | β barrel + bhl | II | L15, L20 | — | | | | |
| L22 | 134 | αβ + bhl + Nt | I, II, III, IV | L17, L32 | L22 | + | ± | | − |
| L23 | 95 | αβ + bhl | I, III | L29 | L23 | + | − | | |
| L24 | 115 | β barrel + ext. + Nt + C | I | — | L24 | + | D | − | − |
| | | ext | | | | | longer | | |
| CTC | 253 | β barrel + αβ + α | II, V, 5S, | L16, L11 | — | | | | |
| | | | A-tRNA | | | | | | |
| L27 | 91 | B + Nt + C ext. | II, V, 5S, | L18, L16 | L21e | − | | − | − |
| | | | P-tRNA | | | | | | |
| L28 | 81 | extended α- | I | — | — | | | | |
| | | helix | | | | | | | |
| L29 | 67 | A | I | L23 | L29 | + | | | |
| | | (leucine zipper) | | | | | | | |
| L30 | 55 | αβ | II, 5S | — | L30 | ± | | | |
| L31 | 73 | αβ + bhl-N-ext. | I, III, IV, V, | L9 | L15e | − | | − | − |
| | | E-tRNA | | | | | | | |
| L32 | 60 | B Zn-finger | I, II, IV, VI | L17, L22 | — | | | | |
| | | motif + Nt | | | | | | | |
| L33 | 55 | B + Ct | II, V, E-tRNA | L35 | L44e | + | | | − |
| L34 | 47 | A + Nt | I, II, III | L4 | L37e | − | | − | ± |
| L35 | 66 | A + ext. | I, II, V | L15, L33 | — | | | | |
| L36 | 37 | B Zn-finger | II, V, VI | L6 | — | | | | |
| | | motif | | | | | | | |

[symbol]

According to White et al., 1999. Science 286, 1571.
^{@}Folds are: α - alpha helical domain. β - beta sheet globular domain. αβ - mixed alpha helical and beta sheet, bhl - β hairpin loop;. ext - extended loop. N-ext - extended loops at the N-terminus, C-ext - extended loops at the C-terminus, N-t - extended tails at the N-terminus, C-t - extended tails at the C-terminus.
¹D - domain similarity
²E - extended loop similarity
³Ct - C-terminal tail similarity, °Nt - N-terminal tail similarity
± In globular domains - some similarity. In extended loops - one ext. is similar while the other is different. In tails - one tails is different in length, the other less or no difference.

Several structural studies have been performed on isolated LRS fragments, among them two NMR studies of the vicinity of H80 (Puglisi, E. V. et al, 1997. Nature Struct. Biol. 4, 775) and of H92 (Blanchard, S. C. and Puglisi, J. D., 2001. Proc Natl Acad Sci USA. 98, 3720). The results of the first study do not fit the in situ conformation within D50S, most likely because of the lack of supporting interactions with neighboring features. The second, however, fits rather well, since most of the structure forms a helix. Nevertheless, even in this case, the curvature of the helix and the shape of the stem loop differ from the present observations.

TABLE 5
Characterization of D50S RNA features.

| | | Comparisons with other |
|---|---|---|
| Helix # | Characteristics in D50S | 50S subunits |
| 1 | located between H94 and L13, coaxial with H2 | disordered in Hm |
| 9 | loop shorter than Tt therefore no contact to H54 | |
| | minor groove | |
| Junc | bends toward the lower part of H11 | no bend in Tt, interacts with |
| 11/12 | | Junc H4/H14 |
| 12 | smaller loop due to sequence difference, no | C197 in Hm flips out |
| | equivalence to nt 197 in Hm; | |
| | interacts with H22 via flipped out U206 | |
| 18 | second bulge interacts with minor groove of H7 | Hm: minor/minor interactions |
| | | with H4 |
| 21 | larger loop than in Tt and Hm; folds backwards, | |
| | connecting U400 via Mg; G399 flips out and contacts | |
| | H11 (U177) and H13 (G225) | |
| 25 | all are similar in the lower part, Tt and Dr are shorter | Tt's loop does not contact H46 |
| | than Hm; | minor groove |
| | Dr loop contacts minor groove of H46 | |
| 28 | A624 and A625 flip out | Hm: counterpart (A674) |
| | | interacts with H4 |
| 38 | loop nt 942-946 points backwards; | Hm: counterpart (1027-1033) |
| | nt 893-908 disordered | interact with H45; |
| | | nt 971-998 disordered |
| 39 | U969 flips out towards loop H4 (in 5S rRNA) | |
| | backbone | |
| Junc | Nt 1036-1037 similar to Tt; | Hm: bulge contacts H39, 40, |
| 41/42 | different from Hm (1123-1230). | 72 |
| 43 | angular separation between H43-44 wider than in Tt | Hm: H43/44 disordered. Tt: |
| | | loop E motif |
| Junc | no similarity to Tt and Hm; | Hm: connection to H96 and |
| 26/47 | Dr shorter than Hm | H61 |
| 57 | similar to Tt, loop interacts with H101 backbone | |
| 58 | Dr is similar to Tt from bulge onward but different | Hm: contacts H56 minor |
| | from Hm; | groove. Loop interacts with |
| | interacts with H54 bulge | H34 |
| 59 | slightly rotated compared to Tt; facing the solvent | does not exist in Hm |
| 61 | No equivalent to extra nt U1722 in Hm; | nt U1722 in Hm interacts with |
| | Dr: L17(Ala6) occupies place of G1730 in Hm | helix 59a |
| 63 | shorter but similar to Hm | Tt: minor/minor with H56 |
| 68 | longer loop than Hm, interacts with base-pairs in | |
| | H22, H88 | |
| 69 | interacts with H71, lies on interface | Hm: disordered; |
| | | Tt: contacts H44 of 30S |
| 73 | U2591 different orientation than Hm equivalent | |
| | (C2647); contacts H35 backbone | |
| | Dr: L32-His4 occupies position of Hm C2647 | |
| 77-78 | arm displaced by a 30 degree angle relative to its | Hm: disordered; |
| | position in Tt | Tt: blocks E-RNA passage |
| 79 | longer than Hm, Tt; loop contacts H52 and loop H58 | |
| 84 | | Nt 2339-2343 disordered |
| Junc | U2483 flips out and contacts G2044 at Junc 73/4 | equivalent position of A2551 |
| 89/90 | | in Dr is taken by a pyrimidine |
| | | (U2607) |
| 96 | C2669 faces its third bulge; located opposite to | |
| | U2727 in H, whose place is occupied by G2847 in | |
| | Dr | |
| Junc 99/1 | Nt 2866-70 face the lower part of H98, contacts H94 | Tt: face the backbone of H98, |
| | | points towards H2 |

Dr—D. radiodurans,
Hm—H. marismortui,
Tt—T. thermophilus,
Junc—junction in the secondary map of D50S,
nt—nucleotide

The sarcin/ricin loop (SRL) is a feature that interacts with G domains of elongation factors and which has been found to be essential for elongation factor binding. This loop is located near protein L14 and the site assigned for A-tRNA in the present docking experiments that were based on the structure of the 70S ribosome complex (Yusupov, M. M. et al., 2001. Science 292, 883). Although conformational dynamics of the sarcin-ricin loop are believed to be involved in factor binding, the high resolution structure of this region determined in isolation (Correll, C. C. et al., 1997. Cell 91, 705) matches that seen in the structure D50S.

The conformation of the 5S rRNA in D50S is slightly different from those determined for it and its complexes in isolation (Correll, C. C. et al., 1997. Cell 91, 705; Nakashima, T. et al., 2001. RNA 7, 692; Lu, M. and Steitz, T. A., 2000. Proc Natl Acad Sci USA. 97, 2023; Fedorov, R. et al., 2001. Acta Cryst D. 57, 968). Two of its binding proteins, called L5 and L25 in E. coli, have been extensively studied in isolation (Nakashima et al., 2001) and in complex with RNA fragments representing their binding sites to the 5S molecule (Lu, M. and Steitz, T. A., 2000. Proc Natl Acad Sci USA. 97; Fedorov, R. et al., 2001. Acta Cryst D. 57, 968). It was found that the conformations determined for L5 from Bacillus stearothermophilus (B. stearothermophilus) in isolation (Nakashima, T. et al., 2001. RNA 7, 692) does resemble that seen in D50S, as described in greater detail below.

D50S contains several proteins and RNA features that do not appear in H50S and T50S (Tables 4 and 5), including two Zn-fingers proteins, and proteins L32 and L36. Almost all of the globular domains of the D50S proteins are peripheral and, as in H50S and T30S, most of them have tails and extended loops that permeate the subunit's core. Analysis of the general modes of the RNA-protein interactions within D50S did not reveal striking differences from what was reported for the other ribosomal particles. However, many of the D50S proteins that have counterparts in H50S show significantly different conformations.

Mutations within a single ribosomal protein potentially mediate adaptation from mild to stressful conditions: In D50S, CTC (named after a general shock protein) replaces the 5S rRNA binding proteins L25 in E. coli and its homologue TL5 in T50S. H50S contains neither L25 nor any of its homologues. Within the known members of the CTC protein family, that of D. radiodurans is the longest, containing 253 residues and thus being about 150 residues longer than E. coli L25 and 60 residues longer than T. thermophilus TL5.

The structure of complexes of T. thermophilus TL5 (Fedorov, R. et al., 2001. Acta Cryst D. 57, 968) and E. coli L25 (Lu, M. and Steitz, T. A., 2000. Proc Natl Acad Sci USA. 97) with RNA fragments corresponding to their 5S rRNA binding regions (40 and 18 nucleotides long, respectively) have been determined at high resolution. Comparisons between them showed that the structure of the N-terminal domain of TL5 is similar to that of the entire L25. Protein CTC has three domains; the N-terminus is similar to the entire L25 and to the N-terminus of TL5 and the middle domain is similar to the C-terminal domain of TL5. However, the relative orientation of the N— terminal and of the middle CTC domains differs from that determined for the two domains of TL5 in isolation. The third domain of CTC, the C-terminal, is composed of three long α-helices connected by a pointed end, bearing some resemblance to structural motif seen in some small ribosomal subunit proteins.

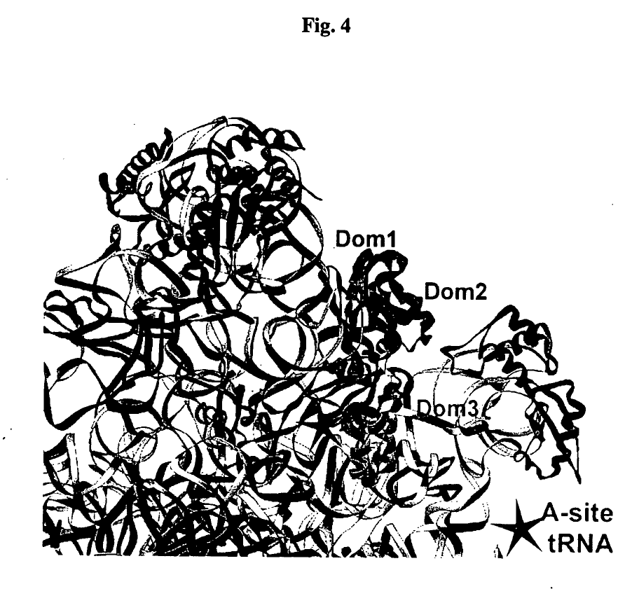

As mentioned above, the N-terminal domain of CTC is located on the solvent side of D50S (FIG. 4), at the presumed position of L25 in E. coli. The middle domain fills the space between the 5S and the L11 arm, and interacts with H38, the helix that forms the intersubunit bridge called Bla (see below). The interactions with H38 and the partial wrapping of the central protuberance (CP) of the subunit, are likely to provide additional stability, consistent with the fact that these two domains are almost identical to the substitute for protein L25 (protein TL5) in the ribosome of T. thermophilus.

The C-terminal domain is placed at the rim of the intersubunit interface. Docking the tRNA molecules, as seen in the 5.5 Å structure of the T70S complex (Yusupov, M. M. et al., 2001. Science 292, 883), showed that the C-terminal domain of CTC reaches the A-site and restricts the space available for the tRNA molecules. The somewhat lower quality of the electron density map of this domain hints at its inherent flexibility and indicates that it may serve as an A-site regulator and may also have some influence on the processing of mRNA. In addition, the C-terminal domain of CTC interacts with the A-finger. This interaction, the manipulation of the binding of tRNA at the A-site, the influence on the mRNA progression and the enhanced stability of the CP caused by CTC may be parts of the mechanisms that D. radiodurans developed for survival under extremely stressful conditions.

Features Involved in Ribosomal Functions:

The L1 stalk: The L1 stalk includes helices H75-H78 and protein L1, a feature that was identified as a translational receptor binding mRNA (Nikonov, S. et al., 1996. Embo J. 15, 1350). Its absence has a negative effect on the rate of protein synthesis (Subramanian, A. R., and Dabbs, E. R., 1980. Eur J Biochem 112, 425). In the complex of T70S with all three tRNA molecules, the L1 stalk interacts with the elbow of E-tRNA and the exit path for the E-tRNA is blocked by proteins L1 from the large subunit and S7 from the small one (Yusupov, M. M. et al., 2001. Science 292, 883). Consequently it was suggested that the release of the deacylated tRNA requires that one or both of these features move. Movement of the L1 arm was also associated with the binding of EF2 in yeast (Gomez-Lorenzo, M. G., 2000. Embo J 19, 2710). In H50S, the entire L1 arm is disordered and therefore could not be traced in the electron density map (Ban, N. et al., 2000. Science 289, 905), an additional hint of its inherent flexibility.

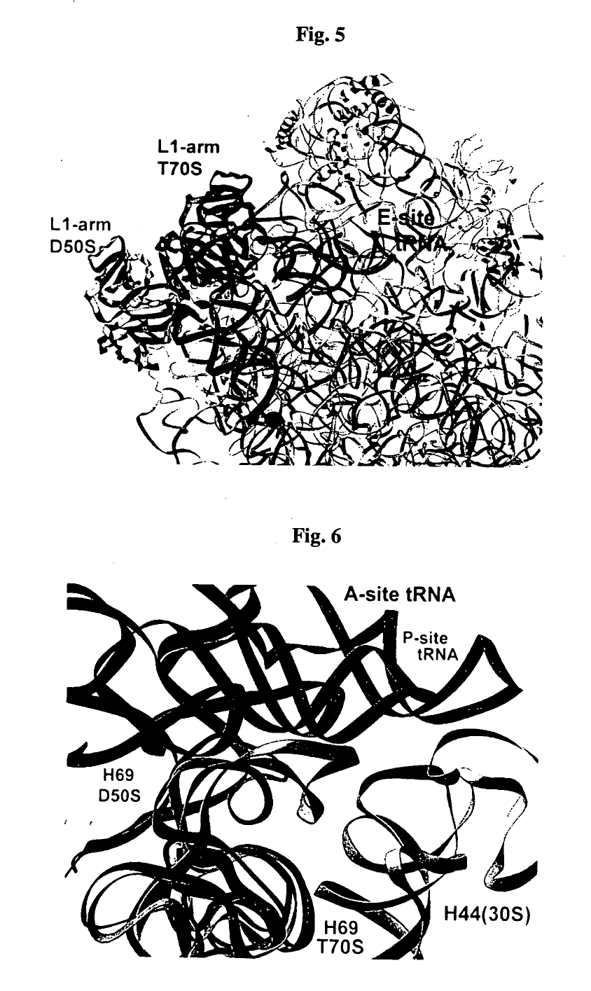

In D50S the L1 arm is tilted about 30 degrees away from it corresponding position in T70S, so that the distance between the outermost surface points of the L1 arm in the two positions is over 30 Å (FIG. 5). The orientation of the L1 arm in D50S allows the location of protein L1 so that it does not block the presumed exit path of the E-site tRNA. Hence, it is likely that the mobility of the L1 arm is utilized for facilitating the release of E-site tRNA. Superposition of the structure of D50S on the LRS of the T70S ribosome suggests definition of a pivot point for a possible rotation of the L1 arm.

It has been suggested that a head-platform concerted motion in the small subunit may assist the exit of the E-site tRNA as well as the translocated mRNA (Pioletti, M. et al., 2001. Embo J. 20, 1829). The present analysis adds to this putative mechanism the exiting E-tRNA and a swinging intersubunit bridge. The 16S RNA and the 30S proteins do not block this path and the movements of the head and the platform may assist the progression of the mRNA together with the E-site tRNA towards the exit site. Density was identified in the current map of D50S that may accommodate a large part of protein L1.

The L7/L12 arm and the GTPase center: A major protruding region of domain II, that connects the solvent region with the front surface of the LRS consists of helices H42, H43 and H44 and the internal complex of L12 and L10. This stalk is involved in the contacts with the translocational factors and in factor-dependent GTPase activity (Chandra Sanyal S. and Liljas A., 2000. Curr Opin Struct Biol 10, 633). Like other functionally important features, the entire L7/12 stalk is disordered in the H50S structure (Ban, N. et al., 2000. Science 289, 905; Cundliffe, E. in The Ribosome: Structure, Function and Evolution (eds. Hill, W. E. et al.) 479-490 (ASM, Washington, D.C., 1990)) but is well ordered in T70S (Yusupov, M. M. et al., 2001. Science 292, 883). In D50S the RNA portion of this domain appears to be ordered, but the two proteins less so, although density that can host a large part of them has been identified. The location of part of this arm (H43) within D50S is somewhat shifted (by 3-4 Å) compared to its position within T70S. Consequently, the conformation of the entire arm in D50S is slightly less compact than its conformation in T70S.

One of the proteins associated with the L7/L12 arm, protein L11, appears in the structure of D50S. L11 together with the 23S rRNA stretch that binds it (the end of H42, H43 and H44), are associated with elongation factor and GTPase activities (Cundliffe, E. et al., 1979. J Mol Biol 132, 235). This highly conserved region is the target for the antibiotic thiostrepton, and it has been shown that cells acquire resistance to this antibiotic by deleting protein L11 from their ribosomes. Large ribosomal subunits lacking protein L11 do not undergo major conformational changes (Franceschi, F. et al., 1994. Syst. & App. Microbiology 16, 697), but cease to bind thiostrepton. Complexes containing L11, or one of its domains together with fragments mimicking the RNA stretch binding it, were subjected to crystallographic and NMR studies (Wimberly, B. T. et al., 1999. Cell 97, 491; GuhaThakurta, D., and Draper, D. E., 2000. J Mol Biol. 295, 569). Interestingly, whereas the structures of protein L11 determined in these studies were similar to that found within the ribosomal particle, there is less resemblance between the structures of the RNA fragments and that determined in situ.

Analysis of disorder in intersubunit bridges of unbound 50S subunit: Several features, known to form intersubunit bridges are exceptionally well resolved in the map of D50S. These include helices H38 and H69, and proteins L5, L14 and L19. Helix H38, the longest stem in the large subunit, has a significant functional relevance as its upper half is the sole component forming the intersubunit bridge termed “B1a bridge” or “A-finger”. In addition, one of its conserved internal loops interacts with the D- and T-loops of A-tRNA (Yusupov, M. M. et al., 2001. Science 292, 883). In D50S it originates on the solvent side of the LRS, makes a sharp bend, and emerges between domain V and the 5S rRNA at the interface surface. Its location and orientation allow its contacts with protein S13.

The orientation of H69 with its universally conserved stem-loop in D50S is somewhat different than that seen in T70S. Both lie on the surface of the intersubunit interface but, in the 70S ribosome, H69 stretches towards the small subunit whereas in the free 50S subunit it makes more contacts with the large subunit (H71) so that the distance between the tips of their stem-loops is about 13.5 Å.

Comparison of the two orientations of H69 (FIG. 6) suggested that a small rotation of H69 in the free 50S subunit is sufficient for turning this helix into bridging position so that it can interact with the small subunit near the decoding center in Helix H44. In this position H69 can also contact the A- and P-site tRNA molecules and be proximal to elongation factor EF-G in the post-translocation state (Yusupov, M. M. et al., 2001. Science 292, 883). Although it seems that H69 undergoes only subtle conformational rearrangements between the free and the bound orientations, it is clear that the displacement and the rotation of a massive helix such as H69 requires a high level of inherent flexibility. This may explain why in the high resolution structure of H50S, which was determined at far from physiological conditions, a distinct disadvantage, H69 is disordered (Ban, N. et al., 2000. Science 289, 905).

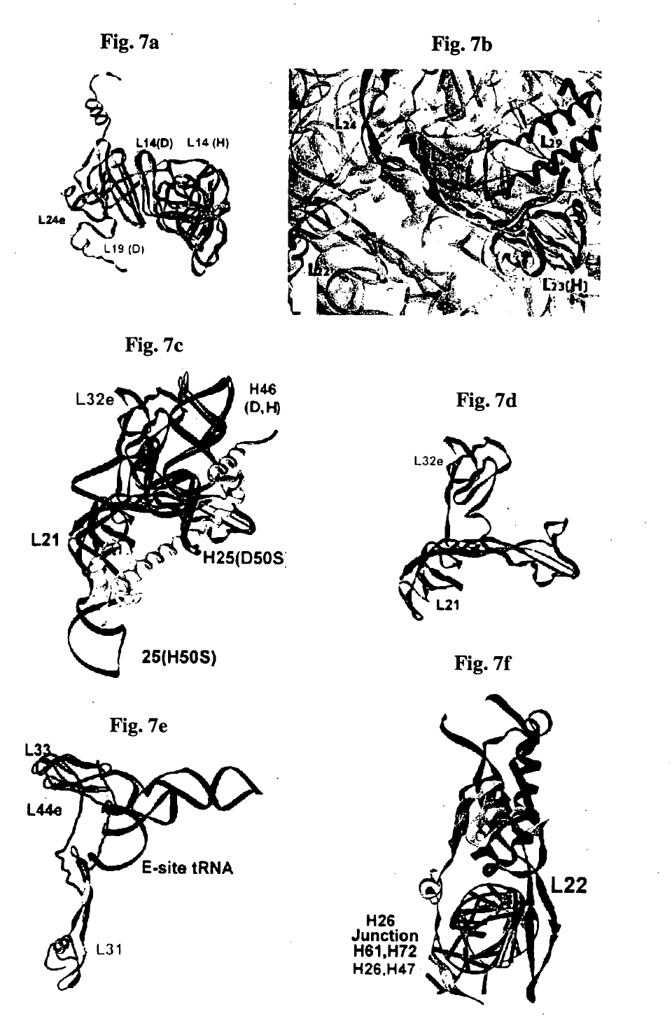

Proteins L14 and L19 form an extended inter-protein beta sheet composed of two β-hairpin loops of L14 and two of L19 (FIG. 7a). In H50S, there is no L19 but, L24e, although different in shape and smaller in size, is located at the same position and forms a similar β-sheet element. Both L14 and L19 are directly involved in intersubunit bridges. L19 is known to make contacts with the penultimate stem of the small subunit, at bridge B6. L14 contacts helix H14 of the 16S RNA to form bridge B8. It is likely, therefore, that the structural element produced by L14 and its counterpart (L19 or L24e) has functional relevance in the construction of these two bridges. In D50S, these proteins, together with protein L3, form one of the two intimately connected protein clusters, consistent with the large number of such cross-links reported (Walleczek, J. et al, 1989. Biochemistry 28, 4099). This clustering may enhance the stability of the structural features required for the intersubunit bridges.

Protein L5, together with S13 which is located in the head of the small subunit, form the only intersubunit bridge (B1b) which is composed exclusively of proteins, (Yusupov, M. M. et al., 2001. Science 292, 883). The entire domain of L5 which is involved in this bridge, like almost all of the RNA features forming bridges with the small subunit, that appear to be fully ordered in T70S and almost so in D50S, are missing in H50S. Additional RNA features that are involved in intersubunit contacts are helices H62, H64, H69 and the lateral arm composed of H68-H71. All are present in the D50S structure in a fashion that allows their interactions with the small subunit and have similar conformations as those seen in T70S (Yusupov, M. M. et al., 2001. Science 292, 883).

Based on the disorder observed in almost all functional features of H50S, it was assumed that most of the features involved in subunit function and in mediating intersubunit contacts are disordered in free LRSs and become stabilized in the 70S ribosome, (Yusupov, M. M. et al., 2001. Science 292, 883). The finding that many of the disordered features in H50S are ordered in D50S indicates that the H50S crystal structure contain features that flex more than those in that of D50S. Thus, it is likely that structures derived from crystallized H50S subunits (Franceschi, F. et al., 1994. Syst. & App. Microbiology 16, 697) represent conformations attained under environmental conditions close to those suitable for selective detachment of the proteins missing in this structure and hence that structural information obtained according to the present invention is significantly more accurate than that obtained for H50S.

Evolutionary Implications:

The nascent-protein exit tunnel become tighter with evolution: More than three decades ago biochemical studies showed that the most newly synthesized amino acids of nascent proteins are masked by the ribosome (Malkin, L. I., and Rich, A., 1967. J Mol Biol. 26, 329; Sabatini, D. D. and Blobel, G., 1970. J Cell Biol 45, 146). A feature which may account for this masking was first seen as a narrow elongated region in images reconstructed at very low resolution (60 Å) in 80S ribosomes from chick embryos (Milligan, R A. and Unwin P N., 1986. Nature 319, 693) and at 45 Å in images of 50S subunits of B. stearothermophilus (Yonath, A. et al., 1987. Science 236, 813). Despite such low resolutions, these studies showed that this tunnel spans the large subunit from the location assumed to be the peptidyl transferase site to its lower part, and that it is about 100 Å in length and 15 Å in diameter, as subsequently confirmed at high resolution in H50S (Nissen, P. et al., 2000. Science 289, 920) and in D50S.

The structural features building the walls of the tunnel, their chemical composition and their “nonstick” character in H50S are described in (Nissen, P. et al., 2000. Science 289, 920). Although the same gross characteristics were identified in D50S, namely a lack of well-defined structural motifs, large patches of hydrophobic surfaces and low polarity, on average, the tunnel in D50S was strikingly wider than in H50S. This widening effect is caused by missing segments, such as the loop (residues 72-77) of protein L4 that in H50S penetrates into the tunnel and by several nucleotides that flip into the tunnel, or by the lower exposure of nucleotides, such as A2581 in D50S, compared to A2637, counterpart thereof, in H50S.

The exit of the tunnel is located at the bottom of the ribosomal particle. The tunnel in D50S is composed of domains I and III, and several proteins including L4, L22-L24 and L29. In H50S, however, L31e and L39e, two proteins that do not exist in D50S, are also part of the lower section of the tunnel and cause its tightening. Of interest is L39e, a small protein having an extended non-globular conformation, which penetrates into the RNA features lining the walls of the tunnel in that region. This protein replaces L23 in D50S and, since it is built of an extended tail, it can penetrate deeper into the tunnel walls than the loop of L23 in D50S. The globular domain of protein L23 in D50S, is similar to that of L23 in H50S, and both are positioned in the same location. However, the halophilic L23 has a very short loop compared to H23 in D50S and, in H50S, protein L39e occupies the space taken by the extended tail of L23 in D50S.

L39e is present in archaea and eukaryotes, but not in eubacteria. Thus, it seems that with the increase in cellular complication, and perhaps as a consequence of the high salinity, a tighter control on the tunnel's exit was required, and two proteins, HL23 and L39e, replace single one. So far there are no indications for a connection between this replacement and evolution. Nevertheless, it is evident that a protein in this delicate position may mediate interaction between the ribosome and other cellular components, evolving further to act as a hook for the ribosome on the ER membrane. A high resolution structure of a eukaryotic ribosome, bound to the ER membrane, should provide an answer to these open questions.

Evolving Structural Elements:

Helix H25: Helix H25 displays the greatest sequence diversity among eubacterial and halophilic large subunits. It contains 27 nucleotides in D50S and 74 in H50S (FIG. 7c). It lies on the solvent side of the subunit and, in D50S, the region that is occupied by this helix in H50S hosts proteins L20 and L21. These two proteins exist in many eubacterial ribosomes but not in that of H. marismortui which evolved later than D. radiodurans. Protein L21 has a small β-barrel-like domain that is connected to an extended loop. Protein L20, in contrast, is built of a long α-helical extension with hardly any globular domain. Its shape and location make it a perfect candidate for its being a protein having a role in RNA organization. This may explain why L20 is one of the early assembly proteins and why can it take over the role of L24 in mutants lacking the latter.

The replacement of proteins by an RNA helix should be rather surprising, since in this way the ribosome could have lost two strong structure-stabilizing elements. However, in this case, regardless of the effects of the extension of helix H25 in archaea and eukaryotes, this did not reduce stabilization of this region since protein L32e has looped tails having sufficient length to compensate for many of the contacts made by the tail of L20 and the loop of L21 (FIGS. 7c-d). It is therefore likely that the loop of L32e organizes the RNA environment in H50S in a fashion similar to the loop of L21 in D50S. The globular domains of proteins L32e and L21 appear to be similar and it is likely that L21 and L32e are indeed evolutionarily related. The globular domain of L32e is rotated by 180 degrees, relative to that of L21, around an axis defined by its tail, and the unoccupied space in H50S corresponding to the location of the globular domain of L21 is occupied by the extension of H25.

The “protein-tweezers” motif: Among the novel protein structures of D50S are two Zn-finger proteins; L32 and L36 that do not exist in H50S and have no replacements or counterparts therein. The position occupied by L32 in D50S overlaps that hosting the loop of L22 in H50S and, in D50S, L32 and L22 form a tweezers-like motif possibly clamping interactions between domains II, III and IV (junctions H26/H47 and H61/H72) (FIG. 7f). These two proteins interact extensively with protein L17, an additional novel protein that occupies the location of L31e in H50S, and the entire region seems to be highly stabilized. The question, still to be answered is: why, with evolution, was a protein replaced by a loop of another one even though this replacement seems to cause partial loss of stability of a well organized structural motif The E-site tRNA: The E-site tRNA may interact, in D50S to the end of the extended loop of protein L31. In H50S, the region interacting with E-site RNA is provided by the extended loop of L44e. These two proteins are located at opposite sides of the location of the E-site tRNA, yet the interactions occur at approximately the same place, via their extended loops (FIG. 7e). In D50S, protein L33, which has no extended loop, occupies the space taken by the globular domain of L44e in H50S, and the globular domains of both are rather similar. These complicated rearrangements may indicate that with evolution the ribosome preserved the configurations and locations of the features involved in the peptide bond formation.

Helix H30b: Helix H30b, which does not exist in D50S, is located on the solvent surface in H50S and makes extensive contacts with protein L18e, a protein which does not exist in D50S, and with the lower part of H38. Protein L18e, in turn, connects H30b to H27 and to the loop of H45 and interacts with proteins L4 and L15. This RNA-protein network seems to be rather rigid and its strategic location may indicate that it protects the ribosomal surface from the increasing complexity of the environment.

Concluding remarks: The LRS has a compact structure, its core is built of well-packed interwoven RNA features and it is known to have less conformational variability than the small subunit. Nevertheless, it assumes conformations which can be correlated to the functional activity of the ribosome. Analysis of results obtained while reducing the present invention to practice support linkage of the functional activity of the ribosome and the flexibility of its features. Based on comparison between the structures of free D50S and that of bound T50S, it is suggested that the ribosome utilizes the inherent flexibility of its features for facilitating specific tasks. Remarkable examples of such characteristics are displayed by helix H69, which creates the 50S hook in the decoding region of the small subunit, and the entire L1 arm, which produces the revolving gate for exiting tRNA molecules.

The striking difference between the conservation of the rRNA fold and the significant diversity of ribosomal proteins indicates that the latter do not only have roles in stabilizing rRNA conformation, but also play a role in binding of factors and substrates and in enhancing intersubunit association. The extended protein termini and the long protein loops are mainly buried within the ribosomal particle and thus are trapped in distinct conformations. However, those which are pointing outside, such as protein S18 in the small subunit (Pioletti, M. et al., 2001. Embo J. 20, 1829), the loop of L5 and the N-terminus of L27, maintain a high level of flexibility and are available to interact, to bind and to enhance the placement of factors and substrates. It is therefore conceivable that in these cases the diversity of ribosomal proteins is linked to the evolution of the interacting components.

Remarkable preservation of structural motifs was observed in ribosomal proteins despite their overall conformational and sequence differences. The L14/L19 inter-protein β-sheet (FIG. 6) shows how functional requirements can be satisfied in evolving ribosomes. Similarly, addition of a protein for creating a tighter tunnel opening was identified as the mechanism employed for reducing freedom of movement of nascent proteins in the increasingly complex environment of higher organisms.

The unique three-domain structure of CTC and the topology of these domains in D50S (FIG. 4) may indicate that ribosomes of a stress-resistant bacterium control the incorporation of amino acids into growing chains by restricting the space allocated for the A-site tRNA. The positioning of a two-domain homologous protein (TL5) in T50S suggests a mechanism for stabilizing ribosomal function elevated temperatures. In addition, D. radiodurans has evolved a third domain in its LRS enabling it to survive under extreme conditions.

Summary: The present results describe the 3.0 Å resolution structure of the LRS of the gram-positive mesophilic bacterium D. radiodurans (D50S). The RNA folds of D50S and 50S from Haloarcula marismortui (H50S) are similar, yet the functionally relevant features of D50S are ordered, in contrast to the disorder observed, presumably due to crystal environment, in the structure of unbound H50S. Analysis revealed replacement of a single D50S protein by two H50S proteins, while tightening the nascent protein tunnel, and indicated strategies that may partially account for survival under stressful conditions. The present analysis confirms that utilization of inherent flexibility for functional tasks is a common ribosomal strategy, and suggest how the L1-arm facilitates the exit of tRNA and how H69 creates the intersubunit bridge to the decoding center.

Thus, while reducing the present invention to practice, the present inventors have generated the first essentially complete model of the high resolution three-dimensional atomic structure of a bacterial LRS which also represents the first such model of a eubacterial LRS. Such a model, therefore, constitutes a dramatic breakthrough in the art, representing the culmination of decades of intensive research aimed at elucidating the extremely complex three-dimensional atomic structure and vital functional mechanisms of the ribosome. As such, the present ribosomal structure model is far superior to all prior art ribosome structure models and provides a critical and potent tool for enabling the rational design or identification of bacterial antibiotics, an urgent medical imperative, particularly in light of the current global epidemics of diseases associated with antibiotic resistant strains of bacteria. The present model also constitutes a potent means enabling the rational design or identification of LRSs having desired characteristics, such as, for example, conferring enhanced protein production capacity for example, for production of recombinant proteins. Also, importantly, the present model constitutes a powerful means for facilitating the elucidation of the vital and universal biological process of protein translation performed by the ribosome.

### Example 2

### Growth of Antibiotic-Large Ribosomal Subunit Complex Crystals and Generation of High Resolution Three-Dimensional Atomic Structure Models of the Interaction of Antibiotics with the Large Ribosomal Subunit

The LRS is the functional binding target for a wide range of antibiotics. As such, models of the structural and functional atomic interactions between antibiotics and the LRS are urgently required since, for example, these would constitute an indispensable and powerful tool for the rational design or selection of antibiotics or of ribosomes having desired characteristics, as described above. In particular, the ability to rationally design or select antibiotics is of paramount medical importance due to currently expanding global epidemics of increasing numbers of lethal diseases caused by antibiotic resistant strains of pathogenic microorganisms. However, all prior art approaches have failed to produce satisfactory high resolution three-dimensional atomic models of the structural and functional interactions between antibiotics and the LRS. Thus, in order to fulfill this urgent need, high resolution three-dimensional atomic structure models of the LRS complexed to the antibiotics chloramphenicol, clindamycin, clarithromycin, erythromycin and roxithromycin were generated while reducing this aspect of the present invention to practice, as follows.

Materials and Methods:

Base Numbering: Bases of the 23S rRNA sequence of D. radiodurans and of the corresponding E. coli sequence are numbered with “Dr” and “Ec” appended as a suffix to the base number for respective identification thereof.

Cell culture: D. radiodurans cells were cultured as recommended by the American Tissue Type Culture Collection (ATCC), using ATCC medium 679 with minor modifications.

Growth of D50S crystals: D. radiodurans LRSs were isolated, and crystals of D50S belonging to the space-group 1222 were grown as described in Example 1, above. Co-crystallization of D50S with antibiotics was carried out in the presence of 0.8-3.5 mM of the antibiotics chloramphenicol, clindamycin, erythromycin, and roxithromycin. Co-crystallization of D50S with clarithromycin was achieved by soaking D50S crystals in solutions containing 0.01 mM of this antibiotic.

X-ray diffraction: Data were collected at 85 K from shock-frozen crystals with a bright SR beam at ID19 at APS/ANL, ID14/2 and 4 at ESRF/EMBL, and at BW6 at DESY. Data were recorded on MAR345, Quantum 4, or APS-CCD detectors and processed with HKL2000 (Otwinowski, Z. and Minor, W., 1997. Macromolecular Crystallography, Pt A 276, 307).

Placements and refinement: The 3.1 Å structure of D50S described in Example 1, above, was refined against the structure factor amplitudes of each of the antibiotic-D50S complexes, using rigid body refinement as implemented in CNS (Brunger, A. T. et al., 1998. Acta Crystallographica Section D-Biological Crystallography 54, 905). SigmaA-weighted difference maps were used for the initial manual placement of the antibiotics. Each of the D50S-antibiotic models was further refined using REFMAC software (Murshudov, G. N. et al., 1999. Acta Cryst section D55, 247). For the calculation of free R-factor a subset of reflections (10% of the data) was omitted from the refinement. The structure coordinates of the antibiotic-D50S complexes were submitted to the PDB under accession numbers 1JZX, 1JZY, 1JZZ, 1K00, and 1K01.

Coordinates and Figures: Coordinates of chloramphenicol, clindamycin, erythromycin, roxithromycin, and clarithromycin were taken from Cambridge Structural Database and antibiotic structures were modeled into the difference density based on their crystal structure. Figures were produced using RIBBONS (Carson, M., 1997. Macromolecular Crystallography, Pt B 277, 493) or MOLSCRIPT (Kraulis, P. J., 1991. Journal of Applied Crystallography 24, 946) software.

Experimental Results:

Crystallographic data for antibiotic-D50S complexes were generated and are listed in Table 6.

TABLE 6
Antibiotic-D50S complex crystallographic data.

| Co-complexed | Resolution | Rsym | Completeness | | Unit cell | R/R_{free} |
|---|---|---|---|---|---|---|
| antibiotic | (Å) | (%) | (%) | <I/sig(I)> | dimensions (Å) | (%) |
| clindamycin | 35-3.1 | 11.8 (57.0) | 94.4 (82.2) | 6.5 (2.1) | 170.286 × 410.134 × 697.201 | 27.1/33.9 |
| erythromycin | 35-3.4 | 14.4 (51.7) | 67.0 (64.1) | 6.2 (1.5) | 169.194 × 409.975 × 695.049 | 29.6/33.1 |
| clarithromycin | 50-3.5 | 9.8 (50.6) | 85.2 (78.1) | 8.1 (2.1) | 169.871 × 412.705 × 697.008 | 29.1/33.3 |
| roxithromycin | 50-3.8 | 10.7 (24.4) | 64.8 (66.8) | 6.2 (2.2) | 170.357 × 410.713 × 694.810 | 24.7/32.0 |
| chloramphenicol | 25-3.5 | 13.9 (60.7) | 62.8 (54.5) | 7.5 (2.0) | 171.066 × 409.312 × 696.946 | 29.1/33.1 |

Three-dimensional atomic structures of antibiotic-LRS interactions: The three-dimensional atomic structure of portions of the LRSs in crystallized chloramphenicol-, clindamycin-, clarithromycin-, erythromycin-, and roxithromycin-LRS complexes are defined by atomic structure coordinates (D. radiodurans numbering system) set forth in Tables 7, 8, 9, 10 and 11, respectively (refer to enclosed CD-ROM for Tables), as follows:

23S rRNA: atom coordinates 1-59533;

ribosomal protein L4: atom coordinates 59534-59731;

ribosomal protein L22: atom coordinates 59535-59862; and

ribosomal protein L32: atom coordinates 59863-59921.

The three-dimensional atomic structure of the antibiotics, or portions thereof, in crystallized chloramphenicol-, clindamycin-, clarithromycin-, erythromycin-, and roxithromycin-LRS complexes are defined by HETATM coordinates 59925-59944 set forth in Table 7, HETATM coordinates 59922-59948 set forth in Table 8, HETATM coordinates 59922-59973 set forth in Table 9, HETATM coordinates 59922-59972 set forth in Table 10, and HETATM coordinates 59922-59979 set forth in Table 11, respectively (refer to enclosed CD-ROM for Tables).

The three-dimensional atomic positioning of Mg2+ ions associated with crystallized chloramphenicol-, clindamycin-, clarithromycin-, erythromycin-, and roxithromycin-LRS complexes are defined by HETATM coordinates 59922-59924 set forth in Table 7, HETATM coordinates 59949-59950 set forth in Table 8, HETATM coordinates 59974-59975 set forth in Table 9, HETATM coordinates 59973-59974 set forth in Table 10, and HETATM coordinates 59980-59981 set forth in Table 11, respectively (refer to enclosed CD-ROM for Tables).

The three-dimensional atomic structures of the portions of antibiotic-LRS complexes comprising the antibiotic and the 23S rRNA nucleotides located within a 20 Å-radius sphere centered on an atom of the antibiotic in crystallized chloramphenicol-, clindamycin-, clarithromycin-, erythromycin-, and roxithromycin-LRS complexes are defined by the structural coordinates (D. radiodurans numbering system) set forth in Tables 12, 13, 14, 15, and 16, respectively (refer to enclosed CD-ROM for Tables). The HETATM coordinates in Tables 12-16 define the three-dimensional atomic structures of their respective antibiotics, as described above, and the non-HETATM coordinates in these tables define the three-dimensional atomic structure of the 23S rRNA nucleotides located within the aforementioned 20 Å-radius sphere.

The electron density maps of the antibiotic-LRS complexes enabled unambiguous determination of the binding sites of the five antibiotics. The chemical nature of the antibiotic-D50S interactions could largely be deduced from the mode of binding. Based on these maps and on the available biochemical and functional data, the structural basis for the modes of action of the antibiotics chloramphenicol, clindamycin, erythromycin, clarithromycin, and roxithromycin is proposed.

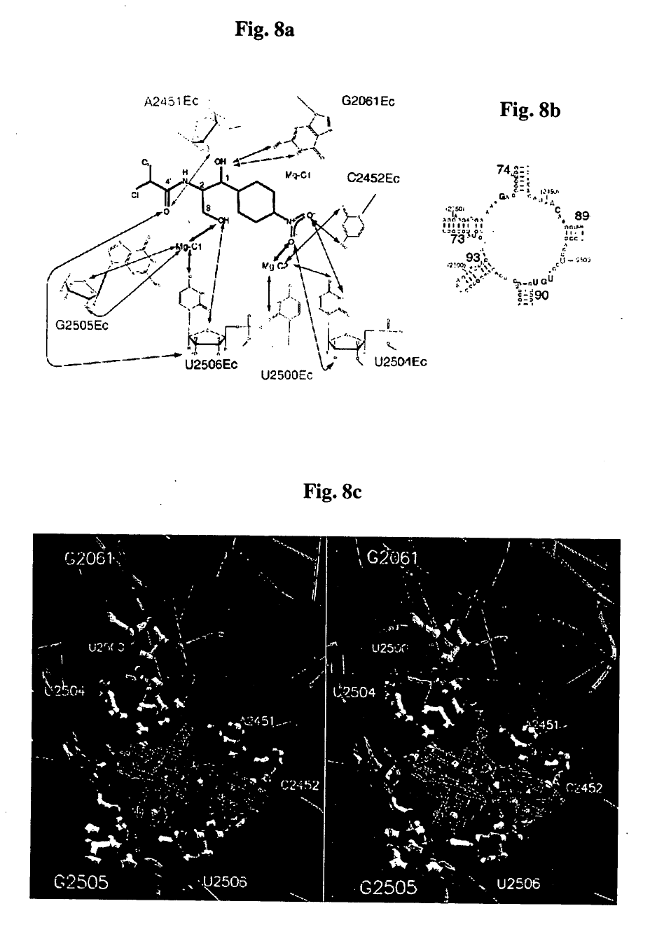

All of the antibiotics analyzed were found to target D50S subunit at the peptidyl transferase cavity and were found to interact exclusively with specific nucleotides that have been assigned to a multi-branched loop of domain V of the 23S rRNA in the two-dimensional structure. Nucleotides of 23S rRNA interacting with chloramphenicol (nucleotides 2044, 2430, 2431, 2479 and 2483-2485), clindamycin (nucleotides 2040-2042, 2044, 2482, 2484 and 2590) and all three macrolides (nucleotides 2040-2042, 2045, 2484, 2588 and 2589) are shown in FIGS. 8a, 9a and 10a, respectively. These findings explain previous mutational and footprinting data (reviewed in Spahn, C. M. T. and Prescott, C. D., 1996. Journal of Molecular Medicine-Jmm 74, 423; Vester, B. and Garrett, R. A., 1988. EMBO Journal 7, 3577; Polacek, N. in RNA-Binding Antibiotics (eds. Schroeder, R. & Wallis, M. G.) (Eurekah.Com, Incorporated, Georgetown., 2000)). None of the antibiotics examined showed any direct interaction with ribosomal proteins. Furthermore, binding of these antibiotics did not result in any significant conformational change of the peptidyl transferase cavity.

Chloramphenicol: At its single binding site, chloramphenicol targets the PTC mainly via hydrogen bond interactions. Chloramphenicol contains several reactive groups capable of forming hydrogen bonds, including the oxygen atoms of the para-nitro (p-NO₂) group, the 1OH and 30OH groups and the 4′ carboxyl group.

One of the oxygen atoms of the p-NO₂ group of chloramphenicol is in a position to form hydrogen bonds with N1 of U2483Dr (U2504Ec) and N4 of C2431Dr (C2452Ec) which have been shown to be involved in chloramphenicol resistance (Vester, B. and Garrett, R. A., 1988. EMBO Journal 7, 3577). The other oxygen atom of the p-NO₂ group interacts with O2′ of U2483Dr (U2504Ec) (FIGS. 8a-b).

The 1OH group of chloramphenicol is located at hydrogen bonding distance (about 4 Å) from N1 and N2 of G2044Dr (G2061Ec) of the 23S rRNA. This nucleotide has been implicated in chloramphenicol resistance in rat mitochondria (Vester, B. and Garrett, R. A., 1988. EMBO Journal 7, 3577) and a mutation of the neighboring nucleotide, A2062Ec, confers resistance to chloramphenicol in H. halobium (Mankin, A. S. and Garrett, R. A., 1991. J Bacteriol. 173, 3559).

The 3OH group of chloramphenicol is fundamental for its activity. The most common chloramphenicol resistance mechanisms involve either acetylation or phosphorylation of this OH group, a modification which renders chloramphenicol inactive (Izard, T. and Ellis, J., 2000. Embo Journal 19, 2690; Shaw, W. V. and Leslie, A. G. W., 1991. Annu. Rev. Biophys. Biophys. Chem. 20, 363). The 3OH group is within hydrogen bonding distance to 4′O of U2485Dr (U2506Ec). The 3OH group of chloramphenicol is also involved in interactions coordinated via a hydrated Mg2+ ion (Mg-Cl, see following section).

The 4′ carbonyl group of chloramphenicol could potentially form a hydrogen bond with the 2′OH of U2485Dr (U2506Ec) and the 2′OH of A2430Dr (A2451Ec), as it is located at a distance of about 4.3 Å from these positions (see FIG. 8a). These findings are consistent with mutations of A2430Dr (A2451Ec) resulting in chloramphenicol resistance (Thompson, J. et al., 2001. Proc Natl Acad Sci USA. 98, 9002; Vester, B. and Garrett, R. A., 1988. EMBO Journal 7, 3577). A stereo view showing the chloramphenicol binding site at the peptidyl transferase cavity of D50S is shown in FIG. 8c.

Magnesium ion-antibiotic interactions: In addition to the hydrogen-bonds between chloramphenicol and 23S rRNA residues, two hydrated Mg2+ ions are involved in chloramphenicol binding, Mg-C1 and Mg-C2, which are not present in the native D50S structure, nor in the complexes of D50S with the other analyzed antibiotics. Thus, their presence at these particular locations depends on chloramphenicol binding.

Mg-C1 mediates the interaction of the 3OH group of chloramphenicol with the O4 atom of U2485Dr (U2506Ec) and with the 2′OH group and 4′O atom of G2484Dr (G2505Ec). Studies have suggested that both of these nucleotides are protected by chloramphenicol (Rodriguez-Fonseca, C., Amils, R. and Garrett, R., 1995. Journal of Molecular Biology 247, 224). Mg-C2 mediates the interaction of one of the oxygen atoms of the p-NO₂ group with the O2 of U2479Dr (U2500Ec), O4 U2483Dr (U2504Ec), and O2 of C2431Dr (C2452Ec) via a salt bridge. This interaction further stabilizes the interaction of chloramphenicol with the peptidyl transferase cavity. The presence of Mg2+ appears crucial for its interaction with and inhibition of the ribosome.

Interestingly, the Mg2+ ion (Mg101) found overlapping with the chloramphenicol location in the native structure is not observed in the chloramphenicol-50S complex, suggesting that the coordinating effect of chloramphenicol is sufficient to maintain the local structure of the 50S subunit in the absence of Mg101. The displacement of Mg101 by chloramphenicol and the coordinating effects of Mg-C1 and Mg-C2 in the presence of chloramphenicol, could provide a partial explanation as to why chloramphenicol, in spite of being a relatively small molecule, has been chemically footprinted to many different positions on the peptidyl transferase ring.

Generation of new metal ion binding sites due to antibiotic-binding as observed for chloramphenicol, may explain the mode of action and/or binding of other drugs as well and may be used as a tool in rational drug design.

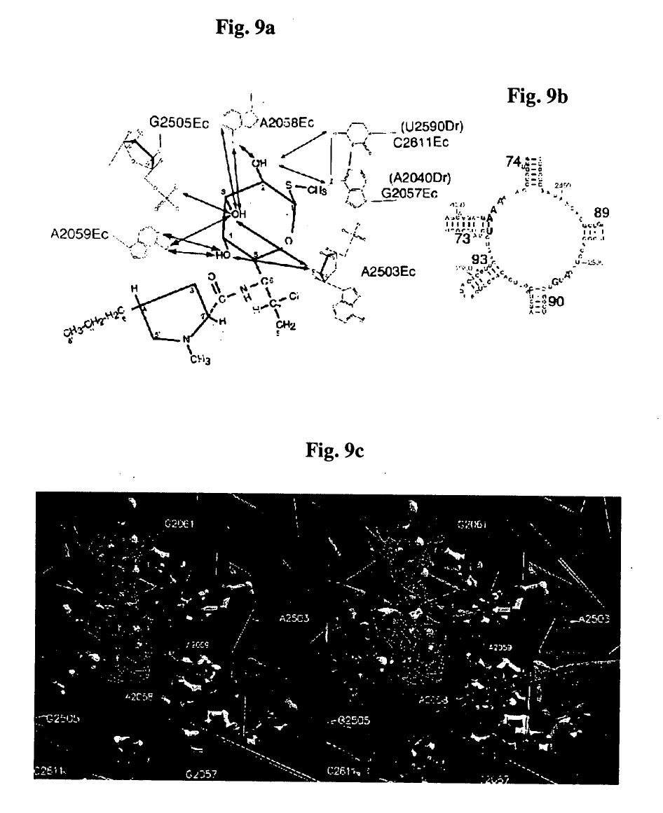

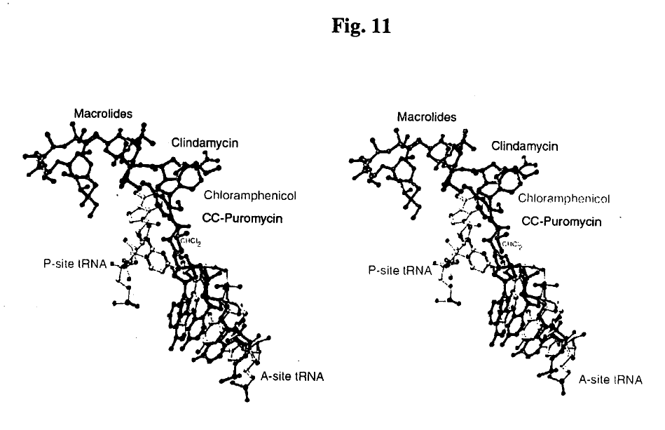

Clindamycin: Although the binding site for the lincosamide clindamycin in the PTC is different from that of chloramphenicol, it appears to be partially overlapping (FIGS. 9a and 11). Novel Mg2+ ions involved in the binding of clindamycin were not identified, however, as observed for chloramphenicol, the binding of clindamycin displaced Mg101.

Clindamycin has three hydroxyl groups in its sugar moiety that can participate in hydrogen bond formation (see FIGS. 9a and 9b). The 2OH of clindamycin appears to form a hydrogen bond with N6 of A2041Dr (A2058Ec). A2041Dr (A2058Ec) is the pivotal nucleotide for the binding of lincosamide antibiotics (Douthwaite, S., 1992. Nucleic Acids Research 20, 4717). Although the 2OH group is less than 4.5 Å away from N6 of A2040Dr (G2057Ec) and O4 of U2590Dr (C2611Ec), additional hydrogen bonds to these nucleotides are unlikely because mutations of A2040Dr (G2057Ec) and U2590Dr (C2611Ec) are thought to alter only the conformation of the 23S rRNA and thus affect nucleotide A2041Dr (G2058Ec) (see FIG. 9b). A stereo view showing the clindamycin binding site at the peptidyl transferase cavity of D. radiodurans is shown in FIG. 9c.

The 3OH group interacts with N6 of nucleotide A2041Dr (A2058Ec) and non-bridging phosphate-oxygens of G2484Dr (G2505Ec). The distances from these moieties to the 3OH group are compatible with hydrogen bond formation. Thus, N6 of A2041Dr (A2058Ec) can interact with both the 2OH and 3OH groups of clindamycin. These structural data explain in the most straightforward way why A2041Dr (A2058Ec) mutations confer resistance. The hydrogen bond to N6 of nucleotide A2041Dr (A2058Ec) can also explain why the dimethylation of the N6 group, which disrupts the hydrogen bonds, causes resistance to lincosamides (Ross, J. I. et al., 1997. Antimicrobial Agents & Chemotherapy 41, 1162). The 3OH group of clindamycin can additionally interact with N1 of A2041Dr (A2058Ec), N6 of A2042 (A2059Ec), and the 2OH of A2482Dr (A2503Ec).

The 4OH group of clindamycin is close enough to the 2′OH of A2482Dr (A2503Ec) and to N6 and N1 of A2042 (A2059Ec) to form hydrogen bonds. This interaction explains why mutations in A2042 (A2059Ec) cause clindamycin resistance in several bacterial pathogens (Ross, J. I. et al., 1997. Antimicrobial Agents & Chemotherapy 41, 1162).

The 8′ carbon of clindamycin points towards the puromycin binding site, and is located about 2.5 Å from the N3 of C2431Dr (C2452Ec). The sulfur atom of clindamycin is located about 3 Å from base G2484Dr (G2505Ec) of the 23S rRNA. Although both of these groups cannot form hydrogen bonds, possible interactions, such as van der Waals or hydrophobic interactions, between these groups and nucleotides of the 23S rRNA may be expected.

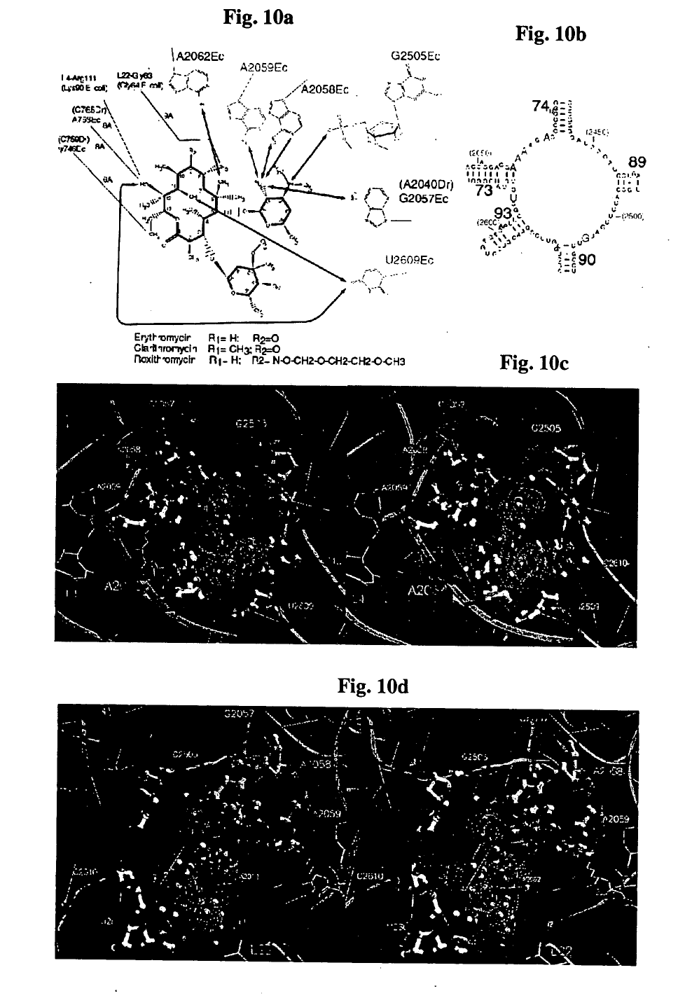

Macrolide antibiotics (erythromycin, clarithromycin, roxithronzycin): As was found to be the case for chloramphenicol and clindamycin, the binding site of the macrolides is composed exclusively of 23S rRNA and does not involve any interactions with ribosomal proteins (FIGS. 10a-d). The three macrolides analyzed were found to bind to a single site, at the entrance of the tunnel in D50S. The erythromycin and clarithromycin binding sites in the D50S peptidyl transferase cavity were found to be identical (FIG. 10c). The roxithromycin binding site at the peptidyl transferase cavity of D50S is shown in FIG. 10d. Their binding contacts clearly differ from those of chloramphenicol, but overlap to a large extent with those of clindamycin (see FIG. 11).

Most of the 14-member ring macrolides, which includes erythromycin and its related compounds, have three structural components: the lactone ring, the desosamine sugar, and the cladinose sugar. The reactive groups of the desosamine sugar and the lactone ring mediate all the hydrogen-bond interactions of erythromycin, clarithromycin, and roxithromycin with the peptidyl transferase cavity.

In all the macrolides examined, the 2′OH group of the desosamine sugar appears to form hydrogen bonds with three positions: N6 and N1 of A2041Dr (A2058Ec) and N6 of A2042Dr (A2059Ec).

These structural results explain not only the genetic studies involving 23S rRNA mutations in macrolide resistance, but also most of the results of macrolide modification in structure activity relation (SAR) studies. The hydrogen bonds between the 2′OH and N1 and N6 of A2041Dr (A2058Ec) explain why this nucleotide is essential for macrolide binding and also shed light on the two most common ribosomal resistance mechanisms against macrolides; the N6 di-methylation of A2041Dr (A2058Ec) by Erm-family methylases (reviewed in Weisblum, B., 1995. Antimicrobial Agents & Chemotherapy 39, 577) and the product of rRNA mutations changing nucleotide identity at this position (Sigmund, C. D. et al., 1984. Nucleic Acids Research 12, 4653). The di-methylation of the N6 group would not only add a bulky substituent causing steric hindrance for the binding, but would prevent the formation of hydrogen bonds to the 2′OH group. The mode of interactions proposed by the present structure implies that a mutation at A2041Dr (A2058Ec) to a nucleotide other than adenine would disrupt the hydrogen bonding pattern, therefore impairing binding and rendering bacteria antibiotic-resistant. A2041Dr (A2058Ec) is one of the few nucleotides of the peptidyl transferase ring which is not conserved among all phylogenetic domains. Sequence comparisons show that mitochondrial and cytoplasmic rRNAs of higher eukaryotes have a guanosine at position 2058Ec of the LRS RNA (Bottger, E. C. et al., 2001. Embo Reports 2, 318). Therefore, the proposed mode of interaction explains the selectivity of macrolides for bacterial ribosomes.

These results also explain the importance of the 2′OH group of the desosamine sugar for erythromycin binding known from SAR studies (Mao, J. C.-H. and Puttermann, M., 1969. Journal of Molecular Biology 44, 347) since changing the 2′OH group will not allow the suggested mode of interaction to take place. The 2′OH of the desosamine sugar is located about 4 Å away from N6 of A2040Dr (G2057Ec), a nucleotide forming the last base pair of helix 73. Although an interaction with this group cannot be ruled out, this base pair is likely engaged in maintaining the proper conformation of A2041Dr (A2058Ec), rather than interacting directly with the macrolides.

The dimethylamino group of the desosamine sugar exists in both protonated (greater than 96% to less than or equal to 98%) and neutral (2-4%) form. This group, if protonated, could interact via ionic interactions in a pH dependent manner with the backbone oxygen of G2484Dr (G2505Ec) (see FIG. 10a). At physiological pH, both species appear to be able to bind ribosomes with the same kinetics (Goldman, R. C. et al., 1990. Antimicrobial Agents & Chemotherapy 34, 426). However, the only study that revealed a strong correlation between pH and potency of inhibition of in-vitro protein synthesis for the macrolide erythromycin concludes that the neutral macrolide form was the inhibitory species. If the neutral macrolide form is in fact the inhibitory species, a hydrogen-bond between the dimethylamine of the macrolides and the nucleotide G2484Dr (G2505Ec) would not be formed.

The structural information presented herein suggest that it would be of interest to derivatize macrolides at the dimethylamine position. Such modifications could improve binding and in addition, could result in inhibition of peptidyl transferase activity. One of the few macrolides able to inhibit peptidyl transferase is tylosin. Tylosin, which contains a mycarose moiety, has been shown to protect A2506Ec, the neighboring nucleotide of G2484Dr (G2505Ec), from chemical modification (Poulsen, S. M., Kofoed, C. and Vester, B., 2000. Journal of Molecular Biology 304, 471). Similarly, modification of the dimethylamine group to a reactive group that would interact with G2484Dr (G2505Ec) in a pH independent manner may lead to more effective macrolides and structurally related compounds.

Only three of the hydroxyl moieties of the lactone ring are within hydrogen bonding distance to the 23S rRNA. The 6-OH group is within hydrogen bonding distance to N6 of A2045Dr (A2062Ec) and likely interacts with it. Although this group has been substituted by a metoxy group to improve acid stability in clarithromycin, the oxygen of the metoxy group is still at hydrogen bonding distance to N6 of A2045Dr (A2062Ec). The presently disclosed structure explains why the chemical footprinting experiments implicate this nucleotide in macrolide binding (Hansen, L. H., Mauvais, P. and Douthwaite, S., 1999. Molecular Microbiology 31, 623).

The 11-OH and 12-OH groups of the lactone group may hydrogen-bond with the O4 of U2588Dr (U2609Ec). Hydrogen bonds at these positions could explain why substitutions of any of these two hydroxyl groups cause a moderate decrease in binding, as would be expected for groups hydrogen-bonding with the same 23S rRNA nucleotide (Mao, J. C.-H. and Puttermann, M., 1969. Journal of Molecular Biology 44, 347).

The reactive groups of the cladinose sugar are not involved in hydrogen bond interactions with the 23S rRNA. Cladinose dispensability was confirmed by SAR studies (Mao, J. C.-H. and Puttermann, M., 1969. Journal of Molecular Biology 44, 347), showing that the 4″-OH is dispensable for the binding. A closely related group of antibiotics, the ketolides, which bind more tightly than macrolides to ribosomes, do not have a cladinose sugar. Moreover, the Kd of RU56006, a derivative of erythromycin lacking the cladinose sugar, is of the same order of magnitude as that of erythromycin (Hansen, L. H., Mauvais, P. and Douthwaite, S., 1999. Molecular Microbiology 31, 623).

Studies have shown that the tip of helix 35 in domain II of 23S rRNA has been implicated in the binding of erythromycin (Hansen, L. H., Mauvais, P. and Douthwaite, S., 1999. Molecular Microbiology 31, 623; Xiong, L. Q., Shah, S., Mauvais, P. and Mankin, A. S., 1999. Molecular Microbiology 31, 633). Although the presently disclosed structure does not suggest direct interaction between helix 35 and erythromycin, the distance between the OH-11 of the lactone ring and the N4 of C765Dr (A752Ec), a nucleotide proposed to interact with erythromycin, is 8.5 Å (Hansen, L. H., Mauvais, P. and Douthwaite, S., 1999. Molecular Microbiology 31, 623; Xiong, L. Q., Shah, S., Mauvais, P. and Mankin, A. S., 1999. Molecular Microbiology 31, 633), whereas the CH3 group branching from position 13 of the lactone ring is located about 4.5 Å from the O2 of C759Dr (G745Ec) (see FIG. 10a). Therefore, hydrophobic, van der Waals, or ion coordinated interactions between the lactone ring of macrolides and this loop of the 23S rRNA cannot be ruled out. In this case, footprinting effects at A752Ec might be of an allosteric nature.

The two ribosomal proteins that have been implicated in erythromycin resistance are L4 and L22 (Wittmann, H. G. et al., 1973. Molecular & General Genetics 127, 175). The closest distance of erythromycin (12-OH) to L4 (Arg111Dr/Lys90Ec) is 8 Å, whereas the closest distance from (8-CH₃) to L22 (Gly63Dr/Gly64Ec) is 9 Å. These distances are more than would be expected for any meaningful chemical interaction. Therefore, the macrolide resistance acquired by mutations in these two proteins is probably the product of an indirect effect that is produced by a perturbation of the 23S rRNA due to the mutated proteins (Gregory, S. T. & Dahlberg, A. E., 1999. Journal of Molecular Biology 289, 827).

The principal difference between the three macrolides analyzed in this study is seen at the 9 keto group of the lactone ring. Although there is no difference in the binding mode of erythromycin and clarithromycin, there is a difference in roxithromycin where an etheroxime chain substitutes the 9 keto group. The present electron density map clearly shows a small part of this etheroxime chain pointing towards the inside of the tunnel. This part of the chain is not involved in the interaction between roxithromycin and 23S rRNA or ribosomal proteins (see FIG. 10c).

Overlapping binding sites: The present results show that a subset of the 23S rRNA nucleotides involved in the hydrogen bond interactions with chloramphenicol is also involved in interactions with clindamycin. This subset consists of G2044Dr (G2061Ec) and G2484Dr (G2505Ec), whose contact with chloramphenicol is mediated via a Mg2+ ion. Both nucleotides have previously been shown by chemical footprinting to be protected by binding of chloramphenicol or clindamycin (Polacek, N. in RNA-Binding Antibiotics (eds. Schroeder, R. & Wallis, M. G.) (Eurekah.Com, Incorporated, Georgetown., 2000)) and a mutation at position G2061Ec has also been reported to confer resistance to chloramphenicol (Vester, B. & Garrett, R. A., 1988. EMBO Journal 7, 3577).

The interaction of the 23S rRNA with clindamycin and with the macrolides also involves some common nucleotide moieties. These are N6 of A2041Dr (A2058Ec), N6 of A2042Dr (A2059Ec), and the non-bridging phosphate oxygen of U2484Dr (U2505Ec). These positions are targeted by the sugar moiety of clindamycin and the desosamine sugar of the macrolides. These two sugars are located at almost identical locations in the structure of the 50S-clindamycin and 50S-macrolide complexes. The overlapping of binding sites may explain why clindamycin and macrolides bind competitively to the ribosome and why most RNA mutations conferring resistance to macrolides also confer resistance to lincosamides (Fournet, M. P. et al., 1987. J Pharm. Pharmacol. 39, 319).

Nucleotide G2484Dr (G2505Ec) is targeted by all antibiotics tested in this study. The importance of this nucleotide position has been previously established (Saarma, U. et al., 1998. Rna-a Publication of the Rna Society 4, 189). Although this nucleotide has been shown to be protected from chemical modification upon chloramphenicol, lincosamide, or macrolide binding (Rodriguez-Fonseca, C. et al., 1995. Journal of Molecular Biology 247, 224; Moazed, D. and Noller, H. F., 1987. Biochemie 69, 879; Douthwaite, S., 1992. Nucleic Acids Research 20, 4717), no mutations of G2484Dr (G2505Ec) conferring resistance to these antibiotics have been reported. In addition, G2484Dr (G2505Ec) is also one of the nucleotides protected upon binding of peptidyl-tRNA (Moazed, D. and Noller, H. F., 1991. Proc Natl Acad Sci USA. 88, 3725). The identity of this nucleotide is important for protein synthesis, albeit not for ribosome-antibiotic interactions where the position of its backbone oxygen or the 2′OH of the sugar appear to be the essential requirement.

Thus, the characterization of overlapping binding sites provided by the present studies indicates the essential 23S rRNA nucleotides which must be targeted by antibiotics in order to inhibit ribosomal function. Such characterization is extremely valuable for the rational design of combined antibiotic therapies and, moreover, can open the door for the design of hybrid antibiotics comprising novel combinations of ribosomal binding sites.

Mechanism of antibiotic action: The relative binding sites for chloramphenicol, clindamycin, and erythromycin in 23S rRNA with respect to the A-site substrate analog CC-puromycin (Nissen, P. et al., 2000. Science 289, 920) and the 3′end of P- and A-site tRNAs (Yusupov, M. M. et al., 2001. Science 292, 883) docked in the present structure are shown in FIG. 11. From these sites, it can be seen that chloramphenicol acts as a competitor of puromycin and thus, as an A-site inhibitor, consistent with previous findings (Vazquez, D. Inhibitors of protein synthesis (Springer Verlag, Berlin, Germany, 1975)). In contrast to puromycin, which acts as a structural analog of the 3′-end aminoacyl tRNA, the location of chloramphenicol in the present structure suggests that this drug may act by interfering with the positioning of the aminoacyl moiety in the A-site. Thus, chloramphenicol may physically prevent the formation of the transition state during peptide bond formation. In addition, the dichloromethyl moiety of chloramphenicol, a moiety shown to be important for chloramphenicol activity (Vince, R. et al., 1975. Antimicrobial Agents & Chemotherapy 8, 439), is close enough to the amino acceptor group of CC-puromycin (FIG. 11). The presence of such an electronegative group in the neighborhood of the amino acceptor could also hamper peptide bond formation.

Clindamycin clearly bridges the binding site of chloramphenicol and that of the macrolides (FIG. 11) and overlaps directly with the A- and P-sites. Thus, its binding position provides a structural basis for its hybrid A-site and P-site specificity (Kalliaraftopoulos, S. et al., 1994. Molecular Pharmacology 46, 1009). Furthermore, clindamycin has the capacity to interfere with the positioning of the aminoacyl group at the A-site and the peptidyl group at the P-site while also sterically blocking progression of the nascent peptide towards the tunnel.

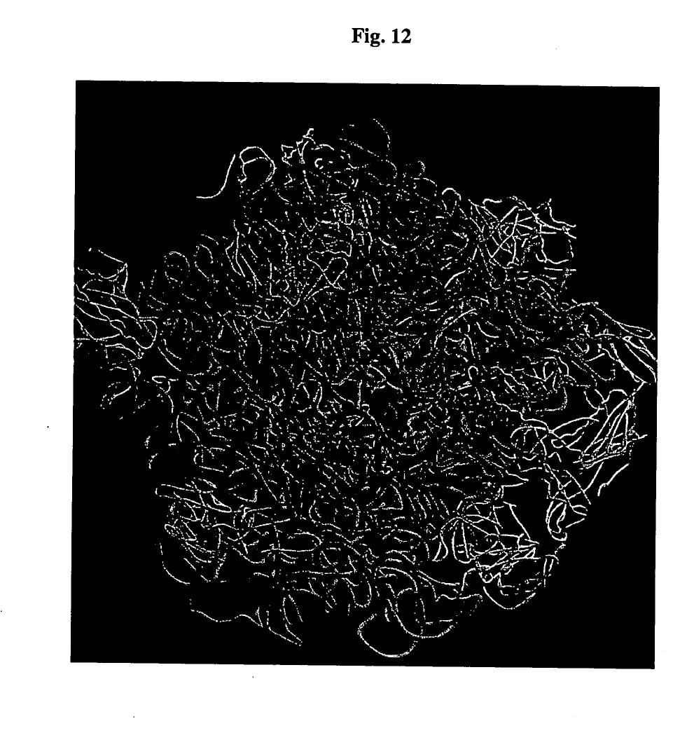

Macrolides, on the other hand, are thought to block the progression of the nascent peptide within the tunnel (Vazquez, D. Inhibitors of protein synthesis (Springer Verlag, Berlin, Germany, 1975)) and, indeed, the present structure shows erythromycin as being located at the entrance of the tunnel (FIG. 12). The macrolide binding site is located at a position that can allow the formation of 6-8 peptide bonds before the nascent protein chain reaches the macrolide binding site. Once macrolides are bound, they reduce the diameter of the tunnel from the original 18-19 Å to less than 10 Å. However, since the space not occupied by erythromycin hosts a hydrated Mg2+ ion, the passage available for the nascent protein is effectively reduced to 6-7 Å in diameter. Moreover, in order to reach this narrow passage the nascent peptide needs to progress in a diagonal direction, thus imposing further limitations on the growing protein chain. These structural results are consistent with previous biochemical findings, showing that peptides of up to 8 residues can be produced by erythromycin-bound ribosomes (Tenson, T. et al., 1996. Proc Natl Acad Sci USA. 93, 5641). It is possible that ribosomes could escape the inhibitory effects of macrolides if proteins with leader sequences specifically capable of threading their way through the narrowed tunnel are translated.

Overall, the binding sites of the antibiotics analyzed in the present study suggest that their inhibitory action is not only determined by their interaction with specific nucleotides, some of them shown to be essential for peptidyl transferase activity and/or A- and/or P-tRNA binding. These antibiotics could also inhibit peptidyl transferase activity by interfering with the proper positioning and movement of the tRNAs in the peptidyl transferase cavity. This steric hindrance may be direct, as in the case of chloramphenicol or indirect as in the case of the three macrolides. In addition to causing steric hindrance, antibiotic-binding may physically link regions known to be essential for the proper positioning of the A- and P-tRNAs and thus prevent the conformational flexibility needed for protein biosynthesis.

The overall interaction of each analyzed antibiotic, together with the lack of any major conformational changes upon antibiotic-binding to the ribosome, supports the theory whereby the PTC evolved as a template for the proper binding of the activated substrates, the A-tRNA and the P-tRNA (Polacek, N. et al., 2001. Nature 411, 498). However, the presence of a catalytic nucleotide cannot be completely excluded.

Conclusion: The present analyses reveal at atomic resolution, for the first time, the structural and functional interactions involved in the binding of chloramphenicol, clindamycin, and the three macrolides erythromycin, clarithromycin, and roxithromycin to a bacterial ribosome and provide a wealth of novel and valuable information regarding the functional and structural basis of antibiotic resistance. As such, the present three-dimensional atomic structure models are very clearly advantageous over prior art models of antibiotic-ribosome interaction. Such models can be used to elucidate the mechanisms whereby ribosomes perform the crucial biological process of protein translation and the mechanisms whereby antibiotics inhibit ribosome function. Crucially, these models constitute a powerful tool for the rational design or selection of novel antibiotics. This is of critical importance, particularly due to current epidemics of diseases caused by antibiotic resistant or multi-resistant strains of bacterial pathogens. Furthermore, the models of the present invention provide much novel and valuable information regarding the mechanisms of ribosome function per se. As such, these models can therefore be utilized to rationally design or select ribosomes having desired characteristics, such as enhanced protein production capacity, which can be used to enhance recombinant protein production. Thus, the unique antibiotic-LRS complex models of the present invention can be advantageously applied in a broad range of biomedical, pharmacological, industrial and scientific applications.

### Example 3

### High Resolution Three-Dimensional Atomic Structure Models of the Interaction of Ketolide and Azalide Antibiotics with the Large Ribosomal Subunit-Elucidation of Mechanisns of Activity Against Macrolide Resistant Bacteria

Currently expanding epidemics of diseases caused by antibiotic resistant bacterial strains have generated an urgent need for novel and improved antibiotics. Whereas certain antibiotics, such as macrolides, have been modified to antibiotics, such as ketolides and azalides, displaying partial activity against bacteria resistant against antibiotics such as macrolide antibiotics, such ketolide and azalide antibiotics nevertheless exhibit suboptimal efficiency, and the mechanisms whereby these exert their partial activity against antibiotic resistant bacteria remain poorly characterized. Ideally, in order to develop or identify optimal antibiotics, the atomic interactions of ribosomal subunits in complex with antibiotics must be elucidated via X-ray crystallography, however, prior art three-dimensional atomic structures of such complexes have not provided information useful for designing optimal antibiotics in many cases, nor for elucidating the mechanisms whereby modified antibiotics, such as ketolides and azalides, display partial activity against antibiotic-resistant bacteria. In order to overcome such limitations, the present inventors have generated crystals of the eubacterial large ribosomal subunit in complex with the azalide azithromycin or the ketolide ABT-773, have used such crystals to solve the three-dimensional atomic structure of the interaction between the eubacterial LRS and such antibiotics, and have analyzed such structures, as follows.

Materials and Methods:

Crystallization: Crystals of the large ribosomal subunit of D. radiodurans (D50S) were generated as described in Example 1 above (Harms J. et al., 2001. Cell 107, 679-688; Schlüenzen et al., 2001. Nature 413, 814-821). Co-crystallization of D50S with the antibiotic azithromycin (CP-62,993 [9-deoxy-9A-methyl-9A-aza-9A-homoerythromycin], Pliva, Croatia) or ABT-773 (Abbott Laboratories) was achieved using a 7-fold excess of antibiotic relative to D50S in the crystallization solution.

X-ray diffraction: Data were collected at 85 K from shock-frozen crystals using a synchrotron radiation beam at ID 19 at Argonne Photon Source/Argonne National Laboratory (APS/ANL) and ID14/2, ID14/4 at the European Synchrotron Radiation Facility/European Molecular Biology Laboratory (ESRF/EMBL). Data were recorded on Quantum 4 or APS-CCD detectors and processed with HKL2000 (Otwinowski, Z. and Minor, W., 1997. Macromolecular Crystallography Pt A. 276, 307) and the CCP4 suite (Bailey, S., 1994. Acta Cryst D. 50, 760).

Localization and refinement: The 3.1 Å structure of D50S (PDB accession code 1LNR) was refined against the structure factor amplitudes of each of the two D50S-antibiotic complexes, using rigid body refinement as implemented in CNS (Brunger, A. T. et al., 1998. Acta Crystallographica Section D-Biological Crystallography 54, 905-921). For the calculation of the free R-factor, 7% of the data were omitted during refinement. The positions of the antibiotics were readily determined from sigmaA-weighted difference maps. The superior quality of the difference maps, showing the hole in lactone ring, unambiguously revealed the position and orientation of the lactone ring. The electron density for the secondary binding site of azithromycin, as described below, indicated a small conformational change in the lactone ring, but the observed differences were considerably smaller than the average coordinate error (about 0.7 Å, according to Luzzati plots), therefore modeling of the presumed conformational change was therefore not attempted.

Coordinates and generation of atomic structures: Atomic structure coordinates of ABT-773 and azithromycin were provided by Abbott Laboratories and Pliva, Croatia, respectively. Three-dimensional atomic structures were generated from coordinates using RIBBONS software (Carson, 1997. Macromolecular Crystallography, Pt B 277, 493-505). The two-dimensional figures displaying the D50S-antibiotic interactions were generated with LIGPLOT software (Wallace et al., 1995. Protein Engineering 8, 127-134).

Nucleotide and amino acid coordinate numbering: Nucleotide coordinates of 23S rRNA numbered according to the D. radiodurans or E. coli coordinate numbering scheme are indicated with the suffix “DR” or “EC”, respectively. Amino acid coordinates of D50S polypeptides are numbered according to the D. radiodurans amino acid residue numbering scheme.

Experimental Results:

The crystal structures of the D50S ribosomal subunit in complex with azithromycin and ABT-773 diffracted to resolutions of 3.2 Å and 3.5 Å, respectively (Table 17).

Atomic structure of D50S-ABT-773 and D50S-azithromycin complexes: The three-dimensional atomic structure of D50S in complex with ABT-773 or azithromycin was found to be defined by the sets of pdb atom coordinates set forth in Table 18 or Table 19, respectively (refer to enclosed CD-ROM). Structure coordinates for proteins in these tables refer to alpha-carbon atoms of amino acid residues, and the chain ID corresponding to constituents of these complexes used in these tables are specified in Table 20.

TABLE 17
Statistics of data collection.

| | | Complete- | | | | R- |
|---|---|---|---|---|---|---|
| Co-crystallized | Resolution* | ness | Rsym | | Rf | free |
| antibiotic | (Å) | (%) | (%) | I/σ(I) | (%) | (%) |
| ABT-773 | 30.0-3.50 | 91.8 | 13.1 | 7.5 | 28.5 | 31.3 |
| | (3.56-3.50) | (88.9) | (35.8) | (2.1) | | |
| Azithromycin | 30.0-3.20 | 86.6 | 11.0 | 9.0 | 27.9 | 30.4 |
| | (3.25-3.20) | (83.4) | (33.9) | (1.9) | | |

*Numbers in brackets denote the highest resolution bin.

TABLE 20
Chain ID's corresponding to constituents of D50S
complexes used in Tables 18-19.

| Constituent | Chain ID |
|---|---|
| 23S rRNA | 0 |
| 5S rRNA | 9 |
| ribosomal proteins L2-L6 | A-E |
| ribosomal protein L9 | F |
| ribosomal protein L11 | G |
| ribosomal proteins L13-L24 | H-S |
| ribosomal protein CTC | T |
| ribosomal proteins L27-L32 | U-Z |
| ABT-773 | ABT |
| azithromycin | AZI |

The electron-density maps generated allowed unambiguous determination of the binding sites of both ABT-773 and azithromycin with the LRS. The superior quality of the maps correlates with the high affinity of both drugs for the D50S subunit. The interactions of ABT-773 with specific nucleotides of the multi-branched loop of domain V were found to be similar to those of erythromycin, but additional contacts with domains II and IV may lead to tighter binding.

Azithromycin was unexpectedly found to display a secondary binding site. The primary site was found to be located in a position and orientation quite similar to that observed for erythromycin or for ABT-773, and the second molecule was found to be in direct contact with the primary binding site, with both sites blocking the path of the nascent chain. The secondary site directly interacts with ribosomal protein L4 in a manner which presumably makes this site highly specific for D. radiodurans, as explained hereinbelow.

The results relating to both azithromycin and ABT-773 were found to be consistent with the majority of mutational and footprinting data (Garza-Ramos et al., 2001. Journal of Bacteriology 183, 6898-6907; Weisblum, 1998. Drug Resistance Updates 1, 29-41), thus explaining the different modes of interactions and differences in activity against certain macrolide-lincosamide-streptogramin B (MLSB) resistant phenotypes.

Atomic structure of the ABT-773 binding site of D50S: Three-dimensional atomic structures of the portion of D50S-ABT-773 complex comprising all of ABT-773 and portions of D50S contained within a 20 Å-radius sphere centered on ABT-773 (coordinates 44, 127, 121) were found to be defined by the subset of Table 18-derived coordinates (refer to enclosed CD-ROM) set forth in Table 21 (refer to enclosed CD-ROM).

Analysis of the D50S-ABT-773 complex atomic structure contained in the above-described 20 Å-radius sphere demonstrated that ABT-773 interacts with the LRS via 23S rRNA nucleotides having coordinates C803, C1773, A2040, A2041, A2042, A2045, G2484, U2588, C2589, and U2590.

Atomic structure of the azithtromycin binding site of D50S: Three-dimensional atomic structures of the portion of D50S-azithromycin complex comprising all of azithromycin and portions of D50S contained within a 20 Å-radius sphere centered on azithromycin (coordinates 42, 120, 130) were found to be defined by the subset of Table 19-derived coordinates (refer to enclosed CD-ROM) set forth in Table 22 (refer to enclosed CD-ROM).

Analysis of the D50S-azithromycin complex atomic structure contained in the above-described 20 Å-radius sphere demonstrated that azithromycin interacts with the LRS via: 23S rRNA nucleotides having coordinates A764, A2041, A2042, A2045, A2482, G2484, C2565, U2588, C2589, and U2590; ribosomal protein L4 amino acid residues having coordinates Y59, G60, G63, and T64; and ribosomal protein L22 amino acid residue having the coordinate R111.

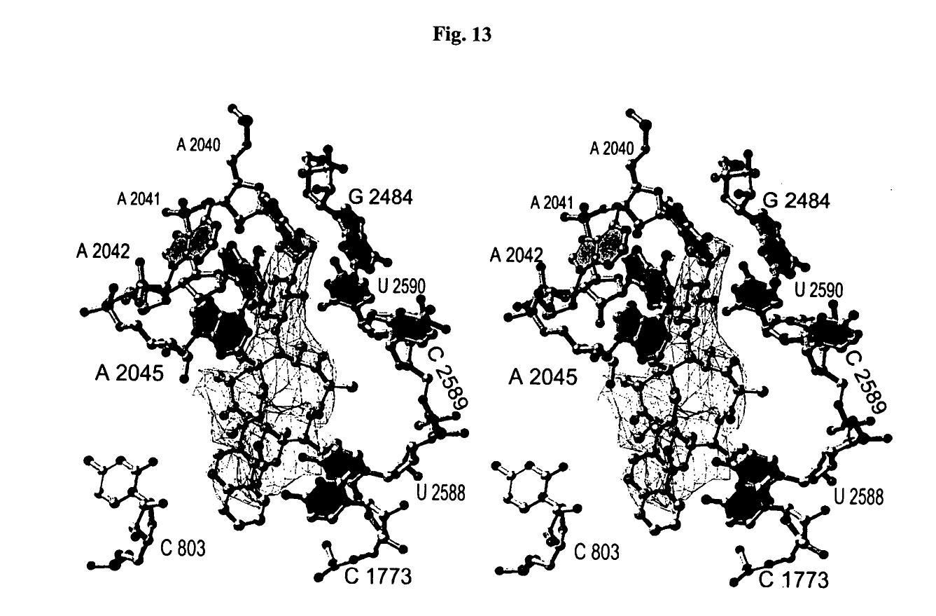

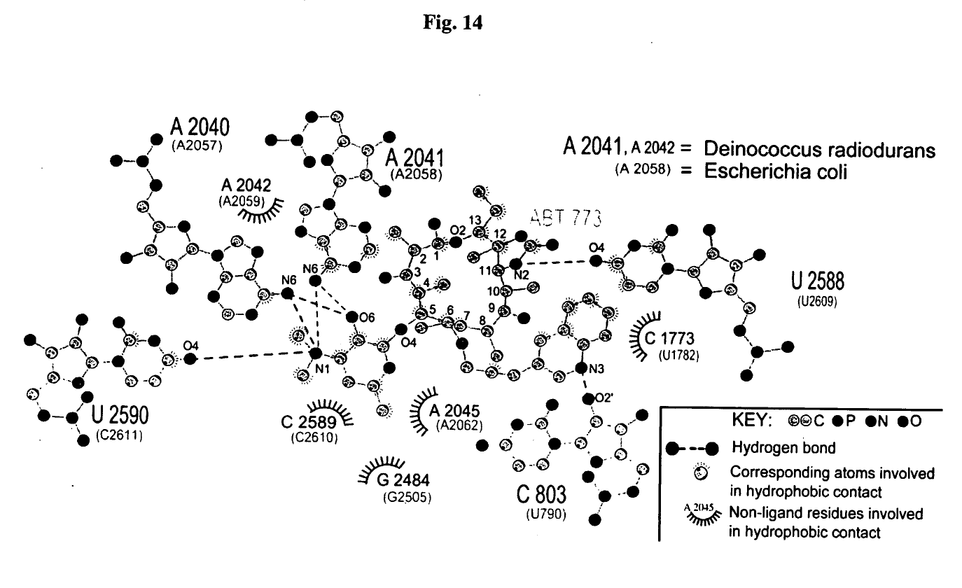

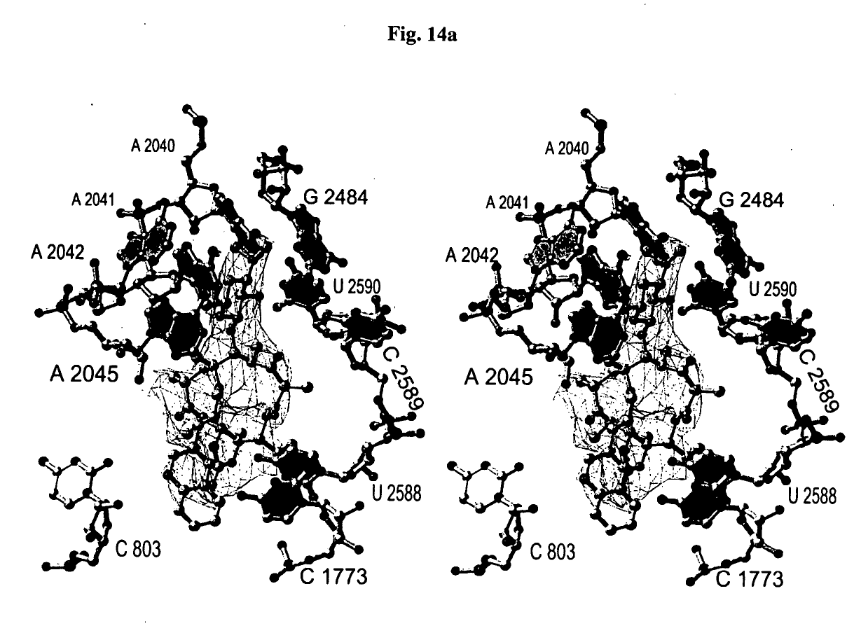

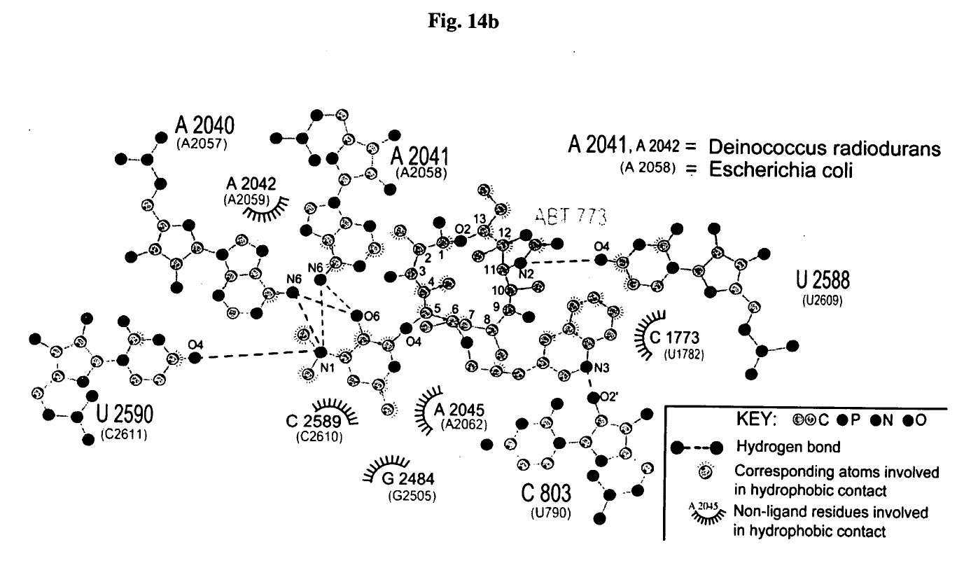

ABT-773: The interactions between ABT-773 and the LRS are shown in FIGS. 13 and 14. The position of ABT-773 appears displaced by about 3 Å compared to the position observed for the macrolides of the erythromycin class, while still leading to the same inhibitory mechanism, namely blockage of the path of the nascent chain through the LRS. The positional shift seems to be induced by the interactions of 23S rRNA with the different functional groups of ABT-773. Both erythromycin modifications, the quinollyallyl and the carbamate groups enhance binding of ABT-773 through the formation of hydrogen bonds and hydrophobic interactions, but the quinollyallyl group does not seem to be the major determinant of the interaction of ABT-773 with 23S rRNA.

The quinollyallyl group is tethered to the macrolide skeleton through a rather flexible linker, which allows the moiety to occupy different spatial positions. The inherent flexibility is reflected by the electron density of the quinollyallyl group, which appears to be less well resolved (FIG. 13). The position of the quinollyallyl best fitting the electron density, allows N3 of the quinollyallyl to form a hydrogen bond with O2′ of C803DR (U790EC) of domain II (FIGS. 13-14 and 15b). This is in agreement with biochemical experiments indicating that domain II might be involved in the binding of some of the ketolides (Garza-Ramos et al., 2001. Journal of Bacteriology 183, 6898-6907; Xiong et al., 1999. Molecular Microbiology 31, 633-639). The quinollyallyl group might stabilize the binding of ABT-773 but does not seem to be a major determinant of the interaction of ABT-773 with 23S rRNA. The rRNA base A752EC (C765DR) was shown to be protected upon binding of ketolides carrying C-11,12 extensions (Hansen et al., 1999. Molecular Microbiology 31, 623-631). C765DR is in the vicinity of the binding site of ABT-773, but does not appear to contribute to the binding of the drug. However, exchanging C765DR to A765DR would possibly create a direct contact between A765DR and ABT-773, consistent with biochemical data (Douthwaite et al., 2000. Molecular Microbiology 36, 183-192). Nevertheless, it is questionable if this interaction further enhances the affinity of ABT-773 for 23S rRNA, a doubt which is also supported by the susceptibility of Heliobacterpylori (C752EC) to ketolides.

The mutation U754EC to A754EC (G767DR) in domain II has been shown to confer resistance to low levels of the ketolide HMR3647 (Xiong et al., 1999. Molecular Microbiology 31, 633-639). Since neither 754EC nor 756EC have been found to be directly involved in binding of ABT-773 or any of the macrolides studied so far, as described above in Example 2 (Schlüenzen et al., 2001. Nature 413, 814-821), it seems that the effect of mutations of these bases on the binding of ketolides is mediated through small conformational shifts affecting the loop connecting helices H35a and H32.

Domain IV, which has not previously been reported to be involved in binding of the ketolides, contributes to the positioning of the quinollyallyl group through hydrophobic interactions of C1773DR (U1782EC). The interactions of the quinollyallyl group with the 23S rRNA seems to be completely independent of the gender of the two rRNA bases involved. Mutations in either C803DR or C1773DR are hence not expected to affect binding of ABT-773. This agrees with the observation that E. coli, possessing uridines in both positions (U/U), and pathogens such as Heliobacter pylori (U/U) or Haemophilus influenzae (A/C) are equally susceptible to ABT-773 (Andrews et al., 2000. Journal of Antimicrobial Chemotherapy 46, 1017-1022; Credito et al., 2001. Antimicrobial Agents and Chemotherapy 45, 67-72).

U2588DR (U2609EC) of domain V of 23S rRNA forms a network of hydrophobic interactions with the carbamate group of ABT-773. Together with the hydrogen bond between O4 of U2588DR and N2 of the carbamate group, these interactions seem to be the main contributors for the enhancement of the binding of ABT-773 to the ribosome, compared to other 14-membered macrolides, rendering the cladinose sugar dispensable. This is in accord with the observation that the mutation U2609EC to C2609EC, which yields ABT-773- and telithromycin-resistant phenotypes, slightly increased the sensitivity to macrolides such as erythromycin and azithromycin, which both lack the carbamate group. This was previously shown by Garza-Ramos and co-workers (Garza-Ramos et al., 2001. Journal of Bacteriology 183, 6898-6907), who have further proposed that the enhanced potency of ABT-773 correlates with stronger interactions of the drug with domain II of 23S rRNA through the quinollyallyl group and with domain V through the carbamate group.

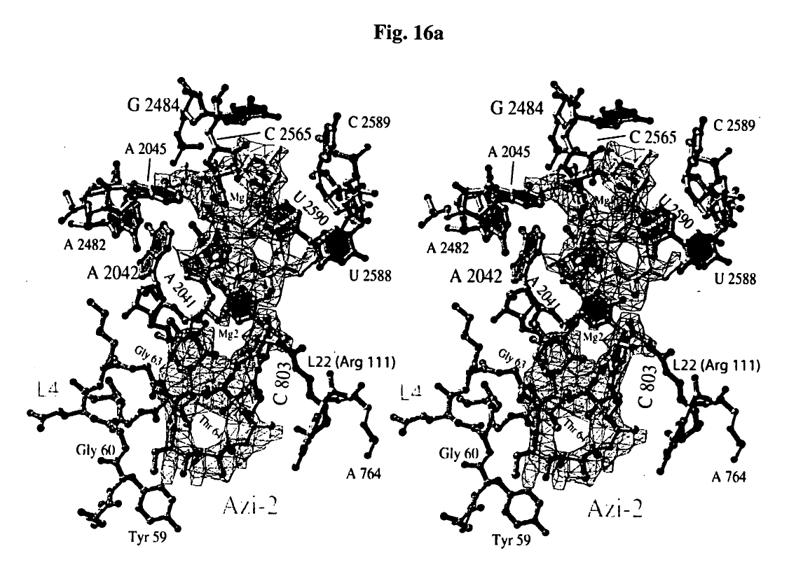

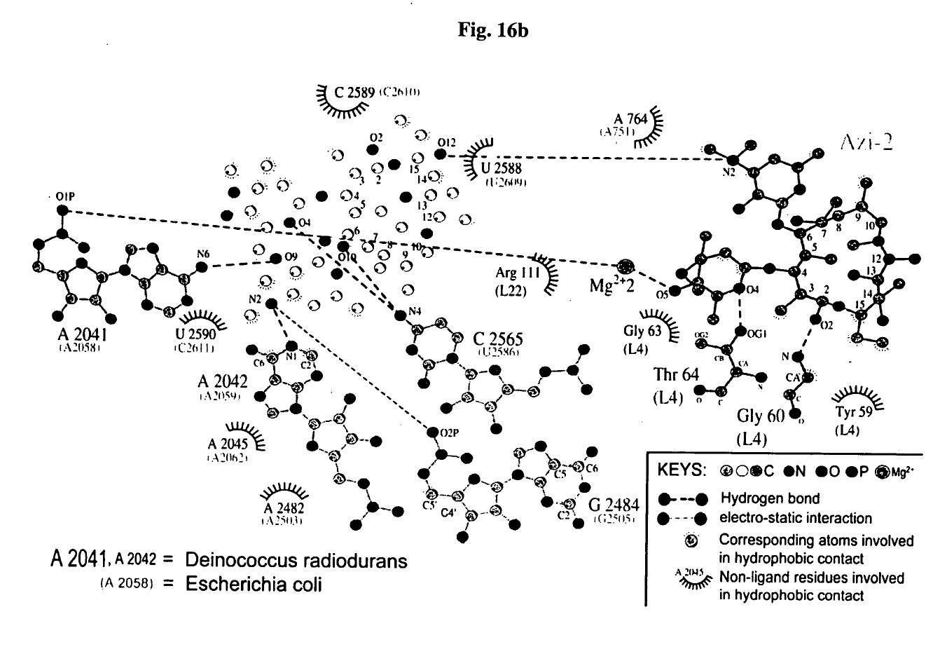

Azithromycin: Analysis of the electron density of the LRS-azithromycin complex (FIG. 16a) unambiguously demonstrated that the mode of binding of azithromycin to the LRS differs significantly from the binding of the other macrolides studied so far, as described above in Example 2. Two azithromycin binding sites were unexpectedly found in the tunnel of the 50S subunit, leading to a more extended conformational change in the 23S rRNA.

The primary binding site of azithromycin (AZI) is located in a position rather close to the location of the other macrolides analyzed, as described in Example 2 above. In particular the lactone ring lies in the same plane as the lactone rings of all other macrolides studied. The small shift of AZI of about 2 Å compared to erythromycin leads to small alterations in the interactions. Bases 2045DR and U2588DR (U2609EC) contribute to the binding of AZ1 through hydrophobic interactions FIG. 16b. A putative Mg2+ ion (Mg1), which is probably coordinated through water molecules, might contribute to the interactions through hydrogen-bonds with the cladinose-sugar and the lactone ring. Since water molecules cannot be detected, the potential hydrogen-bonds involving Mg1 have been omitted from FIG. 16b and Table 23. The lactone ring itself makes less extensive hydrophobic interactions than ABT-773 or the secondary binding site of azithromycin (AZ2). The lower contribution of the lactone ring to the stability of the AZ1 position is reflected by the less well resolved electron density for this part of the model.

TABLE 23
Interactions of 23S rRNA nucleotides with structural components of macrolide antibiotics*.

| | Structural component of macrolide antibiotic | | | | | |
|---|---|---|---|---|---|---|
| | | | | | cyclic | |
| | Desosamine | | Cladinose | etheroxime | carbamate | |
| Antibiotic | sugar | Lactone ring | sugar | chain | group | Quinollallyl |
| ABT-773 | A2041 A2040 | U2588 A2041 | — | — | C1773 | C1773 C803 |
| | A2042 A2045 | C2589 A2042 | | | U2588 | |
| | G2484 C2589 | | | | | |
| | U2590 | | | | | |
| AZI | A2041 A2042 | U2588 C2565 | A2042 C2565 | — | — | — |
| | A2045 A2482 | C2589 Azi² | C2589 U2590 | | | |
| | G2484 | | | | | |
| ERY | A2041 A2040 | U2588 A2042 | A2042 A2045 | — | — | — |
| | A2045 A2482 | C2589 A2045 | C2589 | | | |
| | G2484 | U2590 | | | | |
| ROX | A2041 A2040 | U2588 A2042 | A2042 A2045 | C1773 A2045 | — | — |
| | A2045 A2482 | C2589 A2045 | C2589 | | | |
| | G2484 | U2590 | | | | |

*Interactions which are common among all macrolides investigated are shown in bold, regardless of the type of interaction.
Azi²—second azithromycin molecule,
AZI—azithromycin,
ERY—erythromycin,
ROX—roxithromycin.

Surprisingly, the nitrogen inserted into the lactone ring does not directly contribute to the enhanced affinity of azithromycin. It seems however, that this modification alters the conformation of the lactone ring sufficiently to induce novel contacts to the magnesium ion and C2565DR, which appear to be the major causes of the enhanced affinity of azithromycin.

Contrary to AZI, which interacts exclusively with domains IV and V of 23S rRNA, the lactone ring of AZ2 makes a slightly stronger contribution of hydrophobic interactions which also involve L4, L22 and domain II of 23S rRNA. The main source of interactions originates from hydrogen bonds between Thr64 and Gly6O of the loop of L4 and another putative magnesium ion. The short distance between Gly6O and AZ2 indicates that any other residue in this position will at least alter the positioning of AZ2, and will probably inhibit the binding of AZ2 to the secondary site. However, sequence alignment against all available bacterial ribosomal L4 proteins revealed no other bacterial organism with a glycine present in this position. According to the sequence alignment, 94% of the L4 sequences carry either a Lys or Arg in this position, the remaining 6% were Pro or Ala residues. Hence, the occurrence of a secondary binding site, seems to be highly specific for D. radiodurans. This observation is also supported by kinetic analysis of the binding of azithromycin in E. coli (Dinos et al., 2001. Molecular Pharmacology 59, 1441-1445), which did not reveal a pattern as expected for double binding sites, though it was shown earlier, that macrolides such as erythromycin have the potential to bind to ribosomal proteins L5, L21 and L4 (Suryanarayana, 1983. Biochemistry International 7, 719-725).

Furthermore, AZ2 makes a direct contact to AZ1 through a hydrogen bond between its desosamine-sugar and O1 in the lactone ring of AZ1. This kind of contact will probably not be possible with either 14- or 16-membered macrolides, which further restricts the selectivity of the secondary site to D. radiodurans and 15-membered macrolides.

Due to space constraints, it seems unlikely that AZ2 will be able to bind after AZ1 is in place. The density for the lactone ring of AZ2 was considerably better resolved than that for the lactone ring of AZ1, possibly indicating that, in this particular environment, the affinity for the secondary site might be higher than for the primary site.

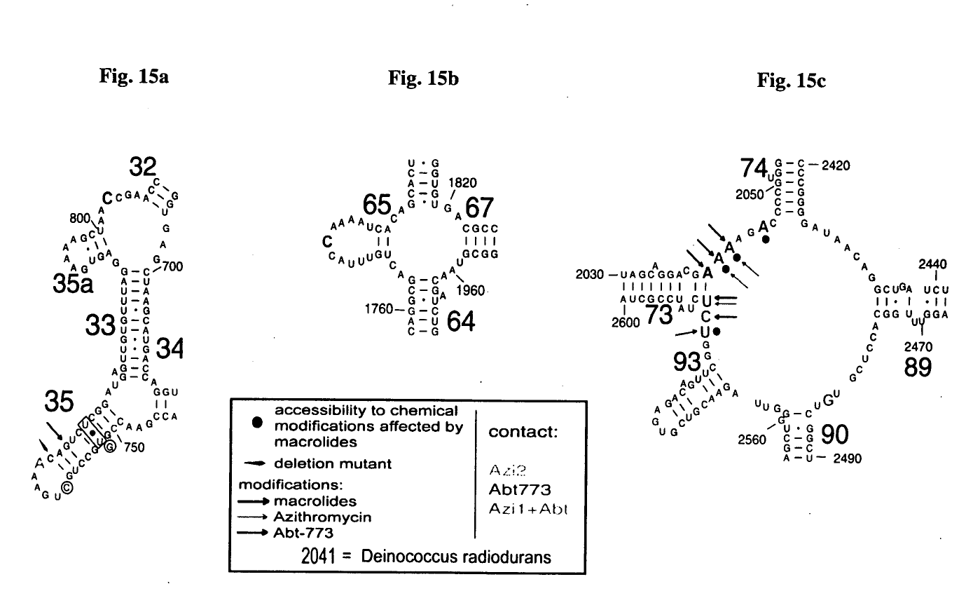

The majority of the observed remaining interactions of 23S rRNA with ABT-773, and the primary binding site of azithromycin, exclusively involving bases of the PTR, agree well with the results obtained for erythromycin, roxithromycin and clarithromycin described in Example 2 above (Schlüenzen et al., 2001. Nature 413, 814-821). As shown in FIGS. 15a-c, the interactions of ABT-773 and AZ1 correlate extremely well with macrolide and ketolide resistances, as well as with changes in the accessibility of the 23S rRNA to chemical modification (Weisblum, 1998. Drug Resistance Updates 1, 29-41).

Summary: Analysis of the crystal structures of D50S-azithromycin or D50S-ABT-773 complexes indicated that despite differences in the number and nature of their contacts with the ribosome, both compounds exert their antimicrobial activity by blocking the protein exit tunnel. In contrast to all macrolides studied so far, two molecules of azithromycin were found to bind simultaneously to the tunnel. The additional molecule also interacts with two proteins, L4 and L22, both implicated in macrolide resistance. The atomic structures described above enabled elucidation of the mechanisms underlying the enhanced activity of ketolides and azalides against specific macrolide-resistant bacteria. The present results confirm the results described in Example 2, above, relating to the interaction of macrolides with the eubacterial LRS, and extend the findings to two relatively novel classes of antibiotics, the ketolides and the azalides. These results emphasize the importance of the lactone ring for the proper positioning of the corresponding drug, and demonstrate that, nevertheless, the flexibility of the rRNA-bases interacting with the antibiotics, and also the flexibility of the moieties of the antibiotics involved in interactions with the ribosome, can compensate to some extent for modifications or alterations. The observed differences between the binding modes of macrolides, azalides and ketolides revealed the contributions of the chemical modifications of the macrolides, based on their enhanced binding properties of these compounds as well as the resistances towards these antibiotics.

Conclusion: The present analyses reveal at atomic resolution, for the first time, the structural and functional interactions involved in the binding of ketolides and azalides to the eubacterial LRS and provide a wealth of novel and valuable information regarding the functional and structural basis of the partial activity of such antibiotics against antibiotic resistant strains of bacteria. As such, the present three-dimensional atomic structure models are very clearly advantageous over prior art, being unique relative to the prior art. Such models can provide valuable information for elucidating the mechanisms of protein synthesis, and the mechanisms whereby azalide and ketolide antibiotics inhibit ribosome function. Importantly, these models constitute a powerful tool for the rational design or selection of novel antibiotics effective against antibiotic resistant strains of bacteria. This is of particular relevance in light of current epidemics of diseases caused by antibiotic resistant bacteria. Furthermore, the above-described models of the present invention provide much novel and valuable information regarding the basic mechanisms of ribosome function. As such, these models can therefore be utilized to facilitate the design or selection of ribosomes having desired properties, such as enhanced protein production capacity, which can be used to enhance recombinant protein production. Thus, the above-described atomic structure models of the present invention can be advantageously applied in a broad range of biomedical, pharmacological, industrial and scientific applications.

### Example 4

### High Resolution Three-Dimensional Atomic Structure Models of the Interaction of Puromzycin and Sparsomycin with the Large Ribosomal Subunit: Structural Basis for Peptidyl Transferase Activity and Inhibition of Protein Synthesis by Puromycin and Sparsomycin

Many diseases caused by bacterial pathogens, including diseases caused by antibiotic resistant bacteria have led to an urgent need for novel and improved antibiotics effective against such pathogens. In order to rationally design or identify such antibiotics, it would be ideal to obtain models of the atomic interactions of ribosomal subunits in complex with protein synthesis inhibitors, such as sparsomycin, and/or ribosomal substrate analogs, such as tRNA analogs, via X-ray crystallography. Such models could also be used to for facilitating rational design or identification of ribosomes having desired properties. However, prior art three-dimensional atomic structures of such complexes have not provided information useful for designing optimal antibiotics. In order to overcome this limitation of the prior art, the present inventors have generated crystals of the eubacterial LRS in complex with sparsomycin and/or tRNA substrate analogs, and have used such crystals to solve the three-dimensional atomic structural basis of the interaction between the eubacterial LRS and such compounds, as follows.

Materials and Methods:

For better understanding of the mechanism of peptidyl transferase activity and of the dynamic aspects associated with it, the three-dimensional structures of the LRS in complex with the following tRNA substrate analogs and/or the antibiotic sparsomycin were determined:

(i) D50S complexed with a puromycin-conjugated substrate analog mimicking the tRNA acceptor stem (ASM),

(ii) D50S complexed with a puromycin-conjugated substrate analog mimicking the CCA 3′ end of the tRNA acceptor stem (ACCP),

(iii) D50S complexed with the universal ribosome inhibitor sparsomycin, or

(iv) D50S complexed with a combination of ASM and sparsomycin (ASMS).

The structures of these complexes were analyzed in order to elucidate of the modes of interactions between the ribosome, the substrate analogs and the ribosomal inhibitors; illuminate elements of flexibility within the peptidyl transferase cavity, including those facilitating the interplay between the A- and P-sites; elucidate the principles of the action of puromycin and sparsomycin; and identify feature that contribute to the dynamics of translocation.

Nucleotide and helix numbering: Nucleotides and helical features are numbered according to E. coli, unless specified otherwise.

Crystallization: D50S crystals were grown as described in Example 1 above (Harms J. et al., 2001. Cell 107, 679-688).

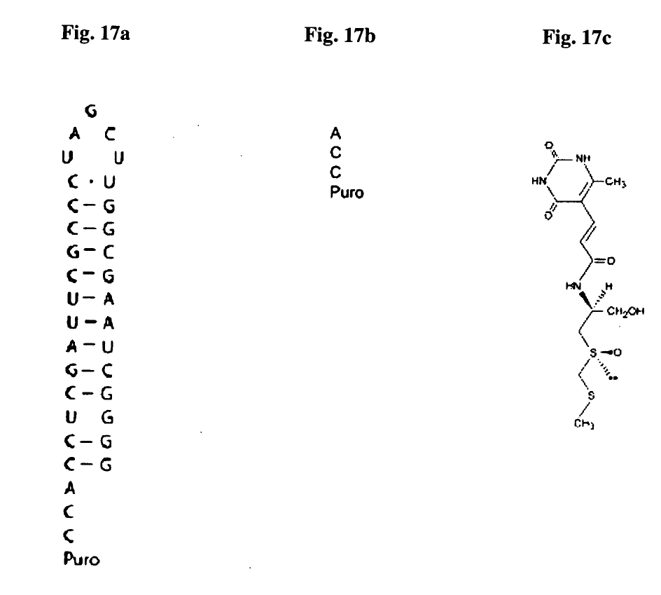

D50S-ASM crystals were obtained by soaking D50S crystals in solution containing 0.025 mM of ASM, a puromycin-coupled 35-nucleotide RNA molecule having the sequence: 5′-GGGGCUAAGCGGUUCGAUCCCGCUUAGCUCCACC-Puro (SEQ ID NO: 1; FIG. 17a). The 3′ end of the RNA molecule was coupled via a phosphodiester bond to the 5′ OH of the N6-dimethyl moiety of puromycin.

D50S-ACCP crystals were obtained by soaking D50S crystals in solutions containing 0.0125 mM of ACCP, which is a puromycin-coupled RNA molecule having the sequence 5′-ACC-Puro (SEQ ID NO: 2; FIG. 17b). The terminal C of the RNA molecule was coupled via a phosphodiester bond to the 5′ OH of the N6-dimethyl moiety of puromycin.

D50S-sparsomycin crystals were generated by co-crystallization of D50S in the presence of a 10-fold excess of sparsomycin (FIG. 17c).

D50S-ASMS crystals were obtained by soaking D50S-sparsomycin crystals in a solution containing 0.025 mM ASM.

X-ray diffraction: Data were collected at 85 K from shock-frozen crystals using a bright SR beam at ID19 at APS/SBC/ANL. Data were recorded on Quantum 4 or APS-CCD detectors and processed with HKL2000 software (Otwinowski and Minor, 1997. Macromolecular Crystallography, Pt A 276, 307-26).

Placements and refinement: Using the 3.0 Å structure of D50S (PDB entry 1LNR) as a starting model, the 3.0 Å structure of D50S was refined against the structure factor amplitudes of each of the complexes, using rigid body refinement as implemented in CNS (Brunger et al., 1998. Acta Cryst D-Biol. Cryst 54, 905-21). SigmaA-weighted difference maps were used for the initial manual placement of substrate analogs or sparsomycin. The coordinates of sparsomycin were obtained energy minimization using the Discover module of INSIGHT II software (Accelrys Inc., San Diego, Calif.). Each of the D50S-ligand models was further refined in CNS.

For the calculation of free R-factor, a subset of reflections (10% of the data) was omitted from the refinement.

Experimental Results:

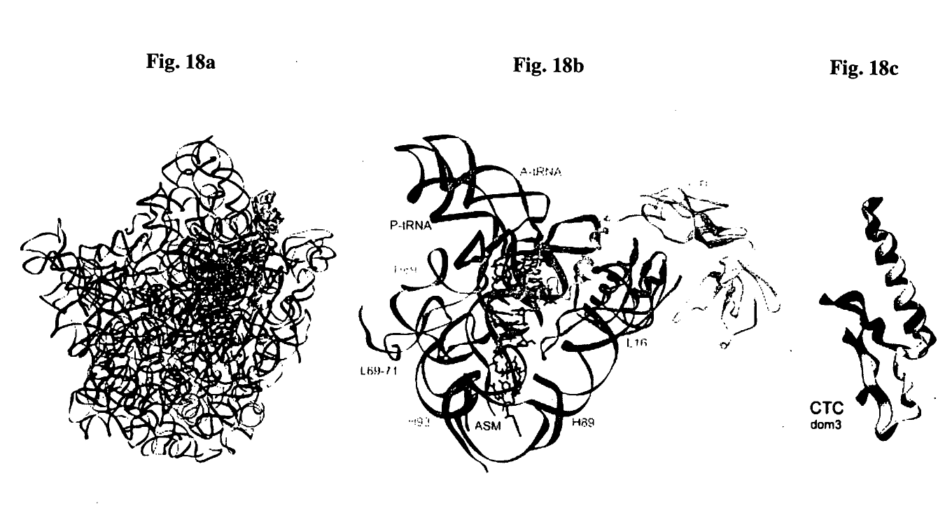

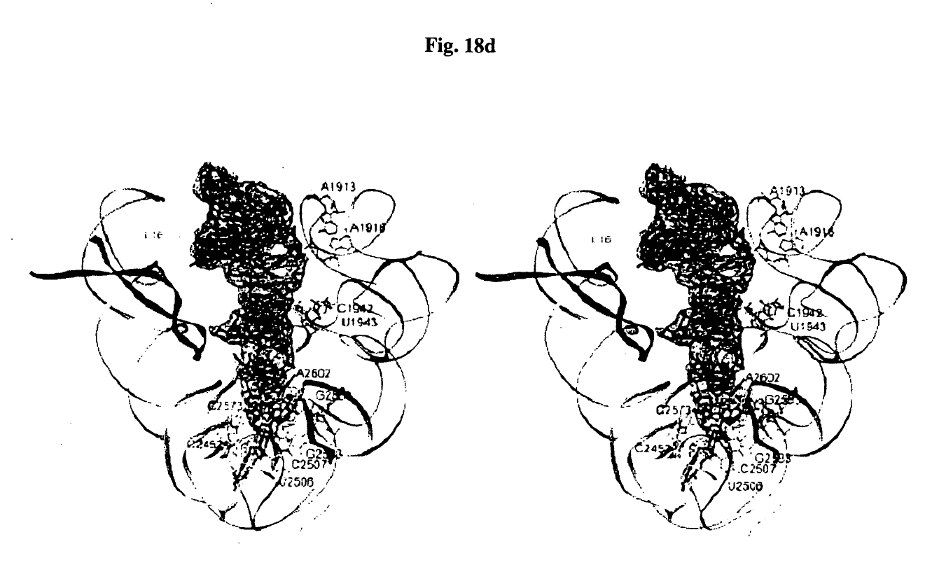

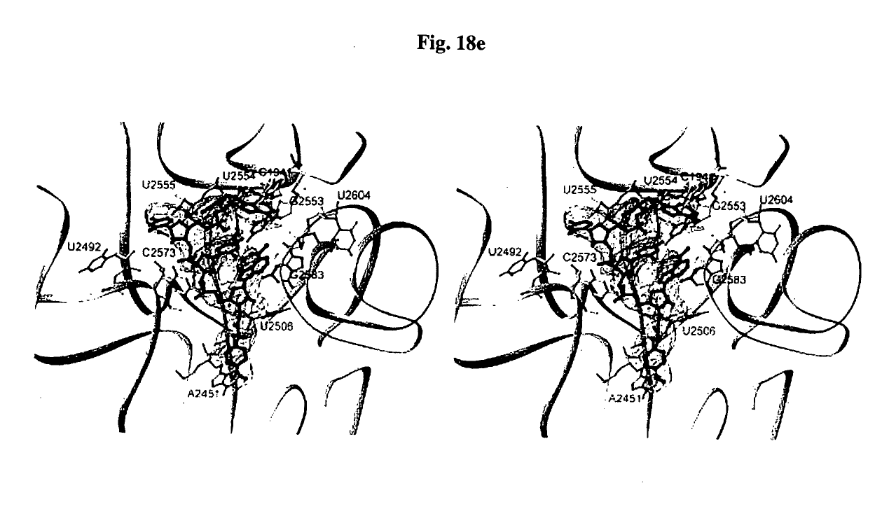

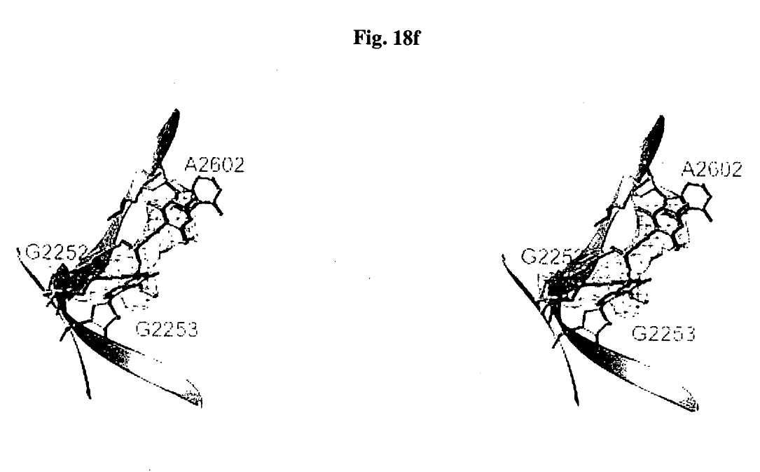

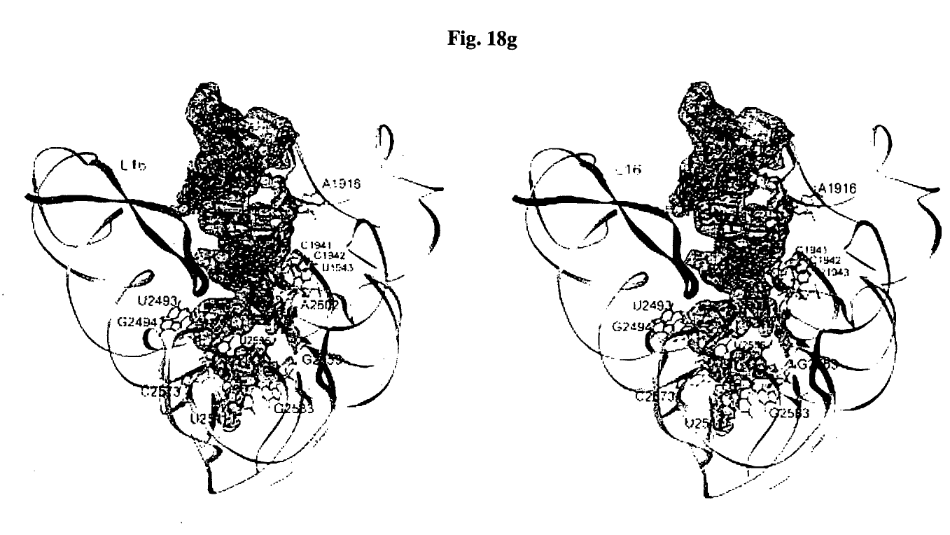

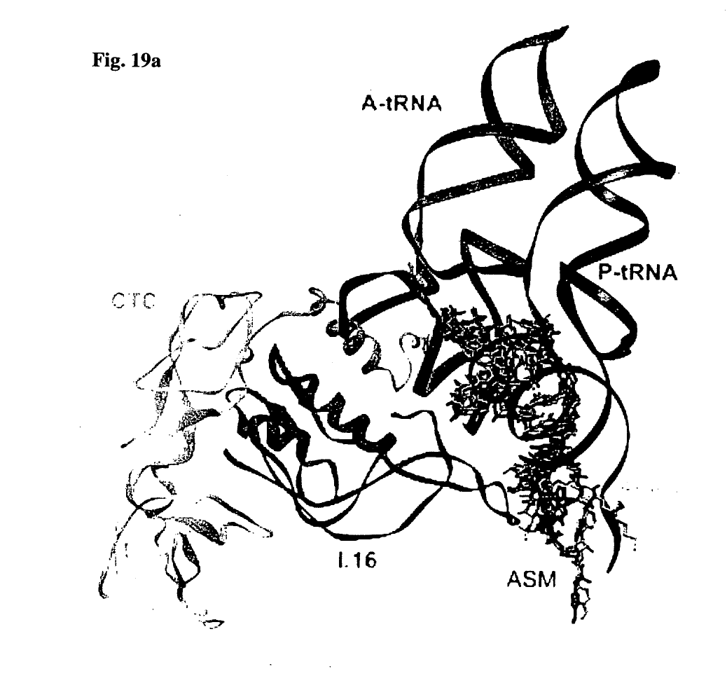

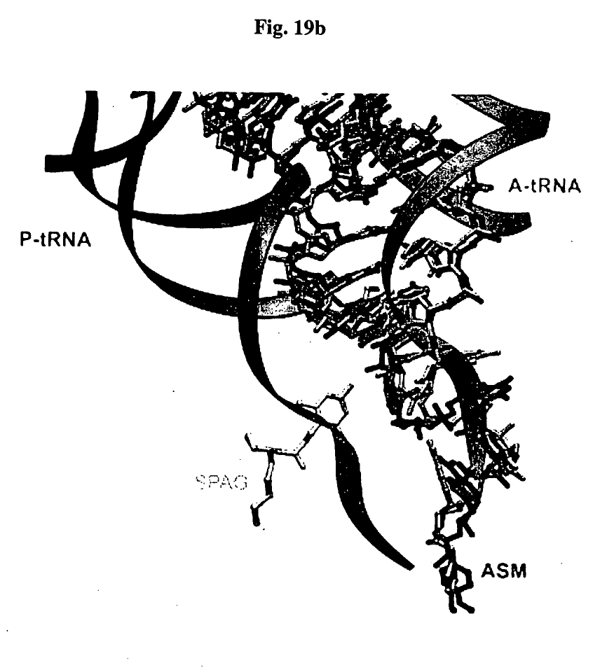

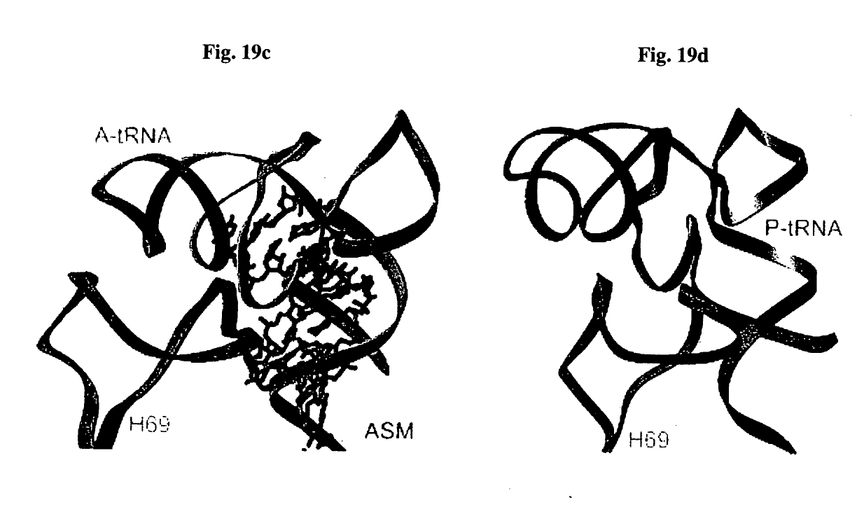

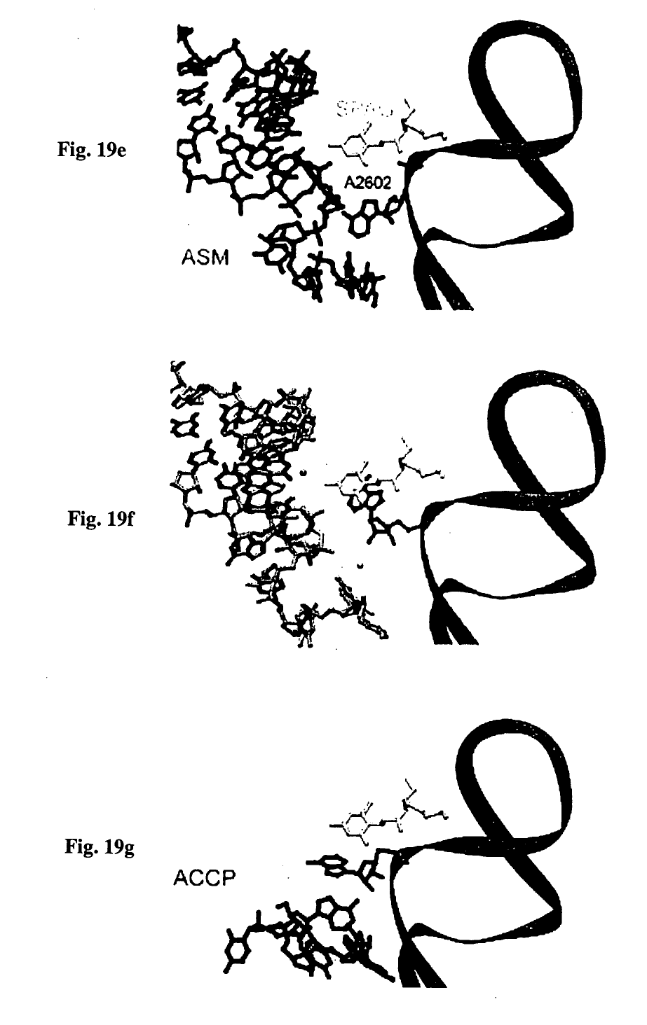

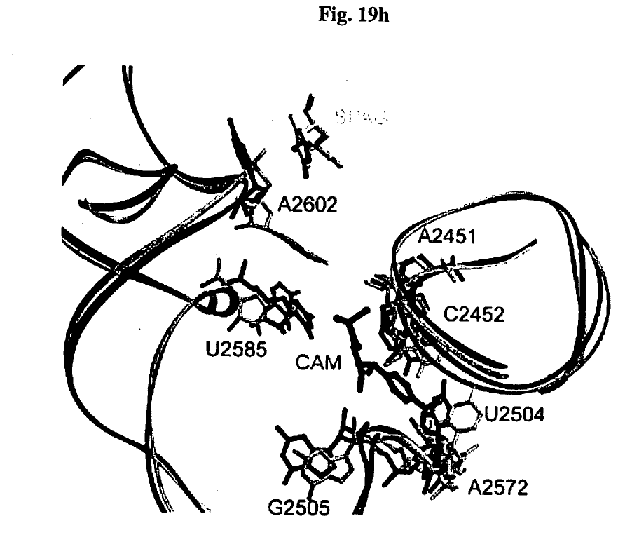

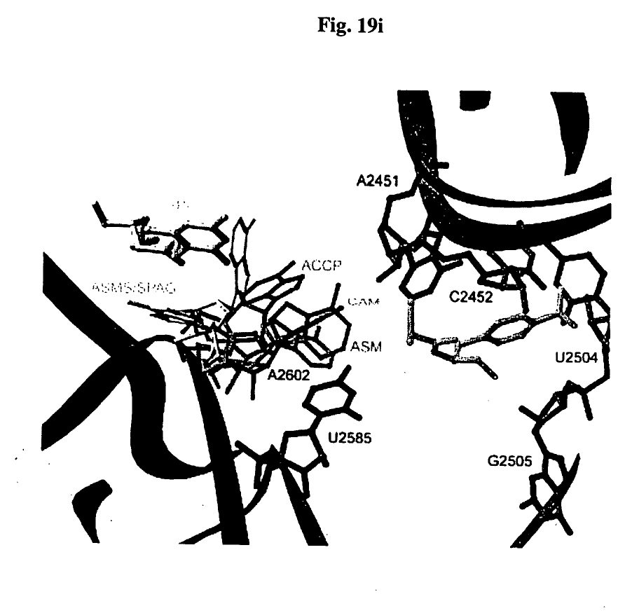

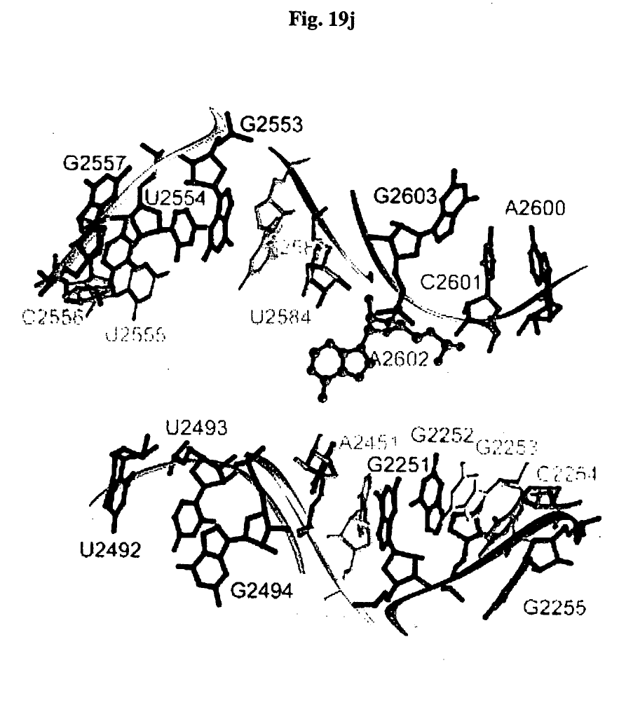

Table 24 shows the summary of the results obtained from crystallographic analysis of crystals of D50S in complex with ASM, ACCP, sparsomycin, and ASMS. The binding modes of these ligands are shown in FIGS. 18a-g and FIGS. 19a-j, as further described below.

TABLE 24
Crystallographic statistics of data collection and refinement*.

| Co- | | | | | | | | Cell |
|---|---|---|---|---|---|---|---|---|
| crystallized | Resolution | Completeness | | | No. of | R factor | R free | dimensions |
| compound(s) | (Å)^{¶} | (%) | I/Isig(I) | Rsym | crystals | (%) | (%) | (Å) |
| ASM | 50.0-3.50 | 99.1 | 4.6 | 12.5 | 12 | 0.2441 | 0.3035 | 169.9 × 409.9 × 695.9 |
| | 3.63-3.50 | 96.8 | 4.1 | 38.4 | | | | |
| ACCP | 50.0-3.70 | 97.8 | 7.9 | 15.6 | 4 | 0.2838 | 0.3355 | 169.9 × 410.4 × 697.1 |
| | 3.76-3.70 | 97.3 | 1.8 | 39.3 | | | | |
| Sparsomycin | 40.0-3.70 | 81.1 | 5.1 | 12.6 | 3 | 0.2710 | 0.3176 | 169.1 × 409.9 × 696.3 |
| | 3.76-3.70 | 80.0 | 1.7 | 37.0 | | | | |
| ASMS | 50.0-3.60 | 95.2 | 5.8 | 16.9 | 7 | 0.2841 | 0.3268 | 169.6 × 409.4 × 695.1 |
| | 3.66-3.60 | 93.8 | 1.7 | 35.6 | | | | |

*Average standard deviations for bond lengths are 0.005 Å and 0.97 degrees for bond angles. Because of the high completeness, refinement was carried out using the data between 8 Å and the last shell.
^{¶}Numbers in the second row describe the highest resolution bin.

Atomic structure of D50S-ACCP, -ASM, -ASMS, and -sparsomycin complexes: The three-dimensional atomic structure of D50S in complex with ACCP, ASM, ASMS or sparsomycin was found to be defined by the sets of pdb atom coordinates set forth in Tables 25-28, respectively (refer to enclosed CD-ROM). Structure coordinates for proteins in these tables refer to alpha-carbon atoms of amino acid residues, and the nomenclature of constituents of these complexes used in these tables are specified in Table 29.

TABLE 29
Nomenclature of constituents of D50S complexes
used in Tables 25-28.

| Constituent | Nomenclature |
|---|---|
| ribosomal proteins L2-L6 | L02-L06 |
| ribosomal protein L9 | L09 |
| ribosomal protein L11 | L11 |
| ribosomal proteins L13-L24 | L13-L24 |
| ribosomal protein CTC | CTC |
| ribosomal proteins L27-L36 | L27-L36 |
| 23S rRNA | ARNA |
| 5S rRNA | BRNA |
| ACCP | ACCP |
| ASM | ASM |
| ASMS | ASM*, SPAR** |
| Sparsomycin | SPAR |
| Mg2+ ions | MGMG |
| Zn2+ ions | ZNZN |

*ASM—ASM,
**SPAR—sparsomycin

Atomic structure of the ACCP binding site of D50S: Three-dimensional atomic structures of the portion of D50S-ACCP complex comprising all of ACCP and portions of D50S contained within a 20 Å-radius sphere centered on ACCP (coordinates 69, 121, 115) were found to be defined by the subset of Table 25-derived coordinates (refer to enclosed CD-ROM) set forth in Table 30 (refer to enclosed CD-ROM).

Analysis of the D50S-ACCP complex atomic structure contained in the above-described 40 Å-radius sphere demonstrated that ACCP interacts with the LRS via nine 23S rRNA nucleotides having coordinates C1924, A2430, U2485, G2532, U2533, U2534, C2552, G2562, and U2583 (according to D. radiodurans numbering).

Atomic structure of the ASM binding site of D50S: Three-dimensional atomic structures of the portion of D50S-ASM complex comprising all of ASM and portions of D50S contained within a 40 Å-radius sphere centered on ASM (coordinates: 83, 120, 104) were found to be defined by the subset of Table 26-derived coordinates (refer to enclosed CD-ROM) set forth in Table 31 (refer to enclosed CD-ROM).

Analysis of the D50S-ASM complex atomic structure contained in the above-described 40 Å-radius sphere demonstrated that ASM interacts with the LRS via 23S rRNA nucleotides having coordinates C1892, A1896, A1899, C1925, U1926, A2431, U2485, C2486, G2532, U2534, C2552, G2562, and A2581; and via ribosomal protein L16 amino acid residues having coordinates 79-81 (according to D. radiodurans numbering).

Atomic structure of the ASMS binding site of D50S: Three-dimensional atomic structures of the portion of D50S-ASMS complex comprising all of ASMS and portions of D50S contained within a 40 Å-radius sphere centered on ASMS (coordinates: 83, 120, 104) were found to be defined by the subset of Table 27-derived coordinates (refer to enclosed CD-ROM) set forth in Table 32 (refer to enclosed CD-ROM).

Analysis of the D50S-ASMS complex atomic structure obtained contained in the above-described 40 Å-radius sphere demonstrated that ASMS interacts with the LRS via 23S rRNA nucleotides having coordinates A1899, C1924, U1926, A2430, U2472, U2485, C2486, G2532, U2534, C2552, G2562, U2563, and A2581, and via ribosomal protein L16 amino acid residues having coordinates 79-81 (according to D. radiodurans numbering); and via Mg2+ ions having the atom coordinates 79393 and 79394 (MG 101 and MG 102, respectively).

Atomic structure of the sparsomycin binding site of D50S: Three-dimensional atomic structures of the portion of D50S-sparsomycin complex comprising all of sparsomycin and portions of D50S contained within a 20 Å-radius sphere centered on sparsomycin (coordinates 69, 129, 105) were found to be defined by the subset of Table 28-derived coordinates (refer to enclosed CD-ROM) set forth in Table 33 (refer to enclosed CD-ROM).

Analysis of the D50S-sparsomycin complex atomic structure contained in the above-described 20 Å-radius sphere demonstrated that sparsomycin interacts with the LRS via the 23S rRNA nucleotide having the coordinate A2581 (according to D. radiodurans numbering).

Binding modes for long and short tRNA mimics: Experiments aimed at binding tRNA molecules to D50S within the crystals led to a severe decrease in resolution, presumably because of steric reasons. However, only a minor resolution decrease was observed in crystals of D50S that were soaked in solutions containing ASM, designed to mimic the acceptor stem of tRNA, which is the part of the tRNA molecule that interacts with the A-site in the LRS. The electron density map of this complex allowed us to define the PT cavity in D50S and to unambiguously localize 25 of the 35 nucleotides of ASM and determine their interactions with the ribosome.

The PTC of D50S and the positioning of ASM within it are shown in FIGS. 18a-b. The walls of the PT cavity are composed of several RNA features. One of them is the flexible helix H69 that forms the B2a bridge, as described in Example 1, above (Harms J. et al., 2001. Cell 107, 679-688; Yusupov et al., 2001. Science 292, 883-96). The features adjacent to H69 at the cavity surface are helices H68, H70 and H71 and H89. The latter forms one of the long walls of the A-site. Consistent with earlier findings (Eckerman and Symons, 1978. Eur J Biochem 82, 225-34; Vazquez, 1979. Mol Biol Biochem Biophys. 30, 1-312; Hummel and Bock, 1987. Nucleic Acids Res 15, 2431-43; Vester and Garrett, 1988. EMBO Journal 7, 3577-88; Mankin and Garrett, 1991. J Bacteriol 173, 3559-63; Aagaard et al., 1994. J Bacteriol 176, 7744-7), the bottom of the cavity is a hydrophobic pocket, built of the backbone of nucleotides 2505-2506 and the bases of 2503-2504, 2451 and 2063.

Analysis of the interactions that ASM makes with the 23s rRNA (Table 34) indicated that the helical stem of ASM interacts with the extended loop of protein L16, and that H69 packs groove-to-backbone with it. Hence it seems that H69 and protein L16 are the key factors influencing the positioning of ASM within the PTC. Interestingly, the main chain of protein CTC does not interact directly with the tRNA mimic, although its conformation underwent substantial rearrangements (FIG. 18c) as a result of the binding of the tRNA mimic, presumably to avoid short contacts.

To correlate the binding modes of ASM in the large subunit with those of the A-site tRNA in the whole ribosome, the coordinates of the three tRNA molecules were docked onto the D50S structure according to their orientation within the 5.5 Å resolution structure of T70S (Yusupov et al., 2001. Science 292, 883-96). The docking placed the 3′ end of the A-site tRNA in a location almost overlapping that of the 3′ end of ASM, so that both 3′ ends interact with the H69-H71 loop (also called the conserved 1942 loop). However, compared to the position of the acceptor arm of A-tRNA in T70S, the helical part of ASM is displaced towards the P-site. Thus, although H89 that runs nearly parallel to the A-site tRNA in T70S and interacts with it, is proximate to ASM, ASM interacts mostly with H69 (Table 34 and FIG. 18d).

TABLE 34
D50S-ASM interactions.

| | | Helix/ | rRNA/Protein | | |
|---|---|---|---|---|---|
| | ASM | loop | D. radiodurans | E. coli | Comments |
| 1. | G 01 | | L16, amino | amino | Hydrophilic with |
| | | | acids 80-81 | acids | protein loop. |
| | | | | 58-60 | Missing in H50S |
| 2. | C 05 O2′ | H69 | A 1899 O1P | A1916 | Missing in H50S |
| 3. | U 06 O2P | H69 | A 1896 N6 | A1913 | Missing in H50S |
| 4. | C 21 O2′ | H69 | C 1892 O2′ | C1909 | Missing in H50S |
| 5. | U 29 O2′ | L69-71 | U 1926 O2P | U1943 | Missing in H50S |
| 6. | U 29 O2 | L69-71 | C 1925 O2′ | C1942 | Missing in H50S |
| 7. | C 30 O2′ | L69-71 | C 1925 O4′ | C1942 | Missing in H50S |
| 8. | A 32 O2′ | H93 | A 2581 N6 | A2602 | |
| 9. | C 33 N4 | H92 | U 2534 O4 | U2555 | |
| 10. | C 34 N4 | H92 | G 2532 O6 | G2553 | BP |
| 11. | C 34 N3 | H92 | G 2532 N1 | G2553 | BP |
| 12. | C 34 O2 | H92 | G 2532 N2 | G2553 | BP |
| 13. | C 34 O2 | H90 | C 2486 O2′ | C2507 | |
| 14. | C 34 O2′ | H90 | C 2486 O3′ | C2507 | |
| 15. | C 34 O3′ | H90 | C 2552 N4 | C2573 | Missing in H50S* |
| 16. | PPU 35 O2′ | L89-90 | U 2485 O2′ | U2506 | |
| 17. | PPU 35 O2′ | L89-90 | U 2485 O2′ | U2506 | |
| 18. | PPU 35 O2′ | L90-93 | G 2562 N2 | G2583 | |
| 19. | PPU 35 N3′ | L89-90 | U 2485 O2′ | U2506 | |
| 20. | PPU 35 N3 | L90-93 | G 2562 N2 | G2583 | |
| 21. | PPU 35 N1 | L90-93 | G 2562 O2′ | G2583 | |
| 22. | PPU 35 - | L74-89 | C 2431 | C2452 | Stacking |
| | tyrosine ring | | | | |

Contacts missing in H50S due to the disorder of the tRNA mini-helix are noted.
The * shows contacts missing in H50S although this part is seen in the map

Puromycin binds weakly to the ribosome, but can be induced to bind with significant affinity thereto in the presence of over 30% of low molecular weight alcohols. Nevertheless, ACCP was unexpectedly found to bind well to D50S. Such binding may be due to the solution used for stabilizing the D50S complex crystals which contains ethanol and ethylhexandiol and may also be due to ACCP itself, since it is slightly longer than puromycin. Table 35and FIG. 18e show that all of the interactions of ACCP with D50S exclusively involve 23s rRNA bases, consistent with results of crosslinking by UV irradiation (Kirillov et al., 1999. RNA 5, 1003-13) but not with the indications for proximity of proteins L27 and L33 to the 3′ ends of the tRNAs (Wower et al., 1995. Cell Biol 73, 1041-7).

TABLE 35
D50S-ACCP interactions**.

| | | | rRNA/Protein | | |
|---|---|---|---|---|---|
| | ACCP | Helix/loop | D. radiodurans | E. coli | Comments |
| 1. | A 32 N7 | L69-71 | C 1924 O2′ | C1941 | |
| 2. | A 32 N6 | H93 | U 2583 O1P | U2604 | |
| 3. | A 32 O2′ | H92 | U 2533 O4 | U2554 | |
| 4. | C 33 O1P | H92 | U 2533 N3 | U2554 | |
| 5. | C 33 N4 | H92 | U 2534 O2′ | U2555 | |
| 6. | C 34 N4 | H92 | G 2532 O6 | G2553 | BP |
| 7. | C 34 N3 | H92 | G 2532 N1 | G2553 | BP |
| 8. | C 34 O2 | H92 | G 2532 N2 | G2553 | BP |
| 9. | C 34 O2′ | H90 | C 2552 N4 | C2573 | |
| 10. | PPU 35 O2′ | L89-90 | U 2485 O2 | U2506 | |
| 11. | PPU 35 N3 | L90-93 | G 2562 N2 | G2583 | |
| 12. | PPU 35 N1 | L90-93 | G 2562 O2′ | G2583 | |
| 13. | PPU 35 N3′ | L89-90 | U 2485 O2′ | U2506 | |
| 14. | PPU 35 O | L74-89 | A 2430 O2′ | A2451 | |

**The numbering of the ACCP bases is according to the ASM numbering scheme.

The present results show that ACCP binds to the A-site (FIG. 18e), and hence is expected to make the same interactions with the PTC as ASM does. Nevertheless, only 7 out of the 14 interactions of ACCP appear to be with the same bases that interact with the 3′ end of ASM. These include the base pairing between G2553 and C34, the contacts between C33 and U2555, and the contacts of the puromycin entity with G2583 and U2506. However, despite exploiting the same bases, differences in the nature of the contacts were observed even among these. The largest variation are in the contacts made by G2583 since it lies almost perpendicular to the puromycin end of ASM, but packs side-to-side with the same moiety of ACCP. Nucleotide C2573 interacts with both compounds, but in a slightly different fashion. Four nucleotides, namely C1941, A2451, U2554 and U2604, make contacts with ACCP but not with the 3′ end of ASM, and three nucleotides, C2507, G2452, and A2602, interact only with the 3′ end of ASM.

The Watson-Crick base-pair between C34 of ASM and of ACCP (corresponding to C75 of the A-site tRNA) and G2553 of the A-loop, appears to be a common feature in ASM, ACCP and ASMS (see below and in FIG. 18g) complexes with D50S, in the tRNA complex with T70S (Yusupov et al., 2001. Science 292, 883-96), and in the liganded H50S (Nissen et al., 2000. Science 289, 920-30; Schmeing et al., 2002. Nat Struct Biol 9, 225-30). In contrast, the contacts made by C33 of ASM and of ACCP, and U2555 of D50S are not seen in the crystal structures of the liganded H50S because the corresponding base (C74 in tRNA) is stacked to U2554. Furthermore, A76 of the CCA end of the H50S ligand induces shifts in three bases, U2585, U2584 and G2583 of H50S, whereas the base in the equivalent position in both ASM (Table 34) and ACCP (Table 35), called PPU35, does not induce rearrangements and makes different contacts. Interestingly, A2451, which was implicated as a direct participant in the ribosomal catalytic mechanism (Nissen et al., 2001. Proc Natl Acad Sci U S A. 98, 4899-903) or as facilitating it (Ramakrishnan, 2002. Cell 108, 557-72), based on its location in the PTC of H50S, interacts with ACCP in the D50S system, but not with the corresponding base of ASM.

TABLE 36
D50S-ASMS interactions.

| | | | rRNA/Protein | | |
|---|---|---|---|---|---|
| | ASMS | Helix/loop | D. radiodurans | E. coli | Comments |
| 1. | G 01 | | L16 aa80-8160) | 59-60 | |
| 2. | G 02 N3 | | Mg 101 | Mg 101 | |
| 3. | C 05 O2′ | H69 | A 1899 O1P | A1916 | |
| 4. | U 29 O2′ | L69-71 | U 1926 O1P | U1943 | |
| 5. | C 30 O2′ | L69-71 | C 1924 O2′ | C1941 | |
| 6. | C 31 O2′ | H93 | A 2581 N3 | A2602 | |
| 7. | C 31 O2 | | Mg 101 | Mg 101 | |
| 8. | C 33 O2′ | H89 | U 2472 O3′ | U2493 | |
| 9. | C 33 N4 | H92 | U 2534 O4 | U2555 | |
| 10. | C 34 N4 | H92 | G 2532 O6 | G2553 | BP |
| 11. | C 34 N3 | H92 | G 2532 N1 | G2553 | BP |
| 12. | C 34 O2 | H92 | G 2532 N2 | G2553 | BP |
| 13. | C 34 O2 | H90 | C 2486 O2′ | C2507 | |
| 14. | C 34 O2′ | H90 | C 2486 O2′ | C2507 | |
| 15. | C 34 O3′ | H90 | C 2552 N4 | C2573 | |
| 16. | PPU 35 O2′ | L90-93 | U 2485 O2 | U2508 | |
| 17. | PPU 35 O2′ | L90-93 | U 2563 O2′ | U2582 | |
| 18. | PPU 35 N3 | L90-93 | G 2562 N2 | G2583 | |
| 19. | PPU 35 O5′ | H90 | C 2552 N4 | C2573 | |
| 20. | PPU 35 O | L74-89 | A 2430 O2′ | A2451 | |
| 21. | PPU 35 N | L74-89 | A 2430 O2′ | A2451 | |
| 22. | PPU 35 N | L74-89 | A 2430 N3 | A2451 | |

The position of ASM in D50S is similar, but not identical, to that of the acceptor stem of the A-site tRNA in the 5.5 Å structure of T70S (Yusupov et al., 2001. Science 292, 883-96). The reasons for this may reflect the difference between tRNA binding to unbound large ribosomal subunit and to assembled ribosome, in which the tRNA also makes substantial contacts with the small subunit, or to the differences in A-site binding in the absence of P-site substrate (Green et al., 1998. Science 280, 286-9). Alternatively, the position of ASM may indicate the existence of an additional binding mode, similar to the suggested “hybrid mode”, in which the movement of the acceptor stem is uncoupled from that of the rest of the tRNA (Moazed and Noller, 1991. Proc Natl Acad Sci U S A. 88, 3725-8).

The PTC tolerates various binding modes, but precise positioning is required for protein biosynthesis: The PTC is highly conserved, nevertheless some diversity was observed in its structure in the different crystal systems. The overall structure of the cavity hosting the PT activity in the liganded D50S is similar to that seen in the native D50S structure, as described in Example 1, above (Harms J. et al., 2001. Cell 107, 679-688), in the antibiotic bound D50S structures, as described in Example 2, above (Schlüenzen et al., 2001. Nature 413, 814-21) and in the complexed 70S ribosome (Yusupov et al., 2001. Science 292, 883-96). It also resembles the PT cavity of H50S, but to a lesser extent. The orientations of both the conserved and variable bases of the PTC seem to depend on several parameters; among them is the functional state of the ribosome. Thus, the conformation of the key nucleotides A2451, U2506, U2585 and A2602, in the complex of T70S with three tRNAs differs significantly from the conformations seen in two complexes of the LRS from H50S with compounds believed to be substrate or transition-state analogs (Yusupov et al., 2001. Science 292, 883-96). Also, the PTC of H50S undergoes notable conformational changes upon binding ligands (Nissen et al., 2000. Science 289, 920-30; Schmeing et al., 2002. Nat Struct Biol 9, 225-30). These include the ordering of the base corresponding to A2602, which is disordered in the 2.4 Å structure of H50S, as are most of the functionally relevant features in this structure (Ban et al., 2000. Science 289, 905-20). Upon binding ligands, A2602 becomes positioned between the A-site and the P-site, while G2583, U2584, and U2585 are shifted (Nissen et al., 2000. Science 289, 920-30). Interestingly, the overall conformation of the rRNA backbone in the vicinity of the 3′-CCA end of P-site tRNA in D50S is closer to that seen in H50S than to that of T70S/tRNA complex, presumably because the functional state of the large subunit, namely being unbound or assembled into 70S ribosome, governs the conformation of the P-site.

The different orientations of the PTC may reflect the flexibility needed for the formation of the peptide bond. However, functional needs may not be the sole reasons for the conformation differences seen in the PTC. Phylogenetic variations may also cause diversity. Thus, altered chemical reactivities of the PTC in E. coli compared to Haloferax gibbonsii have been observed (Rodriguez-Fonseca et al., 2000. Rna 6, 744-54), consistent with the distinct differences in the orientations of some of the nucleotides, detected by the superposition of the structures of H50S (Ban et al., 2000. Science 289, 905-20) and D50S, as described in previous Example 1 above (Harms J. et al., 2001. Cell 107, 679-688). In addition to the functional and phylogenetic aspects, varying conformations of the PTC were observed in E. coli ribosomes under different chemical conditions. Over three decades ago, Elson and coworkers (Miskin et al., 1968. Biochem Biophys Res Commun 33, 551-7) correlated the functional activity of E. coli ribosomes with pH and relative salt concentrations. These results were confirmed recently in experiments showing alterations in dimethyl sulfate (DMS) reactivity within the PTC as a function of pH changes, between pH 6.5, a value close to the pH of H50S crystals, and pH 8.5, a value close to that of D50S crystals (Bayfield et al., 2001. Proc Natl Acad Sci U S A. 98, 10096-101), which led to the suggestion that conformational rearrangements in the structure of the LRS may occur upon pH shift (Xiong et al., 2001. Rna 7, 1365-9).

Diversity in binding modes of different A-site tRNA analogs may also be connected to the nature of the analog, and the differences in positioning of different analogs appear to be correlated with the amount of support given to them by the PTC. ASM is held in its position mostly by the interactions that its helical part makes with H69, the loop between H69 and H71 and protein L16 (FIGS. 18d and 19a). It appears that these contacts play a major role in the precise positioning of ASM, since the various binding modes were detected for the CCA ends of ASM and ACCP and substrate analogs of H50S (Nissen et al., 2000. Science 289, 920-30; Schmeing et al., 2002. Nat Struct Biol 9, 225-30), although all make comparable amount of interactions with their corresponding PTCs. Hence it appears that the lower part of the PTC can tolerate several binding modes that resemble each other, but are not necessarily identical to the precise orientation leading to efficient protein biosynthesis. Consistent with the findings that although most of the interactions of the ACCP with the ribosome are with universally conserved nucleotides, altered reactivities were observed for puromycin in eubacteria and archaea (Rodriguez-Fonseca et al., 1995. J Mol Biol 247, 224-35).

Variation in binding modes were observed even within the subgroup of short tRNA analogs. Comparison of the positioning of the tRNA acceptor stem mimic with those of ACCP and the smaller substrate analogs used so far (Nissen et al., 2000. Science 289, 920-30; Schmeing et al., 2002. Nat Struct Biol 9, 225-30), indicated that the smaller analogs can bind to the PTC in various orientations even under close to physiological conditions. Interestingly, despite its many interactions to the active site, the crystals of the ACCP complex diffract to slightly lower resolution than those of ASM and ASMS (Table 24), consistent with its weak binding in solution and with the variability of the binding modes of the short mimics. The ability of puromycin to bind to the A-site in various modes, in contrast to the tRNA molecules that should be precisely oriented, may indicate that puromycin's mode of action is closer to that of an antibiotic agent, designed to somehow block the protein-biosynthesis process, rather than a substrate designed to perform an accurate reaction. Consequently, a straightforward extension of the mode of reaction was performed by short substrate analogs in suggesting the mechanism for peptide bond formation is only partially justified.

So far, the only proposed detailed mechanism of bond formation was based solely on the binding modes of the 3′ end of an A-site t-RNA mimic together with a short intermediate-state analog (Nissen et al., 2000. Science 289, 920-30). According to this proposed mechanism, the universally conserved nucleotide A2451 participates directly in catalysis of peptide bond formation in a reaction requiring a rather high pK. This has been challenged on biochemical grounds (Barta et al., 2001. Science 291, 203a) and mutants of the putative catalytic bases were shown to be functionally active in-vitro (Polacek et al., 2001. Nature 411, 498-501; Thompson et al., 2001. Proc Natl Acad Sci U S A. 98, 9002-7). Furthermore, the pK required for the catalysis was shown to be associated with the conformation of the PTC in inactive ribosomes (Bayfield et al., 2001. Proc Natl Acad Sci U S A. 98, 10096-101; Miskin et al., 1968. Biochem Biophys Res Commun 33, 551-7). It is conceivable that among the various conformations of the PTC, some may be less suitable for efficient production of proteins, but still may allow for single peptide bond formation. For instance, it was shown that a modified version of the “fragment reaction” led to the creation of a single-peptide bond within H50S crystals at lower than the physiological salt concentration (1.5 M versus 2.5 M), and that KCl, the main constituent of H. marismortui intracellular solution, could be replaced by NaCl. However, conditions that are rather close to the in-vivo environment (2.1 M NH₄Cl), or to the conditions optimized for protein biosynthesis in-vitro (Shevack et al., 1985. FEBS Letters 184), were required for performing a reaction resembling protein biosynthesis, using polyU as mRNA. Similar dependence on pH was reported, Thus, the fragment reaction could be carried out within the H50S crystals, at pH 5.4-6, whereas for obtaining meaningful results in the control experiment, significantly higher pH (pH 7.1 to pH 8.3) was required (Schmeing et al., 2002. Nat Struct Biol 9, 225-30).

H69, an intersubunit bridge involved in substrate binding and in tRNA translocation: The B2a bridge is the bridge that interacts simultaneously with the decoding site in the small subunit and with the PTC in the large one, thus connecting the two active centers of the ribosome. This intersubunit bridge is composed of helix H69, which is connects to H68 and H71. In the structure of D50S, it is located on the 50S intersubunit interface (Harms J. et al., 2001. Cell 107, 679-688; Yonath, 2002. Annu Rev Biophys Biomol Struct 31, 257-73) and for creating the intersubunit bridge it stretches towards the small subunit. Within the 70S ribosome, H69 interacts with both the A- and the P-site tRNAs, whereas H89 interacts extensively with the A-site tRNA (Yusupov et al., 2001. Science 292, 883-96). In the complex of D50S with ASM, most of the contacts between the helical stem of the ASM are with H69. The crucial contribution of H69 to the proper placement of the tRNA mimic is also reflected by the disorder of the helical stem of the tRNA mimic that was bound to H50S crystals, in which H69 itself is disordered (Ban et al., 2000. Science 289, 905-20; Nissen et al., 2000. Science 289, 920-30).

The two most important functional centers, the decoding site in the small subunit and the PTC in the large one, appear to be separated from the other centers involved in translocation. Therefore, for efficient translocation, a sophisticated signaling over long distances is requires. Hence, the structural elements participating in translocation should be the movable ribosomal features that may interact simultaneously with some or all of the involved centers. H69, its extension, H71 and the loop connecting them, are situated between the decoding site and the PTC and, owing to its flexibility, it may be the right candidate to provide the machinery needed for the transmission of signals (Harms J. et al., 2001. Cell 107, 679-688; Yonath, 2002. Annu Rev Biophys Biomol Struct 31, 257-73). The inherent flexibility of H69 and its proximity to both the A- and the P-sites, suggest that besides acting as an intersubunit bridge and a signal transmitter, H69 may also participate in translocation events. The specific conformations of H69 in D50S and T50S, and their modes of binding the A-site tRNA to T70S and the A-site mimic to D50S, implicated H69 as a carrier of the helical part of the A-site tRNA into the P-site (FIGS. 19c-d). Interestingly, mapping of the E. coli modified nucleotides known to be important for the function of the LRS (Green and Noller, 1996. RNA 2, 1011-21; Triman, 1996. Nucleic Acids Res 24, 169-71) onto the D50S structure, showed clustering of the positions corresponding to these nucleotides in the vicinity of the active site of D. radiodurans as well as in H69. The location of the latter on the stem loop of H69 intersubunit bridge in the assembled ribosome led us to suggest that the modified bases play a role in the bridging events.

Sparsomycin inhibits protein synthesis by triggering major conformational alterations within the PTC: The co-crystals of the universal antibiotic sparsomycin with D50S showed almost quantitative binding, although they did not contain N-blocked aminoacyl-tRNA at the P-site, a compound that was implied to enhance sparsomycin binding (Lazaro et al., 1991. Biochemistry 30, 9642-8; Porse et al., 1999. Proc Natl Acad Sci U S A. 96, 9003-8). As with ACCP and the PTC antibiotics described in Example 2 above (Schlüenzen et al., 2001. Nature 413, 814-21), namely chloramphenicol and clindamycin, the binding site of sparsomycin is composed exclusively of 23s rRNA. However, whereas the most antibiotics make ample contacts with the ribosome, sparsomycin creates only a few interactions, rationalizing the lack of clear footprints (Moazed and Noller, 1991. Proc Natl Acad Sci U S A. 88, 3725-8). In fact, stacking interactions between the modified uracil ring of sparsomycin and the highly conserved base A2602 appear to be its only well defined contacts with the ribosome (FIGS. 18f, and 19h). Hence, this suggests that these contacts carry the main inhibitory effect of sparsomycin, consistent with the crosslinking of A2602 by derivatized sparsomycin (Porse et al., 1999. Proc Natl Acad Sci U S A. 96, 9003-8) as well as with the universality of sparsomycin. Also consistent with the present results is the fact that A2602 is the only base that was crosslinked by [¹²⁵I]-labeled sparsomycin, and that the formation of this crosslink was found to be inhibited by antibiotics that prevent peptide bond formation in-vitro (Porse et al., 1999. Proc Natl Acad Sci U S A. 96, 9003-8). In addition, the present results are in accord with biochemical studies showing that derivatization of the uracil ring caused drug inactivation, whereas its second potentially reactive moieties, the sulfoxy group, could be altered with a minor loss of its inhibitory activity (Porse et al., 1999. Proc Natl Acad Sci U S A. 96, 9003-8; van den Broek et al., 1989. J Med Chem. 32, 2002-15). They also explain why substitution of sparsomycin's terminal methyl group by benzyl, phenol-alanine and similar moieties do not decrease the yields of A2602 crosslinking (Lazaro et al., 1996. J Mol Biol 261, 231-8; Porse et al., 1999. Proc Natl Acad Sci U S A. 96, 9003-8).

In its single binding site, sparsomycin is close to two universally conserved guanosine residues, G2252 and G2253 (Gutell et al., 1993. Nucleic Acids Res 21, 3055-74). However, even the closest distances between the O23 and O7 of sparsomycin and N2 of G2252 and G2253, respectively, are too long (4.4-4.7 Å) for hydrogen bonds or van der Waals contacts. However, the proximity of sparsomycin to them may influence protein biosynthesis, since direct involvement of G2252 and G2253 in peptide bond formation has been shown previously (Moazed and Noller, 1989. Cell 57, 585-97). G2252 was implicated in forming a Watson-Crick base pair with C74 of peptidyl-tRNA and mutations at G2253 result in a dominant lethal phenotype (Green et al., 1997. J Mol Biol 266, 40-50).

In contrast to the minor conformational changes induced by the antibiotics studied so far, as described above in Example 2 above (Schlüenzen et al., 2001. Nature 413, 814-21), sparsomycin appears to significantly alter the conformation of both the P— and the A-sites. A2602 is the base that undergoes the most noticeable conformational rearrangements (FIG. 19e) upon sparsomycin binding. Additional nucleotides that are located far from sparsomycin but their conformation is altered are 2438, 2451, 2499, 2500, and 2584, all implicated in A-site binding, and their mutations show modest resistance to sparsomycin (Tan et al., 1996. Journal of Molecular Biology 261, 222-30). Hence, the binding mode of sparsomycin indicates that sparsomycin inhibits protein biosynthesis mainly by introducing substantial alterations of the conformation of the PTC.

The stacking interactions between sparsomycin and A2602, may be sufficient for its firm attachment as long as the ribosome or its large subunit are not actively involved in protein biosynthesis, or in the crystals, owing to the limited mobility of crystalline materials. At the same time, the limited contacts between sparsomycin and the large subunit rationalize the weak binding of sparsomycin to the ribosome (Monro et al., 1968. Proc Natl Acad Sci U S A. 61, 1042-9; Vazquez, 1979. Mol Biol Biochem Biophys. 30, 1-312; Lazaro et al., 1991. Biochemistry 30, 9642-8; Moazed and Noller, 1991. Proc Natl Acad Sci U S A. 88, 3725-8; Tan et al., 1996. Journal of Molecular Biology 261, 222-30; Porse et al., 1999. Proc Natl Acad Sci U S A. 96, 9003-8). Destabilization of sparsomycin binding during protein biosynthesis may also be correlated to changes in the orientation of sparsomycin's counterpart, nucleotide A2602, which was implicated to play an active role in protein biosynthesis (Porse et al., 1999. Proc Natl Acad Sci U S A. 96, 9003-8). Additional interactions with P-site substrates like N-blocked aminoacyl-tRNA that is known to increase the accessibility of nucleotide A2602 (Porse et al., 1999. Proc Natl Acad Sci U S A. 96, 9003-8 and references therein), should lead to tighter binding. Furthermore, the enhancement of sparsomycin binding by N-blocked aminoacyl-tRNA may indicate that sparsomycin may inhibit protein biosynthesis not only by altering the conformation of the PTC, but also by blocking the P-site and by trapping non-productive intermediate-state compounds.

Analysis of the present results indicated that chloramphenicol and sparsomycin do not share overlapping positions, but seem to compete with each other in inhibiting peptide bond formation (Lazaro et al., 1991. Biochemistry 30, 9642-8; Tan et al., 1996. Journal of Molecular Biology 261, 222-30). However, the base of A2602 in sparsomycin is flipped by 180 degrees compared with its position in the presence of chloramphenicol (FIGS. 19h-i), implicating this base as the trigger of the competition between sparsomycin and chloramphenicol. The conformational rearrangements within the A-site, induced by sparsomycin binding, may be responsible for the weak enhancement of sparsomycin resistance by single-site mutations at A-site positions 2438, 2451, 2499 and 2500 (Tan et al., 1996. Journal of Molecular Biology 261, 222-30). Thus, the conformational rearrangements observed crystallographically rationalize the influence of sparsomycin on A-site binding and explain why sparsomycin was considered to be an A-site ligand. The conformational changes and the mode of sparsomycin binding can also be partially correlated to the results of kinetic measurements that suggested that sparsomycin acts first as a competitive inhibitor and later participates in the formation of an inactive complex [(Cundliffe, E. (1981) Antibiotic inhibitors of ribosome function, E. F. Gale, E. Cundliffe, P. E. Reynolds, M. H. Richmond and M. H. Waring, eds. (London, New York, Sydney, Toronto: Wiley); Theocharis and Coutsogeorgopoulos, 1992. Biochemistry 31, 5861-8; Tan et al., 1996. Journal of Molecular Biology 261, 222-30].

Interplay of the A-site and the P-site: FIG. 18b shows the location of ASM in D50S crystals that were grown in the presence of sparsomycin (ASMS). The orientations of ASM, ASMS, and ACCP within the PTC indicated notable variations in their binding modes (Tables 34-36, respectively, and FIGS. 19e-g, respectively). Compared with the positioning of ASM, ASMS is slightly twisted and placed somewhat closer to the P-site (FIG. 19b and FIG. 19d). In its position ASMS interacts with protein L16, but loses one of the contacts that ASM makes with this protein. Altogether, ASM and ASMS make the same number of contacts with the 23s rRNA. Nevertheless, ASMS maintains only 8 out of the 22 interactions that ASM makes with the 23s rRNA within the PTC. Two additional contacts are made between ASMS and bases that interact with ASM, but in a different fashion. Among the contacts common to ASM and ASMS is the pairing of C34 with G2553. Interactions unique to ASMS are with H89 and the loop between H89 and H74. In addition to the interactions of ASMS with the 23s rRNA and protein L16, it makes three hydrogen bonds with a putative hydrated Mg2+ ion (Table 36 and FIGS. 19e-f), located close to its CCA end. This Mg2+ ion with the water molecules bound to it, is seen clearly in the ASMS map, whereas the same position in the ASM map contains only blurred and fragmented features. The contribution of this Mg2+ ion to peptide bond formation needs still to be investigated, but its appearance as a consequence of P-site occupation may hint at its functional relevance.

It is assumed that the differences between the binding modes of ASM and ASMS result from alterations in the PTC. Since ASMS crystals were obtained by soaking co-crystals of D50S and sparsomycin, and since sparsomycin was shown to trigger conformational changes in the PTC, it seems that these were sufficient to modify the binding mode of the A-site substrate analogs, hence suggesting interplay between the A-site and the P-site. These results are consistent with the cooperative interactions between P-site and A-site bound tRNAs which have been previously reported [Pestka, S. (1969) Translocation, aminoacryl-oligonucleotides, and antibiotic action. Cold Spring Harb Symp Quant Biol 34, 395-410; Hishizawa and Pestka, 1971. Arch Biochem Biophys 147, 624-31; Ulbrich et al., 1978. Arch Biochem Biophys 190, 149-54; Green et al., 1998. Science 280, 286-9; Kirillov et al., 1999. RNA 5, 1003-13]. Based on the binding modes of ASM in the presence and absence of sparsomycin, it is concluded that P-site occupation, even by an inhibitor, governs the positioning at the A-site.

A2602 is the feature that undergoes the largest conformational changes upon binding of substrates or inhibitors. As a consequence it has different orientations in each of the complexes of D50S and A-site substrate analogs or inhibitors described herein (FIGS. 19e-g) or elsewhere. In native D50S and in ASM bound structures, A2602 has similar conformations. In both it is located in the middle of the PTC, within contact distance of the ends of the docked A- and P-tRNAs. In the ACCP bound structure the orientation of A2602 is slightly different, but it still points into the PTC. The orientation of A2602 is significantly different in P-site occupied D50S. In both sparsomycin and ASMS structures, A2602 points away from the PTC toward the sparsomycin and acquires an orientation, distinctly different from all of the other orientations observed so far. Interestingly, the conformations of A2602 in the two liganded H50S structures (Nissen et al., 2000. Science 289, 920-30; Schmeing et al., 2002. Nat Struct Biol 9, 225-30) are close, albeit not similar, to those seen for D50S complexes. Combining all structures it seems that the limits of the rotation that A2602 undergoes are its locations in the presence of sparsomycin, which is related to that of chloramphenicol by a flip of 180 degrees (FIGS. 19h-i), an A-site antibiotic. In all other D50S and H50S structures A2602 is located between these two extreme points.

A2602 is the only nucleotide in the PTC that shows such striking diversity. This great variability suggest that A2602 may act as an important A-P-switch, in accord with the suggestion that A2602 and its induced conformational switch play a role in peptidyl transferase activity (Porse et al., 1999. Proc Natl Acad Sci U S A. 96, 9003-8). It is likely that it plays a dynamic role in translocation, working in concert with H69. While the latter assists the translocation near the subunit interface, the A2602 serves as a local switch within the PTC.

Two-fold rotation and active discrimination by the exit tunnel: A pseudo two-fold rotational axis within the peptidyl transferase cavity was identified that relates two groups of nine nucleotides (FIG. 19i). In each group, five belong to A- or to P-loops. Conformation, rather than the type of the base, is related by the pseudo two-fold symmetry. This local two-fold symmetry at the PTC of D50S is consistent with the observation that the CCAs bound in the A-and P-sites are related by a two-fold axis (Nissen et al., 2000. Science 289, 920-30). The local two-fold symmetry provides similar, albeit not identical, environments for the CCA termini, to allow for a smooth translocation with minor rearrangements (Yusupov et al., 2001. Science 292, 883-96) and without being exposed to large energetic differentials.

The peptidyl transferase reaction is exothermic, progressing downhill energetically, however, translocation of the tRNA-mRNA complex involves disruption of existing interactions in one site and the establishment of new interactions in the next site. Owing to the local two-fold symmetry, the environments of the A- and P-sites are similar. Nevertheless, the environment of the 3′ ends of the two tRNAs are somewhat different. In T70S crystals, the P-site tRNA seems to make more interactions with the P-loop than the A-site with the A-loop (Yusupov et al., 2001. Science 292, 883-96). In the liganded H50S crystals, the A- and the P-site tRNA make the same number of contacts with the PTC, but the P-site tRNA makes two base pairs whereas the. A-site tRNA is involved in only one base pair (Nissen et al., 2000. Science 289, 920-30). Hence, in both systems the progression from the A- to the P-site would be energetically favored and should enhance the contacts between the tRNA and the 23s rRNA.

The observation of a two fold symmetry between the A- and P-sites' 3′ tRNA termini implies that regardless of the translocation mechanism, the CCA end of the A-site tRNA bearing the newly formed polypeptide should rotate by approximately 180 degrees on its way from the A- to the P-site. This rotation may be triggered by the creation of the new peptide bond, and can occur, in principle, when the helical part of the tRNA is either at the A-site, or during its translocation to the P-site or after the tRNA reaches the P-site. In order to exclude non-permitted rotations due to space constrains, the three possibilities for rotation were modeled by the present inventors (Agmon et al., to be published). Starting from the location of the tRNA mimic (ASM) in D50S, it was found that a 180 degree rotation of its ACCA end together with the base bound to it, can occur while the helical part of ASM is at the A-site without steric hindrance. Furthermore, it was found that in ASM, the P-O3′ bond connecting bases 31 and 32 of the tRNA mimic, just above the ACCA terminal, is almost overlapping the local two-fold axis. Therefore the ACCA-peptidyl rotation may occur around this bond while the tRNA is at a location similar to that of ASM that may represent an intermediate hybrid state (Moazed and Noller, 1989. Nature 342, 142-8).

Nucleotides U2585 and A2602 are located approximately on the local two-fold axis, and U2585 is situated directly below A2602, in the direction of the protein exit tunnel. This construction hints that the extremely flexible nucleotide A2602 may play a dynamic role in coordinating the tRNA motions, and U2585 may assist in guiding the ACCA during the rotation and in transmitting messages from the tunnel wall to the PTC. This suggested rotation-translation motion could provide benefits not only for translocation but also for the progression of the nascent protein through the tunnel, since it may create a screw motion requiring less force than straight pushing. As the walls of the exit tunnel have bumps and grooves and as its diameter is not uniform, the progression of the nascent protein through the tunnel cannot be approximated to a smooth object progressing along smooth walls. Thus, although the tunnel is of a “non-stick” nature, as described in Example 1 above (Harms J. et al., 2001. Cell 107, 679-688) and elsewhere (Nissen et al., 2000. Science 289, 920-30), the growing proteins move at times through narrow paths, so that their side chains may exercise significant friction and may require assistance. Besides assisting the efficient movement of the nascent protein, the screw motion may be of advantage for active discrimination by the tunnel. This tunnel was recently suggested not to be completely passive, but to function as a discriminating gate (Gabashvili et al., 2001. Mol Cell 8, 181-8; Tenson and Mankin, 2001. Peptides 22, 1661-8; Nakatogawa and Ito, 2002. Cell 108, 629-36; Tenson and Ehrenberg, 2002. Cell 108, 591-4). This gating process can be related the nascent protein in eubacterial ribosomes, or be factor dependent in mammalian ribosomes (Walter and Johnson, 1994. Annu Rev Cell Biol 10, 87-119). The screw movement is suggested to direct the entrance of the nascent protein into the tunnel and assist its progression. As the nucleotides involved in generating this movement are situated in positions that allow them to respond to signaling from specific sequences of the nascent proteins, the may assist the gating procedures.

Base A2602 universality: Since no mutations of A2602 have so far been identified in-vivo, it was thought that mutations of A2602 produce dominant lethal phenotypes that escape selection (Porse et al., 1999. Proc Natl Acad Sci U S A. 96, 9003-8). Indeed, in its suggested task as a conformational switch, A2602 appears to be a pivotal nucleotide for the rotation of the 3′ ends and for the translocation of the tRNA molecules from the A- to P-sites, thus playing a major role in translocation and in the two-fold rotation. For the latter task, its identity seems to be less relevant. In addition, both the movement from A- to P-and the rotation of the CCA-peptidyl moiety may occur independently of A2602, since even slight tension originated by the newly formed peptide bond while at the A-site, may be sufficient to trigger translocation. In the termination step, however, after the production of the final peptide bond, the A-site should be empty, hence the contribution of A2602 may become crucial. This is consistent with the observation that mutations of A2602 cause severe compromise of the peptidyl release activity (Polacek and Mankin, private communication). From this point of view, it is conceivable that deletion or mutation of 2602 will hardly affect single bond formation in processes similar to the fragment reaction, since the small tRNA analogs may bind to the PTC in various orientations, and since no translocation follows this reaction.

Summary: High-resolution crystal structures of D50S in complex with short or long tRNA acceptor-stem mimics showed that precise positioning of long substrate analogs is determined by interactions of their helical stems with 23s rRNA and a ribosomal protein. They also indicated that the B2a intersubunit bridge, in concert with flexible nucleotides within the PTC, assist translocation. Comparing the binding modes of acceptor-stem mimics to free or P-site occupied subunits illuminated dynamic aspects of peptide bond formation, including conformational interplay between A- and P-sites. Analysis of the present results showed that sparsomycin introduces alterations in the PTC, thereby rationalizing controversies concerning the sparsomycin binding site in the LRS and its apparent dependence on P-site occupation.

Conclusion: The above-described results provide, for the first time, high resolution three-dimensional atomic structure models of the interaction of a eubacterial LRS with sparsomycin and/or tRNA acceptor stem mimics. Such models enabled elucidation of the mechanisms of action of the protein synthesis inhibitors puromycin and sparsomycin. These results furthermore provide a wealth of novel information describing structural aspects of the role of the ribosome in peptide bond formation. As such, the present three-dimensional atomic structure models are clearly superior to all prior art atomic structure models of complexes of the LRS and protein synthesis inhibitors. The three-dimensional structure models generated by the present invention can be advantageously utilized to design or identify novel or enhanced antibiotics. Such models can also be utilize in the rational design or identification of ribosomes displaying desired characteristics.

### Example 5

### High Resolution Three-Dimensional Atomic Structure Models of the Interaction of Troleandomycin with the Large Ribosomal Subunit: Elucidation of the Structural Basis for the Dynamic Role of the Ribosomal Tunnel in Cellular Regulation

Due the large number of debilitating and/or lethal diseases caused by bacterial pathogens for which no satisfactory treatments exist, including increasingly frequent diseases caused by antibiotic resistant bacteria, there is an urgent need for novel and enhanced antibiotics. Ideally, three-dimensional atomic structure models of ribosomes complexed with antibiotics, such as troleandomycin, are required in order to facilitate the rational design or selection of antibiotics effective against such pathogenic bacteria. Such models can further be employed to facilitate the rational design or selection of ribosomes displaying desired characteristics, such as the ability to enhance recombinant protein production. However, all prior art approaches have failed to produce satisfactory high resolution three-dimensional atomic models of the structural and functional interactions between antibiotics such as troleandomycin and the eubacterial LRS, or of the structure and function of the ribosome itself Thus, in order to overcome these limitations of the prior art, high resolution three-dimensional atomic structure models of the eubacterial LRS complexed to the antibiotic troleandomycin were generated and analyzed, as follows.

Materials and Methods:

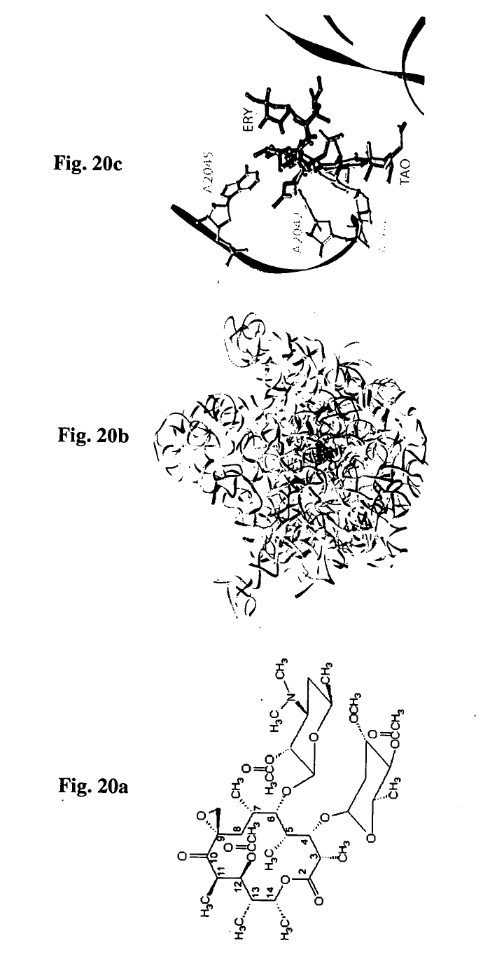

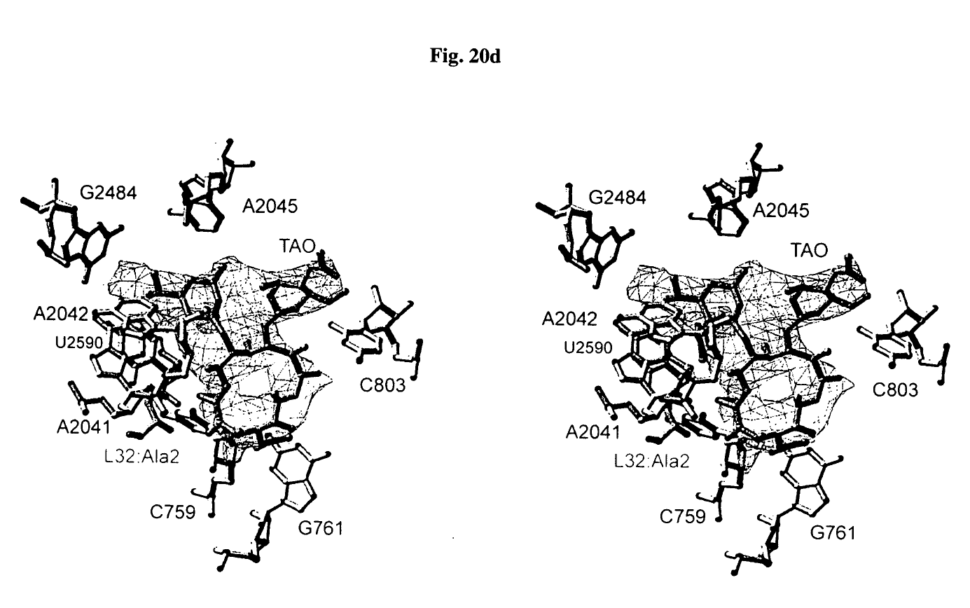

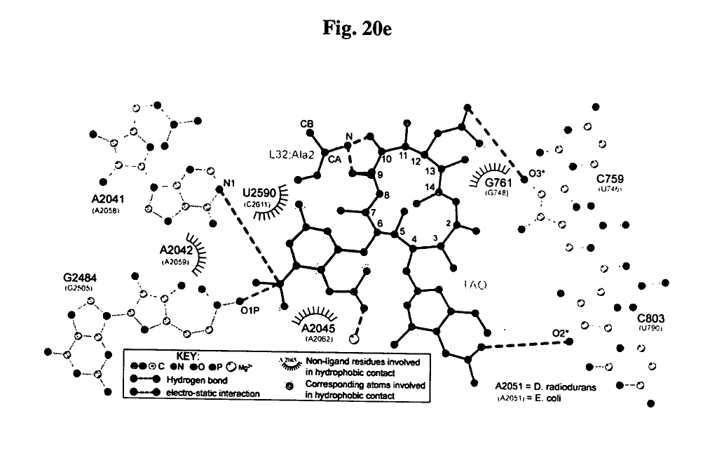

Crystallization: Crystals of D50S were generated as described in Example 1, above (Harms J. et al., 2001. Cell 107, 679-88) and soaked in solutions containing 0.1 mM of troleandomycin (Sigma; FIG. 20a) to generate D50S-troleandomycin complex crystals.

Crystallography: Data were collected at 100 K at ID29/ESRF/EMBL and ID19/APS/ANL, using Quantuum-210 and APS detectors, respectively. Data were processed with DENZO/SCALEPACK and HKL2000 (Otwinowski Z. and Minor W., 1997. Macromolecular Crystallogr A 276, 307-26). Overall and group rigid body refinements were performed with CNS software (Brunger A. T. et al., 1998. Acta Crystallogr D Biol Crystallogr 54, 905-21). Solvent flattened (Fo-Fc) and (2Fo-Fc) maps, calculated with SOLOMON software (Abrahams J. P. and De Graaff R. A., 1998. Curr Opin Struct Biol 8, 601-5), were used for placement of troleandomycin and modeling of the swung L22 conformation (Jones T. A. et al., 1991. Acta Crystallogr A 47, 110-9). Further refinement was carried out using CNS. The nascent polypeptide chain was modeled using O software (Jones T. A. et al., 1991. Acta Crystallogr A 47, 110-9). After modeling, Φ and Ψ angles fell mainly in the beta-sheet region of the Ramachandran plot. Hinge regions were determined from Cα-based dihedral angles (Flocco M. M. and Mowbray S. L., 1995. Protein Sci 4, 2118-22). Figures were produced using RIBBONS (Carson M., 1997. Macromolecular Crystallography, Pt B 277, 493-505) and LIGPLOT (Wallace A. C. et al., 1995. Protein Engineering 8, 127-134) softwares. Unless otherwise stated, nucleotide and amino acid residue coordinates of D50S RNA and polypeptide constituents, respectively, are numbered according to D. radiodurans.

Experimental Results:

Crystals of D50S in complex with troleandomycin, diffracting to higher than 3.4 Å were generated and used to produce electron density maps thereof (Table 37 and FIGS. 20b-e), thereby allowing the definition of the binding site of troleandomycin in the LRS.

TABLE 37
Crystallographic data*.

| | |
|---|---|
| Space group | I222 |
| Wavelength (Å) | 1.038 |
| Unit cell parameters (Å) | 170.3 × 411.1 × 695.5 |
| Resolution range (Å) | 20-3.4 |
| Mosaicity (°) | 0.35 |
| Number of unique reflections | 291,247 |
| Completeness (%) | 88.1 (84.5) |
| Rmerge (%) | 9.9 (43.2) |
| <I>/<σ(I)> | 5.4 (1.4) |
| R-factor (%) | 26.2 |
| R-free (%) | 31.0 |
| R-factor/R-free (%) | 5 |
| Bond lengths (Å) | 0.0073 |
| Bond length r.m.s. deviation from ideality (Å) | 0.005 |
| Bond angles (degrees) | 1.31 |
| Bond angle r.m.s. deviation from ideality | 0.003 degree |

*Values in parentheses refer to the highest resolution shell (3.46-3.4 Å)

Atomic structure of D50S-troleandomycin complex: The three-dimensional atomic structure of D50S in complex with troleandomycin was found to be defined by the sets of pdb atom coordinates set forth in Table 38 (refer to enclosed CD-ROM). Structure coordinates for proteins in these tables refer to alpha-carbon atoms of amino acid residues, and the nomenclature of constituents of these complexes used in these tables are specified in Table 39.

TABLE 39
Nomenclature of constituents of D50S-
troleandomycin complex used in Table 38.

| Constituent | Nomenclature |
|---|---|
| ribosomal proteins L2-L6 | L02-L06 |
| ribosomal protein L9 | L09 |
| ribosomal protein L11 | L11 |
| ribosomal proteins L13-L24 | L13-L24 |
| ribosomal protein CTC | CTC |
| ribosomal proteins L27-L36 | L27-L36 |
| 23S rRNA | 23S |
| 5S rRNA | 5S |
| troleandomycin | TROL |
| Mg2+ ions | MGMG |

Atomic structure of the troleandomycin binding site of D50S: Three-dimensional atomic structures of the portion of D50S-troleandomycin complex comprising all of troleandomycin and portions of D50S contained within a 20 Å-radius sphere centered on troleandomycin (coordinates 41, 132, 123) were found to be defined by the subset of Table 38-derived coordinates (refer to enclosed CD-ROM) set forth in Table 40 (refer to enclosed CD-ROM).

Analysis of the D50S-troleandomycin complex atomic structure contained in the above-described 20 Å-radius sphere demonstrated that troleandomycin interacts with the LRS via: 23S rRNA nucleotides having coordinates C759, G761, C803, A2041, A2042, A2045, G2484, and U2590; a ribosomal protein L32 amino acid residue having the amino acid residue coordinate Ala2; and the Mg2+ ion having the magnesium ion coordinate mg1.

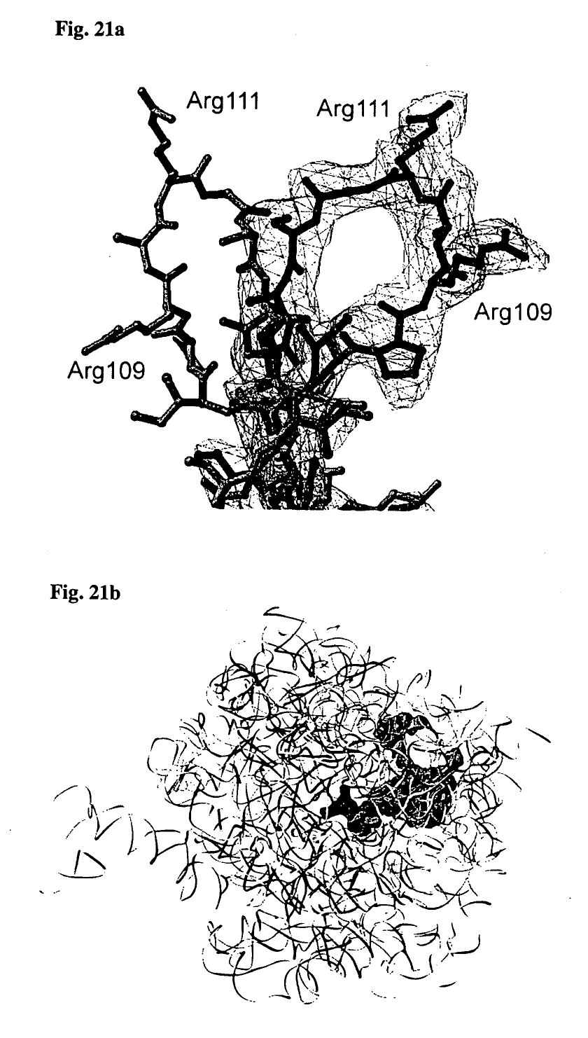

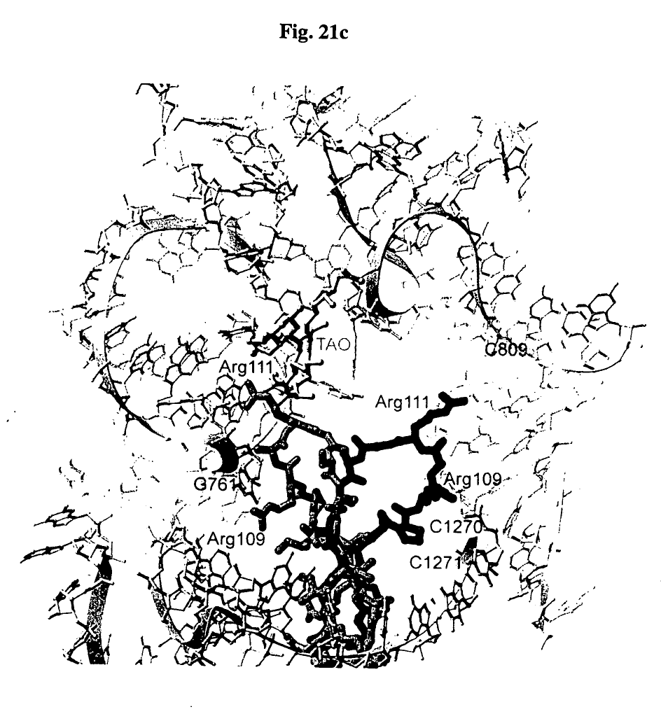

Troleandomycin induces modulation of the tunnel shape: The solvent-flattened difference Fourier map, obtained using the 3.0 Å structure of D50S described in Example 1, above (PDB 1LNR), contained two regions of positive density, and troleandomycin was placed unambiguously in one of them. The second positive density region, located deeper in the tunnel, was clearly interpreted as a novel conformation of the beta-hairpin of protein L22 (FIGS. 21a-c). This, together with a negative difference electron density at the location of the tip of L22 hairpin in D50S native structure, indicate that troleandomycin binding triggered striking conformational changes of this protein. To verify this unexpected result, two independent sets of data were used for constructing electron density maps. Both maps showed clearly troleandomycin binding site and the new conformation of L22 (referred to herein as the “swung” conformation).

As do other 14-member macrolides, troleandomycin binds to the LRS at a single site close to the tunnel entrance. It is, however, located somewhat deeper in the tunnel than erythromycin, so that its center of mass is displaced by 7.1 Å from that of erythromycin (FIGS. 21a-c). Whereas erythromycin is nearly perpendicular to the tunnel's wall, troleandomycin's lactone ring is tilted and forms a 20 degree angle with the tunnel wall, presumably due to its extended conformation and bulkier moieties. In its binding mode, all the functional groups of troleandomycin interact with the ribosome, whereas only the lactone ring and desosamine sugar are involved in erythromycin binding.

Common to troleandomycin and erythromycin are interactions with domain V of the rRNA (FIGS. 20a-e), as described in Example 2, above (Schlüenzen F. et al., Nature 413, 814-21). In particular, the desosamine sugars of both interact with the eubacterial adenine A2041 (A2058 in E. coli). This nucleotide was implicated in macrolide resistance and selectivity via mechanisms based on addition of bulky substituents to its base, either by di-methylation by Erm methylases (reviewed in Gasc J. C. et al., 1991. J Antibiot 44, 313-30) or by mutation to guanine, the nucleotide commonly found in eukaryotes and archaea at this position (reviewed in Weisblum B., 1995. Antimicrob Agents Chemother 39, 577-85). Unique for troleandomycin, and never observed so far for 14-member macrolides, as described above in Example 2 (Schlüenzen F. et al., 2001. Nature 413, 814-21), are its interactions with a protein, L32. Furthermore, the cladinose sugar of erythromycin is hardly involved in contacts with the ribosome, whereas in troleandomycin it interacts with the hairpin of H35 in domain II of the rRNA, previously implicated in binding of erythromycin, and in mutations conferring low resistance levels to it (Hansen L. H., P. et al., 1999. Mol Microbiol 31, 623-31).

In the complex of D50S with troleandomycin, the conformation of the tip of the beta-hairpin of protein L22, composed of 11 residues, differs strikingly from that observed in the D50S structure. This region contains two highly conserved arginines, Arg109 and Arg111, and an invariant glycine, Gly110. Superposition of troleandomycin's location on the D50S structure revealed that its lactone-ring acetyl occupies the space originally used by Arg111 (FIGS. 21a-c). In the D50S structure, the Arg111 side-chain is embedded in a narrow groove formed by nucleotides C759-762 and A764 which limit the space available for conformational rearrangements of Arg111. Consequently the binding of troleandomycin triggers global conformational rearrangements, flipping the tip of the L22 beta-hairpin around two hinges (residues 105-107 and 113-115) towards the other side of the tunnel, so that the distance between the backbone atoms of the native and the swung arginines is about 13 Å (FIGS. 21a-c).

Both the native and the swung conformations of the L22 beta-hairpin are stabilized mainly by electrostatic interactions and hydrogen bonds with the backbone of rRNA. In the native conformation, Arg109 interacts with G761 whereas Arg111 lines the tunnel wall. In the swung conformation, Arg109 interacts with C1270 and C1271 and Arg111 forms electrostatic interactions with C809. These two highly conserved arginines may be considered as a “double-hook” anchoring both native and swung conformations and modulating the switch between the two.

A deletion of three residues (82-84) of L22 leads to erythromycin resistance in E. coli (Wittmann H. G. et al., 1973. Mol Gen Genet 127, 175-89; Gregory S. T. and Dahlberg A. E., 1999. J Mol Biol 289, 827-34). An increase in the tunnel diameter, proportional to the size of the deleted residues, was detected in L22 mutated ribosomes by cryo-electron microscopy, and correlated with erythromycin resistance (Gabashvili I. S. et al., 2001. Mol Cell 8, 181-8). The distance between the erythromycin binding site and the L22 mutations and their proximity to the swinging tip of the L22 hairpin suggests, however, an indirect effect, as proposed earlier based on chemical probing (Gregory S. T. and Dahlberg A. E., 1999. J Mol Biol 289, 827-34). The present results indicate that structurally it is conceivable that a deletion adjacent to the hinge region could alter the conformation of the beta-hairpin tip in the region contacting the wall of the tunnel, and that these rearrangements could propagate and modify the shape of erythromycin binding site.

Regulatory role of the nascent protein exit tunnel: The observed swing of the tip of L22 beta-hairpin indicates its intrinsic conformational mobility. Since the swung conformation severely restricts the space available for the passage of nascent proteins through the tunnel, and since L22 double-hook is highly conserved, it is logical to link the swing of L22 with the putative regulatory role assigned to the tunnel. L22 is hereby proposed to constitute a main player in this task, with its double-hook acting as a conformational switch and providing the molecular tool for the gating and discriminative properties of the ribosome tunnel.

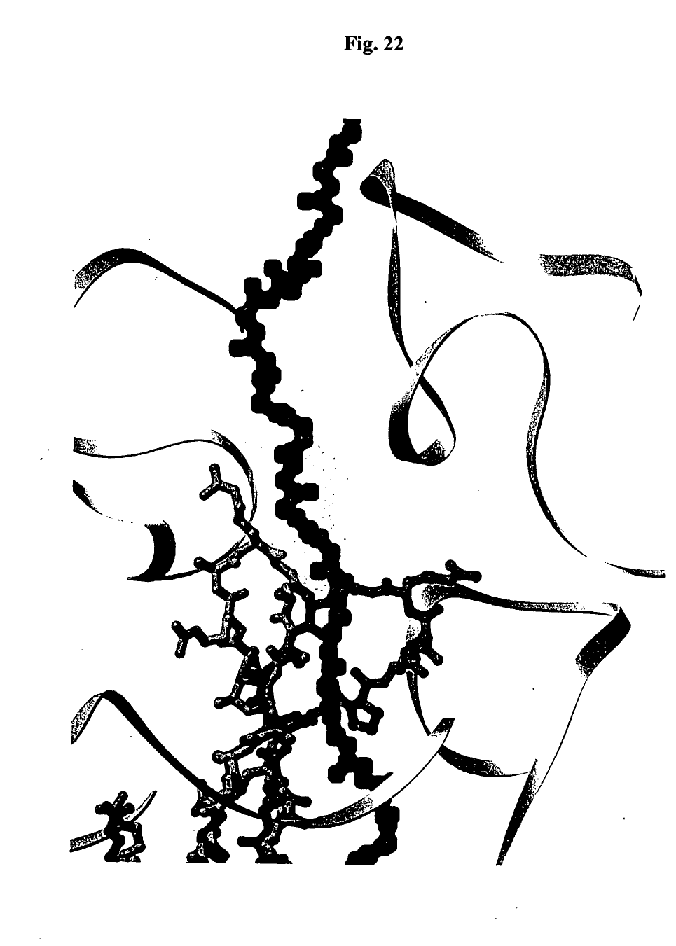

The FXXXXWIXXXXGIRAGP motif of SecM was shown to induce elongation arrest while this protein is being formed (Nakatogawa H. and Ito K., 2002. Cell 108, 629-36). Due to the conformational rigidity of proline, once Pro166 is incorporated into the nascent chain of SecM, the hydrophobic residues Trp155 and Ile156 may trigger a swing in a manner similar to troleandomycin, freeing space for the bulky side chains, but simultaneously sterically blocking progression of the nascent chain in the tunnel. Modeling of a poly-glycine nascent chain, kinked to comply with the curvature of the tunnel confirms that when Pro166 is placed at the entrance to the tunnel, below the active site, the two hydrophobic residues of the nascent chain, Trp155 and Ile156, indeed reach the tip of L22 hairpin and may collide with it (FIG. 22). In principle, nascent chains can use their flexibility to progress smoothly through the tunnel even in the proximity of the L22 beta-hairpin, as presumably happens when residues with bulky side-chains are incorporated into nascent proteins. However, the inherent rigidity of the proline that is located at the narrow entrance to the tunnel should hinder possible adjustments of the nascent chain. Consistently, Pro166, Trp155 and Ile156 were shown to be essential for elongation arrest (Nakatogawa H. and Ito K., 2002. Cell 108, 629-36).

The mechanism suggested by the present results is consistent with all known ribosomal arrest-suppressing mutations (Nakatogawa H. and Ito K., 2002. Cell 108, 629-36). The Gly91 to Ser91, Ala93 to Thr93, and Ala93 to Val93 mutations in L22 of E. coli introduce bulkier residues which, in the presence of the nascent chain, may destabilize the swung conformation. Inserting an adenine into the region forming the groove caging Arg111 in the native structure (A749-A753 in E. coli) should increase stabilization of this conformation and hamper the swing. Arrest suppression by the A2058 mutation (in E. coli) may act by relieving the space restrictions around the proline, such that the nascent chain can progress without colliding with the L22 hairpin.

The outstanding role of L22 and the conservation of its beta-hairpin size and sequence, suggest the discriminating mechanism to be universal. In prokaryotes, the L22 structure appears to be designed for its gating role. Precise positioning of the L22 hairpin stem, required for accurate swinging and anchoring of the double-hook, is presumably achieved by the pronounced positive surface charges of this region (Unge J. et al., 1998. Structure 6, 1577-86). At the same time, possible interactions with cellular factors that may trigger allosteric signaling may be mediated by the negatively charged surface of L22 at the tunnel opening. Of possible significance is the observation that the region of L22 positioned at the tunnel opening, and the patch interacting with rRNA show higher sequence variability in eukaryotes, presumably reflecting the higher complexity of these cells.

In summary, these results rationalize previous observations showing that the elongation process maybe sequence dependent; indicate that alterations between conformational states can take place within the tunnel; reveal that the tunnel may act as a control gate and illuminate its regulatory role.

Summary: A novel mode of action of an antibiotic agent was revealed in the crystal structure of a LRS in complex with troleandomycin. In addition to steric blockage of the ribosomal exit tunnel, troleandomycin triggers a striking conformational rearrangement of ribosomal protein L22, flipping the tip of its highly conserved beta-hairpin, so that it reaches the other side of the tunnel wall. This modulation of the tunnel shape provides the first structural insight into its dynamics. The existence of dynamic features within the ribosomal tunnel and its ability to oscillate between conformations, the known dependence of elongation arrest on sequence motifs within nascent peptides, the correlation between arrest-suppression mutants and the features involved in L22 gating, indicate that the tunnel is involved in sequence discrimination and may play active roles in regulation of intracellular processes. The presently above-described results therefore provide the structural basis for the roles of the tunnel in regulating intracellular events.

The crystal structure of D50S in complex with troleandomycin adds new insights into the mechanisms of action of macrolides. It indicates that macrolides drugs can hamper the progression of nascent chains in various modes, and shows the first case of an antibiotic blocking the tunnel both physically and by inducing conformational alterations in a ribosomal protein. This protein, L22, exhibits an intrinsic conformational mobility and contains a conserved double-hook feature, capable of interacting with two tunnel walls, thus creating a revolving gate within the tunnel. The observation of a dual inhibitory mechanism should open the door for the design of new potent antibiotics.

Conclusion: The above described results provide for the first time atomic resolution models of the structural and functional interactions involved in the binding of troleandomycin to a bacterial ribosome and provide much novel and valuable information regarding the functional and structural basis of the function of an antibiotic such as troleandomycin, as well as of ribosomal function itself. As such, the present three-dimensional atomic structure models are unique and advantageous relative to prior art models of antibiotic-ribosome interaction. Critically, such models constitute a potent means of rationally designing or selecting novel antibiotics. Such models can furthermore be utilized to facilitate rational design or selection of ribosomes having desired characteristics, such as enhanced protein production capacity. Thus, the presently described troleandomycin-LRS complex models are of significant utility in a broad range of biomedical, pharmacological, and industrial applications.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. A composition-of-matter comprising a crystallized complex of an antibiotic bound to a large ribosomal subunit of a eubacterium.

2. The composition-of-matter of claim 1, wherein said eubacterium is D. radiodurans.

3. The composition-of-matter of claim 1, wherein said eubacterium is a gram-positive bacterium.

4. The composition-of-matter of claim 1, wherein said eubacterium is a coccus.

5. The composition-of-matter of claim 1, wherein said eubacterium is a Deinococcus-Thermophilus group bacterium.

6. The composition-of-matter of claim 1, wherein said antibiotic is clindamycin and whereas said crystallized complex is characterized by unit cell dimensions of a=170.286±10 Å, b=410.134±15 Å and c=697.201±25 Å.

7. The composition-of-matter of claim 1, wherein said antibiotic is erythromycin and whereas said crystallized complex is characterized by unit cell dimensions of a=169.194±10 Å, b=409.975±15 Å and c=695.049±25 Å.

8. The composition-of-matter of claim 1, wherein said antibiotic is clarithromycin and whereas said crystallized complex is characterized by unit cell dimensions of a=169.871±10 Å, b=412.705±15 Å and c=697.008±25 Å.

9. The composition-of-matter of claim 1, wherein said antibiotic is roxithromycin and whereas said crystallized complex is characterized by unit cell dimensions of a=170.357±10 Å, b=410.713±15 Å and c=694.810±25 Å.

10. The composition-of-matter of claim 1, wherein said antibiotic is chloramphenicol and whereas said crystallized complex is characterized by unit cell dimensions of a=171.066±10 Å, b=409.312±15 Å and c=696.946±25 Å.

11. The composition-of-matter of claim 1, wherein said antibiotic is ACCP and whereas said crystallized complex is characterized by unit cell dimensions of a=169.9 Å, b=410.4 and c=697.1 Å.

12. The composition-of-matter of claim 1, wherein said antibiotic is ASM and whereas said crystallized complex is characterized by unit cell dimensions of a=169.9 Å, b=409.9 Å and c=695.9 Å.

13. The composition-of-matter of claim 1, wherein said antibiotic is ASMS and whereas said crystallized complex is characterized by unit cell dimensions of a=169.6 Å, b=409.4 Å and c=695.1 Å.

14. The composition-of-matter of claim 1, wherein said antibiotic is sparsomycin and whereas said crystallized complex is characterized by unit cell dimensions of a=169.1 Å, b=409.9 Å and c=696.3 Å.

15. The composition-of-matter of claim 1, wherein said antibiotic is troleandomycin and whereas said crystallized complex is characterized by unit cell dimensions of a=170.3 Å, b=411.1 Å and c=695.5 Å.

16. The composition-of-matter of claim 1, wherein said crystallized complex is characterized by having a crystal space group of I222.

17. The composition-of-matter of claim 1, wherein said antibiotic is selected from the group consisting of chloramphenicol, a lincosamide antibiotic, a macrolide antibiotic, a puromycin conjugate, ASMS, and sparsomycin.

18. The composition-of-matter of claim 17, wherein said lincosamide antibiotic is clindamycin.

19. The composition-of-matter of claim 17, wherein said macrolide antibiotic is selected from the group consisting of erythromycin, clarithromycin, roxithromycin, troleandomycin, a ketolide antibiotic and an azalide antibiotic.

20. The composition-of-matter of claim 19, wherein said ketolide antibiotic is ABT-773.

21. The composition-of-matter of claim 19, wherein said azalide antibiotic is azithromycin.

22. The composition-of-matter of claim 17, wherein said puromycin conjugate is ACCP or ASM.

23. The composition-of-matter of claim 1, wherein said antibiotic is chloramphenicol and whereas said large ribosomal subunit comprises a nucleic acid molecule, a segment of which including nucleotides being associated with said chloramphenicol, wherein a three-dimensional atomic structure of said nucleotides is defined by the set of structure coordinates corresponding to nucleotide coordinates 2044, 2430, 2431, 2479 and 2483-2485 set forth in Table 7.

24. The composition-of-matter of claim 1, wherein said antibiotic is chloramphenicol and whereas said large ribosomal subunit comprises a nucleic acid molecule, a segment of which including nucleotides being associated with said chloramphenicol, wherein a three-dimensional atomic structure of said nucleotides is defined by a set of structure coordinates having a root mean square deviation of not more than 2.0 Å from the set of structure coordinates corresponding to nucleotide coordinates 2044, 2430, 2431, 2479 and 2483-2485 set forth in Table 7.

25. The composition-of-matter of claim 23, wherein a three-dimensional atomic structure of said segment is defined by the set of structure coordinates corresponding to nucleotide coordinates 2044-2485 set forth in Table 7.

26. The composition-of-matter of claim 24, wherein a three-dimensional atomic structure of said segment is defined by a set of structure coordinates having a root mean square deviation of not more than 2.0 Å from the set of structure coordinates corresponding to nucleotide coordinates 2044-2485 set forth in Table 7.

27. The composition-of-matter of claim 1, wherein said antibiotic is chloramphenicol and whereas a three-dimensional atomic structure of said chloramphenicol is defined by the set of structure coordinates corresponding to HETATM coordinates 59925-59944 set forth in Table 7.

28. The composition-of-matter of claim 1, wherein said antibiotic is chloramphenicol and whereas a three-dimensional atomic structure of said chloramphenicol is defined by a set of structure coordinates having a root mean square deviation of not more than 2.0 Å from the set of structure coordinates corresponding to HETATM coordinates 59925-59944 set forth in Table 7.

29. The composition-of-matter of claim 1, wherein said antibiotic is clindamycin and whereas said large ribosomal subunit comprises a nucleic acid molecule, a segment of which including nucleotides being associated with said clindamycin, wherein a three-dimensional atomic structure of said nucleotides is defined by the set of structure coordinates corresponding to nucleotide coordinates 2040-2042, 2044, 2482, 2484 and 2590 set forth in Table 8.

30. The composition-of-matter of claim 1, wherein said antibiotic is clindamycin and whereas said large ribosomal subunit comprises a nucleic acid molecule, a segment of which including nucleotides being associated with said clindamycin, wherein a three-dimensional atomic structure of said nucleotides is defined by a set of structure coordinates having a root mean square deviation of not more than 2.0 Å from the set of structure coordinates corresponding to nucleotide coordinates 2040-2042, 2044, 2482, 2484 and 2590 set forth in Table 8.

31. The composition-of-matter of claim 29, wherein a three-dimensional atomic structure of said segment is defined by the set of structure coordinates corresponding to nucleotide coordinates 2040-2590 set forth in Table 8.

32. The composition-of-matter of claim 30, wherein a three-dimensional atomic structure of said segment is defined by a set of structure coordinates having a root mean square deviation of not more than 2.0 Å from the set of structure coordinates corresponding to nucleotide coordinates 2040-2590 set forth in Table 8.

33. The composition-of-matter of claim 1, wherein said antibiotic is clindamycin and whereas a three-dimensional atomic structure of said clindamycin is defined by the set of structure coordinates corresponding to HETATM coordinates 59922-59948 set forth in Table 8.

34. The composition-of-matter of claim 1, wherein said antibiotic is clindamycin and whereas a three-dimensional atomic structure of said clindamycin is defined by a set of structure coordinates having a root mean square deviation of not more than 2.0 Å from the set of structure coordinates corresponding to HETATM coordinates 59922-59948 set forth in Table 8.

35. The composition-of-matter of claim 1, wherein said antibiotic is clarithromycin and whereas said large ribosomal subunit comprises a nucleic acid molecule, a segment of which including nucleotides being associated with said clarithromycin, wherein a three-dimensional atomic structure of said nucleotides is defined by the set of structure coordinates corresponding to nucleotide coordinates 2040-2042, 2045, 2484, 2588 and 2589 set forth in Table 9.

36. The composition-of-matter of claim 1, wherein said antibiotic is clarithromycin and whereas said large ribosomal subunit comprises a nucleic acid molecule, a segment of which including nucleotides being associated with said clarithromycin, wherein a three-dimensional atomic structure of said nucleotides is defined by a set of structure coordinates having a root mean square deviation of not more than 2.0 Å from the set of structure coordinates corresponding to nucleotide coordinates 2040-2042, 2045, 2484, 2588 and 2589 set forth in Table 9.

37. The composition-of-matter of claim 35, wherein a three-dimensional atomic structure of said segment is defined by the set of structure coordinates corresponding to nucleotide coordinates 2040-2589 set forth in Table 9.

38. The composition-of-matter of claim 36, wherein a three-dimensional atomic structure of said segment is defined by a set of structure coordinates having a root mean square deviation of not more than 2.0 Å from the set of structure coordinates corresponding to nucleotide coordinates 2040-2589 set forth in Table 9.

39. The composition-of-matter of claim 1, wherein said antibiotic is clarithromycin and whereas a three-dimensional atomic structure of said clarithromycin is defined by the set of structure coordinates corresponding to HETATM coordinates 59922-59973 set forth in Table 9.

40. The composition-of-matter of claim 1, wherein said antibiotic is clarithromycin and whereas a three-dimensional atomic structure of said clarithromycin is defined by a set of structure coordinates having a root mean square deviation of not more than 2.0 Å from the set of structure coordinates corresponding to HETATM coordinates 59922-59973 set forth in Table 9.

41. The composition-of-matter of claim 1, wherein said antibiotic is erythromycin and whereas said large ribosomal subunit comprises a nucleic acid molecule, a segment of which including nucleotides being associated with said erythromycin, wherein a three-dimensional atomic structure of said nucleotides is defined by the set of structure coordinates corresponding to nucleotide coordinates 2040-2042, 2045, 2484, 2588 and 2589 set forth in Table 10.

42. The composition-of-matter of claim 1, wherein said antibiotic is erythromycin and whereas said large ribosomal subunit comprises a nucleic acid molecule, a segment of which including nucleotides being associated with said erythromycin, wherein a three-dimensional atomic structure of said nucleotides is defined by a set of structure coordinates having a root mean square deviation of not more than 2.0 Å from the set of structure coordinates corresponding to nucleotide coordinates 2040-2042, 2045, 2484, 2588 and 2589 set forth in Table 10.

43. The composition-of-matter of claim 41, wherein a three-dimensional atomic structure of said segment is defined by the set of structure coordinates corresponding to nucleotide coordinates 2040-2589 set forth in Table 10.

44. The composition-of-matter of claim 42, wherein a three-dimensional atomic structure of said segment is defined by a set of structure coordinates having a root mean square deviation of not more than 2.0 Å from the set of structure coordinates corresponding to nucleotide coordinates 2040-2589 set forth in Table 10.

45. The composition-of-matter of claim 1, wherein said antibiotic is erythromycin and whereas a three-dimensional atomic structure of said erythromycin is defined by the set of structure coordinates corresponding to HETATM coordinates 59922-59972 set forth in Table 10.

46. The composition-of-matter of claim 1, wherein said antibiotic is erythromycin and whereas a three-dimensional atomic structure of said erythromycin is defined by a set of structure coordinates having a root mean square deviation of not more than 2.0 Å from the set of structure coordinates corresponding to HETATM coordinates 59922-59972 set forth in Table 10.

47. The composition-of-matter of claim 1, wherein said antibiotic is roxithromycin and whereas said large ribosomal subunit comprises a nucleic acid molecule, a segment of which including nucleotides being associated with said roxithromycin, wherein a three-dimensional atomic structure of said nucleotides is defined by the set of structure coordinates corresponding to nucleotide coordinates 2040-2042, 2045, 2484, 2588 and 2589 set forth in Table 11.

48. The composition-of-matter of claim 1, wherein said antibiotic is roxithromycin and whereas said large ribosomal subunit comprises a nucleic acid molecule, a segment of which including nucleotides being associated with said roxithromycin, wherein a three-dimensional atomic structure of said nucleotides is defined by a set of structure coordinates having a root mean square deviation of not more than 2.0 Å from the set of structure coordinates corresponding to nucleotide coordinates 2040-2042, 2045, 2484, 2588 and 2589 set forth in Table 11.

49. The composition-of-matter of claim 47, wherein a three-dimensional atomic structure of said segment is defined by the set of structure coordinates corresponding to nucleotide coordinates 2040-2589 set forth in Table 11.

50. The composition-of-matter of claim 48, wherein a three-dimensional atomic structure of said segment is defined by a set of structure coordinates having a root mean square deviation of not more than 2.0 Å from the set of structure coordinates corresponding to nucleotide coordinates 2040-2589 set forth in Table 11.

51. The composition-of-matter of claim 1, wherein said antibiotic is roxithromycin and whereas a three-dimensional atomic structure of said roxithromycin is defined by the set of structure coordinates corresponding to HETATM coordinates 59922-59979 set forth in Table 11.

52. The composition-of-matter of claim 1, wherein said antibiotic is roxithromycin and whereas a three-dimensional atomic structure of said roxithromycin is defined by a set of structure coordinates having a root mean square deviation of not more than 2.0 Å from the set of structure coordinates corresponding to HETATM coordinates 59922-59979 set forth in Table 11.

53. The composition-of-matter of claim 1, wherein said antibiotic is ABT-773 and whereas said large ribosomal subunit comprises a nucleic acid molecule, a segment of which including nucleotides being associated with said ABT-773, wherein a three-dimensional atomic structure of said nucleotides is defined by the set of structure coordinates corresponding to nucleotide coordinates 803, 1773, 2040-2042, 2045, 2484, and 2588-2590 set forth in Table 18.

54. The composition-of-matter of claim 1, wherein said antibiotic is ABT-773 and whereas said large ribosomal subunit comprises a nucleic acid molecule, a segment of which including nucleotides being associated with said ABT-773, wherein a three-dimensional atomic structure of said nucleotides is defined by a set of structure coordinates having a root mean square deviation of not more than 2.0 Å from the set of structure coordinates corresponding to nucleotide coordinates 803, 1773, 2040-2042, 2045, 2484, and 2588-2590 set forth in Table 18.

55. The composition-of-matter of claim 53, wherein a three-dimensional atomic structure of said segment is defined by the set of structure coordinates corresponding to nucleotide coordinates 803-2590 set forth in Table 18.

56. The composition-of-matter of claim 54, wherein a three-dimensional atomic structure of said segment is defined by a set of structure coordinates having a root mean square deviation of not more than 2.0 Å from the set of structure coordinates corresponding to nucleotide coordinates 803-2590 set forth in Table 18.

57. The composition-of-matter of claim 1, wherein said antibiotic is ABT-773 and whereas a three-dimensional atomic structure of said ABT-773 is defined by the set of structure coordinates corresponding to HETATM coordinates 1-55 set forth in Table 18.

58. The composition-of-matter of claim 1, wherein said antibiotic is ABT-773 and whereas a three-dimensional atomic structure of said ABT-773 is defined by a set of structure coordinates having a root mean square deviation of not more than 2.0 Å from the set of structure coordinates corresponding to HETATM coordinates 1-55 set forth in Table 18.

59. The composition-of-matter of claim 1, wherein said antibiotic is azithromycin and whereas said large ribosomal subunit comprises a nucleic acid molecule, a segment of which including nucleotides being associated with said azithromycin, wherein a three-dimensional atomic structure of said nucleotides is defined by the set of structure coordinates corresponding to nucleotide coordinates 764, 2041, 2042, 2045, A2482, 2484, 2565, and 2588-2590 set forth in Table 19.

60. The composition-of-matter of claim 1, wherein said antibiotic is azithromycin and whereas said large ribosomal subunit comprises a nucleic acid molecule, a segment of which including nucleotides being associated with said azithromycin, wherein a three-dimensional atomic structure of said nucleotides is defined by a set of structure coordinates having a root mean square deviation of not more than 2.0 Å from the set of structure coordinates corresponding to nucleotide coordinates 764, 2041, 2042, 2045, A2482, 2484, 2565, and 2588-2590 set forth in Table 19.

61. The composition-of-matter of claim 1, wherein said antibiotic is azithromycin and whereas said large ribosomal subunit comprises amino acid residues being associated with said azithromycin, wherein a three-dimensional atomic structure of said amino acid residues is defined by the set of structure coordinates corresponding to amino acid residue coordinates Y59, G60, G63, T64 and R111 set forth in Table 19.

62. The composition-of-matter of claim 1, wherein said antibiotic is azithromycin and whereas said large ribosomal subunit comprises amino acid residues being associated with said azithromycin, wherein a three-dimensional atomic structure of said amino acid residues is defined by a set of structure coordinates having a root mean square deviation of not more than 2.0 Å from the set of structure coordinates corresponding to amino acid residue coordinates Y59, G60, G63, T64 and R111 set forth in Table 19.

63. The composition-of-matter of claim 59, wherein a three-dimensional atomic structure of said segment is defined by the set of structure coordinates corresponding to nucleotide coordinates 764-2590 set forth in Table 19.

64. The composition-of-matter of claim 60, wherein a three-dimensional atomic structure of said segment is defined by a set of structure coordinates having a root mean square deviation of not more than 2.0 Å from the set of structure coordinates corresponding to nucleotide coordinates 764-2590 set forth in Table 19.

65. The composition-of-matter of claim 1, wherein said antibiotic is azithromycin and whereas a three-dimensional atomic structure of said azithromycin is defined by the set of structure coordinates corresponding to HETATM coordinates 79705-79808 set forth in Table 19.

66. The composition-of-matter of claim 1, wherein said antibiotic is azithromycin and whereas a three-dimensional atomic structure of said azithromycin is defined by a set of structure coordinates having a root mean square deviation of not more than 2.0 Å from the set of structure coordinates corresponding to HETATM coordinates 79705-79808 set forth in Table 19.

67. The composition-of-matter of claim 1, wherein said antibiotic is ACCP and whereas said large ribosomal subunit comprises a nucleic acid molecule, a segment of which including nucleotides being associated with said ACCP, wherein a three-dimensional atomic structure of said nucleotides is defined by the set of structure coordinates corresponding to nucleotide coordinates 1924, 2430, 2485, 2532-2534, 2552, 2562, and 2583 set forth in Table 20.

68. The composition-of-matter of claim 1, wherein said antibiotic is ACCP and whereas said large ribosomal subunit comprises a nucleic acid molecule, a segment of which including nucleotides being associated with said ACCP, wherein a three-dimensional atomic structure of said nucleotides is defined by a set of structure coordinates having a root mean square deviation of not more than 2.0 Å from the set of structure coordinates corresponding to nucleotide coordinates 1924, 2430, 2485, 2532-2534, 2552, 2562, and 2583 set forth in Table 20.

69. The composition-of-matter of claim 67, wherein a three-dimensional atomic structure of said segment is defined by the set of structure coordinates corresponding to nucleotide coordinates 1924-2583 set forth in Table 20.

70. The composition-of-matter of claim 68, wherein a three-dimensional atomic structure of said segment is defined by a set of structure coordinates having a root mean square deviation of not more than 2.0 Å from the set of structure coordinates corresponding to nucleotide coordinates 1924-2583 set forth in Table 20.

71. The composition-of-matter of claim 1, wherein said antibiotic is ACCP and whereas a three-dimensional atomic structure of said ACCP is defined by the set of structure coordinates corresponding to atom coordinates 78760-78855 set forth in Table 20.

72. The composition-of-matter of claim 1, wherein said antibiotic is ACCP and whereas a three-dimensional atomic structure of said ACCP is defined by a set of structure coordinates having a root mean square deviation of not more than 2.0 Å from the set of structure coordinates corresponding to atom coordinates 78760-78855 set forth in Table 20.

73. The composition-of-matter of claim 1, wherein said antibiotic is ASM and whereas said large ribosomal subunit comprises: a nucleic acid molecule, a segment of which including nucleotides being associated with said ASM, wherein a three-dimensional atomic structure of said nucleotides is defined by the set of structure coordinates corresponding to nucleotide coordinates 1892, 1896, 1899, 1925, 1926, 2431, 2485, 2486, 2532, 2534, 2552, 2562, and 2581 set forth in Table 21; and a polypeptide including amino acid residues being associated with said ASM, wherein a three-dimensional atomic structure of said amino acid residues is defined by the set of structure coordinates corresponding to amino acid residue coordinates 79-81 set forth in Table 21.

74. The composition-of-matter of claim 1, wherein said antibiotic is ASM and whereas said large ribosomal subunit comprises: a nucleic acid molecule, a segment of which including nucleotides being associated with said ASM, wherein a three-dimensional atomic structure of said nucleotides is defined by a set of structure coordinates having a root mean square deviation of not more than 2.0 Å from the set of structure coordinates corresponding to nucleotide coordinates 1892, 1896, 1899, 1925, 1926, 2431, 2485, 2486, 2532, 2534, 2552, 2562, and 2581 set forth in Table 21; and a polypeptide including amino acid residues being associated with said ASM, wherein a three-dimensional atomic structure of said amino acid residues is defined by a set of structure coordinates having a root mean square deviation of not more than 2.0 Å from the set of structure coordinates corresponding to amino acid residue coordinates 79-81 set forth in Table 21.

75. The composition-of-matter of claim 73, wherein a three-dimensional atomic structure of said segment is defined by the set of structure coordinates corresponding to nucleotide coordinates 1892-2581 set forth in Table 21.

76. The composition-of-matter of claim 74, wherein a three-dimensional atomic structure of said segment is defined by a set of structure coordinates having a root mean square deviation of not more than 2.0 Å from the set of structure coordinates corresponding to nucleotide coordinates 1892-2581 set forth in Table 21.

77. The composition-of-matter of claim 1, wherein said antibiotic is ASM and whereas a three-dimensional atomic structure of said ASM is defined by the set of structure coordinates corresponding to atom coordinates 78747-79289 set forth in Table 21.

78. The composition-of-matter of claim 1, wherein said antibiotic is ASM and whereas a three-dimensional atomic structure of said ASM is defined by a set of structure coordinates having a root mean square deviation of not more than 2.0 Å from the set of structure coordinates corresponding to atom coordinates 78747-79289 set forth in Table 21.

79. The composition-of-matter of claim 1, wherein said antibiotic is ASMS and whereas said large ribosomal subunit comprises: a nucleic acid molecule, a segment of which including nucleotides being associated with said ASMS, wherein a three-dimensional atomic structure of said nucleotides is defined by the set of structure coordinates corresponding to nucleotide coordinates 1899, 1924, 1926, 2430, 2472, 2485, 2486, 2532, 2534, 2552, 2562, 2563, and 2581; set forth in Table 22; a polypeptide including amino acid residues being associated with said ASM, wherein a three-dimensional atomic structure of said amino acid residues is defined by the set of structure coordinates corresponding to amino acid residue coordinates 79-81 set forth in Table 22; and magnesium ions being associated with said ASM, wherein a three-dimensional positioning of said magnesium ions is defined by the set of structure coordinates corresponding to atom coordinates 79393 and 79394 set forth in Table 22.

80. The composition-of-matter of claim 1, wherein said antibiotic is ASMS and whereas said large ribosomal subunit comprises a nucleic acid molecule, a segment of which including nucleotides being associated with said ASMS, wherein a three-dimensional atomic structure of said nucleotides is defined by a set of structure coordinates having a root mean square deviation of not more than 2.0 Å from the set of structure coordinates corresponding to nucleotide coordinates 1899, 1924, 1926, 2430, 2472, 2485, 2486, 2532, 2534, 2552, 2562, 2563, and 2581 set forth in Table 22; a polypeptide including amino acid residues being associated with said ASM, wherein a three-dimensional atomic structure of said amino acid residues is defined by a set of structure coordinates having a root mean square deviation of not more than 2.0 Å from the set of structure coordinates corresponding to amino acid residue coordinates 79-81 set forth in Table 22; and magnesium ions being associated with said ASM, wherein a three-dimensional positioning of said magnesium ions is defined by a set of structure coordinates having a root mean square deviation of not more than 2.0 Å from the set of structure coordinates corresponding to atom coordinates 79393 and 79394 set forth in Table 22.

81. The composition-of-matter of claim 79, wherein a three-dimensional atomic structure of said segment is defined by the set of structure coordinates corresponding to nucleotide coordinates 1924-2581 set forth in Table 22.

82. The composition-of-matter of claim 80, wherein a three-dimensional atomic structure of said segment is defined by a set of structure coordinates having a root mean square deviation of not more than 2.0 Å from the set of structure coordinates corresponding to nucleotide coordinates 1924-2581 set forth in Table 22.

83. The composition-of-matter of claim 1, wherein said antibiotic is ASMS and whereas a three-dimensional atomic structure of said ASMS is defined by the set of structure coordinates corresponding to atom coordinates 78758-79322 set forth in Table 22.

84. The composition-of-matter of claim 1, wherein said antibiotic is ASMS and whereas a three-dimensional atomic structure of said ASMS is defined by a set of structure coordinates having a root mean square deviation of not more than 2.0 Å from the set of structure coordinates corresponding to coordinates 78758-79322 set forth in Table 22.

85. The composition-of-matter of claim 1, wherein said antibiotic is sparsomycin and whereas said large ribosomal subunit comprises a nucleic acid molecule, a nucleotide of which being associated with said sparsomycin, wherein a three-dimensional atomic structure of said nucleotide is defined by a set of structure coordinates corresponding to nucleotide coordinate 2581 set forth in Table 23.

86. The composition-of-matter of claim 1, wherein said antibiotic is sparsomycin and whereas said large ribosomal subunit comprises a nucleic acid molecule, a nucleotide of which being associated with said sparsomycin, wherein a three-dimensional atomic structure of said nucleotide is defined by a set of structure coordinates having a root mean square deviation of not more than 2.0 Å from the structure coordinate corresponding to nucleotide coordinate 2581 set forth in Table 23.

87. The composition-of-matter of claim 1, wherein said antibiotic is sparsomycin and whereas a three-dimensional atomic structure of said sparsomycin is defined by the set of structure coordinates corresponding to atom coordinates 78757-78778 set forth in Table 23.

88. The composition-of-matter of claim 1, wherein said antibiotic is sparsomycin and whereas a three-dimensional atomic structure of said sparsomycin is defined by a set of structure coordinates having a root mean square deviation of not more than 2.0 Å from the set of structure coordinates corresponding to atom coordinates 78757-78778 set forth in Table 23.

89. The composition-of-matter of claim 1, wherein said antibiotic is troleandomycin and whereas said large ribosomal subunit comprises a nucleic acid molecule, a segment of which including nucleotides being associated with said troleandomycin, wherein a three-dimensional atomic structure of said nucleotides is defined by the set of structure coordinates corresponding to nucleotide coordinates 759, 761, 803, 2041, 2042, 2045, 2484, and 2590 set forth in Table 38.

90. The composition-of-matter of claim 1, wherein said antibiotic is troleandomycin and whereas said large ribosomal subunit comprises a nucleic acid molecule, a segment of which including nucleotides being associated with said troleandomycin, wherein a three-dimensional atomic structure of said nucleotides is defined by a set of structure coordinates having a root mean square deviation of not more than 2.0 Å from the set of structure coordinates corresponding to nucleotide coordinates 759, 761, 803, 2041, 2042, 2045, 2484, and 2590 set forth in Table 38.

91. The composition-of-matter of claim 89, wherein a three-dimensional atomic structure of said segment is defined by the set of structure coordinates corresponding to nucleotide coordinates 759-2590 set forth in Table 38.

92. The composition-of-matter of claim 90, wherein a three-dimensional atomic structure of said segment is defined by a set of structure coordinates having a root mean square deviation of not more than 2.0 Å from the set of structure coordinates corresponding to nucleotide coordinates 759-2590 set forth in Table 38.

93. The composition-of-matter of claim 1, wherein said antibiotic is troleandomycin and whereas said large ribosomal subunit comprises an amino acid residue being associated with said troleandomycin, wherein a three-dimensional atomic structure of said amino acid residue is defined by the set of structure coordinates corresponding to amino acid residue coordinate Ala2 set forth in Table 38.

94. The composition-of-matter of claim 1, wherein said antibiotic is troleandomycin and whereas said large ribosomal subunit comprises an amino acid residue being associated with said troleandomycin, wherein a three-dimensional atomic structure of said nucleotides is defined by a set of structure coordinates having a root mean square deviation of not more than 2.0 Å from the set of structure coordinates corresponding to amino acid residue coordinate Ala2 set forth in Table 38.

95. The composition-of-matter of claim 1, wherein said antibiotic is troleandomycin and whereas a three-dimensional atomic structure of said troleandomycin is defined by the set of structure coordinates corresponding to atom coordinates 1-57 set forth in Table 38.

96. The composition-of-matter of claim 1, wherein said antibiotic is troleandomycin and whereas a three-dimensional atomic structure of said troleandomycin is defined by a set of structure coordinates having a root mean square deviation of not more than 2.0 Å from the set of structure coordinates corresponding to atom coordinates 1-57 set forth in Table 38.

97. A composition-of-matter comprising a crystallized LRS of a eubacterium.

98. The composition-of-matter of claim 97, wherein said eubacterium is D. radiodurans.

99. The composition-of-matter of claim 97, wherein said eubacterium is a gram-positive bacterium.

100. The composition-of-matter of claim 97, wherein said eubacterium is a coccus.

101. The composition-of-matter of claim 97, wherein said eubacterium is a Deinococcus-Thermophilus group bacterium.

102. The composition-of-matter of claim 97, wherein said crystallized large ribosomal subunit is characterized by unit cell dimensions of a=170.827±10 Å, b=409.430±15 AÅ and c=695.597±25 Å.

103. The composition-of-matter of claim 97, wherein said crystallized large ribosomal subunit is characterized by having a crystal space group of I222.

104. The composition-of-matter of claim 97, wherein a three-dimensional atomic structure of at least a portion of said crystallized large ribosomal subunit is defined by a set of structure coordinates corresponding to a set of coordinates set forth in Table 3, said set of coordinates set forth in Table 3 being selected from the group consisting of:
nucleotide coordinates 2044, 2430, 2431, 2479 and 2483-2485;
nucleotide coordinates 2044-2485;
nucleotide coordinates 2040-2042, 2044, 2482, 2484 and 2590;
nucleotide coordinates 2040-2590;
nucleotide coordinates 2040-2042, 2045, 2484, 2588 and 2589;
nucleotide coordinates 2040-2589;
atom coordinates 1-59360;
atom coordinates 59361-61880;
atom coordinates 1-61880;
atom coordinates 61881-62151;
atom coordinates 62152-62357;
atom coordinates 62358-62555;
atom coordinates 62556-62734;
atom coordinates 62735-62912;
atom coordinates 62913-62965;
atom coordinates 62966-63109;
atom coordinates 63110-63253;
atom coordinates 63254-63386;
atom coordinates 63387-63528;
atom coordinates 63529-63653;
atom coordinates 63654-63768;
atom coordinates 63769-63880;
atom coordinates 63881-64006;
atom coordinates 64007-64122;
atom coordinates 64123-64223;
atom coordinates 64224-64354;
atom coordinates 64355-64448;
atom coordinates 64449-64561;
atom coordinates 64562-64785;
atom coordinates 64786-64872;
atom coordinates 64873-64889;
atom coordinates 64890-64955;
atom coordinates 64956-65011;
atom coordinates 65012-65085;
atom coordinates 65086-65144;
atom coordinates 65145-65198;
atom coordinates 65199-65245;
atom coordinates 65246-65309;
atom coordinates 65310-65345;
atom coordinates 61881-65345; and
atom coordinates 1-65345.

105. The composition-of-matter of claim 97, wherein a three-dimensional atomic structure of at least a portion of said crystallized large ribosomal subunit is defined by a set of structure coordinates having a root mean square deviation of not more than 2.0 Å from a set of structure coordinates corresponding to a set of coordinates set forth in Table 3, said set of coordinates set forth in Table 3 being selected from the consisting of:
nucleotide coordinates 2044, 2430, 2431, 2479 and 2483-2485;
nucleotide coordinates 2044-2485;
nucleotide coordinates 2040-2042, 2044, 2482, 2484 and 2590;
nucleotide coordinates 2040-2590;
nucleotide coordinates 2040-2042, 2045, 2484, 2588 and 2589;
nucleotide coordinates 2040-2589;
atom coordinates 1-59360;
atom coordinates 59361-61880;
atom coordinates 1-61880;
atom coordinates 61881-62151;
atom coordinates 62152-62357;
atom coordinates 62358-62555;
atom coordinates 62556-62734;
atom coordinates 62735-62912;
atom coordinates 62913-62965;
atom coordinates 62966-63109;
atom coordinates 63110-63253;
atom coordinates 63254-63386;
atom coordinates 63387-63528;
atom coordinates 63529-63653;
atom coordinates 63654-63768;
atom coordinates 63769-63880;
atom coordinates 63881-64006;
atom coordinates 64007-64122;
atom coordinates 64123-64223;
atom coordinates 64224-64354;
atom coordinates 64355-64448;
atom coordinates 64449-64561;
atom coordinates 64562-64785;
atom coordinates 64786-64872;
atom coordinates 64873-64889;
atom coordinates 64890-64955;
atom coordinates 64956-65011;
atom coordinates 65012-65085;
atom coordinates 65086-65144;
atom coordinates 65145-65198;
atom coordinates 65199-65245;
atom coordinates 65246-65309;
atom coordinates 65310-65345;
atom coordinates 61881-65345; and
atom coordinates 1-65345.

106. The composition-of-matter of claim 97, wherein said crystallized large ribosomal subunit comprises a nucleic acid molecule, a segment of which including nucleotides or amino acid residues being capable of specifically associating with an antibiotic selected from the group consisting of chloramphenicol, a lincosamide antibiotic, a macrolide antibiotic, a puromycin conjugate, ASMS, and sparsomycin.

107. The composition-of-matter of claim 106, wherein said lincosamide antibiotic is clindamycin.

108. The composition-of-matter of claim 106, wherein said macrolide antibiotic is selected from the group consisting of erythromycin, clarithromycin, roxithromycin, troleandomycin, a ketolide antibiotic and an azalide antibiotic.

109. The composition-of-matter of claim 108, wherein said ketolide antibiotic is ABT-773.

110. The composition-of-matter of claim 108, wherein said azalide antibiotic is azithromycin.

111. The composition-of-matter of claim 106, wherein said puromycin conjugate is ACCP or ASM.

112. The composition-of-matter of claim 106, wherein a three-dimensional atomic structure of said nucleic acid molecule is defined by the set of structure coordinates corresponding to atom coordinates 1-59360 set forth in Table 3.

113. The composition-of-matter of claim 106, wherein said antibiotic is chloramphenicol and whereas a three-dimensional atomic structure of said nucleotides being capable of specifically associating with said chloramphenicol is defined by the set of structure coordinates corresponding to nucleotide coordinates 2044, 2430, 2431, 2479 and 2483-2485 set forth in Table 3.

114. The composition-of-matter of claim 106, wherein said antibiotic is chloramphenicol and whereas a three-dimensional atomic structure of said nucleotides being capable of specifically associating with said chloramphenicol is defined by a set of structure coordinates having a root mean square deviation of not more than 2.0 Å from the set of structure coordinates corresponding to nucleotide coordinates 2044, 2430, 2431, 2479 and 2483-2485 set forth in Table 3.

115. The composition-of-matter of claim 106, wherein said antibiotic is chloramphenicol and whereas a three-dimensional atomic structure of said segment including said nucleotides being capable of specifically associating with said chloramphenicol is defined by the set of structure coordinates corresponding to nucleotide coordinates 2044-2485 set forth in Table 3.

116. The composition-of-matter of claim 106, wherein said antibiotic is chloramphenicol and whereas a three-dimensional atomic structure of said segment including said nucleotides being capable of specifically associating with said chloramphenicol is defined by a set of structure coordinates having a root mean square deviation of not more than 2.0 Å from the set of structure coordinates corresponding to nucleotide coordinates 2044-2485 set forth in Table 3.

117. The composition-of-matter of claim 106, wherein said antibiotic is clindamycin and whereas a three-dimensional atomic structure of said nucleotides being capable of specifically associating with said clindamycin is defined by the set of structure coordinates corresponding to nucleotide coordinates 2040-2042, 2044, 2482, 2484 and 2590 set forth in Table 3.

118. The composition-of-matter of claim 106, wherein said antibiotic is clindamycin and whereas a three-dimensional atomic structure of said nucleotides being capable of specifically associating with said clindamycin is defined by a set of structure coordinates having a root mean square deviation of not more than 2.0 Å from the set of structure coordinates corresponding to nucleotide coordinates 2040-2042, 2044, 2482, 2484 and 2590 set forth in Table 3.

119. The composition-of-matter of claim 106, wherein said antibiotic is clindamycin and whereas a three-dimensional atomic structure of said segment including said nucleotides being capable of specifically associating with said clindamycin is defined by the set of structure coordinates corresponding to nucleotide coordinates 2040-2590 set forth in Table 3.

120. The composition-of-matter of claim 106, wherein said antibiotic is clindamycin and whereas a three-dimensional atomic structure of said segment including said nucleotides being capable of specifically associating with said clindamycin is defined by a set of structure coordinates having a root mean square deviation of not more than 2.0 Å from the set of structure coordinates corresponding to nucleotide coordinates 2040-2590 set forth in Table 3.

121. The composition-of-matter of claim 106, wherein said antibiotic is clarithromycin, erythromycin or roxithromycin, and whereas a three-dimensional atomic structure of said nucleotides being capable of specifically associating with said antibiotic is defined by the set of structure coordinates corresponding to nucleotide coordinates 2040-2042, 2045, 2484, 2588 and 2589 set forth in Table 3.

122. The composition-of-matter of claim 106, wherein said antibiotic is clarithromycin, erythromycin or roxithromycin, and whereas a three-dimensional atomic structure of said nucleotides being capable of specifically associating with said antibiotic is defined by a set of structure coordinates having a root mean square deviation of not more than 2.0 Å from the set of structure coordinates corresponding to nucleotide coordinates 2040-2042, 2045, 2484, 2588 and 2589 set forth in Table 3.

123. The composition-of-matter of claim 106, wherein said antibiotic is clarithromycin, erythromycin or roxithromycin, and whereas a three-dimensional atomic structure of said segment including said nucleotides being capable of specifically associating with said antibiotic is defined by the set of structure coordinates corresponding to nucleotide coordinates 2040-2589 set forth in Table 3.

124. The composition-of-matter of claim 106, wherein said antibiotic is clarithromycin, erythromycin or roxithromycin, and whereas a three-dimensional atomic structure of said segment including said nucleotides being capable of specifically associating with said antibiotic is defined by a set of structure coordinates having a root mean square deviation of not more than 2.0 Å from the set of structure coordinates corresponding to nucleotide coordinates 2040-2589 set forth in Table 3.

125. The composition-of-matter of claim 106, wherein said antibiotic is ABT-773, and whereas a three-dimensional atomic structure of said nucleotides being capable of specifically associating with said antibiotic is defined by the set of structure coordinates corresponding to nucleotide coordinates 803, 1773, 2040-2042, 2045, 2484, and 2588-2590 set forth in Table 18.

126. The composition-of-matter of claim 106, wherein said antibiotic is ABT-773, and whereas a three-dimensional atomic structure of said nucleotides being capable of specifically associating with said antibiotic is defined by a set of structure coordinates having a root mean square deviation of not more than 2.0 Å from the set of structure coordinates corresponding to nucleotide coordinates 803, 1773, 2040-2042, 2045, 2484, and 2588-2590 set forth in Table 18.

127. The composition-of-matter of claim 106, wherein said antibiotic is ABT-773, and whereas a three-dimensional atomic structure of said segment including said nucleotides being capable of specifically associating with said antibiotic is defined by the set of structure coordinates corresponding to nucleotide coordinates 803-2590 set forth in Table 18.

128. The composition-of-matter of claim 106, wherein said antibiotic is ABT-773, and whereas a three-dimensional atomic structure of said segment including said amino acid residues being capable of specifically associating with said antibiotic is defined by a set of structure coordinates having a root mean square deviation of not more than 2.0 Å from the set of structure coordinates corresponding to amino acid residue coordinates 803-2590 set forth in Table 18.

129. The composition-of-matter of claim 106, wherein said antibiotic is azithromycin, and whereas a three-dimensional atomic structure of said nucleotides being capable of specifically associating with said antibiotic is defined by the set of structure coordinates corresponding to nucleotide coordinates 764, 2041, 2042, 2045, A2482, 2484, 2565, and 2588-2590 set forth in Table 19.

130. The composition-of-matter of claim 106, wherein said antibiotic is azithromycin, and whereas a three-dimensional atomic structure of said nucleotides being capable of specifically associating with said antibiotic is defined by a set of structure coordinates having a root mean square deviation of not more than 2.0 Å from the set of structure coordinates corresponding to nucleotide coordinates 764, 2041, 2042, 2045, A2482, 2484, 2565, and 2588-2590 set forth in Table 19.

131. The composition-of-matter of claim 106, wherein said antibiotic is azithromycin, and whereas a three-dimensional atomic structure of said segment including said nucleotides being capable of specifically associating with said antibiotic is defined by the set of structure coordinates corresponding to nucleotide coordinates 764-2590 set forth in Table 19.

132. The composition-of-matter of claim 106, wherein said antibiotic is azithromycin, and whereas a three-dimensional atomic structure of said segment including said nucleotides being capable of specifically associating with said antibiotic is defined by a set of structure coordinates having a root mean square deviation of not more than 2.0 Å from the set of structure coordinates corresponding to nucleotide coordinates 764-2590 set forth in Table 19.

133. The composition-of-matter of claim 106, wherein said antibiotic is azithromycin, and whereas a three-dimensional atomic structure of said segment including said amino acid residues being capable of specifically associating with said antibiotic is defined by the set of structure coordinates corresponding to amino acid residue coordinates Y59, G60, G63, T64 and R111 set forth in Table 19.

134. The composition-of-matter of claim 106, wherein said antibiotic is azithromycin, and whereas a three-dimensional atomic structure of said segment including said amino acid residues being capable of specifically associating with said antibiotic is defined by a set of structure coordinates having a root mean square deviation of not more than 2.0 Å from the set of structure coordinates corresponding to amino acid residue coordinates Y59, G60, G63, T64 and R111 set forth in Table 19.

135. The composition-of-matter of claim 106, wherein said antibiotic is ACCP, and whereas a three-dimensional atomic structure of said nucleotides being capable of specifically associating with said antibiotic is defined by the set of structure coordinates corresponding to nucleotide coordinates 1924, 2430, 2485, 2532, 2533, 2534, 2552, 2562, and 2583 set forth in Table 20.

136. The composition-of-matter of claim 106, wherein said antibiotic is ACCP, and whereas a three-dimensional atomic structure of said nucleotides being capable of specifically associating with said antibiotic is defined by a set of structure coordinates having a root mean square deviation of not more than 2.0 Å from the set of structure coordinates corresponding to nucleotide coordinates 1924, 2430, 2485, 2532, 2533, 2534, 2552, 2562, and 2583 set forth in Table 20.

137. The composition-of-matter of claim 106, wherein said antibiotic is ACCP, and whereas a three-dimensional atomic structure of said segment including said nucleotides being capable of specifically associating with said antibiotic is defined by the set of structure coordinates corresponding to nucleotide coordinates 1924-2583 set forth in Table 20.

138. The composition-of-matter of claim 106, wherein said antibiotic is ACCP, and whereas a three-dimensional atomic structure of said segment including said nucleotides being capable of specifically associating with said antibiotic is defined by a set of structure coordinates having a root mean square deviation of not more than 2.0 Å from the set of structure coordinates corresponding to nucleotide coordinates 1924-2583 set forth in Table 20.

139. The composition-of-matter of claim 106, wherein said antibiotic is ASM, and whereas a three-dimensional atomic structure of said nucleotides being capable of specifically associating with said antibiotic is defined by the set of structure coordinates corresponding to nucleotide coordinates 1892, 1896, 1899, 1925, 1926, 2431, 2485, 2486, 2532, 2534, 2552, 2562, and 2581 set forth in Table 21.

140. The composition-of-matter of claim 106, wherein said antibiotic is ASM, and whereas a three-dimensional atomic structure of said nucleotides being capable of specifically associating with said antibiotic is defined by a set of structure coordinates having a root mean square deviation of not more than 2.0 Å from the set of structure coordinates corresponding to nucleotide coordinates 1892, 1896, 1899, 1925, 1926, 2431, 2485, 2486, 2532, 2534, 2552, 2562, and 2581 set forth in Table 21.

141. The composition-of-matter of claim 106, wherein said antibiotic is ASM, and whereas a three-dimensional atomic structure of said segment including said nucleotides being capable of specifically associating with said antibiotic is defined by the set of structure coordinates corresponding to nucleotide coordinates 1892-2581 set forth in Table 21.

142. The composition-of-matter of claim 106, wherein said antibiotic is ASM, and whereas a three-dimensional atomic structure of said segment including said nucleotides being capable of specifically associating with said antibiotic is defined by a set of structure coordinates having a root mean square deviation of not more than 2.0 Å from the set of structure coordinates corresponding to nucleotide coordinates 1892-2581 set forth in Table 21.

143. The composition-of-matter of claim 106, wherein said antibiotic is ASM, and whereas a three-dimensional atomic structure of said segment including said amino acid residues being capable of specifically associating with said antibiotic is defined by the set of structure coordinates corresponding to amino acid residue coordinates 79-81 set forth in Table 21.

144. The composition-of-matter of claim 106, wherein said antibiotic is ASM, and whereas a three-dimensional atomic structure of said segment including said amino acid residues being capable of specifically associating with said antibiotic is defined by a set of structure coordinates having a root mean square deviation of not more than 2.0 Å from the set of structure coordinates corresponding to amino acid residue coordinates 79-81 set forth in Table 21.

145. The composition-of-matter of claim 106, wherein said antibiotic is ASMS, and whereas a three-dimensional atomic structure of said nucleotides being capable of specifically associating with said antibiotic is defined by the set of structure coordinates corresponding to nucleotide coordinates 1899, 1924, 1926, 2430, 2472, 2485, 2486, 2532, 2534, 2552, 2562, 2563, and 2581 set forth in Table 22.

146. The composition-of-matter of claim 106, wherein said antibiotic is ASMS, and whereas a three-dimensional atomic structure of said nucleotides being capable of specifically associating with said antibiotic is defined by a set of structure coordinates having a root mean square deviation of not more than 2.0 Å from the set of structure coordinates corresponding to nucleotide coordinates 1899, 1924, 1926, 2430, 2472, 2485, 2486, 2532, 2534, 2552, 2562, 2563, and 2581 set forth in Table 22.

147. The composition-of-matter of claim 106, wherein said antibiotic is ASMS, and whereas a three-dimensional atomic structure of said segment including said nucleotides being capable of specifically associating with said antibiotic is defined by the set of structure coordinates corresponding to nucleotide coordinates 1899-2581 set forth in Table 22.

148. The composition-of-matter of claim 106, wherein said antibiotic is ASMS, and whereas a three-dimensional atomic structure of said segment including said nucleotides being capable of specifically associating with said antibiotic is defined by a set of structure coordinates having a root mean square deviation of not more than 2.0 Å from the set of structure coordinates corresponding to nucleotide coordinates 1899-2581 set forth in Table 22.

149. The composition-of-matter of claim 106, wherein said antibiotic is ASMS, and whereas a three-dimensional atomic structure of said segment including said amino acid residues being capable of specifically associating with said antibiotic is defined by the set of structure coordinates corresponding to amino acid residue coordinates 79-81 set forth in Table 22.

150. The composition-of-matter of claim 106, wherein said antibiotic is ASMS, and whereas a three-dimensional atomic structure of said segment including said amino acid residues being capable of specifically associating with said antibiotic is defined by a set of structure coordinates having a root mean square deviation of not more than 2.0 Å from the set of structure coordinates corresponding to amino acid residue coordinates 79-81 set forth in Table 22.

151. The composition-of-matter of claim 106, wherein said antibiotic is sparsomycin, and whereas a three-dimensional atomic structure of said nucleotides being capable of specifically associating with said antibiotic is defined by the set of structure coordinates corresponding to nucleotide coordinate 2581 set forth in Table 23.

152. The composition-of-matter of claim 106, wherein said antibiotic is sparsomycin, and whereas a three-dimensional atomic structure of said nucleotides being capable of specifically associating with said antibiotic is defined by a set of structure coordinates having a root mean square deviation of not more than 2.0 Å from the set of structure coordinates corresponding to nucleotide coordinate 2581 set forth in Table 23.

153. The composition-of-matter of claim 106, wherein said antibiotic is troleandomycin, and whereas a three-dimensional atomic structure of said nucleotides being capable of specifically associating with said antibiotic is defined by the set of structure coordinates corresponding to nucleotide coordinates 759, 761, 803, 2041, 2042, 2045, 2484, and 2590 set forth in Table 38.

154. The composition-of-matter of claim 106, wherein said antibiotic is troleandomycin, and whereas a three-dimensional atomic structure of said nucleotides being capable of specifically associating with said antibiotic is defined by a set of structure coordinates having a root mean square deviation of not more than 2.0 Å from the set of structure coordinates corresponding to nucleotide coordinates 759, 761, 803, 2041, 2042, 2045, 2484, and 2590 set forth in Table 38.

155. The composition-of-matter of claim 106, wherein said antibiotic is troleandomycin, and whereas a three-dimensional atomic structure of said segment including said nucleotides being capable of specifically associating with said antibiotic is defined by the set of structure coordinates corresponding to nucleotide coordinates 759-2590 set forth in Table 38.

156. The composition-of-matter of claim 106, wherein said antibiotic is troleandomycin, and whereas a three-dimensional atomic structure of said segment including said nucleotides being capable of specifically associating with said antibiotic is defined by a set of structure coordinates having a root mean square deviation of not more than 2.0 Å from the set of structure coordinates corresponding to nucleotide coordinates 759-2590 set forth in Table 38.

157. The composition-of-matter of claim 106, wherein said antibiotic is troleandomycin, and whereas a three-dimensional atomic structure of said segment including said amino acid residues being capable of specifically associating with said antibiotic is defined by the set of structure coordinates corresponding to amino acid residue coordinate Ala2 set forth in Table 38.

158. The composition-of-matter of claim 106, wherein said antibiotic is troleandomycin, and whereas a three-dimensional atomic structure of said segment including said amino acid residues being capable of specifically associating with said antibiotic is defined by a set of structure coordinates having a root mean square deviation of not more than 2.0 Å from the set of structure coordinates corresponding to amino acid residue coordinate Ala2 set forth in Table 38.

159. A method of identifying a putative antibiotic comprising:
(a) obtaining a set of structure coordinates defining a three-dimensional atomic structure of a crystallized antibiotic-binding pocket of a large ribosomal subunit of a eubacterium; and
(b) computationally screening a plurality of compounds for a compound capable of specifically binding said antibiotic-binding pocket,
thereby identifying the putative antibiotic.

160. The method of claim 159, further comprising:
(i) contacting the putative antibiotic with said antibiotic-binding pocket; and
(ii) detecting specific binding of the putative antibiotic to said antibiotic-binding pocket, thereby qualifying the putative antibiotic.

161. The method of claim 159, wherein step (a) is effected by co-crystallizing at least said antibiotic-binding pocket with an antibiotic.

162. The method of claim 159, wherein said eubacterium is D. radiodurans.

163. The method of claim 159, wherein said eubacterium is a gram-positive bacterium.

164. The method of claim 159, wherein said eubacterium is a coccus.

165. The method of claim 159, wherein said eubacterium is a Deinococcus-Thermophilus group bacterium.

166. The method of claim 159, wherein said antibiotic-binding pocket is a clindamycin-binding pocket and whereas said structure coordinates define said three-dimensional atomic structure at a resolution higher than or equal to 3.1 Å.

167. The method of claim 159, wherein said antibiotic-binding pocket is an erythromycin-binding pocket and whereas said structure coordinates define said three-dimensional atomic structure at a resolution higher than or equal to 3.4 Å.

168. The method of claim 159, wherein said antibiotic-binding pocket is a clarithromycin-binding pocket and whereas said structure coordinates define said three-dimensional atomic structure at a resolution higher than or equal to 3.5 Å.

169. The method of claim 159, wherein said antibiotic-binding pocket is a roxithromycin-binding pocket and whereas said structure coordinates define said three-dimensional atomic structure at a resolution higher than or equal to 3.8 Å.

170. The method of claim 159, wherein said antibiotic-binding pocket is a chloramphenicol-binding pocket and whereas said structure coordinates define said three-dimensional atomic structure at a resolution higher than or equal to 3.5 Å.

171. The method of claim 159, wherein said antibiotic-binding pocket is an ABT-773-binding pocket and whereas said structure coordinates define said three-dimensional atomic structure at a resolution higher than or equal to 3.5 Å.

172. The method of claim 159, wherein said antibiotic-binding pocket is an azithromycin-binding pocket and whereas said structure coordinates define said three-dimensional atomic structure at a resolution higher than or equal to 3.2 Å.

173. The method of claim 159, wherein said antibiotic-binding pocket is an ACCP-binding pocket and whereas said structure coordinates define said three-dimensional atomic structure at a resolution higher than or equal to 3.7 Å.

174. The method of claim 159, wherein said antibiotic-binding pocket is a puromycin conjugate-binding pocket and whereas said structure coordinates define said three-dimensional atomic structure at a resolution higher than or equal to 3.5 Å.

175. The method of claim 174, wherein said puromycin conjugate is ASM.

176. The method of claim 159, wherein said antibiotic-binding pocket is an ASMS-binding pocket and whereas said structure coordinates define said three-dimensional atomic structure at a resolution higher than or equal to 3.6 Å.

177. The method of claim 159, wherein said antibiotic-binding pocket is a sparsomycin-binding pocket and whereas said structure coordinates define said three-dimensional atomic structure at a resolution higher than or equal to 3.7 Å.

178. The method of claim 159, wherein said antibiotic-binding pocket is a troleandomycin-binding pocket and whereas said structure coordinates define said three-dimensional atomic structure at a resolution higher than or equal to 3.4 Å.

179. The method of claim 159, wherein said antibiotic-binding pocket is selected from the group consisting of a chloramphenicol-specific antibiotic-binding pocket, a lincosamide antibiotic-specific antibiotic-binding pocket, a macrolide antibiotic-specific antibiotic-binding pocket, a puromycin conjugate-specific antibiotic-binding pocket, and a sparsomycin-specific antibiotic-binding pocket.

180. The method of claim 179, wherein said lincosamide-specific binding pocket is a clindamycin-specific antibiotic-binding pocket.

181. The method of claim 179, wherein said macrolide antibiotic-specific binding pocket is an erythromycin-specific antibiotic-binding pocket, a roxithromycin-specific antibiotic-binding pocket, a troleandomycin-specific antibiotic-binding pocket, a ketolide antibiotic-specific binding pocket, and an azalide antibiotic-specific binding pocket.

182. The method of claim 181, wherein said ketolide-specific binding pocket is an ABT-773-specific antibiotic-binding pocket.

183. The method of claim 181, wherein said azalide-specific binding pocket is an azithromycin-specific antibiotic-binding pocket.

184. The method of claim 179, wherein said puromycin conjugate-specific antibiotic-binding pocket is an ACCP-specific antibiotic-binding pocket or an ASM-specific antibiotic-binding pocket.

185. The method of claim 159, wherein the antibiotic comprises at least two non-covalently associated molecules.

186. The method of claim 159, wherein said antibiotic-binding pocket forms a part of a component of said large ribosomal subunit selected from the group consisting of a polynucleotide component, a polypeptide component and a magnesium ion component.

187. A computing platform for generating a three-dimensional model of at least a portion of a large ribosomal subunit of a eubacterium, the computing platform comprising:
(a) a data-storage device storing data comprising a set of structure coordinates defining at least a portion of a three-dimensional structure of the large ribosomal subunit; and
(b) a processing unit being for generating the three-dimensional model from said data stored in said data-storage device.

188. The computing platform of claim 187, further comprising a display being for displaying the three-dimensional model generated by said processing unit.

189. The computing platform of claim 187, wherein the eubacterium is D. radiodurans.

190. The computing platform of claim 187, wherein the eubacterium is a gram-positive bacterium.

191. The computing platform of claim 187, wherein the eubacterium is a coccus.

192. The computing platform of claim 187, wherein the eubacterium is a Deinococcus-Thermophilus group bacterium.

193. The computing platform of claim 187, wherein said set of structure coordinates define said portion of a three-dimensional structure of a large ribosomal subunit at a resolution higher than or equal to a resolution selected from the group consisting of 5.4 Å, 5.3 Å, 5.2 Å, 5.1 Å, 5.0 Å, 4.9 Å, 4.8 Å, 4.7 Å, 4.6 Å, 4.5 Å, 4.4 Å, 4.3 Å, 4.2 Å, 4.1 Å, 4.0 Å, 3.9 Å, 3.8 Å, 3.7 Å, 3.6 Å, 3.5 Å, 3.4 Å, 3.3 Å, 3.2 Å and 3.1 Å.

194. The computing platform of claim 187, wherein said set of structure coordinates define said portion of a three-dimensional structure of the large ribosomal subunit at a resolution higher than or equal to 3.1 Å.

195. The computing platform of claim 187, wherein said set of structure coordinates defining at least a portion of a three-dimensional structure of the large ribosomal subunit is a set of structure coordinates corresponding to a set of coordinates set forth in Table 3, said set of coordinates set forth in Table 3 being selected from the group consisting of:
nucleotide coordinates 2044, 2430, 2431, 2479 and 2483-2485;
nucleotide coordinates 2044-2485;
nucleotide coordinates 2040-2042, 2044, 2482, 2484 and 2590;
nucleotide coordinates 2040-2590;
nucleotide coordinates 2040-2042, 2045, 2484, 2588 and 2589;
nucleotide coordinates 2040-2589;
atom coordinates 1-59360;
atom coordinates 59361-61880;
atom coordinates 1-61880;
atom coordinates 61881-62151;
atom coordinates 62152-62357;
atom coordinates 62358-62555;
atom coordinates 62556-62734;
atom coordinates 62735-62912;
atom coordinates 62913-62965;
atom coordinates 62966-63109;
atom coordinates 63110-63253;
atom coordinates 63254-63386;
atom coordinates 63387-63528;
atom coordinates 63529-63653;
atom coordinates 63654-63768;
atom coordinates 63769-63880;
atom coordinates 63881-64006;
atom coordinates 64007-64122;
atom coordinates 64123-64223;
atom coordinates 64224-64354;
atom coordinates 64355-64448;
atom coordinates 64449-64561;
atom coordinates 64562-64785;
atom coordinates 64786-64872;
atom coordinates 64873-64889;
atom coordinates 64890-64955;
atom coordinates 64956-65011;
atom coordinates 65012-65085;
atom coordinates 65086-65144;
atom coordinates 65145-65198;
atom coordinates 65199-65245;
atom coordinates 65246-65309;
atom coordinates 65310-65345;
atom coordinates 61881-65345; and
atom coordinates 1-65345.

196. The computing platform of claim 187, wherein said set of structure coordinates defining at least a portion of a three-dimensional structure of the large ribosomal subunit is a set of structure coordinates having a root mean square deviation of not more than 2.0 Å from a set of structure coordinates corresponding to a set of coordinates set forth in Table 3, said set of coordinates set forth in Table 3 being selected from the group consisting of:
nucleotide coordinates 2044, 2430, 2431, 2479 and 2483-2485;
nucleotide coordinates 2044-2485;
nucleotide coordinates 2040-2042, 2044, 2482, 2484 and 2590;
nucleotide coordinates 2040-2590;
nucleotide coordinates 2040-2042, 2045, 2484, 2588 and 2589;
nucleotide coordinates 2040-2589;
atom coordinates 1-59360;
atom coordinates 59361-61880;
atom coordinates 1-61880;
atom coordinates 61881-62151;
atom coordinates 62152-62357;
atom coordinates 62358-62555;
atom coordinates 62556-62734;
atom coordinates 62735-62912;
atom coordinates 62913-62965;
atom coordinates 62966-63109;
atom coordinates 63110-63253;
atom coordinates 63254-63386;
atom coordinates 63387-63528;
atom coordinates 63529-63653;
atom coordinates 63654-63768;
atom coordinates 63769-63880;
atom coordinates 63881-64006;
atom coordinates 64007-64122;
atom coordinates 64123-64223;
atom coordinates 64224-64354;
atom coordinates 64355-64448;
atom coordinates 64449-64561;
atom coordinates 64562-64785;
atom coordinates 64786-64872;
atom coordinates 64873-64889;
atom coordinates 64890-64955;
atom coordinates 64956-65011;
atom coordinates 65012-65085;
atom coordinates 65086-65144;
atom coordinates 65145-65198;
atom coordinates 65199-65245;
atom coordinates 65246-65309;
atom coordinates 65310-65345;
atom coordinates 61881-65345; and
atom coordinates 1-65345.

197. A computing platform for generating a three-dimensional model of at least a portion of a complex of an antibiotic and a large ribosomal subunit of a eubacterium, the computing platform comprising:
(a) a data-storage device storing data comprising a set of structure coordinates defining at least a portion of a three-dimensional structure of the complex of an antibiotic and a large ribosomal subunit; and
(b) a processing unit being for generating the three-dimensional model from said data stored in said data-storage device.

198. The computing platform of claim 197, further comprising a display being for displaying the three-dimensional model generated by said processing unit.

199. The computing platform of claim 197, wherein the eubacterium is D. radiodurans.

200. The computing platform of claim 197, wherein the eubacterium is a gram-positive bacterium.

201. The computing platform of claim 197, wherein the eubacterium is a coccus.

202. The computing platform of claim 197, wherein the eubacterium is a Deinococcus-Thermophilus group bacterium.

203. The computing platform of claim 197, wherein the antibiotic is clindamycin and whereas said set of structure coordinates define said portion of a three-dimensional structure at a resolution higher than or equal to 3.1 Å.

204. The computing platform of claim 197, wherein the antibiotic is erythromycin and whereas said set of structure coordinates define said portion of a three-dimensional structure at a resolution higher than or equal to of 3.4 Å.

205. The computing platform of claim 197, wherein the antibiotic is clarithromycin and whereas said set of structure coordinates define said portion of a three-dimensional structure at a resolution higher than or equal to 3.5 Å.

206. The computing platform of claim 197, wherein the antibiotic is roxithromycin and whereas said set of structure coordinates define said portion of a three-dimensional structure at a resolution higher than or equal to 3.8 Å.

207. The computing platform of claim 197, wherein the antibiotic is chloramphenicol and whereas said set of structure coordinates define said portion of a three-dimensional structure at a resolution higher than or equal to 3.5 Å.

208. The computing platform of claim 197, wherein the antibiotic is ABT-773 and whereas said set of structure coordinates define said portion of a three-dimensional structure at a resolution higher than or equal to 3.5 Å.

209. The computing platform of claim 197, wherein the antibiotic is azithromycin and whereas said set of structure coordinates define said portion of a three-dimensional structure at a resolution higher than or equal to 3.2 Å.

210. The computing platform of claim 197, wherein the antibiotic is ACCP and whereas said set of structure coordinates define said portion of a three-dimensional structure at a resolution higher than or equal to 3.7 Å.

211. The computing platform of claim 197, wherein the antibiotic is ASM and whereas said set of structure coordinates define said portion of a three-dimensional structure at a resolution higher than or equal to 3.5 Å.

212. The computing platform of claim 197, wherein the antibiotic is ASMS and whereas said set of structure coordinates define said portion of a three-dimensional structure at a resolution higher than or equal to 3.6 Å.

213. The computing platform of claim 197, wherein the antibiotic is sparsomycin and whereas said set of structure coordinates define said portion of a three-dimensional structure at a resolution higher than or equal to 3.7 Å.

214. The computing platform of claim 197, wherein the antibiotic is troleandomycin and whereas said set of structure coordinates define said portion of a three-dimensional structure at a resolution higher than or equal to 3.4 Å.

215. The computing platform of claim 197, wherein the antibiotic is selected from the group consisting of chloramphenicol, a lincosamide antibiotic, a macrolide antibiotic, a puromycin conjugate, ASMS, and sparsomycin.

216. The computing platform of claim 215, wherein said lincosamide antibiotic is clindamycin.

217. The computing platform of claim 215, wherein said macrolide antibiotic is selected from the group consisting of erythromycin, clarithromycin, roxithromycin, troleandomycin, a ketolide antibiotic and an azalide antibiotic.

218. The computing platform of claim 217, wherein said ketolide antibiotic is ABT-773.

219. The computing platform of claim 217, wherein said azalide antibiotic is azithromycin.

220. The computing platform of claim 215, wherein said puromycin conjugate is ACCP or ASM.

221. The computing platform of claim 197, wherein the antibiotic is chloramphenicol and whereas said set of structure coordinates defining at least a portion of a three-dimensional structure of the complex of said chloramphenicol and said large ribosomal subunit corresponds to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

222. The computing platform of claim 197, wherein the antibiotic is clindamycin and whereas said set of structure coordinates defining at least a portion of a three-dimensional structure of the complex of said clindamycin and said large ribosomal subunit corresponds to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

223. The computing platform of claim 197, wherein the antibiotic is clarithromycin and whereas said set of structure coordinates defining at least a portion of a three-dimensional structure of the complex of said clarithromycin and said large ribosomal subunit corresponds to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

224. The computing platform of claim 197, wherein the antibiotic is erythromycin and whereas said set of structure coordinates defining at least a portion of a three-dimensional structure of the complex of said erythromycin and said large ribosomal subunit corresponds to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

225. The computing platform of claim 197, wherein the antibiotic is roxithromycin and whereas said set of structure coordinates defining at least a portion of a three-dimensional structure of the complex of said roxithromycin and said large ribosomal subunit corresponds to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

226. The computing platform of claim 197, wherein the antibiotic is ABT-773 and whereas said set of structure coordinates defining at least a portion of a three-dimensional structure of the complex of said ABT-773 and said large ribosomal subunit corresponds to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

227. The computing platform of claim 197, wherein the antibiotic is azithromycin and whereas said set of structure coordinates defining at least a portion of a three-dimensional structure of the complex of said azithromycin and said large ribosomal subunit corresponds to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

228. The computing platform of claim 197, wherein the antibiotic is ACCP and whereas said set of structure coordinates defining at least a portion of a three-dimensional structure of the complex of said ACCP and said large ribosomal subunit corresponds to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

229. The computing platform of claim 197, wherein the antibiotic is ASM and whereas said set of structure coordinates defining at least a portion of a three-dimensional structure of the complex of said ASM and said large ribosomal subunit corresponds to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

230. The computing platform of claim 197, wherein the antibiotic is ASMS and whereas said set of structure coordinates defining at least a portion of a three-dimensional structure of the complex of said ASMS and said large ribosomal subunit corresponds to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

231. The computing platform of claim 197, wherein the antibiotic is sparsomycin and whereas said set of structure coordinates defining at least a portion of a three-dimensional structure of the complex of said sparsomycin and said large ribosomal subunit corresponds to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

232. The computing platform of claim 197, wherein the antibiotic is troleandomycin and whereas said set of structure coordinates defining at least a portion of a three-dimensional structure of the complex of said troleandomycin and said large ribosomal subunit corresponds to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

233. The computing platform of claim 197, wherein the antibiotic is chloramphenicol and whereas said set of structure coordinates defining at least a portion of a three-dimensional structure of the complex of said chloramphenicol and said large ribosomal subunit is a set of structure coordinates having a root mean square deviation of not more than 2.0 Å from a set of structure coordinates corresponding to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

234. The computing platform of claim 197, wherein the antibiotic is clindamycin and whereas said set of structure coordinates defining at least a portion of a three-dimensional structure of the complex of said clindamycin and said large ribosomal subunit is a set of structure coordinates having a root mean square deviation of not more than 2.0 Å from a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

235. The computing platform of claim 197, wherein the antibiotic is clarithromycin and whereas said set of structure coordinates defining at least a portion of a three-dimensional structure of the complex of said clarithromycin and said large ribosomal subunit is a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

236. The computing platform of claim 197, wherein the antibiotic is erythromycin and whereas said set of structure coordinates defining at least a portion of a three-dimensional structure of the complex of said erythromycin and said large ribosomal subunit is a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

237. The computing platform of claim 197, wherein the antibiotic is roxithromycin and whereas said set of structure coordinates defining at least a portion of a three-dimensional structure of the complex of said roxithromycin and said large ribosomal subunit is a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

238. The computing platform of claim 197, wherein the antibiotic is ABT-773 and whereas said set of structure coordinates defining at least a portion of a three-dimensional structure of the complex of said ABT-773 and said large ribosomal subunit is a set of structure coordinates having a root mean square deviation of not more than 2.0 Å from a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

239. The computing platform of claim 197, wherein the antibiotic is azithromycin and whereas said set of structure coordinates defining at least a portion of a three-dimensional structure of the complex of said azithromycin and said large ribosomal subunit is a set of structure coordinates having a root mean square deviation of not more than 2.0 Å from a set of structure coordinates corresponding to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

240. The computing platform of claim 197, wherein the antibiotic is ACCP and whereas said set of structure coordinates defining at least a portion of a three-dimensional structure of the complex of said ACCP and said large ribosomal subunit is a set of structure coordinates having a root mean square deviation of not more than 2.0 Å from a set of structure coordinates corresponding to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

241. The computing platform of claim 197, wherein the antibiotic is ASM and whereas said set of structure coordinates defining at least a portion of a three-dimensional structure of the complex of said ASM and said large ribosomal subunit is a set of structure coordinates having a root mean square deviation of not more than 2.0 Å from a set of structure coordinates corresponding to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

242. The computing platform of claim 197, wherein the antibiotic is ASMS and whereas said set of structure coordinates defining at least a portion of a three-dimensional structure of the complex of said ASMS and said large ribosomal subunit is a set of structure coordinates having a root mean square deviation of not more than 2.0 Å from a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

243. The computing platform of claim 197, wherein the antibiotic is sparsomycin and whereas said set of structure coordinates defining at least a portion of a three-dimensional structure of the complex of said sparsomycin and said large ribosomal subunit is a set of structure coordinates having a root mean square deviation of not more than 2.0 Å from a set of structure coordinates corresponding to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

244. The computing platform of claim 197, wherein the antibiotic is troleandomycin and whereas said set of structure coordinates defining at least a portion of a three-dimensional structure of the complex of said troleandomycin and said large ribosomal subunit is a set of structure coordinates having a root mean square deviation of not more than 2.0 Å from a set of structure coordinates corresponding to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

245. A computer generated model representing at least a portion of a large ribosomal subunit of a eubacterium, the computer generated model having a three-dimensional atomic structure defined by a set of structure coordinates corresponding to a set of coordinates set forth in Table 3, the set of coordinates set forth in Table 3 being selected from the group consisting of:
nucleotide coordinates 2044, 2430, 2431, 2479 and 2483-2485;
nucleotide coordinates 2044-2485;
nucleotide coordinates 2040-2042, 2044, 2482, 2484 and 2590;
nucleotide coordinates 2040-2590;
nucleotide coordinates 2040-2042, 2045, 2484, 2588 and 2589;
nucleotide coordinates 2040-2589;
atom coordinates 1-59360;
atom coordinates 59361-61880;
atom coordinates 1-61880;
atom coordinates 61881-62151;
atom coordinates 62152-62357;
atom coordinates 62358-62555;
atom coordinates 62556-62734;
atom coordinates 62735-62912;
atom coordinates 62913-62965;
atom coordinates 62966-63109;
atom coordinates 63110-63253;
atom coordinates 63254-63386;
atom coordinates 63387-63528;
atom coordinates 63529-63653;
atom coordinates 63654-63768;
atom coordinates 63769-63880;
atom coordinates 63881-64006;
atom coordinates 64007-64122;
atom coordinates 64123-64223;
atom coordinates 64224-64354;
atom coordinates 64355-64448;
atom coordinates 64449-64561;
atom coordinates 64562-64785;
atom coordinates 64786-64872;
atom coordinates 64873-64889;
atom coordinates 64890-64955;
atom coordinates 64956-65011;
atom coordinates 65012-65085;
atom coordinates 65086-65144;
atom coordinates 65145-65198;
atom coordinates 65199-65245;
atom coordinates 65246-65309;
atom coordinates 65310-65345;
atom coordinates 61881-65345; and
atom coordinates 1-65345.

246. The computer generated model of claim 245, wherein the eubacterium is D. radiodurans.

247. The computer generated model of claim 245, wherein the eubacterium is a gram-positive bacterium.

248. The computer generated model of claim 245, wherein the eubacterium is a coccus.

249. The computer generated model of claim 245, wherein the eubacterium is a Deinococcus-Thermophilus group bacterium.

250. A computer generated model representing at least a portion of a large ribosomal subunit of a eubacterium, the computer generated model having a three-dimensional atomic structure defined by a set of structure coordinates having a root mean square deviation of not more than 2.0 Å from a set of structure coordinates corresponding to a set of coordinates set forth in Table 3, the set of coordinates set forth in Table 3 being selected from the group consisting of:
nucleotide coordinates 2044, 2430, 2431, 2479 and 2483-2485;
nucleotide coordinates 2044-2485;
nucleotide coordinates 2040-2042, 2044, 2482, 2484 and 2590;
nucleotide coordinates 2040-2590;
nucleotide coordinates 2040-2042, 2045, 2484, 2588 and 2589;
nucleotide coordinates 2040-2589;
atom coordinates 1-59360;
atom coordinates 59361-61880;
atom coordinates 1-61880;
atom coordinates 61881-62151;
atom coordinates 62152-62357;
atom coordinates 62358-62555;
atom coordinates 62556-62734;
atom coordinates 62735-62912;
atom coordinates 62913-62965;
atom coordinates 62966-63109;
atom coordinates 63110-63253;
atom coordinates 63254-63386;
atom coordinates 63387-63528;
atom coordinates 63529-63653;
atom coordinates 63654-63768;
atom coordinates 63769-63880;
atom coordinates 63881-64006;
atom coordinates 64007-64122;
atom coordinates 64123-64223;
atom coordinates 64224-64354;
atom coordinates 64355-64448;
atom coordinates 64449-64561;
atom coordinates 64562-64785;
atom coordinates 64786-64872;
atom coordinates 64873-64889;
atom coordinates 64890-64955;
atom coordinates 64956-65011;
atom coordinates 65012-65085;
atom coordinates 65086-65144;
atom coordinates 65145-65198;
atom coordinates 65199-65245;
atom coordinates 65246-65309;
atom coordinates 65310-65345;
atom coordinates 61881-65345; and
atom coordinates 1-65345.

251. The computer generated model of claim 250, wherein the eubacterium is D. radiodurans.

252. The computer generated model of claim 250, wherein the eubacterium is a gram-positive bacterium.

253. The computer generated model of claim 250, wherein the eubacterium is a coccus.

254. The computer generated model of claim 250, wherein the eubacterium is a Deinococcus-Thermophilus group bacterium.

255. A computer generated model representing at least a portion of a complex of an antibiotic and a large ribosomal subunit of a eubacterium.

256. The computer generated model of claim 255, wherein the eubacterium is D. radiodurans.

257. The computer generated model of claim 255, wherein the eubacterium is a gram-positive bacterium.

258. The computer generated model of claim 255, wherein the eubacterium is a coccus.

259. The computer generated model of claim 255, wherein the eubacterium is a Deinococcus-Thermophilus group bacterium.

260. The computer generated model of claim 255, wherein the antibiotic is selected from the group consisting of chloramphenicol, a lincosamide antibiotic, a macrolide antibiotic, a puromycin conjugate, ASMS, and sparsomycin.

261. The computer generated model of claim 260, wherein said lincosamide antibiotic is clindamycin.

262. The computer generated model of claim 260, wherein said macrolide antibiotic is selected from the group consisting of erythromycin, clarithromycin, roxithromycin, troleandomycin, a ketolide antibiotic and an azalide antibiotic.

263. The computer generated model of claim 262, wherein said ketolide antibiotic is ABT-773.

264. The computer generated model of claim 262, wherein said azalide antibiotic is azithromycin.

265. The computer generated model of claim 260, wherein said puromycin conjugate is ACCP or ASM.

266. The computer generated model of claim 255, wherein the antibiotic is clindamycin and whereas the set of structure coordinates define the three-dimensional structure of the computer generated model at a resolution higher than or equal to 3.1 Å.

267. The computer generated model of claim 255, wherein the antibiotic is erythromycin and whereas the set of structure coordinates define the three-dimensional structure of the computer generated model at a resolution higher than or equal to 3.4 Å.

268. The computer generated model of claim 255, wherein the antibiotic is clarithromycin and whereas the set of structure coordinates define the three-dimensional structure of the computer generated model at a resolution higher than or equal to 3.5 Å.

269. The computer generated model of claim 255, wherein the antibiotic is roxithromycin and whereas the set of structure coordinates define the three-dimensional structure of the computer generated model at a resolution higher than or equal to 3.8 Å.

270. The computer generated model of claim 255, wherein the antibiotic is chloramphenicol and whereas the set of structure coordinates define the three-dimensional structure of the computer generated model at a resolution higher than or equal to 3.5 Å.

271. The computer generated model of claim 255, wherein the antibiotic is chloramphenicol and whereas a three-dimensional atomic structure of the portion of a complex of said chloramphenicol and the large ribosomal subunit is defined by a set of structure coordinates corresponding to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

272. The computer generated model of claim 255, wherein the antibiotic is clindamycin and whereas a three-dimensional atomic structure of the portion of a complex of said clindamycin and the large ribosomal subunit is defined by a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

273. The computer generated model of claim 255, wherein the antibiotic is clarithromycin and whereas a three-dimensional atomic structure of the portion of a complex of said clarithromycin and the large ribosomal subunit is defined by a set of structure coordinates corresponding to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

274. The computer generated model of claim 255, wherein the antibiotic is erythromycin and whereas a three-dimensional atomic structure of the portion of a complex of said erythromycin and the large ribosomal subunit is defined by a set of structure coordinates corresponding to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

275. The computer generated model of claim 255, wherein the antibiotic is roxithromycin and whereas a three-dimensional atomic structure of the portion of a complex of said roxithromycin and the large ribosomal subunit is defined by a set of structure coordinates corresponding to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

276. The computer generated model of claim 255, wherein the antibiotic is ABT-773 and whereas a three-dimensional atomic structure of the portion of a complex of said ABT-773 and the large ribosomal subunit is defined by a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

277. The computer generated model of claim 255, wherein the antibiotic is azithromycin and whereas a three-dimensional atomic structure of the portion of a complex of said azithromycin and the large ribosomal subunit is defined by a set of structure coordinates corresponding to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

278. The computer generated model of claim 255, wherein the antibiotic is ACCP and whereas a three-dimensional atomic structure of the portion of a complex of said ACCP and the large ribosomal subunit is defined by a set of structure coordinates corresponding to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

279. The computer generated model of claim 255, wherein the antibiotic is ASM and whereas a three-dimensional atomic structure of the portion of a complex of said ASM and the large ribosomal subunit is defined by a set of structure coordinates corresponding to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

280. The computer generated model of claim 255, wherein the antibiotic is ASMS and whereas a three-dimensional atomic structure of the portion of a complex of said ASMS and the large ribosomal subunit is defined by a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581 set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

281. The computer generated model of claim 255, wherein the antibiotic is sparsomycin and whereas a three-dimensional atomic structure of the portion of a complex of said sparsomycin and the large ribosomal subunit is defined by a set of structure coordinates corresponding to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

282. The computer generated model of claim 255, wherein the antibiotic is troleandomycin and whereas a three-dimensional atomic structure of the portion of a complex of said troleandomycin and the large ribosomal subunit is defined by a set of structure coordinates corresponding to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

283. The computer generated model of claim 255, wherein the antibiotic is chloramphenicol and whereas a three-dimensional atomic structure of the portion of a complex of said chloramphenicol and the large ribosomal subunit is defined by a set of structure coordinates having a root mean square deviation of not more than 2.0 A from a set of structure coordinates corresponding to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

284. The computer generated model of claim 255, wherein the antibiotic is clindamycin and whereas a three-dimensional atomic structure of the portion of a complex of said clindamycin and the large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

285. The computer generated model of claim 255, wherein the antibiotic is clarithromycin and whereas a three-dimensional atomic structure of the portion of a complex of said clarithromycin and the large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

286. The computer generated model of claim 255, wherein the antibiotic is erythromycin and whereas a three-dimensional atomic structure of the portion of a complex of said erythromycin and the large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

287. The computer generated model of claim 255, wherein the antibiotic is roxithromycin and whereas a three-dimensional atomic structure of the portion of a complex of said roxithromycin and the large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

288. The computer generated model of claim 255, wherein the antibiotic is ABT-773 and whereas a three-dimensional atomic structure of the portion of a complex of said ABT-773 and the large ribosomal subunit is defined by a set of structure coordinates having a root mean square deviation of not more than 2.0 Å from a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

289. The computer generated model of claim 255, wherein the antibiotic is azithromycin and whereas a three-dimensional atomic structure of the portion of a complex of said azithromycin and the large ribosomal subunit is defined by a set of structure coordinates having a root mean square deviation of not more than 2.0 Å from a set of structure coordinates corresponding to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

290. The computer generated model of claim 255, wherein the antibiotic is ACCP and whereas a three-dimensional atomic structure of the portion of a complex of said ACCP and the large ribosomal subunit is defined by a set of structure coordinates having a root mean square deviation of not more than 2.0 Å from a set of structure coordinates corresponding to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

291. The computer generated model of claim 255, wherein the antibiotic is ASM and whereas a three-dimensional atomic structure of the portion of a complex of said ASM and the large ribosomal subunit is defined by a set of structure coordinates having a root mean square deviation of not more than 2.0 Å from a set of structure coordinates corresponding to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

292. The computer generated model of claim 255, wherein the antibiotic is ASMS and whereas a three-dimensional atomic structure of the portion of a complex of said ASMS and the large ribosomal subunit is defined by a set of structure coordinates having a root mean square deviation of not more than 2.0 Å from a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581 set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

293. The computer generated model of claim 255, wherein the antibiotic is sparsomycin and whereas a three-dimensional atomic structure of the portion of a complex of said sparsomycin and the large ribosomal subunit is defined by a set of structure coordinates having a root mean square deviation of not more than 2.0 Å from a set of structure coordinates corresponding to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

294. The computer generated model of claim 255, wherein the antibiotic is troleandomycin and whereas a three-dimensional atomic structure of the portion of a complex of said troleandomycin and the large ribosomal subunit is defined by a set of structure coordinates having a root mean square deviation of not more than 2.0 Å from a set of structure coordinates corresponding to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

295. A computer readable medium comprising, in a retrievable format, data including a set of structure coordinates defining at least a portion of a three-dimensional structure of a crystallized large ribosomal subunit of a eubacterium.

296. The computer readable medium of claim 295, wherein the eubacterium is D. radiodurans.

297. The computer readable medium of claim 295, wherein the eubacterium is a gram-positive bacterium.

298. The computer readable medium of claim 295, wherein the eubacterium is a coccus.

299. The computer readable medium of claim 295, wherein the eubacterium is a Deinococcus-Thermophilus group bacterium.

300. The computer readable medium of claim 295, wherein said set of structure coordinates define said portion of a three-dimensional structure of a crystallized large ribosomal subunit at a resolution higher than or equal to a resolution selected from the group consisting of 5.4 Å, 5.3 Å, 5.2 Å, 5.1 Å, 5.0 Å, 4.9 Å, 4.8 Å, 4.7 Å, 4.6 Å, 4.5 Å, 4.4 Å, 4.3 Å, 4.2 Å, 4.1 Å, 4.0 Å, 3.9 Å, 3.8 Å, 3.7 Å, 3.6 Å, 3.5 Å, 3.4 Å, 3.3 Å, 3.2 Å and 3.1 Å.

301. The computer readable medium of claim 295, wherein said set of structure coordinates define said portion of a three-dimensional structure of a crystallized large ribosomal subunit at a resolution higher than or equal to 3.1 Å.

302. The computer readable medium of claim 295, wherein said structure coordinates defining at least a portion of a three-dimensional structure of a crystallized large ribosomal subunit correspond to a set of coordinates set forth in Table 3, said set of coordinates set forth in Table 3 being selected from the group consisting of:
nucleotide coordinates 2044, 2430, 2431, 2479 and 2483-2485;
nucleotide coordinates 2044-2485;
nucleotide coordinates 2040-2042, 2044, 2482, 2484 and 2590;
nucleotide coordinates 2040-2590;
nucleotide coordinates 2040-2042, 2045, 2484, 2588 and 2589;
nucleotide coordinates 2040-2589;
atom coordinates 1-59360;
atom coordinates 59361-61880;
atom coordinates 1-61880;
atom coordinates 61881-62151;
atom coordinates 62152-62357;
atom coordinates 62358-62555;
atom coordinates 62556-62734;
atom coordinates 62735-62912;
atom coordinates 62913-62965;
atom coordinates 62966-63109;
atom coordinates 63110-63253;
atom coordinates 63254-63386;
atom coordinates 63387-63528;
atom coordinates 63529-63653;
atom coordinates 63654-63768;
atom coordinates 63769-63880;
atom coordinates 63881-64006;
atom coordinates 64007-64122;
atom coordinates 64123-64223;
atom coordinates 64224-64354;
atom coordinates 64355-64448;
atom coordinates 64449-64561;
atom coordinates 64562-64785;
atom coordinates 64786-64872;
atom coordinates 64873-64889;
atom coordinates 64890-64955;
atom coordinates 64956-65011;
atom coordinates 65012-65085;
atom coordinates 65086-65144;
atom coordinates 65145-65198;
atom coordinates 65199-65245;
atom coordinates 65246-65309;
atom coordinates 65310-65345;
atom coordinates 61881-65345; and
atom coordinates 1-65345.

303. The computer readable medium of claim 295, wherein said structure coordinates defining at least a portion of a three-dimensional structure of a crystallized large ribosomal subunit have a root mean square deviation of not more than 2.0 Å from a set of structure coordinates corresponding to a set of coordinates set forth in Table 3, said set of coordinates set forth in Table 3 being selected from the group consisting of:
nucleotide coordinates 2044, 2430, 2431, 2479 and 2483-2485;
nucleotide coordinates 2044-2485;
nucleotide coordinates 2040-2042, 2044, 2482, 2484 and 2590;
nucleotide coordinates 2040-2590;
nucleotide coordinates 2040-2042, 2045, 2484, 2588 and 2589;
nucleotide coordinates 2040-2589;
atom coordinates 1-59360;
atom coordinates 59361-61880;
atom coordinates 1-61880;
atom coordinates 61881-62151;
atom coordinates 62152-62357;
atom coordinates 62358-62555;
atom coordinates 62556-62734;
atom coordinates 62735-62912;
atom coordinates 62913-62965;
atom coordinates 62966-63109;
atom coordinates 63110-63253;
atom coordinates 63254-63386;
atom coordinates 63387-63528;
atom coordinates 63529-63653;
atom coordinates 63654-63768;
atom coordinates 63769-63880;
atom coordinates 63881-64006;
atom coordinates 64007-64122;
atom coordinates 64123-64223;
atom coordinates 64224-64354;
atom coordinates 64355-64448;
atom coordinates 64449-64561;
atom coordinates 64562-64785;
atom coordinates 64786-64872;
atom coordinates 64873-64889;
atom coordinates 64890-64955;
atom coordinates 64956-65011;
atom coordinates 65012-65085;
atom coordinates 65086-65144;
atom coordinates 65145-65198;
atom coordinates 65199-65245;
atom coordinates 65246-65309;
atom coordinates 65310-65345;
atom coordinates 61881-65345; and
atom coordinates 1-65345.

304. A computer readable medium comprising, in a retrievable format, data including a set of structure coordinates defining at least a portion of a three-dimensional structure of a crystallized complex of an antibiotic and a large ribosomal subunit of a eubacterium.

305. The computer readable medium of claim 304, wherein the eubacterium is D. radiodurans.

306. The computer readable medium of claim 304, wherein the eubacterium is a gram-positive bacterium.

307. The computer readable medium of claim 304, wherein the eubacterium is a coccus.

308. The computer readable medium of claim 304, wherein the eubacterium is a Deinococcus-Thermophilus group bacterium.

309. The computer readable medium of claim 304, wherein said antibiotic is selected from the group consisting of chloramphenicol, a lincosamide antibiotic, a macrolide antibiotic, a puromycin conjugate, ASMS, and sparsomycin.

310. The computer readable medium of claim 309, wherein said lincosamide antibiotic is clindamycin.

311. The computer readable medium of claim 309, wherein said macrolide antibiotic is selected from the group consisting of erythromycin, clarithromycin, roxithromycin, troleandomycin, a ketolide antibiotic and an azalide antibiotic.

312. The computer readable medium of claim 311, wherein said ketolide antibiotic is ABT-773.

313. The computer readable medium of claim 311, wherein said azalide antibiotic is azithromycin.

314. The computer readable medium of claim 309, wherein said puromycin conjugate is ACCP or ASM.

315. The computer readable medium of claim 304, wherein said antibiotic is clindamycin and whereas said set of structure coordinates define said portion of a three-dimensional structure of a crystallized complex of an antibiotic and a large ribosomal subunit at a resolution higher than or equal to 3.1 Å.

316. The computer readable medium of claim 304, wherein said antibiotic is erythromycin and whereas said set of structure coordinates define said portion of a three-dimensional structure of a crystallized complex of an antibiotic and a large ribosomal subunit at a resolution higher than or equal to 3.4 Å.

317. The computer readable medium of claim 304, wherein said antibiotic is clarithromycin and whereas said set of structure coordinates define said portion of a three-dimensional structure of a crystallized complex of an antibiotic and a large ribosomal subunit at a resolution higher than or equal to 3.5 Å.

318. The computer readable medium of claim 304, wherein said antibiotic is roxithromycin and whereas said set of structure coordinates define said portion of a three-dimensional structure of a crystallized complex of an antibiotic and a large ribosomal subunit at a resolution higher than or equal to 3.8 Å.

319. The computer readable medium of claim 304, wherein said antibiotic is chloramphenicol and whereas said set of structure coordinates define said portion of a three-dimensional structure of a crystallized complex of an antibiotic and a large ribosomal subunit at a resolution higher than or equal to 3.5 Å.

320. The computer readable medium of claim 304, wherein said antibiotic is ABT-773 and whereas said set of structure coordinates define said portion of a three-dimensional structure of a crystallized complex of an antibiotic and a large ribosomal subunit at a resolution higher than or equal to 3.5 Å.

321. The computer readable medium of claim 304, wherein said antibiotic is azithromycin and whereas said set of structure coordinates define said portion of a three-dimensional structure of a crystallized complex of an antibiotic and a large ribosomal subunit at a resolution higher than or equal to 3.2 Å.

322. The computer readable medium of claim 304, wherein said antibiotic is ACCP and whereas said set of structure coordinates define said portion of a three-dimensional structure of a crystallized complex of an antibiotic and a large ribosomal subunit at a resolution higher than or equal to 3.7 Å.

323. The computer readable medium of claim 304, wherein said antibiotic is ASM and whereas said set of structure coordinates define said portion of a three-dimensional structure of a crystallized complex of an antibiotic and a large ribosomal subunit at a resolution higher than or equal to 3.5 Å.

324. The computer readable medium of claim 304, wherein said antibiotic is ASMS and whereas said set of structure coordinates define said portion of a three-dimensional structure of a crystallized complex of an antibiotic and a large ribosomal subunit at a resolution higher than or equal to 3.6 Å.

325. The computer readable medium of claim 304, wherein said antibiotic is sparsomycin and whereas said set of structure coordinates define said portion of a three-dimensional structure of a crystallized complex of an antibiotic and a large ribosomal subunit at a resolution higher than or equal to 3.7 Å.

326. The computer readable medium of claim 304, wherein said antibiotic is troleandomycin and whereas said set of structure coordinates define said portion of a three-dimensional structure of a crystallized complex of an antibiotic and a large ribosomal subunit at a resolution higher than or equal to 3.4 Å.

327. The computer readable medium of claim 304, wherein said antibiotic is chloramphenicol and whereas said three-dimensional structure of a crystallized complex of an antibiotic and a large ribosomal subunit is defined by a set of structure coordinates corresponding to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

328. The computer readable medium of claim 304, wherein said antibiotic is clindamycin and whereas said three-dimensional structure of a crystallized complex of an antibiotic and a large ribosomal subunit is defined by a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

329. The computer readable medium of claim 304, wherein said antibiotic is clarithromycin and whereas said three-dimensional structure of a crystallized complex of an antibiotic and a large ribosomal subunit is defined by a set of structure coordinates corresponding to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

330. The computer readable medium of claim 304, wherein said antibiotic is erythromycin and whereas said three-dimensional structure of a crystallized complex of an antibiotic and a large ribosomal subunit is defined by a set of structure coordinates corresponding to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

331. The computer readable medium of claim 304, wherein said antibiotic is roxithromycin and whereas said three-dimensional structure of a crystallized complex of an antibiotic and a large ribosomal subunit is defined by a set of structure coordinates corresponding to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

332. The computer readable medium of claim 304, wherein said antibiotic is ABT-773 and whereas said three-dimensional structure of a crystallized complex of an antibiotic and a large ribosomal subunit is defined by a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

333. The computer readable medium of claim 304, wherein said antibiotic is azithromycin and whereas said three-dimensional structure of a crystallized complex of an antibiotic and a large ribosomal subunit is defined by a set of structure coordinates corresponding to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

334. The computer readable medium of claim 304, wherein said antibiotic is ACCP and whereas said three-dimensional structure of a crystallized complex of an antibiotic and a large ribosomal subunit is defined by a set of structure coordinates corresponding to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

335. The computer readable medium of claim 304, wherein said antibiotic is ASM and whereas said three-dimensional structure of a crystallized complex of an antibiotic and a large ribosomal subunit is defined by a set of structure coordinates corresponding to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

336. The computer readable medium of claim 304, wherein said antibiotic is ASMS and whereas said three-dimensional structure of a crystallized complex of an antibiotic and a large ribosomal subunit is defined by a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581 set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

337. The computer readable medium of claim 304, wherein said antibiotic is sparsomycin and whereas said three-dimensional structure of a crystallized complex of an antibiotic and a large ribosomal subunit is defined by a set of structure coordinates corresponding to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

338. The computer readable medium of claim 304, wherein said antibiotic is troleandomycin and whereas said three-dimensional structure of a crystallized complex of an antibiotic and a large ribosomal subunit is defined by a set of structure coordinates corresponding to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

339. The computer readable medium of claim 304, wherein said antibiotic is chloramphenicol and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2044, 2430, 2431, 2479 and 2483-2485 set forth in Table 7; nucleotide coordinates 2044-2485 set forth in Table 7; HETATM coordinates 59925-59944 set forth in Table 7; the set of atom coordinates set forth in Table 7; and the set of atom coordinates set forth in Table 12.

340. The computer readable medium of claim 304, wherein said antibiotic is clindamycin and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2044, 2482, 2484 and 2590 set forth in Table 8; nucleotide coordinates 2040-2590 set forth in Table 8; HETATM coordinates 59922-59948 set forth in Table 8; the set of atom coordinates set forth in Table 8; and the set of atom coordinates set forth in Table 13.

341. The computer readable medium of claim 304, wherein said antibiotic is clarithromycin and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 9; nucleotide coordinates 2040-2589 set forth in Table 9; HETATM coordinates 59922-59973 set forth in Table 9; the set of atom coordinates set forth in Table 9; and the set of atom coordinates set forth in Table 14.

342. The computer readable medium of claim 304, wherein said antibiotic is erythromycin and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 10; nucleotide coordinates 2040-2589 set forth in Table 10; HETATM coordinates 59922-59972 set forth in Table 10; the set of atom coordinates set forth in Table 10; and the set of atom coordinates set forth in Table 15.

343. The computer readable medium of claim 304, wherein said antibiotic is roxithromycin and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 2040-2042, 2045, 2484, 2588 and 2589 set forth in Table 11; nucleotide coordinates 2040-2589 set forth in Table 11; HETATM coordinates 59922-59979 set forth in Table 11; the set of atom coordinates set forth in Table 11; and the set of atom coordinates set forth in Table 16.

344. The computer readable medium of claim 304, wherein said antibiotic is ABT-773 and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 803, 1773, 2040-2042, 2045, 2484, and 2588-2590 set forth in Table 18; nucleotide coordinates 803-2590 set forth in Table 18; HETATM coordinates 1-55 set forth in Table 18; the set of atom coordinates set forth in Table 18; and the set of atom coordinates set forth in Table 21.

345. The computer readable medium of claim 304, wherein said antibiotic is azithromycin and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 764, 2041, 2042, 2045, A2482, 2484, 2565, and 2588-2590 set forth in Table 19; nucleotide coordinates 764-2590 set forth in Table 19; HETATM coordinates 79705-79808 set forth in Table 19; the set of atom coordinates set forth in Table 19; and the set of atom coordinates set forth in 22.

346. The computer readable medium of claim 304, wherein said antibiotic is ACCP and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 1924, 2430, 2485, 2532, 2533, 2534, 2552, 2562, and 2583 set forth in Table 20; nucleotide coordinates 1924-2583 set forth in Table 20; atom coordinates 78760-78855 set forth in Table 20; the set of atom coordinates set forth in Table 20; and the set of atom coordinates set forth in Table 25.

347. The computer readable medium of claim 304, wherein said antibiotic is ASM and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 1892, 1896, 1899, 1925, 1926, 2431, 2485, 2486, 2532, 2534, 2552, 2562, and 2581 set forth in Table 21; nucleotide coordinates 1892-2581 set forth in Table 21; amino acid residue coordinates 79-81 set forth in Table 21; atom coordinates 78747-79289 set forth in Table 21; the set of atom coordinates set forth in Table 21; and the set of atom coordinates set forth in Table 26.

348. The computer readable medium of claim 304, wherein said antibiotic is ASMS and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 1899, 1924, 1926, 2430, 2472, 2485, 2486, 2532, 2534, 2552, 2562, 2563, and 2581 set forth in Table 22; nucleotide coordinates 1899-2581 set forth in Table 22; amino acid residue coordinates 79-81 set forth in Table 22; atom coordinates 79393 and 79394 set forth in Table 22; atom coordinates 78758-79322 set forth in Table 22; the set of atom coordinates set forth in Table 22; and the set of atom coordinates set forth in Table 27.

349. The computer readable medium of claim 304, wherein said antibiotic is sparsomycin and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinate 2581 set forth in Table 23; atom coordinates 78757-78778 set forth in Table 23; the set of atom coordinates set forth in Table 23; and the set of atom coordinates set forth in Table 28.

350. The computer readable medium of claim 304, wherein said antibiotic is troleandomycin and whereas said three-dimensional structure of a crystallized complex of an antibiotic and a large ribosomal subunit is defined by a set of structure coordinates having a root mean square deviation of not more than 2.0 Å from a set of structure coordinates corresponding to a set of coordinates selected from the group consisting of: nucleotide coordinates 759, 761, 803, 2041, 2042, 2045, 2484, and 2590 set forth in Table 38; nucleotide coordinates 759-2590 set forth in Table 38; atom coordinates 1-57 set forth in Table 38; the set of atom coordinates set forth in Table 38; and the set of atom coordinates set forth in Table 40.

351. A method of crystallizing a large ribosomal subunit of a eubacterium comprising:
(a) suspending a purified preparation of the large ribosomal subunit in a crystallization solution, said crystallization solution comprising a buffer component and a volatile component, said volatile component being at a first concentration in the crystallization solution, thereby forming a crystallization mixture; and
(b) equilibrating said crystallization mixture with an equilibration solution, said equilibration solution comprising said buffer component and said volatile component, said volatile component being at a second concentration in the equilibration solution, said second concentration being a fraction of said first concentration,
thereby crystallizing the large ribosomal subunit.

352. The method of claim 351, wherein the eubacterium is D. radiodurans.

353. The method of claim 351, wherein the eubacterium is a gram-positive bacterium.

354. The method of claim 351, wherein the eubacterium is a coccus.

355. The method of claim 351, wherein the eubacterium is a Deinococcus-Thermophilus group bacterium.

356. The method of claim 351, wherein said volatile component is an alcohol component.

357. The method of claim 351, wherein said volatile component comprises at least one monovalent alcohol and at least one polyvalent alcohol.

358. The method of claim 357, wherein the volumetric ratio of said at least one multivalent alcohol to said at least one monovalent alcohol is selected from the range consisting of 1:3.0-1:4.1.

359. The method of claim 357, wherein the volumetric ratio of said at least one multivalent alcohol to said at least one monovalent alcohol is 1:3.5.

360. The method of claim 357, wherein said at least one monovalent alcohol is ethanol.

361. The method of claim 357, wherein said at least one polyvalent alcohol is dimethylhexandiol.

362. The method of claim 351, wherein said first concentration is selected from a range consisting of 0.1-10% (v/v).

363. The method of claim 351, wherein said fraction is selected from a range consisting of 0.33-0.67.

364. The method of claim 351, wherein said fraction is 0.5.

365. The method of claim 351, wherein said buffer component is an optimal buffer for the functional activity of the large ribosomal subunit.

366. The method of claim 351, wherein said buffer component is an aqueous solution comprising:
MgCl₂ in such a quantity as to yield a final concentration of said MgCl₂ in said crystallization solution, said equilibration solution, or both selected from a range consisting of 3-12 mM;
NH₄Cl in such a quantity as to yield a final concentration of said NH₄Cl in said crystallization solution, said equilibration solution, or both selected from a range consisting of 20-70 mM;
KCl in such a quantity as to yield a final concentration of said KCl in said crystallization solution, said equilibration solution, or both selected from a range consisting of 0-15 mM; and
HEPES in such a quantity as to yield a final concentration of said HEPES in said crystallization solution, said equilibration solution, or both selected from a range consisting of 8-20 mM.

367. The method of claim 351, wherein said crystallization solution, said equilibration solution, or both have a pH selected from the range consisting of 6.0-9.0 pH units.

368. The method of claim 351, wherein said equilibrating is effected by vapor diffusion.

369. The method of claim 351, wherein said equilibrating is effected at a temperature selected from a range consisting of 15-25 degrees centigrade.

370. The method of claim 351, wherein said equilibrating is effected at a temperature selected from a range consisting of 17-20 degrees centigrade.

371. The method of claim 351, wherein said equilibrating is effected using a hanging drop of the crystallization mixture.

372. The method of claim 351, wherein said equilibrating is effected using Linbro dishes.

373. The method of claim 351, wherein said crystallization solution, said equilibration solution, or both comprise 10 mM MgCl₂, 60 mM NH₄Cl, 5 mM KCl and 10 mM HEPES.

374. The method of claim 351, wherein said crystallization solution, said equilibration solution, or both have a pH of 7.8.

375. The method of claim 351, wherein said crystallization solution comprises an antibiotic.

376. The method of claim 375, wherein said antibiotic is selected from the group consisting of chloramphenicol, a lincosamide antibiotic, a macrolide antibiotic, a puromycin conjugate, ASMS, and sparsomycin.

377. The method of claim 376, wherein said lincosamide antibiotic is clindamycin.

378. The method of claim 376, wherein said macrolide antibiotic is selected from the group consisting of erythromycin, clarithromycin, roxithromycin, troleandomycin, a ketolide antibiotic and an azalide antibiotic.

379. The method of claim 378, wherein said ketolide antibiotic is ABT-773.

380. The method of claim 378, wherein said azalide antibiotic is azithromycin.

381. The method of claim 376, wherein said puromycin conjugate is ACCP or ASM.

382. The method of claim 375, wherein said antibiotic is selected from the group consisting of chloramphenicol, clindamycin, roxithromycin, and erythromycin, and whereas said crystallization solution comprises said antibiotic at a concentration selected from the range consisting of 0.8-3.5 mM.

383. The method of claim 375, wherein said antibiotic is ABT-773 or azithromycin, and wherein the concentration of said antibiotic in said crystallization solution is about 5.5 to 8.5 times higher than the concentration of said large ribosomal subunit in said crystallization solution.

384. The method of claim 375, wherein said antibiotic is sparsomycin, and wherein the concentration of said antibiotic in said crystallization solution is about 8 to 12 times higher than the concentration of said large ribosomal subunit in said crystallization solution.

385. The method of claim 351, further comprising soaking the crystallized ribosomal subunit in a soaking solution containing an antibiotic.

386. The method of claim 385, wherein said antibiotic is clarithromycin, and whereas said soaking solution comprises said antibiotic at a concentration selected from the range consisting of 0.004-0.025 mM.

387. The method of claim 385, wherein said soaking solution comprises said antibiotic at a concentration of 0.01 mM.

388. The method of claim 385, wherein said antibiotic is ACCP, and whereas said soaking solution comprises said antibiotic at a concentration selected from the range consisting of 0.0150-0.0100 mM.

389. The method of claim 385, wherein said antibiotic is ASM, and whereas said soaking solution comprises said antibiotic at a concentration selected from the range consisting of 0.020-0.030 mM.

390. The method of claim 385, wherein said antibiotic is troleandomycin, and whereas said soaking solution comprises said antibiotic at a concentration selected from the range consisting of 0.080-0.120 mM.

391. The method of claim 385, wherein said antibiotic is ASMS, further wherein the crystallized ribosomal subunit is co-crystallized with sparsomycin, and whereas said soaking solution comprises ASM at a concentration selected from the range consisting of 0.020-0.030 mM.


SEQUENCE LISTING (2 sequences)

SEQ ID NO: 1 - RNA, 34 nt, Artificial sequence
  single strand RNA oligonucleotide
ggggcuaagcgguucgaucccgcuuagcuccacc

SEQ ID NO: 2 - RNA, 3 nt, Artificial sequence
  single strand RNA oligonucleotide
acc